SEQUENCE LISTING (1 sequence)

SEQ ID NO: 1 - DNA, 166962 bp, unidentified
  source 1..166962: mol_type = genomic DNA
atttattaatatttctattactattgaaaattatggcattttctatgttgaccaatacatgaaaatttcatttttcccaaataagactggtgttggatattgggaaagtcatgtttctgaattaaatgaaagtgaatatgttagtacacatgaaaagtttttagactttttatatcatgctgatctaactagtcattatatagatattcatgaatttaaaaagatgatggagaaagtattccaagcatactgtttacttagataactgatatcctctatgctttaagatagatcttcaaatattatgatataatagatctatgaattgagctaagaggtgaaaatgtcagaaactaagcctaaatataattacgtaaacaataaagagcttttacaagctattattgattggaaaacagaattagcaaataataaagacccaaataaagtagttcgtcagaatgatactatcggattagccattatgcttattgcagaaggcttatctaaacgtttcaacttttcaggatacacccagtcttggaaacaagaaatgattgcagatggtatagaagcttctattaaggggcttcacaattttgatgaaacgaaatataaaaacccacatgcatatataactcaagcttgttttaatgcattcgtccagcgtattaaaaaagaacgtaaggaagttgcaaagaaatatagttacttcgttcacaatgtctatgacagccgtgacgacgatatggttgcgttagtagatgaaacttttattcaagacatctacgataaaatgacgcattacgaagaatcaacctatagaacaccgggggctgaaaagaaaagtgttgtagacgattctcctagtttggattttttatatgaggctaacgattaacctctccggattcttggaagaaatacctgaagttgaagctattccctatttacttaaaatgtatctcagggaagttttagctcttgacattgatattgatccagaaaatccgtatgataccgcttttaaatctaatggtgtagaattaaactatcggtatcatttaacagatgatgatttttattttatattagagaaataatatgactgataaacccgaaattaatgatgaagtggaaaagcttatttcttctattgaagaaaagaaccgtcttgaagcagaaagaaaagcaaataagttattgtctaaaaacaaacgcgaactgaatcgtctttataagcacgctcagatcgcagctgaaaacaataattttgctcaatacgaatatgctatcaagaaaagtcgggatattctaaaacagccatataacgatgaacttattagtattctttggaagactactagatcacagattgaggatatgattgatgcttacacatgtaaaattcaagcgtcttaaaattaatgcaggatttactgaatctttgaatggtcatctttgtgtgaaaatttctgaaaaagaataccatgatagttcaattaaagaagttaatcctcctattgtaagagcagaccctaatatgaaagtgtgggttgattcttatcaagtcaaaaaatggtggcagttatgaaagatgaacacccagacttctgaaatagattataataaagttcgttcctctaaagaggaaatgatgagactctttaaagagtctcatgataaagctaaagcagaaggaactataacatataagcgtataaaatttagaagctctaatgagcctctatacggtgtattatgcggataggagcttcggctcctatattgctttataaatttttggtaaaataaaccaaaacaaagaggatattaaatgaaagtatgtatttttatggctcgaggtcttgaaggctgtggtgtaactaaattctcactcgagcaacgtgattggtttattaaaaatggtcatgaagtgactttggtttatgctaaagataaatcatttactcgtaattgcgcacatgattataaatcattttcaattccggttttattggcaaaagaatacgataaaacacttaagctggtaaatgattgtgatattctaattatcaattcagttcctgctacttcagttgaagaagacactattaataactataaaaaaattattgataacattaaaccttcggttcgtgttgtagtttatcaacatgaccattcttctctttctttgcgccgaaatttgggattagaagaaactgttcgtcgagctgatgttatttttagccattctgataatggtgattttaataaagttctgatgaaagaatggtatccagaaactgtttctctgtttgatgatattgaagaagcaccgacagtatataactttcagcctcctatggatattgcgaaggttcgatcaacctactggaaagatgtttctgaaattaacatgaatgttaaccgttggattggtcgtacgactacatggaaaggtttttatcagatgtttgattttcatgaaaaacatcttaaacctgcaggactaagtactattatggaaggtctggaacgttctccagcgttcattcctattaaagaaaaaggaattccatacgagtattatcgtcttcatcaagtagaccaaattaaaattgctcctaatttgccaacgcaaattcttgaccgttatgtaaatagcgaaatgcttgaacgtatgagtaaatccggatttggttatcagctgagtaagttggacaaaaaatatctacaacgttctttagaatatactcatctcgagcttggtgcatgtggaacaattccggtattttggaaatctactggcgaaaatttaaaattccgtgttgataatactcctttgacctcgcatgatagcggtatcatttggtttgatgaaaatgacatggaatcaacatttgaacgtattaaagaactgtcatctgaccgaactctttatgaccgcgaacgcgaaaaagcttatgaatttttgtatcagcatcaagattcaagcttctgctttaaagaacagtttgacattattacaaaataaagggcttcggccctttagctttatacagagtttgatataatgatatttcttggatatgtgatactttttcttgcattttatctattcactagagcatgttggattgggttctttagcacaccagatgggtttatttcaataattttattttgcatttcaatgacggttcttgatatatgaaaattttaaatttaggtgattggcatttaggcgttaaagctgatgatgagtggattcgcggtattcaaattgatggaattaagcaagcgatagaatattctaagaaaaatggaattacggtatggattcaatacggtgatatttttgatgtgcgaaaagcgatcacgcataaaactatggaatttgctcgtgaaatagttcaaatgcttgatgatgctggtattaccctacatactattgtaggaaatcacgatcttcactacaaaaatgtaatgcatccaaatgcttctactgagcttttggctaaatatcctaatgttaaagtatatgataagcctactacagtagattttgacggatgtttaattgatttaattccttggatgtgtgaagaaaatactggtgaaattcttgaacacatcaaaacttcatctgcttctttttgtgttggtcactgggaactgaatggattttatttttataaaggaatgaaatctcatggtcttgaacctgatttccttaagacttataaagaagtgtggtctggtcacttccatactatctctgaggctgctaacgttagatatattgggacaccatggacactaactgcaggtgacgagaatgaccctcgtgggttctggatgtttgatacagaaacagaacgaatggaatttattccaaacaatactacctggcatcgtagaattcattatccatttaaaggaaaaattgactataaagattttacaaatctatcagtacgtgttatagtaactgaagtagacaaaaatctaacgaagttcgaatctgaactagaaaaagttgtgcattcattacgagttgtgtcaaagattgataactttgtcgagtcagatgacagtgaagaagttgaagttcaatctcttcagacgttgatggaagaatacattaatgcaattccagacatcactgattctgaccgtgaagcacttattcaatatgcaaatcagttatatgtagaggcaacacaatgacttttgatgaatttaaaaatgttatgatgagtcagcattttgaatgcgaagtaaaagatgatattggtcataaagaaattattgaatattggtttgaaccgctagaggttgaagataattgtattaaaaaggttacggtctgcactgactgggctgtatcttttaacttcaacattttagataatgacacacctaaatcattacgagatatggctgtatcttgtattaaggatgcatattgtgaagttttcgacatttgacattaatgatgaattcatagcaaatattgattataccgaagaagattctagatatgttggaataatttatattacatcaaaaacagcacaaggtgttgtttgcatggctgaatttgatgaatactttttagattatgacgatatgatagaatggtctaaaagatacattaaaaggaatcttttgtgaagaattttaaactaaaccgagttaggtatcaaaatataatgtcagtaggtggaaatcctattgacattcaattagataaggttcaaaaaactcttattactggacgaaatggcggtggtaagtctactatgctagaagccatcacatttgggctttttggtaagccatttcgtgatgtaaagaaaggtcaattaataaacagcacaaataagaaagaacttttagttgaactgtggatggaatatgatgagaaaaagtactatatcaaaagaggacaaaaaccaaacgttttcgaaatcaccgttaacggtacacgtcttaatgaatctgccagcagtaaagatttccaagcagaatttgaacagcttatcggaatgtcatatgccagtttcaagcagattgttgtccttggtacagcagggtatacccctttcatgggtttgtcgacccctgcacgaagaaagcttgtggaagacctgcttgaggtaggaacattagctgaaatggataagcttaataaagcactaatacgtgaattaaattcacaaaaccaagtgtttgatgttaaaaaagatagtattatccaacaaattaaaatatataatgataacgttgaacgccagaaaaaattgtctggtgacaaccttactcgtctgcagaatatgtatgatgatttggcaaaagaagctagaacgctaaaatcggaaatagaagaagctaatgaaagattagttaatattgttttagatgaagacccgactgatgcatttaataaaatcggtcaagaagcatttttaattaaatcaaaaattgactcgtataataaagtcattaatatgtatcacgaaggtggattatgtccaacctgtttgtcacaattaagttccggtgataaagttgtttctaaaattaaagataaagtttctgaatgtacgcattcatttgaacagctttcaacacaccgtgataatttaaaagttcttgttgatgaataccgagataatattaaaacccagcagtcgttggcaaatgatattcgcaataaaaagcaatctctaatcacgacggtagataaagctaaaaaagttaaagcggctatagaaaaagcatcttctgagtttattgaccatgctgatgaaatagcattgcttcaagaagaacttgataaaattgttaagacaaaaactaatttagtaatggaaaaataccaccgaggaattttgactgatatgctcaaagattctggtattaaaggtgctattattaaaaagtacattccattatttaataagcagattaaccattatcttaaaataatggaagcggattatgtgtttacattagatgaagaatttaatgagacaattaaatcccgtggtcgtgaagattttagttatgcttcatttagtcaaggtgaaaaagcacgaattgatattgctcttttatttacttggcgtgatattgctgaaaaagtttcaggtgttaaaataaacacactaattcttgatgaagtttttgattcagcgaccgacgttgaaggtgtaaaagctatttcaactattttagatagtttaaaaaatactaatgtttttgttatttcacatagagaccatgacccacaagcatatggtcagcaccttcaaatgaagaaagttggtcgatttactgtaatggtttaatttataagagattatgctttaatttattagagtataatctctgtggaggaaaaacatggaatattcaactggacaacatctattaactattcctgaaataaaacgatatattctgagaaataatttttctaatgaagagcatatagttactgaatctatgcttaggaatgcatttaaagcagaatatacaaaaataatgtccaatagaaatgaagcttggactgttactgattattatgactaaaggtgtattatgactaaaattactgtgaattatactgttgatgtaaaagatattcagccaaaacacgtgcgttctgactcaaatccgcaaaaccaaaataaaattcgtcgagcatgggttttgtctctttctgataacgcaatggaagttattcagaacaaaattaaatctgcacctgctcgtcatgcgtattatgaagctatcgatcgtgaagtaagtaataaatggattgaactaatgcgcaaacatactacagaatccctaaacgccggtgctaaatttattatgacttcatgtggtgaacgccttgaagatgaatattgcggtaatgcagatgaacgtctaattgttgctgctcaaattgttgccgaaacaatcgcagctgattttaatcgttaattgctttatttaattagttataaaattaaatctcatttgaattgaaggaaattacatgaaactgtctaaagatactactgctctgctgaaaaatttcgctactattaattctggtattatgcttaaatccggtcaatttattatgactcgcgcagttaatggtacaacttatgcggaagcaaatatttctgacgttattgattttgatgtagcaatttacgatttgaacggttttctcggtattctgtctctagttaatgatgatgcagaaatttcccagtcagaagatggtaatattaaaattgctgatgctcgttcaacaattttttggccagcagccgatccgagtacagtagttgctcctaataaaccaattccattcccggtagcatctgttgttactgaaattaaagctgaagaccttcaacagctgttgcgtgtatctcgtggtctgcagattgatacaattgctatcacggtaaaagaaggtaaaatcgtaattaacggttttaataaagtagaagattctgctctgacccgtgttaaatattctttgactcttggtgattatgatggtgaaaatacatttaatttcattatcaatatggcaaatatgaaaatgcaaccaggaaattataaacttctgctctgggcaaaaggtaaacaaggtgctgctaaatttgaaggtgaacacgcgaattatgtagtagctcttgaagctgattctacccacgatttttaatagagggcttcggccctttataatttacactaaaacttgaatgaggaaattatgattaccgtaaatgaaaaagaacacattcttgaacagaaatatcgtccatctactatcgatgaatgtattcttcccgcctttgataaagaaacctttaaatctattacaagtaaaggtaagattccacatattattcttcattctccttctccaggaacaggtaaaacaactgtagcaaaagcattgtgtcatgatgtaaatgctgatatgatgtttgtgaatgggtcagattgtaaaattgatttcgttcgcggtcctttgactaattttgccagcgcagcttcatttgatggtcgtcaaaaagtaatcgttattgatgaatttgaccgttcagggctagcagagtctcagcgacatcttcgttcctttatggaagcttatagttcaaactgtagtattattattactgctaacaatattgatggtattattaaaccacttcagtcacgctgccgagttattacattcggtcaaccgaccgatgaagataaaattgaaatgatgaagcagatgattcgtcgattgactgaaatctgtaagcatgaaggaattgctatagctgatatgaaagttgtagcagctttggttaaaaagaattttcctgattttcgtaaaactattggcgagctcgatagttattcatctaagggtgttttggatgctggtattttatcactggttactaacgatcgtggtgctattgatgatgttcttgagtctctcaaaaataaagatgttaaacaactcagagctttagcaccaaaatatgcagccgattattcgtggttcgtgggtaaacttgccgaagaaatctattcacgtgtaactccacagagtattattcgtatgtacgaaattgtcggcgaaaataatcagtatcatggtattgcagctaatactgaattgcatttagcttatcttttcattcagttagcatgtgaaatgcagtggaagtgatatgagcttatttgaagatgatattcaattaaacgagcatcaagttgcttggtattcaaaagattggacagctgtccaatccgctgctgattcttttaaagaaaaagctgaaaatgagttttttgaaataattggagctattaataataaaactaaatgctctattgctcaaaaagattattcaaaattcatggttgaaaatgcattatcacaatttccagagtgcatgccagctgtatatgctatgaatttaattggatcaggcttaagtgatgaagcccattttaattatctaatggctgcagttcctcgtggtaaaagatatggtaaatgggcaaaactagttgaagattccaccgaagtattgattattaagttacttgctaagcgttatcaagttaatacaaatgatgcaattaactataaatcaattcttactaaaaatggaaaactacctttagtattaaaagaacttaaaggattagtcacggatgattttttgaaagaagtgactaagaacgtaaaagaacagaaacaactcaaaaaactagcattggaatggtaaaatgattgaaattactcttaaaaaacctgaagattttctgaaagtaaaagaaactttgactcgtatgggaattgctaataataaagataaagttctgtatcagtcctgtcatattcttcagaaaaaaggactatactatatcgttcattttaaagaaatgcttcgtatggatggccgtcaagttgaaatgacagaagaagatgaagttcgtcgtgattcgattgcatggctgttagaagattggggactaattgaaatcgttcctggtcaaagaacttttatgaaagatttaactaataacttccgagttatttcttttaaacaaaaacatgaatggaaactcgttcctaaatatacgattggtaattaatatgactgctataactccgcaagaatacatggcgtctcttaaagaaaaatataatctttctgcaacagaaacactttttgatttaccagaaaacctccaattaaaatttcaggtagaatttcaaaaattagttcacccagaacaaaaacattttactgcagtcgttaagtcaattaatgcagatggattgacaattttcacccggcaaatagtactaatttaagcaaggggcttcggccccttatttggagtataatatatcaagagcctaataactcgggctataaactaaggaatatctatgaaagaattttatatctctattgaaacagtcggaaataacattgttgaacgttatattgatgaaaacggaaaggaacgtactcgtgaagtagaatatcttccaactatgtttaggcattgtaaggaagagtcaaaatacaaagacatctacggtaaaaattgcgctcctcaaaaatttccatcaatgaaagatgctcgagattggatgaagcgaatggaagacatcggtctcgaagctctcggtatgaacgattttaaactcgcttatatcagtgatacgtatggttcagaaattgtttatgaccgaaaatttgttcgtgtagctaactgtgacattgaggttactggtgataaatttcctgacccaatgaaagcagaatatgaaattgatgctatcactcattacgattcaattgatgatcgtttttatgttttcgaccttttgaattcaatgtacggttcagtatcaaaatgggatgcaaagttagctgctaagcttgactgtgaaggtggtgatgaagttcctcaagaaattcttgaccgagtaatttatatgccattcgataatgagcgtgatatgctcatggaatatattaatctttgggaacagaaacgacctgctatttttactggttggaatattgagggatttgacgttccgtatatcatgaatcgcgttaaaatggttcttggtgaacgcagtatgaaacgcttctctccaatcggtcgggtaaaatctaaactaattcaaaatatgtacggtagcaaagaaatttattctattgatggcgtatctattcttgattatttagatttgtataagaaattcgcatttactaatttgccgtcattctctttggaatcagttgctcagcatgaaactaaaaagggcaaattaccatacgacggtcctattaataaacttcgtgagactaatcatcaacgatatattagttataacatcattgacgtagaatcagttcaagcaattgataaaattcgcgggtttatcgatctagttttaagtatgtcttattacgctaaaatgcctttttccggtgtaatgagtcctattaaaacttgggatgctattatttttaactcattgaaaggtgaacataaggttattcctcaacaaggttcgcacgttaaacaaagttttccgggtgcatttgtgtttgaacctaaaccaattgctcgtcgatatattatgagttttgacttgacgtctctgtatcctagcattattcgtcaggttaacattagtcctgagactattcgtggtcagtttaaagttcatccaattcatgaatatatcgcaggaacagctcctaaaccgagtgatgaatattcttgttctccgaatggatggatgtacgataagcatcaagaaggtatcattccaaaggaaatcgctaaagtatttttccagcgtaaagattggaaaaagaaaatgttcgctgaagaaatgaatgccgaagctattaaaaagattattatgaaaggcgcagggtcttgttcaactaaacctgaagttaaacgatatgttaagttcagtgatgatttcttaaatgaactatcgaattatactgaatctgttctcaatagtttgattgaagaatgtgaaaaagcggctacacttgctaatacaaatcagctgaaccgtaaaattcttattaacagcctttacggtgctcttggtaatattcatttccgctactatgatttgcgaaatgctactgctattacaatttttggtcaagtcggtattcagtggattgctcgtaaaattaatgagtatctgaataaagtatgtggaactaatggcgaagatttcatcgcagcaggtgatactgattcggtatatgtttgtgtagataaagttattgaaaaagttggtcttgaccgattcaaagagcagaacgatttagttgaattcatgaatcagttcggtaagaaaaagatggaacctatgattgatgttgcatatcgtgagttatgtgattatatgaataaccgcgagcatctgatgcatatggaccgtgaagctatttcttgccctccacttggttcaaagggcgttggtggattttggaaagcgaaaaagcgttatgctctgaacgtttatgatatggaagataagcgatttgctgaaccgcatctaaaaatcatgggtatggaaactcagcagagttcaacgcctaaagcggtccaagaagctctcgaagaaagtattcgtcgtattcttcaggaaggtgaagagtctgtccaagaatattacaagaacttcgagaaagaatatcgtcaacttgactataaagttattgctgaagtaaaaactgcgaacgatatagcgaaatatgatgataaaggttggccaggatttaaatgcccgttccatattcgtggtgtgctaacttatcgtcgagctgttagcggtttaggtgtagctccaattttggatggaaataaggtaatggttcttccattgcgtgaaggaaatccatttggtgataagtgcattgcttggccgtcgggtacagaacttccaaaagaaattcgttctgacgtactgtcttggattgactactcaactttgttccaaaaatcgtttgttaaaccgcttgcgggtatgtgtgaatcggctggtatggactatgaggaaaaagcttcgttagacttcctgtttggctgatagaataaatctagggacctccgggtccctttttcatacaagtaatataaatctatacttatgaaaaacagatgattctggacatttagaattccctaaaaaattttcacaaaactgtttacaagactgttcctctgtggtactatacaactatcaactactgatacagatttggagaatgaaatgaaaattgttatgggatgcttagcagtttgtttagttgcattagcagcagttccatttgttagtgttgaaaatgatactcaacctgtgattaaatctagcaccgttattcacactaatggtaaaatatcagttaaaattgatgataatcttcatgtgaatacaaatggatctttaggtgtgcaaataggtaatacctgcataagcactactggagtaattactacttgcatttgaggaaattattatgaaaatcgctattttgcttattgcattaggtcttactggttgtgtggctcaaggaccggtaataaatcagtctgatgtaggaaaaattgtaaactgttcaagcaaattttataatcctaatgtcaagtgttataaagaagctccaaagcaaacagtagaacaaatgcaggcgaattttgacgaatctattcgtccagatgaatctgctcaagcatatcgtaattcggatgtaattacacgcgaagaaaaaattgaaaactactgcgccgagctttgggcaaattgggctaataattaccaatggcgtaccggtaaaaatgctccgatggagtatgtagtgaattcttataattcatgtgtaaaaaatttgactaagtgaggaaaacatggaaactttagtagcaggttcaatttttatggttttagtttcaggtgtgttggctattattatatacatgcttccatggtttattgccttgatgcgtgggtcaaaatcgacagtaggaatctttttcacatctttactgtttaactggtcaattattggttggtttattacatttatttggtcaattgcgggtgaaactaaaaagtctgcacagccaaatcaggtaattatcatcagagagaaggaatgaaaagcaaaattatagcagtgttgcttttaatcttgatgattataataagtatatactatagtgtaacggttcctcttatgattccagctattattttaggttggagcttattactgttacaagttaaatatgaatgtatcaattgaggtttaaatgattagtgactctatgacagttgaagaaatccgtcttcatttggggcttgcattaaaagaaaaagatttcgtagttgataaaactggtgttaaaactattgaaattattggcgcatcatttgtagcagatgaaccatttatttttggcgctcttaatgatgaatacattcagcgtgaacttgaatggtataaatctaagagcttgtttgttaaagatattccaggtgaaacaccaaagatttggcagcaggtagcatcttctaaaggcgaaattaactcgaattatggttgggctatctggtcagaagataactatgctcaatatgatatgtgtttagctgaacttggtcaaaatcctgattctcgacgtggtatcatgatttatactcgtccatccatgcaatttgactacaataaagatggtatgtcagatttcatgtgtactaatacagtacagtacctgattcgtgataagaaagtcaatgcagtagtaagcatgcgctcaaatgacgtggtcttcggattccgtaatgattatgcatggcaaaaatacgtattagataaattagtatctgatttgaatgcaggcgattcaactcgtcagtataaagcaggttctattatatggaatgtcggaagtcttcatgtatattctcgtcatttttacttggttgatcattggtggaatactggcgaaactcatattgtgaaaaaagattataacggtgagtggaaatgattcaattcgtaattccaagctatcaacgcgtaggggcagtttctgcccttgatatgtttccgactgattatgaaccgcatattgtagtgcgcgaacatgaagaaaaagcttatcatgacgcttatgggtctaaagctaaaattataactattcctgatgatgtaaatggaattgccggtactcgtaaagcaattactgatatgtatgcaggtcaacgaatctggatgattgacgatgatactactattcgtatgagttcaatgcgaaaaagagatgaccgtcgttgtgtggataaagttaatcaattgactcatgaacagttctatgaattgattcaatacgtcgaagatgccatggattgcgggtattatcatggtcatgctcgcctaccaatttttaaaattacttcatcttggggtaattatcgtgaaaattcatatggattcacgaatacatggtatgaccttggaaaacttacgacagaacaaattgggtatggaaaaattgatttgtgcgaagatatgtatgcatttctcaatttaattaatcaaggttatccgcatttggccttgttcaaatatctggttgtatctggaaaagcacaagctcctggcgggtgcagttcaattcgtagtaattctaaacataatagagcgcttgagcaaatcaatagagagtttccagagcaagctcgttggaaaacttctaatattgaaaaacgaaaatcgttgggtgaagaagacgagccattaaaggttcttcgcatgtgcgtttcacgtaaagaaaaatcagaagcatttcataagtttaatgctattcatccaatagcagttgattaatgcctaaatttattgtgttataattactctatctttaaccagtgaggaaaatataatgatgcctatggaaaaaatgaatgtctattgcagatttaaaatcccgtttgattaaagcttccacttctaaaatgactgctgaactgactacatctaaattctttaatgaaaaggatgtaatccgtacaaaaatcccaatgcttaatattgctatttctggtgcgattgatggcggcatgcagtctggtttaactattttcgcagggccttctaaacactttaaatcaaatatgtctttgactatggttgcggcatatttgaacaaatatcctgacgcggtttgtctattctatgatagcgaatttggtattactccagcttatttgcgatccatgggagttgacccggaacgagtaattcatacgccaattcagtcagttgaacagctgaaaattgatatggtgaatcagcttgaagctattgagcgtggtgaaaaggttattgtattcatcgactcaatcggtaatatggcttctaagaaagaaacggaagacgccttgaatgaaaaatctgtggcagatatgactcgtgctaaatcactgaagtcattatttcgtattgttactccttattttagcattaaaaatattccatgcgttgcggttaaccatacaattgaaacaattgaaatgtttagtaaaaccgtgatgacaggtggtacaggcgtaatgtattcggctgataccgtattcattatcggtaagcgtcagattaaagatggttctgatcttcaggggtatcaatttgttctaaatgtagaaaaatctcgtaccgttaaagaaaaaagtaaattctttattgatgttaaatttgacggtggtatcgatccttattctggattgttagatatggctctagaattaggatttgtagtaaaacctaagaatggttggtatgctcgtgaatttcttgacgaagaaaccggcgagatgattcgcgaagaaaaatcttggcgtgcaaaagataccaactgcactacattctggggtcctttatttaagcatcaaccattccgagatgctattaaacgcgcttatcagttaggtgctattgatagtaatgaaattgttgaagctgaagttgatgaattaattaactcaaaggttgaaaaatttaaatctccagaaagtaaaagtaaatcagctgctgatttagaaactgacctcgagcagttaagtgatatggaagaatttaatgaataaagatgatttagatttagatctagaaattatcgatgaatccccctcttcggagggggaagaagaaagaaaagaacgtctttttaatgaatctcttaagataattaaatctgccatggaaaatgttatccaggagattgtcattaaactagaagatggttctacacacattgtgtatgtgacaaaattagattgggttgatggaaaagtcgtaatggactttgctgttcttgaccaagaaagaaaagctgagttagctcctcatgtagaaaaatgtattacaatgcaactacaagatgcatttaataaaaggtcaaagaaaaagtttaaattcttttaaggagtaagtgtggtagaaattattctttctcatctcatatttgatcaagcttatttttcaaaagtttggccatatatggattcagaatattttgaaagtggtccagctaaaaatacgttcaaattaattaaatctcatgttaacgagtaccatagcgttccatctattaatgcgttaaatgttgcattagaaaatagttcatttactgaaacagaatattctggtgtaaaaacacttatttcaaaactagctgattctccggaagaccacagctggttagtaaaagaaacagaaaaatatgttcagcaaagggcgatgtttaatgctacgtctaaaataatcgaaattcaaactaatgctgagcttcctccggaaaaacgaaataagaaaatgccagatgtaggtgctattcctgatatcatgcgtcaagcattatcaatttcatttgatagctacgttggtcatgattggatggatgactacgaagcacgctggctatcttatatgaataaagctcgtaaagttccatttaaactcagaattctaaacaaaattactaaaggcggagctgagacaggaacactgaacgttttaatggctggcgttaacgttggtaaatcattaggactttgttcattggcagcagattatttgcagctcggacataatgttctttacatttccatggaaatggcagaagaagtctgtgctaagcgtattgatgctaatatgcttgatgtttctcttgatgacattgatgatgggcatatttcttacgctgagtataaaggaaaaatggaaaaatggcgtgagaaatctactcttggtcgtttaatcgttaaacagtatcccactggtggagcagacgctaatacattcaggtctctcttgaatgaattgaagctcaaaaagaattttgttccaacgatcattattgtcgactatctaggtatttgtaaatcttgtcgcattagagtttattcagaaaatagttacacaactgttaaagctatcgcagaggaattgcgtgctctggctgttgaaaccgaaactgttctttggactgcagcacaggtcggtaaacaagcttgggactcttccgatgttaacatgagcgatattgcagaatctgctggtcttccagcaacagctgattttatgcttgcggtcattgaaaccgaggagctagcagctgctgaacaacaactcattaagcaaatcaaatcacgatatggtgataagaataagtggaataagtttttgatgggtgttcaaaagggaaatcaaaaatgggtagaaattgaacaagattctaccccaaccgaagtgagtgaagtagcaggttcacaacagattcaggctgaacagaatcgctatcaaagaaatgaatccactcgagctcagttagatgctttggcaaatgaattaaaattttagtttacaaactgacaagactatggtatagtagtcttgttggttaaatgaggagattgttatggaattggtaaaggtagtttttatggggtggtttaagaatgaaaacatgtttactaaagaaatcacaatgatgaaagatgacgttcaatgggctactactcaatatgctgaagttaataaagcattagttaaagctttcattgatgacaagaaagtatgtgaagtagattgccgaggataatatgcatattgttttatttaaacctactccgtataacgtcaggaaaaatactcaattcaaagcacttattgcggatacatgggaattggtgttagatattccagcagaagaaagccctccatttggtcgagtggaatttattaagtttgttgtacgtcctacgaagaggcagattcgtcagtgtaaaaggcattttcgtaagatcgttaagctagagaaacagtttgtaacatgtgattatgcagaagttttaaaatagctgtttacttttgttggaaattgagatactatataaacataaactactgaggagattatcatgaaaaaatttatttttgctacaatttttactttggcttcttgtgctgctcagcctgctatggcaggatatgataaagacttgtgtgaatggtctatgactgcagatcagactgaagttgaaactcaaattgaagcggatattatgaatattgttgagcgcgatcgtcctaaaatgaaagctgaagtgcaaaaacaacttaagtctggtggtgtaatgcagtataattatgttctgtattgcgataaaaacttcaataataaaaatatcgtaaccgaaatcacaggtgaataatatgatttattatatgcacaaaaatccaccgtttaaatacggtaaatttcctgatgcacaatgttataatataacgccgaatgaaaataataacggatatcatattggtgtaatttttgtaattgttaaagataacgaaatcgttgcctgggcagattttaaaggtacaacatatgatgttaatccggtgcctttcacatattacaatttaatggatttggcatatgattataattggtttaatcatgacgcgttggttcatattgaaggtgtaggatttgacatctcttattctagctactcattgtgtcctatgagtcgggctcataaagaacctgaatatttttcatttaggaaacgtgtaaactttaaacgcagcacagagtatgtcggtggaatatttgttaaagataatcgaattactcgcattatttatcctttgggctgtggtcaaaaagatattgatgttgatttagactttactgaaaacatcattaatcatatttcttctgtgtattttgatatcgatcacaaaattgtaatatgtggatatgaattaccatcagaaaaacagtctaaagtaaatgtttctattgacgatcaaatttttaatgcttttatgaataaaggttaatatgtatagttctgaattttcatatttaaaaatggaaaaaatttcatacgattttatagatgaaaaggtttattacagtttccatgaaccacgttttaatagtgaggttgggtttattgtagtaaaagacaatttcattttaaagatatattcggcattaaaggattttcactacgaaaatattaacctaaaatttgataaagaaaacgttcgtaattgtgcagtaacaattacaggaaataaaggcacatgtgttatgctatctgatgaaattaatgatttgctaaatgatgccgaaaaggttgctattccatctattgatgaccaaatttttaatgcttttatgaatagaggttaatatgaaaacatataaagaatttattaaagaagatatggtcgctggagattcaggtggtaatcctgaaaatatctctactggaacaacgtcaggtgctgtagtaaataaaggtcctgaacagattcctaaaaagaagaaagaggaatctaaagaaaaagaagagtaaaaatgtcatcaataccttggattgataatgagtttgcgtatcgtgcattagctcatttacctaaattcacacaagtaaataatagttcaacttttaaattgcggtttagatgccctgtttgtggagattcaaaaaccgatcagaataaagctcgtggatggtattatggtgataataatgaaggaaatattcattgttataactgtaactatcatgcaccaatcggaatatatttaaaggagtttgaacccgatttatatcgtgagtatatctttgaaataagaaaagaaaaaggtaaaagtcgtccagtagaaaaacctaaagaacttcctaaacaacctgagaagaaaataattaaatctcttccgtcatgtgttagattagataaactggcggaagaccatccaattataaaatatgtaaaagctcgttgtattccaaaggataaatggaaatatctttggtttacaactgaatggcctaaattagttaatagcatcgcaccgggaacatacaaaaaggaaatttctgagcctcgtcttgttattccaatttataatgctaacggaaaagctgagtcttttcaaggacgtgcattaaagaaagatgctcctcaaaaatatatcaccattaaagcttatcctgaggcaacaaaaatatatggagtcgaacgagttaaagatggtgatgtatatgttctagaaggacctatagattcactttttattgaaaatggtatagctattacgggtggtcaattagacctagaagttgttccatttaaagatagacgagtgtgggttttagataatgaacctcgtcaccctgacactatcaagcgaatgactaaattagttgatgcaggagaaagggttatgttttgggataaatctccctggaaatcaaaagatgtcaatgatatgattagaaaggaaggtgcaacccctgaacaaattatggaatatatgaaaaataatattgcccaggggttgatggctaaaatgcggttatttaaatatgctaagatttaaatcaacccaactaaagcaaatgctaaatctacaaatgtatcaagagtaactactggaatattaataccatgtgcaattgcaataggtgatagaataaagttccagagtaaaattcctaccatagcagaaatagtaaaagctatacgttttttattaccttttatggcattaacaagtgccattaatttttgtaccatatgtcctccttattgctttatatatttattgtataattaatctactaacccatgaattgaaaggaaaaataatggcttactttaacgaatgttcacaactgattgaaggcgctgataaagctcaaaatgaatactgggatattcttggtgatgaaaaagacccactgcaggttatgcttgatatgcagaaatctttacaggttcgtttagctaaagataaacctgaatataaccgtcatcctgatgatttggctactgcaggtgaagttgttgattggctacgcaatcagaaagattatattgatgatgaattccgtgaacttctgacttctcttggtggtatgagtaatggtgaaaaagacgcatctgctgtatggaaaccttggaagtcgcagcatggtgaacgtcgtgaaactctgattactgatttgtctccgcaagaccagttagaaattaaatttgaaatgattgatattcttcatttcgttctgaatatgttccagggtcttggtcttacagcagaagaaatctttaaactttattacctgaaaaatgctgaaaatttcgcacgtcaagaccgaggttattaatggcttgtttaaataaacgtcaactcaaaaagttgaataagaaaagggcgaaagcccttactgctttattacgtaatgaaataactaaagaaatagaccaagagattattgaacaactctggaaagccgctaaattgtaagttataaatacacctgtaattaaacaataaaggagtttattatgggtggtttcgttaacatcaagacctttactcatccagcaggtgaaggcaaagaagttaaaggtatggaagtttctgtagcatttgatgtatattcaaatgaacaccgtattgcagattctcattatcaaatctttccgtcagaaaaagcagcttattctgatgtagtttccgatgcagcaacttggaaaactaagaatgctgcaatgtttacgcctacagaaataggtggttaataattcaaggactccctagggagtccttttttcattttaccggtttactttccaaaatgagtatggtataataaaattatcttatagaggagagcactatgttaaatcgttggattaaaccaaatgaagatttagatattatcatttcacggcatgtaatgaagaaatatgaactgcagccatggtccacagaagttgttgtgcattcatttatgatgtacgcagatggttctgtcgaatttaatacagagattcgatatgattatggcgagaagcaagttgaattcaagagaggttttttgtagtgtttatctttaattggtttaaaagtttctttacggattttttctctacgactcctggggaaggtgtagttcctatttcaaatgactaccttcctttaactgtagttgagtatgtttatatgggagatggaacagtagaagcagttactatgacttatgaagaagcccaagaatattataaaaatccttggcgatggtcagtacctactacatcatctaatgcacagaatacacagtctagttctgattcatatgatactaatgttcctgttcatgtatggacgggcgattcatgtggaagttcttgtgattctagttgttcatctacatcttgtgattgaggaaaattatggaagcaattttgtttgaaatgtctatcaatagtattagtatggcgtttgtcaaggatgttccaattactgtagcagtaatgattgataaaagatatgacaatgacatgtatctagtagaagattttatttcaatgccagttccagaagatgttgaaataaaacttaaaaagatcggaattattgaaactgtacaaaattctccatttacgtcaattcaagcatttactaaatctaactatattgatgttggtgaagcattttataacaataaaccgctgtccttctattcatatgattcaatatatgattggaaaatagataaaggaaataaattcataattgcggaagaaattgcgctatcatattttattacttctttatggaataaattaactccaaatttgctaaaaattcataaatttgacgatgctcctactattgttttaggtaaaatgaatgaaagtcctaaagaaaatatttgaatggtttaatcgaccaaactcaatgtatattgatgatggttgggttgaacaagcaaataaagaaatgcagaacgagtcagaagaatggatgaaatcaatgattagtgctgagaaagaaaagaaattagaacgctcagcgcttaaattgatgagagacatctatggtgataaatcatgaccaaagatatgacgttagaagaggcaaaggctaaagcaaatgaagccttggatttgcttcttaaaattggcagtaagatgatggaagaaaatgagaagtatatccaggaaaataaaattcctgatggtccattagtaggcaagaggaaaacacatgattgaagtagcaaaacattattcaatagaatttatgtctaaagaaggtaaatcagtaaatacacttgataaaaaatgctcattaattattcctttagcagaaaatccggattttttaattaaagatataaaagaaagaaaatatccagaaaatgttattctaattataaagcatactgaagatattttacagaatactgattcaccgttttcttcttccgaagctttaactattaaaggctataaaagagctcatgaatatggtctttttgacatgtttgaagacgataaagttaaattagcgagtcaaccttctaaaagtaaaacattcattattgaagatattaaagatataaatgcatttgttaagatggtctgggctcattttgatgttggactacgctggagaatgtccgaagaagaaagaaagatcattgaaactaatcgtaaatttggtttttatcgctaggaattaatatggatttatttgagatgttagaagataatcattctacgaatatccagaatgattctagtgattataaaaaagagtaccgtatagtattacagaattatggaattgaagccccagatgctcttctagaagaactagcttcataccatcttgaccctccgccttgggctccctgggcaaaataattcaaaaagttgtttactttcctttctaacgatgatatgatagcttctgaagtatacggaggctatcatgattattaatcctgcagatgttgaacagttatctataaaagctgaaagcgttgattttcaatatgatatgtataaaaaagtctgtgaaaaatttactgactttgagcagtctgttctttggcaatgtatggaagccaaaaagaatgaagctcttcatcagcagttgaataaaatcgttaaaaagcatttaactaaatcaccttatcagctatatcgtggtatatcaaaatcgacaaaagaactcattaaagatttacaagttggagaagtgttttcaacgaacagggtagattcatttactactagtttgcacacagcatgtggtttttcttatgttgagtatttcactgaaataatatttcgcttaaaaactgataaagcttttaattattctgaccatatcagcgatattatactttcttctcctaatactgagtttaagtacacgtatgaagatactggtgggctagattcagagcgtactgataacttaatgatgattgtgcgtgaacaagaatggatgattccaattggaaagtatagaataacttctatttcaaaagaaaaattacacgattcatttggaacatttaaagtttatgatattgaggtagttgaatgaaatactcaataatgcaactaaaagattttaaaataaaatcaatggatgcatcggtgcgggcttctattcgtgaagaattgctttctgaagggtttaatttatctgaaattgaacttttaattcattgtattactaataaaccagacgatcattcttggttaaatgaaatagtcaaatctcgtttggttccaaacgataaacctctttggagaggtgttccagttgagactaaacaggtgttaaatcaaggaattgatattattacatttgataaagtagtatcagcttcatatgataaaaatatagctctacattttgcttctggattagaatacaacacacaagttatttttgaattcaaagctcctatggtgtttaatttccaggagtatgctataaaagctttacgttgtaaagaatatagtccgagttttaagtttccggatagccatcgttatcgtaatatggaattagtttcagatgaacaagaagtaatgataccagctggaagtgtatttagaattgcggatagatacgagtataaaaagtattcaacatacactatctatactcttgattttgaaggatttaatctataatggaaggacttagattcattataccatgaaagttttaaagcatttttcataaagttgtttacaagctgaagtaaaaatgttatagtataagtagttaacccgtccgtgagaaaaatatgaaactgtctaaaaatcaaattcgtaaaattacacgtcgtctagagcacactcaggcatctgctaaaagacgttctaaagattttaacttagacttcaattacattaagaacattttagatcagaaagtttgcgcttactcaggagaaccttttgataatcgtattgaaggagagaaattatcattagaacgttttgataataacgttggatacattaaagggaatgttattgcagtaaagaaaaagtataatacatttcgttctgattatactttagaagagttgattgaaaagcgtgatttatttgctttgcgaattggtcgttcatctgcgaaaaaagttcataaactaaatttagatgaaaagaaatgggctaaaatcaaaaagacttataatcaaattaaagctatacagaaaaaacgtgaaaaccgaattgaacacatttctcagctttctaaatcaaaacagacctctgacattaagctaaggattatagcacttaaagctcgtattgatggttctcgcatagcagaaggcgctgaagttgttaaactgaacgttcttcttaaaggctcggattggaaaattgtgaaaaagttgtcagaagcagaaatgcaatatgatatgtgtgataaaattattcaaggtgtagagcggtatcaaaacttgtcttttattgataaacttaaactgaaaagaggatacccgctaaattgttcaatttttaaacttatccgaggataatatggtttatgtatatgcgatagtttaccgagacaaagacggatttacggcgccagttccacttgatgaacatcgtcctgctgtgttttttgaaagagagattgctgatagagtttttactactctcaaagaccaatataaactagctttaggtatgggaattccaagactagttgagactccacgcaagttttggtttaataaaatagaagttaaacatgttaagcctgatgtagatacacaaagattatatcaacgaattctagatactgggcgtattgttagtattccaattgcagggactttccgatgacatttgatgatttgacagaaggccagaaaaatgcctttaacatcgttatgagggctattaaagaaaagaaacatcatgtaactattaatggacctgctggtactggtaagactactcttactaagttcatcattgaagctttaatatctacgggtgaaactggtatcattttggcagctcctacacatgcagctaaaaagattctttcaaaactgtcggggaaagaagcgagtactattcacagtattcttaaaattaacccagttacgtatgaagaaaacgttctttttgaacaaaaagaagtaccagatttagctaaatgcagggtattaatctgcgacgaagtgtcaatgtatgatagaaagctcttcaaaattttgctttcaactatcccaccgtggtgtactataattggaataggcgataataagcaaattagacctgttgacccaggagaaaatactgcttatatcagtccattctttacacataaagatttttatcagtgtgaacttactgaagttaaacgcagtaatgctcctattattgatgtagctactgatgttcgtaacggtaagtggatttatgataaagttgttgacgggcatggagtacgtggatttactggtgataccgctttacgcgattttatggtaaattatttttcaatcgtcaaatctttagatgatttgtttgaaaatcgcgtaatggcatttacgaataaatctgttgacaagttaaatagcattattcgtaaaaagatttttgaaactgataaagactttattgtcggtgaaattattgtaatgcaagaaccgttaattaaaacatataaaattgatggaaagcctgtatcagaaattatttttaataatggacaattagttcgtattatagaagcagagtatacatcgacgtttgttaaagctcgtggtgttcctggagaatacttaattcgtcattgggatttaacagtagaaacttacggcgatgatgaatattatcgtgaaaagattaaaataatttcatctgatgaagagctatataagtttaacctgtttttaggtaaaacggcagaaacttataaaaattggaataaaggcggaaaagctccgtggagtgatttttgggatgctaaatcacagtttagtaaagtgaaagcacttcctgcatcaacattccataaagctcagggtatgtctgtagaccgtgctttcatttatacgccttgtattcattatgcagatgctgaactagctcagcagcttctttatgttggtgtaacacgtggtcgttatgatgtgttttatgtgtgaggctatatgattaacatcaattcaaaatatttaaatcgtctaatagatggtataaggaagcatactaataagcaagataatctcgatgttatggtaacaggagctgagctccttcataagctttatcttataagtgatactatattagcaattaaacgaattgaaaaacaatcatatcacagtaatacagatacggtaattacactagatgaaagtgtctgtaaattactaattaaatttgaggaagctattcgtggaaataactaaagaccagttttatctgcttcaagataatgtgagcgaaatttatgaaatagcttatagtaaaaatcgtagaaatgtaaaaattgaatctggcaagttaatgcttcaattagaagaaattgaacgagatttaattgcgttagaattcttttgtggtgaagtgaaaactgttacaatcagtgattatgttttaggcgaaattagccatctttataaggcgattattaatgattgaattaagttggtatcagtttaaatctcttatggcaaatgttaaagctgtcattgaagaaaatccaggacctgaaaatgtaactattcgtgaaaaagcttcaaagatagtatacagtcttgaagagatgcaaaaagatattgaatctatggcaaaatttattgatgagcccattaataaagtttatatccaagattatactgtaggtcaaattcgcgatttagcgaggaaagtttaatgtttgattttattatagattttgaaacgatgggaagcggtgaaaaagcagctgttattgatttggctgtaattgcttttgaccctaacccggaagttgttgaaacattcgatgaactagtttcacgtggcattaaaattaaatttgacttaaaaagtcaaaaaggacatcgtctttttactaaaagcactatcgaatggtggaagaatcagtctcctgaagctagaaaaaatattgcaccatcagatgaagatgtaagcactatcgatggtattgcaaaattcaatgattacatcaatgcacataacatcgatccttggaaatctcaaggttggtgccgtggaatgtcgtttgattttccaattttagtcgatctcattcgcgatattcaacgtcttaacggcgtttccgagaatgagcttgatacatttaagttagaaccgtgtaaattctggaatcagcgtgatattcgtaccagaattgaagcacttctccttgttcgtgatatgaccacgtgtcctcttccaaaaggaactttagatggattcgttgcgcatgattccattcatgactgtgcgaaagacatcctgatgatgaagtatgctttgcgctatgctatgggtcttgaagacgctccatcagaggaagaatgcgatcctctgtctcttccaacaaaacgataaaaagttgtttacttcctcggttagttatggtattataacaccatagctactgaggataataaaatgaaaatttatcgtgttgaatcatcatttagtattcttaattatgaagatgctataacaatacgtcgagatctttgtgttcaaataacgccatacaggagtataatggattcatggagcgaagagtggttattacacgtaggttatgacagacctaattttatgcatcatagtaatgataataaaagaattcctcttccgcacgaagacaaactattagttaaaaatgctaatatagcaattaatactaattttgagaaagaagaatatgtaggcgtagatcactatattccaggatggtttatagctctttatcattttgctttcgctagcgaatatgatatgatgagatggttcacacgagaagagcgtgaagaattagcttctaaaggattttatctcgctgtatacgaagtacctgatgaccaagttatcattggcgatcatcaagtaatgttccgcaagtcacatgccgaattaaaaggctttattaccttagattaaggggaaaacatcatgaatatcagtactacagctacttacatcctgaaagacgaaaagcaaatcaactggtatggtgcttcagtgcgagcttatgaaaagttttgtgaaattttaggcgccactcaaggtaatagtttcactattgttaacttcatcaaacaagctgaaggcccttggttattcttagaaattaaaaattcatacggtacaatttctactaccgcccttccaagcaccgactttgaactgttgattgaagaagtagaacccgttaaagacacctgtattgatatggatatcgcttgtatgtacggtactctggttaagcctattgagcctactgatttgttgtgtaaagctgtttcaattcgacgtccgttcgcaggtccagtgagtggatgggtaactgatcagtggattgaagacggtgttgaacttctgaacgttgttcatgcaggtgattttagtgtagttccacgtagtgcagtaattaatatccttaattaagtttactttatagagagcctgttataatggctctcatggagaataaatcttaacaacaaagagagaaaaattatgattactatcggtgaaattaagcgtgtatcgtctgcatctcgttctaaagctgcaggtcgtctagttgaagtggtaggacttcgtactaatggtactggtcgtgttatagaagttaaagttcgaatcattcctagttctggaaaaactgaaactcagtacgcttacgtaagtcctaaatttctggaaactcgtgttagctctcattttgcttctgctgaagaagttgaatccactgagtctaatgcacaatttaaatgggcgttgtgtaaaggcgttgaatttgtaactgatgaagaattcgattatattgatgatttcgctggtccaagtaaaactgataggatgtgtggcttcatcacagaccaatgggttgaagatggtgaaaagctctacaatatcgtattcttaggtgacttccgggttgttgaagaatctgaaatcactcgttatgcttctcctcgtaaagcttaaggaattattatgaatattgtaaatctctctactggtgattacgtatatgtatccgttgcttctcgttctaaagttgcaggaaagctggttgaagttctttctgtaagtgatccagtttttggtcgtaacgaggttaaagttcgtgacgctgatggtaacattggttatgtctcaagtcagttcattgagccagaagaaagcgcatttaaatggaaactttgttcttcagttttagttgtaaaagaccataaattcacaattgcggcggcaacagatggaactccatggacaacgaatcaagtcgctggttttatttcggaccgttgggtagaagatggtgtcaagctctacaatattgtttttgcaggccagttcatggtagttcctgaattccaaattaaaaaatggtgcgaagcagctttagcataaatttgcataaagtttaaaattttttcataaaactatatacatcagtagttgattatggtactatatcaatatcaactactgatacagaaaataacttggagaataaaatggaaaacttaattatcatcgagcaatctttcaacgattatggtatggcttacggttatcgtgcgataatggaagattctcgtggatgtgttatcgatattgctgaatgtaaagatttactgcagcttttgaagattgttcgtaaaaattgggattgtgaaaacattaaagttcgtgttgtaacggaagaagaaactgtttttcatgatgtaaaattcgctaaaggtgctgctactcttcttaaacgcatcgctccactgttcaattaatgaggaaattgtaatgaaacgtaaaattgttcagaattgcactaatgacgaatttgaagatgtattatttgatccaaatctggtagtagttcaaaaggaacatactaataagtttactcacttgacttcggtttatgtgtacgaaaaagttggtgataaacaaccaatttatggtgtgtttcgcgaaattactgaagatggtacaacttattggaaggaaacttattaatggctattaaatttgaagttaataaatggtatcaatttaaaaataaacaagctcaagaagattttatcaaggaccatactgaaaatggaatttatgcacgtcgtcttggtatggaaccttttagagttacagatattgactatcttggtcgtcctactaaacttgtgacatctagtggaaaattggcgttatcttctggtaaagatattttggatgaagattttatctggctttcaacgagtgaagctgcgttctttgatgaagtggaaaatccataccaggcagctgaagagcaagagcaggaagagaaagagcaagaacaaatagaagatttcacagaattcccagtcatgaaagttactattgaaaataatgaacatgcatggtccttgtatcagatgttgaaagcacattttaaggaataattatgccaatgtatgattataaatgccaatccgaagattgcgggcatgaatatgaaaaaattaaaaagatttctgaacgagaaaatgatgtttgccctaaatgtcatcgtttgtctactcgtcggccttctgctcctaagcatgtgaatggcggtttttacgatttacttaaagggtgattatgtttaaaatcggtaagaaatatcgtgttcgcgaaggtgaagaaaagaaatatctactttctgctagtaataggaatggttctattaatgccgtaatatctacaagcgaatttatcgttgaagatatgaaaggtcataatgttacaatgattagtacagcaactagcagaaatgctggaaaaattcttcacagtctgcggagtgaagttttaatttatgatgaagaatttgacttcttcgaagaagttcctgaagattttgattttgaatgcactatcactatgaaatctggcgaccctctttcttttacagttaaagatgaaagaagccgtttgggaattattagtctccttcaagccattaaatttaagtgaaaactatgaaatatattaatcgttctattgcagcattagtattagcagtatctttagtaggatgtactgatgctgataatgcaactaaaattttatcatcgagtggttttacgaatattgaaatcactggatataattggttcggttgctctgaaaatgatttccaacatactggatttcgtgctattggacctactgggcagaaagtagaaggaacagtatgttctgggttgttctttaaggattcaactattcgttttaaataaaaggccttcgggcctttagctttatgattgccggagtataatattcccgaaaccaaacgaggataagtaatgattaagaatgaaattaaaattctgagcgatattgaacatatcaaaaagcgtagtggcatgtatattggctcttctgctaatgaaatgcatgagcgctttctgtttggtaaatgggaaagtgttcagtatgtacctggtcttgttaagcttattgatgaaattatcgataactcagtcgatgaaggtattcgtactaagtttaaattcgcaaataaaattaatgttaccattaaaaataatcaggtgacagttgaagataacggtcgcggtattccacaagcgatggttaaaacacctactggtgaagaaattcctggtccagttgctgcttggactattccaaaagcgggtggtaactttggtgatgataaagaacgtgtcaccggtggtatgaatggtgttggttctagtttaactaacattttttctgtgatgtttgtcggtgaaactggcgatggtcaaaataatattgtagttcgttgttcaaatggcatggaaaataaatcctgggaaactattcctggaaaatggaaaggaactcgtgttactttcattccagattttatgtcatttgaaactaatgagctgtcccaagtttatcttgacattacactagatcgtctccagacacttgctgttgtttatcctgatattcaatttacctttaatggtaaaaaggttcagggcaattttaagaaatatgcacgacagtatgatgaacatgctattgttcaagaacaagaaaattgttctattgctgttggtcgttcaccggatggatttcgtcaattaacttatgtgaataacattcacactaagaatggtggccatcacattgactgtgttatggatgatatttgtgaagaccttattccacaaatcaaacgtaagttcaaaattgatgtgactaaagcacgtgttaaagaatgtttgactatcgttatgtttgttcgtgatatgaaaaacatgcgatttgattctcagactaaagagcgtttgacttctccatttggcgaaattcgtagtcatattcaacttgatgctaaaaagatttcacgcgctattctaaataatgaagctattttgatgccaattattgaagctgccttggcccgtaaattggcagcagaaaaagcggcagaaactaaagcagctaaaaaggcttctaaagctaaggttcataaacatatcaaagcgaatctttgtggtaaagatgctgacactactcttttcttgactgagggtgattctgctatcggatatcttattgatgttcgtgataaagaacttcatggtggttatccattgcgtggtaaagttctcaacagttggggtatgtcatatgctgatatgcttaaaaacaaagaactatttgatatttgcgcaatcactggattagttcttggtgaaaaagctgaaaacttgaattatcataatattgctattatgaccgatgctgaccatgatggtctaggaagcatttatccttctcttcttggattttttagtaattggccagaactgtttgagcaaggacgaattcgctttgtcaaaactcctgtaatcatcgctcaggtcggtaaaaaacaagaatggttttatacagtcgctgaatatgagagtgccaaagatgctctacctaaacatagcatccgttatattaaaggacttggctctttggaaaaatctgaatatcgtgagatgattcaaaatccagtatatgatgttgttaaacttcctgagaactggaaagagctttttgaaatgctcatgggagataatgctgaccttcgtaaagaatggatgagccagtagtttactttaccacaaggatgtggtataattaattgggcaaatgaggatattggaatgaaatcatataaagtaaatttagaactttttgataaagcagttcatcgagaatatagaatcattcaacgctttttcgatatgggagaagccgaagaatttaaaaaccgctttaaagatattagagataaaattcaatccgacaccgcaactaaagacgaactactagaagttgctgaagttattaagcgtaatatgaattaatgaggaaattatgattatcaccactgaaaaagaaacaattcttggtaatggttctaaatcaaaagcatttagcatcacagcatctcctaaagtatttaaaattctgtcgtctgatttgtatacaaacaagattcgcgcagtagtccgtgaattgattaccaacatgattgatgctcatgctcttaatggtaatcctgaaaaatttatcattcaagttcctggacgtttagatccacgatttgtttgccgagattttggtcccggtatgagtgattttgatattcaaggtgatgataattctcctggtttgtataattcatacttcagttcatctaaggctgaatctaacgatttcattggcggatttggtttaggttctaaatctccgtttagttatactgatacatttagtattacttcgtatcataaaggtgaaattcgtggttatgtagcttacatggatggcgatggtccacaaattaaacctacattcgtaaaagaaatgggtccagatgataaaactggtattgaaatcgtagttccagttgaagaaaaagactttagaaactttgcttatgaagtttcttatatcatgcgaccgttcaaagatttggctattattaatggtcttgaccgcgaaattgattattttccggattttgatgactattacggtgtaaatccagaaagatactggccagatcgcggcggattatatgctatctatggcggtattgtatatcctatcgatggtgttattagagaccgtaactggttaagcatccgcaatgaagtgaattacattaagtttccaatgggttcacttgatattgctccatctcgtgaggctctttcactagatgatcgcactcgtaaaaatatcattgaacgagttaaagaactcagtgagaaagcatttaatgaagatgtaaaacgatttaaagaatctacatctcctcgccatacgtatcgcgagttaatgaaaatgggctattctgctcgagattatatgattagtaattcagtcaaattcacgactaaaaatctgtcatataagaagatgcagagcatgtttgaacctgacagtaagttatgtaatgcaggcgtggtgtatgaagtaaatcttgaccctcgactgaaacgcattaagcaaagtcatgaaacttcagccgttgcatcaagttatcgtctgtttggtattaatacaacaaaaattaatatcgttattgataatattaaaaatcgtgttaatattgtccgtggattagcacacgcgttagatgatagtgaatttaataacactttgaatattcatcacaatgagcgtcttctgtttattaatccggaagtagaatcgcagattgatttactccctgatattatggcgatgtttgaaagtgatgaagttaacattcactatttgtcagaaattgaagctttggttaaaagttatattccaaaggtagttaaaagtaaagctcctcgtcctaaagctgctacagcatttaaatttgaaattaaagatgggcgctgggaaaaagaggaactatttacacttacgtcagaagcagatgaaattactggttatgtagcgtatatgcatcgttctgacattttctctatggatggtactacatcgctttgtcatccatctatgaatattttgattcgtatggctaatcttattggcattaatgaattttatgttattcgtccgcttttgcagaaaaaggtaaaagaactcggtcagtgccaatgtatttttgaagctttgcgtgatttatatgtagatgcttttgatgatgtagattatgataagtatgtaggttattcaagttcagctcaacgatatattgataaaattatcaagcatcctgaactagattttatgatgaagtacttcagtacagatgaagtttctgaagaatatacacgactcgctaatatagttagttcattgcgcggtgtatattttaatggtggaaaagataccatcggtcatgacatctggacggtaactaatctttttgatgtattatcaaataatgcttcaaaaaacagtgataaaatggttgctgagtttaccaagaaattccgtattgtttccgacttcatcggatatcgcaactctttaagtgatgatgaagtttctcaaatcgctaaaactatgaaggcccttgcggcctaataaggaaaattatgtacaatattaaatgcctgaccaaaaacgaacaagctgaaattgttaagctgtattcaagtggtaattacacccaacaggaattggctgattggcaaggtgtatcggttgacacaatccgtcgtgttttgaaaaatgctgaagaagctaaacgctctaaagttactattagcggtgatattacagttaaagttaatagcgatgtagttattgctccagttgctaaatctgacattatttggaatgcatctaaaaaattcatttcgattactgttgatggtgtaacttataatgcaacacctaatactcattcaaacttccaggaaattcttaatctgctcgtagcagataagttagaagaagcggcacaaaaaattaatgttcgtcgcgctgttgaaaaatatatttccggtgatgttcgaattgaaggtggaagtttattctatcaaaatattgaattgcgatctggtttggttgatcgtattcttgattcgatggaaaaaggcgaaaactttgaattttattttccgttcttggaaaatctgctggaaaacccaagccagaaagcggtatctcgactctttgatttcttggtagcaaacgatattgaaattacagaagatggttacttctatgcttggaaagtagttcgcagcaattactttgactgtcactcaaatacctttgataacagtcctggtaaagtagttaaaatgccacgtactcgtgtaaatgacgatgatacacaaacttgttctcgtggtctgcatgtgtgttctaaatcttatattcgtcacttcggtggttcaaccagccgagttgtaaaagttaaagttcatcctcgtgatgtagtatcaattccgattgattacaacgacgctaaaatgcgtacctgccaatacgaagtaattgaagacgttactgaacaatttaaataagggcttcggcccttaactaaggaaaattatgttaggttatcaagcacgagtaaaagaagaatacgatcaattaatgctcaaaattaatgcactgagcaaatttttagaaagcgcaaagtttctaacggttagtgcagttgagcaagaactgctgctttcacagttcatctcaatgaaatcttatgctgagtgtctagagaaaagaattgcacaattcaaataaaataagggcttcggtccttttgttttaaggtgaattatgatttattgtatgaatatcggtgattccgacataaaagaaatttctctacatggtaatcatcacgctaatattgtgtactgtaataaatttgagtttggacacgaaaaacttgggtttctttattgcaatgatgttatagtcatggataaaaaggaactcgatgaacttgaccgtgagtctttagatgaaaatgaccgaatttattacggcactcttaaagtatataacgcttatctcagcggtaaaaaggaaagtctaaatgcagaaaacgaatccgggtttacagagactatttcagattccgacatttaccctatcgaacagtgatttgactagtgaaatgaaggtcaaaattgctgatacagcaagatactctttaaaacaaaacccgaaccaggataaagcagaagttattgaaagatgccgtatcgcggtgtacgcagagttttttgtggcagattggctaagcggatatgtcaataaaggccaagaaaatgtcgatgatccgtacacgtatgcatgggatgttttagcgcatccaaaatattgcgggcttcgtgtagaagtcaaaacacatcaaactgactcgcgttggatttcggtaacaacgggatgcagcggagagtatccatatggttctggaataaatttagggcccattctaaatcatcaagtcgctgactgtataattatattcaacactaaagaaattcatccaggtgtcatccagtacactccgaagttcatcggtgacagagaagaccttcgtaaggttgtaagaaaaagcaactacaacggatggtatctttccatttaaaaattttcacaaaacggtttacataccacaaggactgtggtaatatacaactatcaactgatacggatttggagaataaaatgtttactacagctgaattaaaacgagcaaaagctaagaaagggcaaggaaaatataaagctgaattagttaaagaacttcagtttgctgaggctgaattgaattcaatgattattcaaaatgctccagaaactgaaattgctcttaaacgtattgcgaataagtgtcttcgtgatgcaatcgtcgatcttttagcggattattgagtaaaatgaaaatcgttgagattgaactatgagttcattatggtggtgttttgtttggttaattagtattccattaatttgtttaacatttacttttgtgatgaggttattatgaaaatttttaattctgtacttattgcttgcgcgtggtgggttgcacaggtttcagcggtagtagttggtattcacatttattacgaatatttttaaaaaagttgtttacaagactgttcctctgtggtactatacaactatcaactacggaggaacacaaaatgaaaaagattgttaaagctatatggaatgtagttataatactaatagttttgagtatattcccaatcgttttaatgattgatatgttaaacgcttattttggatttatgtgaggaaaatatgaagcgtaaacgcggtgcttttacatttattgaatggtttttcgataatatttttccggctttatttatttttatgctgattttcgccttaggttcagttgtagttggaatctatttgatggcagtagtcggaatggatattcatcaaaatggtttaaaatccgtagttgaaacaatttggaatggtgtaaaatgatgaatttgttgagcggttggttttatattcttatgttttacattggtgcaaattttccatattggatgggatggtcaacaactgcatttggattttatactctttgaggtgaattatgaaaagttttaaagatgtaaaagttggtgaaattttctgtttagataacggtgatcagttaattcgtatttcacctcttaagagcactagcgagaaaccgacagttaacgctactttagcaaataacagtaatgaacgtttctgtattgaaaatgatactgaaacttataccgtagaagagttttgggaattgagcgtcgactgcgacgattaatttaatggccgtgtgtattcatgcggccttggagtagaaaataatttagaggaaattaatatgaaatatatgactgttactgatctgaatgatgcaggtgctactgttattggtacaatcaagggcggtgagtggtttttgggaactccgcataaagatattttatctaaacctggattttactttttagtaagtaaattagatggtcgtccatttagtaatccatgtgtgtcagcacgattttatgtaggtaatcagcgttctaagcaaggattcagtgcagttctaagtcatattcgtcaacgtcggtctcagcttgcacgtactattgcaaataacaatgttccatacacagtgttttatctgcctgcttctaagatgaaacctctgacgacgggatttggaaaaggacagttagctttagcgtttactcgtaatcatcattctgagcatcaaacacttgaagaaatgaaccgtatgttggctgataactttaaattcgttttgcaggcatattaatgagtaatttccataacgaacatgtgatgcagttctatcgtaataattttaaaactaaaggcgtcttcggacgccagtgaggaaaatatgaaatttgtttatggtatagcctttttctgctggtcctttttattcgccggaacagtaataactggagaaacttcagtatttttacaagcaacagctcaaggcgtattagcaattttatcattgttttggctattaaatgaaaatgattaagaaaatcttgggctattcattagcccttgctactttattagtagcactatattatggagtaatgttcggattaattcaagtcgtgcttttcatttctgatgttattatggcactacattcactagtatggtaaatttatgcaactgaataatcgcgatttaaaaagtatcattgataatgaagcattggcttatgctatgtacacggttgaaaatcgtgccattccaaatatgattgacggatttaagccagttcaacgatttgttattgctcgagctcttgatttggcacgaggaaataaagataagtttcacaaactcgcttctattgcaggtggtgtagcggaccttggatatcatcatggtgaaaactctgcgcaagacgcaggtgctttgatggctaacacttggaataataattttcctctgttagatggtcaaggaaactttggttctcgtaccgtccaaaaagcagcagcaagtcgttatatttttgctcgtgtaagtaaaaatttctataacgtatataaagatactgaatatgctccggtacatcaagataaagaacacattccgcctgctttctatttgcctattattcctactgttcttcttaatggtgtttccggtattgcaactggttatgcaacttacattcttcctcatagtgtttcttctgttaagaaagctgtattacaagctcttcaaggaaagaaagtaactaaaccgaaggtagaattcccagaatttcgtggtgaagtcgttgagattgatggccaatatgaaattcgtggaacatataagtttacttcacgaactcaaatgcatatcactgagattccgtataagtatgatcgtgaaacttatgtgagtaaaatcttagacccacttgaagataaaggcttcattacatgggatgatgcttgtggtgagcatggttttggattcaaagttaaattccgtaaagaatattctttgagcgataacgaagaagaacgccatgcaaaaattatgaaagacttcgggttgattgagcgtcgttctcagaatattacggttattaatgagaaaggaaaactacaagtttacgataacgtagttgatttaattaaagactttgttgaagttcgtaaaacttatgtccaaaaacgaattgataacaaaatcaaagaaactgagtcagcttttcgtttagcctttgccaaggcacatttcattaagaaagtaatttcaggtgaaatcgttgtgcagggtaaaactcgcaaagaactgaccgaagaactttcgaaaatcgatatgtattcttcttatgttgataaactagttggaatgaatatttttcatatgacttccgacgaagcaaagaaacttgctgaagaagctaaagctaagaaagaagaaaacgaatattggaaaactactaatgtagttaccgaatacactaaagatttagaggaaatcaaatgagtccattcataggtattacaagcgctgcattagtatccggtagcattttactggcgggtttaggtgttgttccagccgtagcaggaggtcttcttgcgttcggcattcaacgtgttatcatgacagttatcacagtcatgcagtaatttttagggagagccgaggctctcccttttttatttcaaaaatttttttcacaaaacggtttacaaccaaagcatactgtggtactatacaactatcaactactgatacagaattacggagattagaaaatgtctaaagtaacttacatcatcaaagcttctaacgatgttctgaatgaaaaaactgctgcgattttaattactattgctaagaaagatttcattacagctgcagaagttcgtgaggtgcatccagatttaggtaacgcagtagttaatagtaatattggggtgttgattaaaaaaggcctggtggagaaatcaggtgatggattaatcattactggtgaagctcaggatattatttcaaatgcagcaactttatacgcacaggaaaatgctccggaactactgaaaaaacgagcaactcgtaaagctcgtgagattacttctgatatggaagaagataaagacctcatgttaaaacttttagatgaaaatggatttgttcttaaaaaggtcgaaacttatcgtagtaactatctcgctattttagaaaaacgcactcacggaattcgtaattttgaaattaacaacaatggaaatatgcgaatttttggatacaaaatgatggaacatcatattcagaaatttactgatatcggaatgtcatgtaaaatcgctaaaaacggtaatgtgtatcttgacattaaacgctcggaagaaaacattgaagcagtaatcactgtagcatctgaactgtgaggaataaataatgaacaagttagaaattatcaatgaacttcgtcgttgtgcagaacctactcaagagggttgggatatttggtaccatggagcttatcttggaactattgtaaagattaagactggtaaatacatgattattcgtgaaagtaaagatgctccagtaggtattcgcaataatttcatggcagcgataagttcctttacagatgcagcttacgaaatttaccttgccgattataaagaattccaggaatctcaaccagttattcgctcaattggtgttaacaaagctcaacagaaaactttgtggcaacgtattaaaggatggtttaaatgaacccatttatcaatcgtttaaaaatgctgaatgttcctttatctcgtgaaactccagaaagtcttgctgaaaaatttaaagcgcatggttataaatgcacagaagaagatattctaaaagaagttcctgaaatatgttggcagactgcatattgggatgaaaaccaaaagtatcaacgacgaattgtctgcgcggccaaccgttttaaattaaaagatggacgaactcttattattccaggtgctcgtcattattctaaagatatggccgaagttttagatgtagttaaacctcaattagttactcaacaagtttgtgatgatgaccaagggtttattgaccaatatagtaattattggacacgtgaagaagcaatgattattgcaacttacgctggacaagtacgtattgaacggggtggtagtgaaaaagaactttactctgaggacctttactaatgaatattaaaaagtttcaaatcgatggaattatgaatcaaattcaggcactggaatatgccaataaaatgatgtcaactaattggggaatttatgcgaatgagccagcatttaaattttgtgatatggaatttaccaaaaagcttgtaggaaaagattatgtatgcccatttagttctccggtaaatggaatgctaaaacctgctttacgcgatctttatattgcgatgaacgaagaaatgataaaagaattaaaacgtcaactgaaggtgattcaatttggccagggaaattaattcaaaatctgattattttaattctctcaatgataaagataaaaatctaataaggcattttattgttgagatgggatatactgatacacgtgatttaagagaacatatatttgaatgtggtgtagctaaaaagttttcattcacatgcaaatgtttaagagaggtaattcagcactatgaacaatttagtcgcaaaacataattttaataaagcttctgttcataaggataagaagaaagtgtttaaagaatctaatcgcaaacagaaacataaggggaaggtctatgattattgattctcagtctgtggttcaatacacaatcaaaattgatattctagaaaagctatataagtttctaccaaatttataccactcaattgttaatgaattagttgaagaactacatctcgagaataatgatttcttgattggaacttataaagacctctcaaaagcaggatatttttacgtaattccagctccaggaaaaagtattgatgatgtattaaaaactataatgatttatgtccatgattatgaaattgaagattatttcgaatgagtcataatcttgaaaaagtaatcgagcataatgtagctcaggaacgtgagtcgttcaaggaattcgtagaaaaaatttttcaagaaaataacacagaccagtttacaaatcaaacgtctgatgatattataacaaagtcaactaattgagtggtatagttaatgaataaaaatattgatacagttcgtgaaattattactgttgcgtctattttgattaaattttccagagaagatattgttgaaaatcgtgctaattttattgcatttctgaatgagattggagtaacgcatgaaggtagaaaattaaatcagaattcattccgtaaaattgtttctgaattaactcaagaagataagaaaaccctcatcgacgaattcaacgagggttttgagggtgtatatcgatatctagagatgtatacgaacaaataattatttagcccttcctaatattctggccgcctgagaacatattgattcaaggcggtcattacttatatgatcatttctataccagtacatggttatcgttccagcatagatattatccaaattaaaatatggacaactgtacatgtagtttatttcgggagtaggctttttagttggtaaaaaagcaaattttgagttggaataataatgacgtccatttaaatgaactgcatattcatccatagttttatcaacaggatatcctccaagtgatttttcacttattgttgaaggtaattttccttcatatgctataatatcgacaaaatagtttaagtttttagggcggaaagaatacaccgcactaaagtctgcctcagatgatatgtgaactatctggagttgttccagggcgacagattcaaagcgtgcatttctttccttttcaataatttcactgtatgtttcatactttgattgcttatagtactcaaagaaactatctcccctataccaaacaatcgccatcataaacaaaagaattacgacagctacccgggaagcaagaactttcccggtagcgttatctttgaacaagcgatctagaacaccaaatagaatatcagagggcgaaaatgatattctaggtgctgccatagaccctccttttaaggatatttattcaaaaattatactcttgaaccgtatacggctccaacgttttgccatgttggagccgatcctgctacagcagcaccaggatttgtcggaggtgttgaataaccattacctggattagcatattgacctggttgaccaacttcacctccaccgccagcccaatattggccagtattaccaccagcaccaggtgctgttaagctagcatttccaccaggccatcgagcaccattatttccgcctaaaccaaatggacgaccaccgccgcctccacatacatattttcctgcccagttattggcttgcgaatatccaccgccaccgccaccgcctccagcaatagctcccccattattaattcgtaggcgtccacctatatcattttggatacatacaccaccgtcagacgctccaatcgctccagcagcccaaccattcccgccgcgaccatacatcgttacgccgtgtatattcaattgaacatattcattcggtgtatctccgtacatgaagaataaaggaacgtctctagaatatgatactaaatctccagtgatattaaacactataggtgcactacctgcctcaaaacatctatctcggaaccattgaccattgaagttatggtccgctccaagtgtgtgaattacttcaacagatcgacctatcatattactcatccagaacggaatgcccattcttaatcgttgggccgcgccacccatccaattttgtcctgtattaattactgcagacgatcctacccacggtcctgctactgccatataaacctccaaggggccgaagcccctcattttattttaacaatgatttaactaattttttaagctcttcaatctctgacttaagttctgcaatttctgcagtatgctcattgattgcagctgtatttaaaccaattacaccgttatagttcaaacgaagtaaagcttcgccatcagggtcaccttcaactaattcaggcaaaatagcttgaacttcttgagctatcaaaccggcgttaggttcccatttctgattgccttcttcatctaggcctcgcttctgcatataagtgtaaccgttaatttttgaaagtgtttgtgaagcattttcaaatttaacaaggtcttttttaacacgaatatcagaacggacataaacatcgcggacatatgtggaataactgtcattcgcctggacaagatggccgtgagaaataattgctttgccaacttctgtccaaaaagctccatttgaatcccaaaggccaggatgagaatgataagatgaagaccacgaagcaatatgcgctccatttgcaaccatattgattcggccgtcaccaccatgaactaatccagtatcattatcaccaataacaagacacgggccggtccatacagggtcaccaccgccagctgataatcttgctccagcttgaaccatattaggagcatacagaactccattgtggtgaaattcatagatagcttgtgggtcagaaccattttcctggttaccaacacggataatagcctgagcccattggttagtagtgcgtcgcataccaaaatcaacacccgatatgtatccttcattagtaataccagattttcctttgataatcgggtaataactatcattaccgacattgccaaaatctacaaatattggcgcttcttgtcgccattggtttccccaatcaccagcttggtgcgcaacccattgtgtaccgtttatatgaaggcggtctctattaaaattaaatgttccttgaccttgaggagaaagcgatatttcgccgttatttgttatgtatacaccgccttgtgtaatgtaactataaaaacctagaccattgtcgccgccgcctttgcctatataccagttacctgtaccacctacatcactacggatatgtgatgaagcaccttcgcccgcagttaaaatgagagcatcattgcctgccgataaagtcaatttaccagtcatcgtgtcgccttctttagaaacacgagtagctattgcatcgcgaatatttgtggtttctcccgcagaagtaaacgatttccaaatatctgagaaaatattaccattacgagcaatagtagcttcaccgccgccaacttgaacgttatcggggaaattaactgtaccatcagcattaaatcttgatgtatggccttgtgaaccatcttctttcaaatgatgtaaaacaaatgtaccagaattaatttcagtacctaaagaccagacagctttaccgtttaaaaacttctgtttaacaataggataatatttgcttacagcagcatcatcgatttcttggaaaataggagcagcttgtgaatactgaatatcataaccacctgcacccgcgccatgaccagcaaatttaatgtagccatttgcagcaaacgtgttataacttgattctaaatctggaataacaaccttaccagtatccaaagcaatactaaatggacgaagtggaccgatatcaccatttttgccttcaccgtaagcagttggaataatatgaagactaccttctgaacgtctaaaaatggctccataatctgcattccaaatgcgaagagcattcactgctccagatgaaatttcgcctgttgtgtgaactcctctatttccagcagtaacaccagatgattcaaatgaaccgttaacgttcatttctacttcaccagtagtaagtctttgaatatagctcatccagctagttgcatcggaaatttccatgacatttttgcgattaccttcgctaggattagtaaatgtattaccccacacagaaattgtcatgttatccacagaactactacctgttgtgttacctttaaacagagctgtagaagcattatttctaccatcgatattaacagaaccgccaataacatcaatattaccaggagtaactaataaccctccagcagtattaatattagttgttcctcttccgcgaaagtaatggtgatatctaccgccttgataatatcctaaaagagtctgaccatccccaaacgcgttattatcatggtatgtcactacagtagcaagagcataattttcagttggaggagtagttaaatcggttccgttagtagaatatccagctataaattttcttaggctagttgtttcgcttggactaaataaaaacgtttttgtgccattgttaacactgaaataatatccgtcttgatgccatttaaatccagtatcattatctcctaaagcgatactagacggtcctaatattgagtaatccttaggtccgggtgttaaaccgatggaaattgagtgacttgatttaattccgccgctccaagtaaatgaccaaagataagcgtctaatccatctccactataccatgttattccatcattatctttaacttcatggaaaataccagaaccaactgcattacgcataacacgaagatgattaatagcagtagtgctttcagataatggaacattatctgccatcgaatagatgtcataatcatctttaacttttttattggtgataactttattggtataaattaccggagttgatacagatttccacccaaaaacttctggtgaagaaaacaaaccatttccattaaaagcgaaagtgctttctgaaccagctttgtaatcttgaactctgatgttaactacctgtgtagttaaaccgtcattagcaggagaaaaaataataccgcgttcacggtcagctgaatcatgaaaacgaacgtgtgccgttcctgaactttttgtaataagttctccgccttcagtcataatctgcccagcagcaataatatcacgagttactcgagcaataccggttaaattaaaattaccagtctgggtgaatgtaccgtttaaagtataatcgccagtttggttataattgcctttatgaataacattcccgtcaatactaccgcctttagcaaaacctaaatcaataatatttccctggtcatctttagtaaaaagtacacggtcttttaagtttatagccaattcaccttcggctaatactgaagcagctggacgttgacctgccgttttgcttcttttaaattgtatttgttttaaagtagccataagtcctcttaataatagccgaaatcttgaacagaatccttaattacgatttggtcaaatcgcggaacatgcgagggttgagatgcaggattctgtgaaaaaaggtttggcgcagttaaatttcctatcatagtgtctccagagcgtaatactctagagtttgcatttgttgtaacaatatttattgctccatcaacataatcttttctcgtaaggtcataattatttactggtgataacgaagttgcgcgtatttgtgatgcttcaattacaccctttgagctaatatctccgtttcgtgtgttaattactacagttctacctgctgccccttgtgatgatttaaagccgataccataccatgaaacaatatccatatttgtagtatcatatgtagcattatcaccaggaccggcaaaaataccgttgtcacctgacccagcaccaggaacagtcattcctttattaaaaattactttatttaaatatgtaccaccattagctttagaaacgaaatcgttatcagcagcttgtggtttattatattctgaatatattttaaatgatttatagagtacatcgtcaccggctggattcaatggaaaatttccttgatgccaaatgacagagcctccagttgttgaacctacttttaaatcagccattgtatgcccctttattttaatagtatttataaagaaaaagggaacccgaaggctccctcaatttaaacttctctaaattcctgtccaaacactttaccagttttagacgatgcttgcgtaggaagtaccattatatccggaggtgaagcagattcacagatataattaactcgaataccattgacgccaaactcagcaggttttgaaatgccaccatttcttgacacttcagaaaagcttaaatttctcataccgccttgacccgcttgtgcagttctctgtgcatatatcgtaaatccaactgcattttctggaacaactacatagtcttcctttaattcccaagacccagcctgtccagtaaactcagcttgtgtcgaggaaatatatccattcgacgcattataaaaacgaattgaaatattagtagttcccaacgctaataaatcagcgtcagcatatagttgtgctttaagataaagaacatctccaggaattaaattatagtcagaaagtttacttattgcagatgctgtaggtagacgtgcaatttcgttattagttccaccaacagctgacataaattgctcaacgctttcatatgttcttttaggaaatcctgtcgctccaacatcttctaaactatcaaatactacatctaatatagtttgatagtcatctgtgctttttctgttactcagtttaacatgctctagtgcgatagctcttttagaagaagtgtaaaaggcagcatatgatacgtcaaatcttgacaataaagaatcagatggaaaagcagatgttcctgccgttcttaaccaagatactacttcaggaggaaaattaacctttccgctagttaatatagcaacaagtctattgcttgaaaaagaattcatgaaactaacaaaggcagcagatgttgtatcatttgaagtagaaaaaacatatgacttactatcaaccaatgctcctgtagaagggtcaaaaactcttaaatgaagacctgcactaaatgtttgacttcctacaggattatcttgaaatttaacgtatggtcctgcggtagaaagcgggcaagaacccgctatacttattttatatcttactgaattgctttccgataaaaatggcgtttggacatatccttgtccaaactctgccataaatttttccataatacctcttattcaacccattcaaatttaacagttttattcactgggtcaggaataatgcgaacattaccaattcgtaagaaatcacgaatagtaagattacccattgtagcattatcagatggtaaagcaccaatatcagatggttgaggaggattacctccatcaaatacctgaacaaaacttgaccaagaatttttggttttctgccatgtacgtgtccagcgagtggtacgcgcttctggcgttgttggataagtaatccaatcttggtaaagcgaatcaagtgtgttaccaaactgagtcagtgtaccaggagatttaacttcttcgccacgttctaaatatggaagaccagtcacttcattagttttttcaaccattttaaaataacctgggaactggttataagtagctgaatcgttaatatcaattgaccagaatcctacagtatcagatgttggcgcacgtgtatataagtcagatgttttagtaccttgagaacgaattctcgagttaacagttaaaccaccagaatttatagtagcacctttggcgatgattaagctttcaccaatcgttacttgaccagatgcattattaatagctaatgggcgtaatccgttaaatccaccagtctgatcacccgatgcagtaagcataaaattcgttactgatccatcattgcgaacaatgaaaccataatttccgttaatagcacggaaagcatttgcgcttcgagaaataatttcaccagctgcagtgactgaattaccaaatgttgctacgccatttgcattcaatgttcctgaagcattaacatttatcggtgttactgtaccgttgatattaaatgttatatttccggctttattgcgctgagaataaaaatgactagatgtttcatcactaacttcaaatacggtagaacgtgttgtatctaattccccactaaattgatttccccacactctaacagtcatcgtttgggctgggtttgttccagtttgaggtcctttctcaaaaatcaatcgagttgctgttccagtattagaaatagttaatgtactatttgctgaaactgatccaccgaacgtagcagtactagatgatacaagaggggcactcagattcgtttgttgagttaaggttagtgaaccattaaccgtctgcgcaatatccctacgaatgaactgagatgaatctagaccatccaataaattactatctgcagcttttgcttttaatggcaaataatttgctaatacacggtttaattcgtatggtgatactgcatagctatttttctcgtatagctctaagtcctgggtagaaccggctgtatcattaccaacgaatgtaattgaaccagatgaagttttaacaaagcctcttactgcagtagtagctgcccaagtaggttcactctgcacaatccattttaaattttttggagatacagcagtatttgccgatgttccagcaacagtttcggtttgtgttgcaactttaataacaccttcttgtgcttcagtagatttagtacctaaaagctttttaggagttattaaaacattatctaattttccagcagcagcttcaacttgtgtagccacacgaagtgtaccacgttgtgtctcatttgcttcaagaatattaagggtataatggtcccagagagttcctgattcaactaatccagatagagcaacaacagaagtacgatcagtactattaaatctagttttaatttttaatggtgtagagatacgagtatcgtcgacgcctgcgtcgaattcaacttgtgtagcaatttcagctataccacttaaactttcagttgctctacggtcatttaaagttttaggagtgactgcacgagtataatcagttcctgtattaacttcactttgcgtagcaatttcaattaaaccaattcttccatcagttgatgtctttttatgtaacgtttctggcgttacaacagcatttgcccatccttcctgactttgtccagcaattacttcactttcaactgctaaaattactgcaccttgttgtgttggagtagctttatactgatccaaggctttaggtgaaacaactaagttattagtgtttttattataaacatttgtaccatttaattcacggctagaagccggagtagcacctgcagtagatacaaaagttacaataccagataatgattcagaaccttgacgagcttgaagctttttaggagtaatgatggttgtatcatctgttcctgtgtttgtttcttgctgcgtagcaatttcagcgacacccctacgagtttctgtagcagttctttcattcaactttttaggagtgatgataagatcatctgcaaaagagaatgtggtatcctggtttacttgcgcagttgttgctattcttgcaatacctctacgagtttctgtagcagtacgattagctaacgtttctggagtaattgccagttctttttgtggagaattttctagatcagcattagcttgagcctgtgtagctaaagcaattacgcctaatcttgctctagtagaattatttaaagagtctactctttctacagttggaacgttttgctgtacaacccaatattttccatcggaatcttcgatataagcaagctgtaaaactggaacataattggtttcgccattaaaaactaattcttgaacagtcacccatttagtttcaggtgggtattctgaacgttttggaaattgcagcaattgaactgaagaagcaattttatcttcaccggcagctttgattttaactgtttgtccttttctcatataattcatggaaatcttaacagtatcaccgatagaaatatcagttggaagctgaagttcaattgtttgggttgttccattattcgcgccaaataccatgacttcttcatttggacgaatattagaattagtcgttataatgcgtaaacgtgctttactatccccgtcaaacaatctccacaatttctcattatcatcaaacattaagaaaccatcaatagatgtacggccttcaatggaatgagttccaacttcttgtactgaagttgtttcatcatatgtggtaacaattgtgtgataaagtggatttaatttatctaaatcaacaaaattaataatatccccatgattagcaaatctaggaagtttaatattaattgctgaagcagaagtaaatctacgcacgataaaatcgtttgattgcgcttgataaagacttgccggtgttacaactgccgcttctctgctataatcagcaacatacatttgccacagacgattactaaaaattaaaactatctgtgattttggatgagtcattagtactgagcgtacttgttgacctctaaagtttacaatgctttgtaccggagctgtaattaaaacctggttaactccaggttttcctccaatatcttggagaacgatagtatcaccatcaattggagaagatggcaaagtaaatgtaatgtcattacccgctgcggtgttaactgaaatcgattcgcctgattttaattgataaggtcctgacgaaacagttgtccagtttgcatcagtgcgtaacgctctccatcgtacgctattaaaagctcctgctggttttggaatatcatttatagcagcccaaaagcggttatcataaatgattacaaaatcttttaaatatccacgagttggatcatattgttgaactgtgttttcttgaattaagtaatcaacgttaacaccgtcagttcctacggcacgatcagctaaagctacgttgattattttatcaccacctgcgtccagaccatcttctgctctgaactttcttttaatctcggccattctcccgggctcctattgtgttttcaataataagtatttatacttgtttactttaagattcggatggtatataatagaaatctcactaattgaacgaggttcatatggatttagaaatgatgctggatgaagattacaaagaaggaatctgcttaattgactttagtcaaattgcgctttcaactgctttggtaaacttcccagataaagaaaaaattaatttatcaatggttcgtcatttgatattgaactcaattaagtttaatgttaaaaaagcaaaaacgcttggatacactaaaattgtactgtgtattgataacgcgaaatctggatactggcgtcgtgatttcgcgtattattataagaaaaaccgcggaaaagcacgagaagaatctacttgggactgggaaggttattttgaatccagtcataaagttatagatgaattgaaagcttatatgccatacattgttatggatattgataagtatgaagcagatgaccacattgctgttcttgttaaaaagttctctttagaaggacataaaattttaatcatttcgtcggatggtgactttacacagcttcacaaatacccaaatgttaagcaatggtcaccgatgcataagaaatgggttaaaattaaaagcggttctgctgaaattgactgtatgactaaaatccttaaaggcgacaaaaaggataacgttgcttcagttaaagtacgatctgacttttggtttaccaaagttgaaggtgaacgaactccttcaatgaaaacttcaatcgttgaagctattgctaatgaccgtgagcaagctaaggtgcttctcacagaatctgaatataatcgttataaagaaaatttagttctaattgattttgattatattcctgataatattgcttcaaacattgtgaattactataattcatataaattaccaccgcgtggcaaaatttattcatattttgtaaaagcgggtctttctaaattaactaatagcattaatgaattttgaggtgaataatggctaaaaaagaaatggttgaatttgatgaagctatccacggcgaagacttggctaaatttattaaagaagcatctgatcataaactgaaaatttccggttataatgaattgattaaagatattcgaattcgtgccaaagatgaacttggtgttgatggcaagatgtttaatcgtcttttggctctatatcataaagataaccgtgatgtgtttgaagctgaaactgaagaggtagttgaactttatgacacagttttctctaaatgatattcgtccggtcgatgaggccgggctttcagaaaaagaactttcccttaaaaaggaaaaagatgaaatcgcaaagcttcttgatcgacaagaaaatggatttatcattgaaaaaatggtagaagagtttggaatgagttatcttgaagctacgacagcattcttggaagaaaactctattcctgaaactcaatttgctaaatttattccttcgggtataattgaaaaaattcagtcagaagctattgacgaaaatcttttacgtccttctgttgttcgttgtgaaaaaactaatacattagattttctgctatgattaaactccgcatgcctgctggtggtgaaagatacattgatggtaaatcagtttataaattatacttaatgataaaacaacatatgaatggaaagtatgatgttattaagtataattggtgcatgcgggtgtctgatgccgcttatcaaaagcgaagggataagtattttttccagaagttatcagaaaaatataaattaaaggaacttgctttaatctttataagtaatttggttgctaaccaagacgcttggattggcgacatctctgacgctgatgcacttgtgttttatcgtgaatatatcggacgcttaaagcaaattaaatttaagtttgaagaagatattcgcaacatttattattttagtaaaaaagttgaagtttctgcttttaaagaaatctttgagtataatccaaaagttcagtcaagttatatttttaaacttctgcaatcgaatataatttcgtttgagacgtttatcttgcttgattcgtttttaaatataatcgataaacatgatgaacagactgataatttagtctggaataattattctataaagttaaaggcttatagaaaaattttaaatatcgattcacaggaagctaaaaaagtttttattgaaactgtgaaatcttgtaagtattaattgcttattataaatagattataattatctcactgaccagctatgaggtcatacatcgtcatagcaccaactgttaattaaattaaaaaggaaataaaaatgtttaaacgtaaatctactgctgaactcgctgcacaaatggctaaactggctggaaacaaaggtggtttttcttctgaagataaaggcgagtggaaactgaaactcgataatgcgggtaacggtcaagcagtaattcgttttcttccgtctaaaaatgatgaacaagcaccattcgcaattcttgtaaatcacggtttcaagaaaaatggtaaatggtatattgaaaactgttcatctacccacggtgattacgattcttgcccagtatgtcagtatatcagtaaaaatgatttgtacaacactgacaataaagagtacggtcttgttaaacgtaaaacttcttactgggctaacattcttgtagtaaaagacccagctgctccagaaaatgaaggtaaagtatttaaataccgtttcggtaagaaaatctgggataaaatcaatgcaatgattgcagttgatgttgaaatgggtgaaactccggttgatgtaacttgtccgtgggaaggtgctaactttgtactgaaagttaaacaagtttctggttttagtaactacgacgaatctaaattcctgaatcaatctgcgattccaaacattgacgatgaatctttccagaaagaactgttcgaacaaatggttgacctttctgaaatgacttctaaagataaattcaaatcgtttgaagaactgaatactaaattcggtcaagttatgggaactgctgtgatgggcggtgccgctgcaactgctgctaagaaagctgataaagttgctgatgatttggatgcattcaatgttgatgacttcaatacaaaaactgaagatgattttatgagctcaagctctggcagttcatctagtgctgatgacacggacctggatgaccttttgaatgacctttaatagattatattactaattaattggggaccctagaggtcccctttttttatttcaaaaatttttcacaaaacggtttacaagcataaagctttatggtactatacaactatcggcaatactgctgatgattaaagaggaaaataatatggctaaagttgatattgacatcgttgattttgaatatattgaagagattattcgtaatcgttatcctgaacttagtatcacaagtattcatgatgatcccaattattgcaatttttctattgtcattgaaggtcctcttgaagaccttacccgctttatggctaatgaatattgcgatggcatggattctgaagacgcagaattttacatgggactgattgaacaataattatcaagggggctatttgatagccccgttaaaatgaggaaatcaaagtggaagttggaaaaacttatatcattaaccctttatttaaaaatgactttgctaacgagtgccgttgcaatgtaatgatgctaaatttaatagagaagcacggacctagttttgttgtagatgaaatggaaagtgatgattgtgctggtgactgtgtggtacgtgtaactatggaagacggcactgaatacatggcgggtggcccacttgagacgtattttgagatttaccctgaagaattcaaatactttactgaggtaactttagaaaagcctcaatccgctaatgaaggtatagtttctggcgtaacaaaaattcactgcattgttgacgaaaacaatgtagatgaaatcattgaacttttgcgaaaaacttttaaggcctaatatgactaaagatgatttcctggatgcggtagaaaaagctagtgagaaatttgaagtgatgggtgtacgggtccataaagacagaaattacggactcgatattcaaacgtattcgttatggcttgtgggtgaacacagtcttgtaaccagtttagttatgggtgtcgatacctatcctctattggttaagttcagaagtatggaaaaggctactaaagaaatgattaagcttggtaagtggctcgataaaattcaaaaataattgtttacaacggggtagtagtgtgatactattaccctatcaaaactaatggagaaaagaaaatgttcgcaccttatattatggcagcagttatgttggtctgtttatatcttttgattaaagcttgctaaggagaataaaatgagattacaacgccagagtatcaaagattcagaagttagaggtaaatggtattttaatatcatcggtaaagattctgaacttgttgaaaaagctgaacaccttttacgcgatatgggatgggaagatgaatgtgatggatgccctctttatgaggatggagaaagcgcaggattttggatttaccattctgacgtcgagcagtttaaagctgattggaaaattgtgaaaaagtctatttgaggaaaataatatgtctgacattcgtgtagctggaattgctcgagtaagtattcgttgccaacttaaaactgcacccgggacggcttatattaatttatcacacgacctatgttctagtgaaagaccattaactggtgtaattacgttttacgctggagttggcggaaacgaatttactgtaggcgaatttgttggctgtaaattaaaagttcagaagaacgtgttagaactttttagcgatctttatggtgatagtgatgaagtttttgatgaaatctcgcaagcagttaacagaggaatggtaactttagttaaaatgattaaagatagcggatttgcttctggcccctttcagtgggaggtaatatgatttttgtatttgaatttatgaatgatgaattcgattatgcaatttttaacgcattgcataatcctgatttaaatgaatttaaagaaatgttttctgacgctttaagtatgtcagaagaatactgtggagaatgtcaacgtgtttgtgtgacagtttttgaaaacaaagaaaagacatatgaagaattattctttgatgctaataaagccactgaatggtttatcgaaagaggatttgtgtaatgattaaattagtatttgcttatgctccaactaaaacagtcgaaggctttaatgaattagcattcggtttaggtgatgggttaccatggggacgagttaaaaaggacctccagaattttaaagctcgtaccgaaggaacaattatgattatgggtgctaaaacgttccagtcattatctacattacttcccggtcgtagccatattgtggtgtgtgaccttgagcgtgattatcctgtaactaaagacggtgatttagcacatttctatattacttgggcgcaatatataacttacatttctggtggcgaaattcaagtgtcaagccctaatgcaccattcgagactatgcttgatcagaattccaaagtaagtgtaattggaggacccgctctactatatgctgcattaccttatgcggatgaagtagttgtttcccgcatcgttaaaaggcatcgtgttaattcaacggttcagttagatgtaagttttcttgatgatataagcaagcgtgaaatggttgaaacgcattggtataaaatagatgaagtaacaacccttacggaatcagtatataaatgaaataacgcgcggcggaaatatgaattttaattattaccctattctattagaaaaagatgagaaacagccaaagtggcagggtcctcagtttattaaaggtgtctatcaattagtagttcctaaagacaagatttatagcagttgtttcactgaatccgcttgtagtattttcggtaatagttctccgtattggaattttgacataaagctggatagaaatatcgatatttggttgaaagctatggatattggtaatatcacgtttgatgagaataattatcatattattggtcgcttttccaaacgcggtaaagaattatatttcacgcctgaaattgagagaaaatttgatgctaaaccgtattgattatttagcttcataaaatgtattataatttatataaggtactaaatgaaacaataccaagatttaattaaagacatttttgaaaatggctatgaaaccgatgaccgaacaggcacaggaacaattgctttgtttggtactaaattacgctgggatttaactaaaggttttcctgcggtaacaactaagaagctcgcctggaaagcttgcattgctgagctgctttggtttttatcaggaagcacaaatgtcaatgatttacgattaattcagcacgattcattaattcaaggcaaaacagtctgggatgaaaattacgaaaatcaagcaaaagatttaggatatcatagtggtgaacttggtcctatatacggtaaacaatggcgtgatttcggtggtgtggatcaattagttgaaactattgatcgtattaaaaaattaccaactgatcgacgtcaaatcgtttcagcatggaatccagctgaaatcaagcagatggcattaccgccttgtcatatgttctatcagtttaatgtgcgtaatggctatttggatttgcagtggtatcaacgatcagtagatgtttttctcggccttccatttaatattgcatcatatgctgcgttagttcatattgtagctaagatgtgtaatcttactccaggagatttgatattttctggtggtaatactcatatctatatgaatcatgtagaacaatgcaaagaaattttgcgtcgtgaacctaaagagctttgtgaactggtaataggcggattgccttataaattccgttatctttctactaaagaacaattggaatatgttcttaaactcaggcctaaagatttcgttctcaaagattatcagtcccacggtgtcttgaaaggaaaaatggcggtataattttaatttaattgcgaggatatatgattttacgatttaaagatacttctggtgtagttctttttacacttcctaatccaagtgagctagaagttccaggaccaaatcagcctattatcatttatggcaaaaaatattatactcataaaatgactcgtgagtattttgataataaaatttccacagttaaaacttcttctgactgttactacgatattactgttttaacggaagaacaatatgaacacttacaaccgcgcaggccgtatatgccgagtagtgaataaatataaatccgactttgatgttaatattcaccgtggtacattttggggaaattacgtcggtaaagatgctggcagccgggaggctgccattgaattattcaaaaaagattttatacgtcgaattaaatccggagaaataactaaagcacatttagagcctttacgtggaatgaggctaggatgcacatgtaaaccaaagccgtgtcatggtgatataatagctcatatagttaaccgattgtttaaagacgattttcaagttgaggacttatgcaattaattaatgttatcaaaagtagtggtgtttctcagagctttgacccacaaaaaattattaaagttttatcttgggcagctgaaggaacatcagtagatccttatgaattatatgaaaatattaaatcttatctccgtgatggaatgacaactgatgatattcagactattgtcattaaggccgctgcgaattctatttcggttgaagaacctgattatcaatatgtagctgcacgctgtttaatgtttgctcttcgtaagcatgtttatgggcagtatgaaccacgttcatttattgaccatatttcttactgtgtaaatgaaggtaaatacgaccctgaattgttgtcaaaatattctgcagaagaaattacatttttagaatcaaaaattaagcacgagcgggatatggaatttacttattccggggcgatgcaattaaaagaaaaatatctagttaaagataaaaccactggtcaaatttatgaaactccacagtttgcatttatgactattggaatggcattgcatcaagatgaacctgttgatagattaaaacacgttatccgtttttatgaagcagtatctactcgacagatttcattaccaactcctattatggctggttgccgtactccaactcggcagtttagttcatgcgttgttattgaggcaggtgattcattaaagtctatcaataaggcttccgcttcaattgttgaatatatctctaaacgtgctggaattggtattaacgttggtatgattcgtgctgaaggttctaagattggcatgggtgaagtacgccatactggtgttattcctttttggaaacattttcagactgctgttaaatcatgctcacagggtggaattcgtggcggcgctgctactgcttattatcctatttggcatttggaagttgaaaatcttcttgttttgaaaaataacaaaggcgtagaagaaaaccgcattcgtcatatggattatggtgttcaactgaatgatttgatgatggaacgttttggaaagaacgattacattactttgttcagtccccatgaaatgggcggagagctttattattcttattttaaagaccaagaccgtttccgtgaattgtatgaagccgcagaaaaagaccctaatattcgtaaaaagcgtattaaagctcgtgaactatttgaattgctcatgactgaacgctcaggaacagcaaggatttatgtacagttcattgataatacgaataactatactccgtttattcgtgaaaaggcacctattcgtcagagtaacttgtgctgtgaaattgctattccaacaaatgatgtgaatagtcctgatgctgaaattggattgtgtactctctctgcattcgtattagataattttgactggcaagaccaagataaaattaatgaattggcagaagtccaggttcgtgctcttgataatcttttggattaccaaggatatccagttcctgaagcagaaaaagctaaaaagcgtcgtaacctcggtgtaggtgttactaactatgcagcttggttggcaagtaactttgcttcttatgaagatgctaacgatttaacacatgaactatttgagagattacagtatggactcattaaagcatccattaaactcgccaaagaaaaaggaccttgcgaatattattcagacactcgttggtctcgaggcgaattacctatcgactggtacaataaaaagattgaccaaatcgcagctccaaaatacgtttgtgactggtcgtcgctgcgggaagaccttaagctctttggcatccgtaatagcacattatcagcacttatgccatgtgagtcatcttcccaagtttctaacagtacaaacggtatcgagcctccacgtggaccagtctctgttaaagaatcaaaagagggctcctttaatcaagtcgtgcccaatattgagcataacatagacctgtatgattatacgtggaaattagctaagaaaggtaataagccttatcttacgcaagtagctattatgctgaaatgggtatgtcaatcagcttcagcgaatacatattatgacccacagatttttccaaaaggaaaggttccaatgtcaataatgattgatgaccttttgtatttttggtattttggcggaaaaaatttctattatcataatacccgtgacggttctggtactgatgattatgaaatagaaactccaaaagctgaagattgttcatcctgtaaattatgatataatttgactcacggacgagtcactatctattaactaagcggaaaatttatgagcacagtttttaatacaaatccggttgatgttttaaaagaacctatgttcttcggttcaggtcttggtattgcgcgttatgatattcaacgtcataaagtttttgaagatttaaccgaaaagcaattgtcatttttctggcgtcctgaagaagtaaacttgatgatggatgctgcacaatttaacaagcttccccagtatcagcaagatatttttactaataacctgaagtatcaatcacttttagatagcattcagggtcgtgcaccgtctgctgtacttatgtcattaatttcagatccaagccttgatacatgggttgctacatggacttttagtgaaactattcacagtcgttcatatactcatatcatgcgaaatctttatactgatccgtcaaaggtatttgatgagattgtattagataaagctatcatgaaacgtgctgaatctattggtcgttattatgatgatgttattgagaaaactcgtgaatgggaaaacgcaaaagaaatttttaggtgtttttcagagaatgaatatgatttggacgcatattcaaatagccttatgcatgtagaagaatgcaaatacgaactaatgcgttctctttacctttgtctgcatgtaatcaacgcattggaagctattcgtttttatgtatcctttgcatgtactttcaacttccataagaacatggaaatcatggaaggtaatgcgaagattatgaaatttattgcacgtgatgagcagcttcatcttaaaggtacacaatatattattcgtcaacttcaatctggtactgatggcgatgaatgggttaaaattgccaaagagtgtgaacaagaagcagttgatattttcatggaagttaaccgccaagaaaaagattgggcagttcatttatttaaagatggcgatgttcctggattaaatacaaatagcatgtggagctttattgattacttaactgtatctcgtatgaaacagtgtggtcttccatgcccgattaccgatgctccggttaaacatccatatccttggattcgtgaataccttaattctgataatgttcaatccgcgccgcaagaagtagaactgtcatcttatcttgttgcacagattgataatgatgttgatgataaagttatgatgagttttaaaaaatacttttaaggagtgggccgcaaggcccattttattatgaaagaaattgcaacagaatattcatttattaaatatactgagttagaattagacgacaacggaagtataaaacaactatctattccaaataagtataacgtaatttatgctattgctataaatgatgagcttgtttatattggaaaaactaaaaatttacgtaaaagaataaattattatagaactgctattaatcgtaaagacaaaacgtctgattctactaaatctgcattaattcatgctgcgctaaaggaaggaagcaaagttgaattttacgcccgccaatgttttaatctttctatgacaaatgagttaggtacaatgacaatcgcgacgattgacctagaggagccgctattcattaaactgtttaacccgccttggaatattcaacacaagaaaaaatgatgcttccacatggagtgtggtactatattcaaaacacaaaagaggatacacaatgcaagaactttttaacaatttaatggaactatgtaaggattcacagcgtaagtttttttactcggatgatgtaagtgcatctggaagaacttacagaattttctcatataattatgcatcttattctgattggttacttccagacgcattggaatgtcgtggaatcatgtttgaaatggatggagaaaaaccagtaagaattgcctctcgtcccatggaaaagttttttaacttgaacgaaaatccattcacgatgaatatcgatttaaatgatgttgattacattctgacaaaggaagatgggtctttggtatcaacttatttagacggtgatgaaattctgttcaaatcaaagggttcaatcaaatccgaacaggctttaatggctaatggtattttgatgaatattaatcaccatcggttgcgtgatagacttaaagaattagctgaagatggatttactgctaacttcgaattcgtcgctccaactaatagaatcgttcttgcttatcaagagatgaaaatcattttattgaatattcgtgaaaatgaaacaggcgaatacatttcatatgatgatatttataaagatgctgctcttcgtccatatctagttgaacgatacgaaatcgatagccctaaatgggtagaagaagctaaaaatgcggaaaacatcgaaggctatgttgctgtgatgaaagatggttctcattttaaaattaagtctgactggtatgtgtctcttcatagcacaaaaagttcattagataatccagaaaaattgtttaagactattattgatggtgcatcagatgatcttaaagcaatgtatgctgacgatgaatattcatacagaaaaattgaagcatttgaaacgacttatctgaagtacttagaccgagctctgtttttagttcttgactgtcataataagcattgtggtaaggatagaaagacttatgcgatggaagcgcaaggtgttgctaaaggtgttggaatggatcacctgttcggtatcattatgagcctatatcaggggtacgatagtcaagaaaaggtcatgtgcgaaatcgaacagaattttttgaaaaattataaaaaatttatcccagaaggatactaagctgtttacaagtccctcgtgttgtgttatagtagtcttactgacataacatgaggactttatgatggatttgcagcttattactactgaaatggtcgttgaagcatacggtgatactacagatgggatttctgtattcaaaggaaatcgtcgagttggatatatcaccgatcttaagaaagatttagctaagcaagtcaagcggaaaacgaccattaaagaatatcgaaatcgtcgtcttgagcaagcccgtgatatgcttcctgatgcggttgaggagatgaaagtctttttagaaaatcaactcgctaagtatgattgtgaagtgtttattaatcagactcaacctaatgttcatatcaatagctgcaaatgctatatcatcgttaatcctttgacaggaaagcatcgtcttggaattagtaatccaaatcgtagcgcatcggatatggcagaagatgttgcggcgtgctttaaaatttctaaatctccggctgaacatcatattttaattaacggtctttctcaagacgatattatagaggttattaaaactttatgcatgtaagtaattttacagctggattgctattacttgtaatagcatttggcggaacatctattattttaaaaaataaggtagaaagattagaaacgtctgttgtagaaattacaaaaacggccaatgaaaacgccttagcattaaataatttgcgaattcagtataattatattgatgcgatgaataataaaaatcgtgaggcgattgctgctattgagcgtgaaaatgaaaaactgcgcaaagatgcaaagaaggcggatgtggtggctcgtaagccaggattggttgaaaaacaaatcaacaactccttcaacaagttcgcagaagacatccaggacctttctaaatgattaaattatctgcagtaatattatctattggtcttctagttggttgttcgacaaagcctctagaagtaaagaaagaaacagttcatcctaattggcctgtgcaaataaagtcatatgacgaagctaaactatcttggcaagttaaagttattgatggtaaagcttgggtaggtatgccatttgaagattctcaggaatttcgtatttggcttaatgatgtaaaacgatatgtacatgaccagaaaactatgatatgttattatcgtcaagagctaaaagaggataaatgtaaatgatttcatggtatcaatttgaacatctaaaaggattaatttatgaatccgagatggctgcaatgatttatggacgacagattcaacgattagaatctttacctccaactaatgatgttttattagctcaatcacgcgctaatctcaaaaatgaatatcaaaataagtggggtaaagcatctaaagacctgcatgattatattcaatcattggttgagaaaaataaatgaaaactctgttagaacgttatattgaatgttcagaccgttacattgatgcatgccgtgaaaatgtatcaagtattagcgaagacattgaacacgctaaagctttagatgatgcgggtaaagccctgcgaaaagaagcgaaagctcgtgggcttgatatgtatcagcttaaaaatcacatgataaaatttatttcatccaatgttcagagtaaatcagcgaatcaatcaacagctgaattatacaaaggacggcgtgagcataatattcgtattcttgaagttttcttaggaattaaatgatgaaaaagattattttgactgtaggttgtcctggttctggtaaaagcacttgggctcgtgaatttattgctaagaatcctgggttttataatattaatcgtgatgattatcgccagtctgttatggcacacgaagaacgtgatgagtacaaatacaccaagaagaaagaaagcatcgtgacttatatgcaacacgatgcggcacatatgattctctgccaagacggaacgaagggtgtaattgtttcagatactaatctgaatcctgaacgtcgtcttgcctgggaagaatatgctaagcagtggggacaccaagtcgaatatcaagtgttcgatgttccatgggttgaattagttaaacgtaactcaaaacgtggaactaaagcagtaccaattgatgttttacgctcaatgtataaaagcatgcgagagtatctcggtcttccagtatataatgggactcctggtaaaccaaaagcagttatttttgatgttgatggtacactagctaaaatgaatggtcgtggtccttatgaccttggaaaatgcgataccgatgttatcaatcctatggttgttgaactgtctaagatgtatgctcttatgggttatcaaatcgtagtcgtttcaggccgtgaaagtggaactgaagaagacccaacaaaatattatcgtatgacccgtaaatgggttgaggatattgctggcgttccattagttatgcaatgtcaacgcgaacaaggcgatacccgtaaagatgatgtagttaaagaagaaattttctggaaatacatcgcgccgcattttgacgtgaaattagctattgatgaccgaactcaagtagttgaaatgtggcgtcgcatcggtgttgaatgctggcaagtcgcctcgggagatttttaatggctttgcaccatgaaacttgggctattgttattgtaaatagcggtttagttggtactagtaatgggcaattttgtgtatttactagtgaaaatagagcatgggaggaatgtcttaaattaagagaaaagaatcctgatgttgaactagtagtaaagaaaactaaactgcctttaccatggaaaacttatgaataacctagaaaagatttatcatctttgtgataaaattgaaaaagaaaagaaatatctattttgtttatggcctattgttgatggaagagtaggcttagatgttcttgattatgaaacagaagacagagtagatggttcaacttttgataacgctttagatgttattgattggcttgaagaaaattatgtgaggtaaatatgtttccgacttactctaaaatcgtagaagtagtgtttagccaaattatcgctaataatatgtttgaaaagcttgataacgcagccgagcttcgaatccatgctcaagtgactcatgtattgaacactttgcttccagaccaggtggattctgttgccattacgctgtatccaggttccgcgcatatcattgttgtgttcggtcttgatgctgagctagtcatcaaaggcgatattcgttttgaatcgcagacagcagaattcaaagcaatttaatagtttactttacggtagagttgtgatattatagctctaccaaaacaaatgaggaaattgaaatgagcgaatggtttgaagaagataaggtttatcgctttaaagctggatataaagatatttttaatgaaacttgcggggctaataaacgaattgcccagtttattggggaaaattcatttaaagtaaaaatagatcctgcgaaaaatgttattagcattaaacgcgaaattgatgattgttggtataaagctgttgatgtaatgggtgaatcctataaagttagcccgttattttcaattgcttatatgttagaatattcttttttcgaagaagttcaaaaagatgattctgtaagtaaatttgaaattaaaactgataaagaaattaagtggaaagtagtaggtattactggttgtatgttctatatctatgctcaaactgatacgaaggaagaagctaaaaagaaagctctagaatatcttgaagagtatgaagaaggcccggtaatgattactcaagatgctgaattagtttttgtcaaattagttaaaaacgttgaaagtaaggagctgggatcaacatgctaagtgaaaaaccgattactgttaaagaattccaagaaaaagttaagctatttgcacaggaattggtaaataaggtttctgaacgatttcctgaaacatcggttcgtgttattaccgaaactcctcgttcagtattagtaattgtgaatccaggtgatggtgatcaaataacacatctcaaattggattttgatggattagttgaagcacaaagggtgtatggcgtactatgatgaatttaactgatataattgataattgtcttgaaaatgatactggcgatcatagagcgcttgattctgaaacagctcagttcattagaataactttaatgaatgacactctagtaaatagtattcatccttctgtgtatgatgctattattgtgacgaagtatccagttgagcttcacaaaaagatggttggcgcaatttttattgataagaaaaaccgctttaaagatgggcagaatataattagttctgttattaaaagtataactaaacttcgtcacgaaatttatcgtgttgaaactgctaaatctgcttatctggtgattatgaaatgaaagcgagtacagtacttcaaattgcatatttagtatcgcaggaatcaaaatgttgctcctggaaggtaggagcagtaattgaaaagaatggacgtattatttctaccggatataatggttcacctgctggtggtgtgaactgttgtgattatgctgctgagcaaggttggttgttgaataagcctaaacacactatcattcaaggccataaacctgaatgcgtatcatttggttcaactgattgctttgtcttggctaaagaacatcgtagtgctcactctgaatggtcgtctaaaaatgaaattcatgctgaactaaatgcaattttgtttgctgcacgaaatggttcttctattgaaggtgctactatgtatgtaacactttctccttgcccagattgtgcaaaagcgatagctcaatctggtattaaaaaactggtttattgcgaaacatacgataaaaataaacctggctgggatgatattctgcgaaatgcaggtattgaagtgtttaatgttcctaagaaaaacttgaataagttaaactgggaaaatatcaacgaattctgtggtgaataatgaaatttcgtttggtaaagcttacagcaattagttcttattctaacgagaacatctcgtttgctgtagagtataagaaatattttttctctaaatggaaacagtattataaaactgattggactagtattgatagaccatatagttggaaatctgatttagaaaaatgccaaaaattactttccacccttaaagaacgtggaagaactcatattaaaactgtaataggtaaataaatgaaactgacaactgaacagaaagtagcaattcgtgaaattttgaaaactaaattgtccatgggtatttcaaacgtagtttttgaaaagtctgatggtactattcgtactatgaaaggtactcgcgatgctgactttatgccaaccatgcaaactggtaaattgactgaatctactcggaaagaatctactgatatgattccagtatttgatgttgagcttggcgagtggcgaggtttttctattgacaaattgatttctgttaatggtatgaaagttgagcatttactccaatttattggtaaataaatgctttaagaattatttgttattattaattcatctgttaacaaaaaggaaaaacgatgtctgaagtacaacagctaccaattcgtgctgtcggtgaatatgttattttagtttctgaacctgcgcaagccggtgatgaagaagttacagaatcaggacttattatcggtaaacgtgttcaaggtgaagttcctgaactatgtgtagttcactctgtcggtcctgatgttcctgaaggtttctgcgaagttggtgatttgacttctcttccagttggtcaaattcgaaacgttccgcatccttttgtagctctgggtcttaagcagccaaaagaaattaaacaaaaattcgttacctgtcactataaagctattccgtgtctttataagtgatataaataataatatgaattgggtgtcggaataataagttaaccgaacaattctatgtggtagtctacaactgagagatctgtcgaaagaagatgaaattcagaagaacgtgactaccgagttttaatctctaacgagaatttttaaatgattaaacaattacaacacgctcttgaactgcaacgaaacgcatggaataatggtcacgaaaactatggcgcatctattgatgttgaagccgaagctcttgaaatcctgcgttatttcaaacatctgaatcctgctcaaactgcattagctgctgagcttcaggaaaaagatgaacttaagtatgctaagcctctggcttctgctgcacgaaaagcagttcgtcactttgtggtaacactgaagtaatttattggagattcactgccttagtgtgagctaaaatcgaggagccatcgaactgtctgattaatgatttgcgaatcattatagttttaagaccccggcagttttatggtgtacctcttgaatgtgaatgatgacgggtttatggttatcctgtcgttaaatatccaaaacctatgttccccttgagggcttgcgcaggcaatgccaataagtcctgcattttcatttaaaagagaatttataatggcaaaacaagctaaagcaaagaaagcagttgaaaagaaagttggtgattctaaacgcgctggctacaagcgtgggtcgaactctcgtatcaatcaaactgttgagaagatcatgcgccgagcacgtgcggttcttcgagatgatgcttctcgttttggtaagcagaaagcataagttaaggactccttcgggagtccttttttattttccaaagattgcataaagttgtttacagtatggttcctttgtgatagtattatcttacacaaacaaaggagaataaaatgaaaacgattaatctgaacgctgcagttaagactaaatgcttcaatggtaaatatgatgaaactatgtggttcttaatggcaattgaaggtgatattattgaagtagaaacaacagaaggtatgggaacagatttcacctttacaattcaagttcataatttctttactggttggatttatgaattgaatacagtaatcgttggaaaaattgaacaaaatgaattaggcgaatggtattatgttacagctcgccaacgtgccgaacgtttgattgagaagatgaaaaaagttggtaaacttgacatgaagcactggaaagtagtaaaataattgtttactttggtacaggatatgatattatatacctgtaccgcaattaaacatctcggagaataaaaatgaactacatcaactttgaacgtaaatatgtttctaatggtattgcaggttctattgatactatttgcctttggaaacatcaaaatggatcagtatgcgaaattgaacagtatatgactcctaactacgtttatatgcgatttgaaaatggcatcacggtttcaatcacaatggaaggttcaaacttcaaaatcgctctggatgatgattttcgtcaacgcgatttagggacccatccttgctggaatggcgctaatcgtaagcttctggttaaaacttggattcgtcatattttgagtaacagagctaaacctgagcatcttgaagcaatctttgatgtagttcttaacgaatttgatatttaaaataaaacaggggcttcggccccttactgaggaaaatactatgtttatgactacttattttgatacccgcaaaaatttctgcgaagttgttttctctaaggcgcctaaagaccttcctgctcatttgcaacctaccagtgaatcgattaaaaattatgttgatgtggtttgtcctttagaattccgtacagtaaatgggcgtgatactttagctatcgctaaacttaatcgtgaaattgacattgacccttcaattgcgcgtgaaattaatatttctgatattggcggcggtaatgttaaatcacacggctttcagatgaggttctgatgaaattctttttaggtcaaactgttgaattaaagggagttggtatacctggattaatttctaaggttctacctccatttaaatggagtggtgttcaaataaaagaggcttatattgtttcctgggtagatggaaatgaagaccttcgtatgggtgatgaattatctcctatttacggattaaaggaattagtatgaatataattaataagatttttggaattcagtacattaaggtcacatataaagtaacagataaaaatccatatactgatgaacatgaagaaccacaagttaagtctattatattagaaaaggaccctaactggccagttgaatttcgtcttccttgctatggtcattgggctgatgttgaaattataagtattgaaaatgcctgagttagagattagaagcaattttagatggccatcgtgtgcattaagtaatttcgcccaatggccttttgttatggatggtatccaatttggaggtcttgaaggatttctccaaggatgtaaggtgaaaaatgttgaacagcaacgtcgtgtatttgggttatccgggcttgccgcccaacaagctggaaggtcttatgctagagctcaggaccgtgggaccctcttctggctcggagttccgttttcaagatactctccagcgtggaaagaattatacacaaatgcgtattttgaggcagcgatccaaaacaaggggtttcgtgatgccttacaagcctctaaaggaaaaattttgaagcatagcatggctagtggtctaacaaaagatgatacaatactaaccgaagttgaatttattgatgtgttaaacctattaagagactctctatgaagcctactattttgactgatattgatggtgtatgtttaagctggcaatcaggccttccttattttgcccaaaaatataatcttccattagaacatattttaaaaatgattcaggacgaaaaatttatttctcctggtaagctttttaattgcgatgaagaacttggtgttaaattaatcgaaaagtataatcgttcagattttattcgctatttgtctccatataaagatgccctatgtgtaattaacaaattaaaagaagattataattttgtagctgttacagcattgggtgattctattgacgctctgctgaatcgtcaatttaatttgaatgctctttttcctggtgccttctcagaagtattgatgtgtggtcatgattcttcaaaagaagagttgttcaaaaaggcaaaagagaaatataacgtaatttgttatattgacgatctcgctcaccactgcgatcatgcaagtgaaatattaaatgttcctgtttattggatggctcgaggggagcgtgacagtattccaaaaactgcacagcgagtttatacatggaatgatgtagagaataagctttttccatcgaaggaaaataaagaaagttttgatagtgaaaaagttataaaagatgtaattgagaagatgattaaaaacgattcttttccttggaacactacctggagaactcctggatttaatccttataatctatatcatccatatcagacgcatccgtttcagacatggaactatattaaacccggtggcatagagtatttgtataatagacctactagtggtgataatattttccaaggagcattctaatgtttgtcgttcacactatttatgaaaatgaaggtaatactacacgtgattacggtcacgtaaatcaattttttagatgcaatccagaatttcgagctcaaaaagacgaacgaatttttaaaaaatgtgtagagcaaggtttcatttacgtcaagcactggatgcaaggaaataaagttagaaccacgtaccataagtctttgactgagcttaatgatgaattgatttataatagagctgtaaaccaaactctaaaggatgaacaatgattcttaaaattctgaacgaaatagcatctattggttcaactaagcagaagcaagcaattcttgaaaagaataaagataatgaattgcttaaacgagtatatcgtctgacttattctcgtgggttacagtattatatcaagaaatggcctaaacctggtattgctacccagagttttggaatgttgactcttaccgatatgcttgacttcattgaattcacgttagctactcggaaattgaccggaaatgcggcaattgaggaattaactggatatattgctgacggtaaaaaagatgatgttgaagttttgcgtcgggtgatgatgcgagaccttgaatgtggtgcatcagtatctattgcaaacaaagtttggccaggtttaattcctgaacaacctcaaatgctggcaagttcttatgatgaaaaaggcattaataagaatatcaaatttccagcttttgcccagttaaaagctgatggagctcggtgctttgctgaagtcagaggtgatgaattagatgatgttcgtcttttatcacgagctggtaatgaatatctaggattagatcttctcaaggaagagttaatcaaaatgactgcagaagctcgccagattcatccagaaggtgtgttaattgatggcgaattggtataccatgagcaagttgaaaaggaaccagaaggcctagattttctttttgatgcttatcctgaaattagtaaagctaaagaattcgccgaagtagctgaatcacgtactacttctaatggaatcgctaataaatctttaaagggaaccatttctgaaaaagaagcacaatgcatgaagtttcaggtctgggactatgttccattagtagaaatatatggtcttccagcatttcgtttaaaatatgatgtacgtttttctaaactagaacaaatgacatcaggctatgataaagtaattttaattgaaaaccagatagtaaataacctagatgaagctaaggtaatttataaaaagtatattgaccaaggtcttgaaggtattattctcaaaaatatcgatggattatgggaaaatgctcgttcaaaaaatctttataaatttaaagaagtaattgatgttgatttaaaaattgtaggaatttatcctcaccgtaaagaccctactaaagcaggtggtttcattcttgaatcagagtgtggaaaaattaaagtaaatgctggttcaggcttaaaagataaagccggtataaaatcgcatgaacttgatcgtactcgcattatggaaaaccaaaattattatattggaaaaattctagagtgcgaatgcaacggctgggtaaaattagatggacgtactgattacgttaagctatttcttccaattgcaattcgtttacgtgaagataaaactaaagctaatacgttcgaagatgtatttggcgattttcatgaggtaactggtttatgaaagcttacttagaaacaattgtcgtggctcaaaaagaaggtggagatgtttccactgcggtatcgcaggttattcttgaattcccagataaactagcatatgatgaattcacggaaagatttgatccatatgaaaaaggtccaaagtttgaagtatatcgtactcttttagctctctattaattaaaggccttcgggcctttaattttataaatagaataaacactagagaggataacatggaacttattacagaattatttgacgaagatactactcttccgattacaaacttaaatccaaagaagaaaataccacaaattttttcagttcatgttgatgatgcaattgaacaaccaggctttcgtttatgtacctatacatctggaggtgatactaatcgtgatttaaagatgggcgataaaatgatgcacattgttccttttacattaactgctaaaggttcaattgctaaattaaaaggtcttggtccaagcccgattaattatatcaattcagtttttactgttgcaatgcaaacaatgcgtcaatataaaattgatgcctgcatgcttcgtattcttaagtctaaaaccgccggtcaagctcgacaaattcaacttattgctgatagactcatccgcagtcgttcaggtggcagatacgtccttcttaaggaactctgggattacgataaaaagtatgcatatattcttatacatcgtaaaaatgtatcactagaagacattccaggagttccagaaattagcaccgagctctttactaaagttgaatcgaaggtcggtgatgtttatatcaataaagatactggagctcaagtaaccaaaaatgaggcaattgctgcatctattgcacaagaaaatgataaacgttctgaccaagccgtaatcgttaaagttaaaatttcccgtagagcaattgcacaaagtcagtcattggaatcttctagatttgaaagtgaattattccagaagtatgaatctacagcagctaattttaataaaccagctactgcacctttaattcccgaagcagaagaaatgaaaattggaattaattcattagcttctaaaacaaaggcagcaaaaattattgctgaaggaacggcagatgaacttcactatgattataaattctttccaatgagtcaagtcggtgaagtttcagaaaaaattaaagaagtaatttttaatgcaattaaaaatgaaccaactacttcaataaaatgtttagagaaatacgcagcagctgtaaatcaattctttgaagattataaagataaatggcttgctaatcataataaaaatcgtaaaggacaaccagatgaagtctgggaagaaataactaaaaattcctggaacgcagcaaaaactaaattcctcaaacgaatgatttacagtttttctggaattggtgctggtccaatgattgatattactattgcccgcgatggttctaaatatactccatcacaaaagcgtggcattagagaatattgcggttcaggatatactgacatcaataatcttcttttaggtcgttacaatccagaacgatatgatgtaatgaatgaaaaagaaattgaatctgctataaataacttagattcagcttttgaaaatggtgaccgtataccagaaggcattacagtttatcgtgctcaaagtatgactgctcctatatatgaagcgttagttaaaaataaagtgttctacttcagaaattttgtatctacttctttaactcctatcatttttggacgttttggaatcacacatgctggtattggtcttttagaaccagaagctcgcaatgaattaacagttgataaaaatgaagaaggaataactattaatccaaacgaaataagagcgtataaagaaaatcctgaatacgttaaagttcaaataggatgggcaattgatggagctcataaagttaatgttgtatatccaggaagtctcggaatagcaacagaagctgaagttattctaccgcgtggattgatggtcaaagttaataaaataactgatgcttctaataatgacggaacaacatctaataatacaaaactcattcaagctgaagttatgaccacggaagagctcacagaatcggtaatctatgacggagaccatttaatggaaaccggtgaagtagttgcaatgacaggtgatattgaaatagaagacagagttgactttgcatcatttgtttcatcaaacgttaaacagaaagtagaatcatctcttggaattattgcgtcttgcatagatattacaaacatgccttacaagttcgttcaaggataaatcatggaacttattacagaattatttgacggcgcttcggcgccggttgttaacttaaatcctaagcataaaataccacaaatttttgctattcaagccggtgaagaaagcgtgcttcctggatttagattttgtacatacacctctggcggtgatacgaataaaaacgttaagccaggcgacaaaatgatgcatatcgtaatgataggtgttaatgaaaaattatcattagttaagcttaaaaacttgggtggaaatccaattggcgtcattaatgctgtttttgatactgctcttcaaacaatgaaacagtataaaattgatgcatgcctattccgcgtactaaaaagtaaaacaaatggcgctgctcgtcaaatgcaagttattgctgaccgtttagtacgtactaaaggagcaggtcgatatgttcttttaaaggaaatctgggactatgataagaaatatgcatatattatggtttaccgtaaaaatgccaatttagaagacattccaggtgtacctcctatttcaactgagttattcacaaaagttgaatcaaaggttggtgatgtttatgtagacgttaaaacaggtaatgctgttcctaaagctgtcgcagttgctgcttctattgctttagaaaatgataagcgtacggatcaagctgttattcagaaaactaaaattagtcgtcgattagcagcacaagctcaatattctactgttgatgcttcacttcagggtgatagtttcgctgctaagaaatatcaagagtttgaatctaaagttccggtatataaagcagaaggaccaatgaactctggcgttattcaggttggttcaaacttcagcaaaggagctatcggtggtatgaggagtgcttctcgttttaaatctagtgattatgaactagaaagcttccgaaatcatattgcattagcccatgcacgtttacgtgatccatccatcaagttgcagagcgatataacatatcaaagttctcaagaatatttaaagaataaagaattttttgattataaaactgataaaattttaagcgatcttgctgacattaatatttctaatagctttgatgttattaagaaaattatcaatgatttggttaaaggttctaaagctacgccagatgaaaagacagctattattcaatttgtcatgaatggcatttataaattaattgatgaatccgctactcaggcgtatgaatatgcaagcaccgaagtaactccaaaaggattaactcaggctgaatctgacgtaattgaagattattgcgcagattcatatgttgaaatgaactcgttccttttgggtaaaccagattctacccgtgaagaatatatggaacgagctattaaacatattgagacgcttgattctgcattcgctaaaggttcagttcttcctccaggaactacactttaccgtggacaagaagttacttttaaaactttgcgtcataacattgaaaataaaatgttctatttcaagaacttcgtatcgacatcacttaaaccaaatatcttcggtgagcatggtaaaaactatatggctctagatgattccggtgcagtattttctggtgaaggagaaggttccgttgatgcagaagatttgatgcatatgggtagtcattctacatatgctaatgaagatgctgaaactagcgtgggtatggtaattaaaggagctgaacgagttaaagttatcgttccaggtcatttgtcaggatttccatcagaagctgaagttattctaccacgtggaattttactgaagattaataaagtaagtacttactttatgaaagaaactgcttataacaagtatctaatcgaaggtacaatcgttcctccttctgaacaattagaagaatcagtatatgatggcgaccatctaatggaaactggtgaagttcgtccaatggctggatttaatcaattccttgtagaagaatcaaaagaagaggaaaacgaagtttctcaaatcttagcttctttggttaacatcaacggaatgtctaaaaagttcaaaatgtagtttacaagtccctcgtgttgtgttatagtagtcttactgacataacatgaggaacacaaaatgaaatcttctttgcgctttttaggtcaagaactcgtcgttgaaggcgttattcccgctgataacgctttcaatgaagcagtttacgatgaatttatcaaaatttttggaacagataaaaagttcggaatttttccttctgaaaatttttcaaagccagaacagactgaaagtattttccaaggtgtagtaacaggtaaatttgagtcagaagctccggtaaaaattgaggtttatattgaagaaacctctgttgcttcagttgctgcttttatttcattccgtaaataaaaatatggggaccgaaaggtccccattgttatattgctcctaatattttactttgcgaattgacaattcctgtcatagtattaatatttgaaatgcttcctgcagttccccctaatctactaagtcgtgaaagcgaattagatagtccagtaacacctccactatttccgagaacgctttgaataccatttatagtagcagattcaagccattcaagggcagcttgtctatcaactgctccagcctgcatcactctatacgcaaaagtaacgtcaaatgtagttatttggttatctccatcatatgataactcaggagcgctcacagatattggaatacagccagtgaacatcaccgcagtatgaggtaatccattgcgagaatgaagattaacctgaatatctgcctcgacatcttgcggcaaagcacgcaatccagttactgggtcttgaacagcgttaacccagtcttgcattgcacgatagttacaagcttctgaatccattctgaatgaaataaccaaagggtctaattctcttccagttatacgaatattaggagaattatagttccagtcagtttcataggataatctattctctggcatttttacagaatatatcatcaatccagatgagttatatgccatgttaaagaagtcaattaaatatgtaccaactgtaaatgagcctaataaactctgaacagtacgctgactcatagcgccaataagatatttactgactcccgattttcttattagtttttgtgtgccagcggtaattagcgtggtaattccctgattaatatcaccttgagttaatcccaaccaatctgaatttagacccaagttattataagcaaagttactaattgagcttattaacgatgagcttttagttgatggagttgtagcaaaaacacagctgaacatattattacgttggaagtctgcgtttattgcttgattattaaattcctctaaagaatacattagaaagtccccgtatataaagaagcacggtttaacgtaataatctctctcatagtaatctcgagtgtaaaagaacttggaaggttaggagcaatagctaatccattaaagttaccatttggtgttttatcaaatctaatactctgtatttggcatggaccgaatacttcagtctttccatcaaatttagaagttgcaccaaagtttttcaccatccaaattgtcgggtttgaaactacaagaacgttcgttaaactcgatgtcattttttcaaataacgtcttgtttttaatcgcgtcttctggcgttaatggttcaataaatgtagaacgataccactcatctaaatatccttttatttcagcagcatattgagatttaccagtttcaccgtaagaaaaatagttaaaatactgatagatattaataatagccattaaatcttctgttgagcgcggagtcaaatcccaggtgaacacttttgttctattttcagcaccgccatacatacttctagctgttgtatagatctgttcattattatcagccattattccttgtgttatactttcaagcgcaccaaatactgcagttgaagccatattacttagcacaccagtagcagtacctccgccttttgtaataagactatcttgaacatcattaaatctatgtgatgatgtatcgacgtcagatttagatctaggtaaaagaatatttgcaacaggaactttacttattgttcctgaattattatctgatattagtccatttgatagttttgatactgtattactgatagtgtttctggctgtacgtaaaatactcgaagatgaagaagagtagttagatctcatcgttctaagacttccagaatccctagatgacatattgtatgcagtaaataataatccattcttatataggtctgttacctggaagtcccctgtagtgtcattaccactagcacgcccagttggaaactgggctgtgtatgttttagttcctacttctgatttagtactctgtccggctgaaattgtttcaccggactttttaattaaatcagcagttatttctttaacaattgccatattattccttaattaactcccgtcgcaccaaatacaccaggagcagttgtgcttgtgacaggtgtcatattatgaacgacagtatttttcttaataacattattagtattattgattgaaggagatgcttgttgaacaggagcttgttgtgctttgttcttttcaatcacctggacctgttttgcttctggtgatttagcagaagtttcaggtttggcattaggctgatttttcttgagctcttgataagtggcatcaattttagaaaatctagcagcaagttcttttttaaccgctggtgaattatttaaatccggatcattcattcgttttttaaggtcttcatgagcggcttcaattgatttaactgtcgagtctttgctcatatcagctgaatcagcgtatttttcaaaacgaatcatcgcagcacgagcttcattagctttcattaaagcattttttctttcttccggtgaaagttgttttaatttttcttcttctgctgcacgttcttcgtcagtagtcaatgcttctttattatctacaccacgaatccagttagatgcacgagttttccagttcgcaattttgtctaatccttttgctattgggccaagatcatcattcattcgtttatcttgataatttgctactttttcttggtcttctttattaagagatgctccagtagaattttggaaattttctaatgctcttccttctacttcatcagcagtatccttcatgccaggaataactcgaagaattgctgcagataatttagccatacctaattgaataagctctcctaaattataaagaacacttccaagcccttcaacaatagctactgtcaatccgccccaatctccagcttcccaaagttgtttaattttatcaatagaatcaaagatgctctgtaataaaggaccccatgttccggtttcgctagagaatttagtaaaatcagtgctaaataaatcccaggcttttgaaaatttatctgaccagtatttaaaatgaaccatcagcaggtctattccaataacaacagccattatcaatgcaaccattttagcagcttcaatagcagcactgacggtatacttaaatagcatgctagatattttatcagtaattgaaatagatttcttaaatccgaaatcaactgtctttgttaatttatctaaagcttgagataattttaagttaaatgcgtctcttttttgcttctcttctggagactcttgttttggttcaattggctgaggagtagggaaaaaatcagcatcaggatcattattaactgcttcaggagccggtaataaaggacccacagattcagctgtatcatcctcgacgactttaacaggaatagcattttcgactactgctaaactcgttccagtttgctgaattccagctgtctgaattttttgttccagtaaacttgttaatttatctaatttgcttccgagcgattcaccgatttctttattaatattgttgccaatttcaacagtttcagcaattaactcagaaccggcagtagtatcacttactgcgctttcaacgttatcaattgctccaactatttcattcgatttttcttcgacagtttgagctattaattcagaggcagcttgagcatcatccaatttcgtagatatgtcattaagtccagataaagtgttggaagcggatttagccgcttcctgtgttggtttattatctgaaataacttttctacgcatcgtttgcatttcttgtggctttttcattcaaataatccaataatattgccaattccggttattggaccattagggccaggaattgctaaagttgtagaaatatcatttgcccattttaaaacaaatgctggcatctcaagaaaattaatttctttaacttcatcattgaccttaagcaagcatttggataacatatcgcttacagttaaaaattgttcaaattttccaggaggtctaaaataaaatgtatttccttggtattgaaattccaatctttggcatacataaacgtcattaatatcataagtataaccatctatttctttacgagatttaatctttccattaaattctagcaaatgaatagaaacgaaatcaacttctgccggtgataaatttggacaaatagaatcaataagaagctttaaattttcatcaggacctttaacatcttttaaaatgttataatgtttaagacccatcttaggaatagaaacttctttattacttatcggaagaactactttcttcagtggtagtatcagttttaaattcatttttaaccttaactgggtcgattgtttccagtttcgttgcattagtgaacatataaagatgagttacggaattattatttgataattcgtgaataacttcatcaacgtaaaattctgttttaaattggtttttactatcattaaaaataattttaacgccgggagtcaagttaaaattaccgacagtagaacatttagcgtaaccatcatactgcgccatagtctgaagacgaatagcttcttcatatccatttctataagtcatttcagaataagcacctgaccttgacactacaatagagttttcaccctttcctgtagtaatcattggtaatgaagaatctaaaaatgaatgagcatagatagtagcatttttcattggatcacgtttatgcgggtttgatttagtcaaccaaacaaaatcatatgctaatggatatttcaattcttggacgaattgacctattaaagttggctcacctacaatcattggatatggttcttgatttatcatcatatcatagtccatcatgttaactcccatgatgtcttgccatacaaatacgaacttatcgcttcctaccgctagagcaacttctcttacatatgacaaatagttttcaaatgtgctagtccatggaatatcaggaacataagcattaatagcatttatcgctggagttaataatgtacgatcttgataaattacgccaagcatttcctttatagattcaccggcatcagggaaaaatggtctaccaaatttaagattttctatagaatgaatagttcctaattcaatagcaatgatgttatcgccttttgaatctactgacacggaaaaatgcttgcatccataaattcgggttttaacattattaatatcgtttgcattagctacagaaatctgaattatttcatttccatccatttttgtatgaatgtttttagaatcataaaattgtagcattccttcatttcgaccataaagagagtcacgcatagttaatgtggtaatagtagcagctaattcaacaaatctattattactccaagcgtcataactctcaaataatttaacgctgagatttggatatccaggacgttgtaacatactcattgatgtttatccttctcaatcagttttaatacgaatccgcgctcggcaggaatcattttcattattgaatttaagctataattactttttacaagcgtgtgattaatttgataaaaagtaaatatctcatctggattaactaatagcttaaacacatctactatatcagtgtattttttaatgtacttatcacaacacgacatgtgcaatgttaaattaataggattcattgcatcgagaattttttctaatgtttctatctcgatggcgtcaacaagttctatttgacttgattcactaatttccttccaatcataccacatttcatcgacttgaacagaatgaatattttcagtaatcatctttgctttattttcataaaactcagaaggaaaatttaatttaattttaacattagctacatcaaaaataggttcctttaattctttttgatatatttcaaatggaactgtcttttcttttttacattttggacatacaaatgtgactggtactttagttttacctattgaacctacaaatacctgcaaaaatataaatggttgccaagtctttggatagtctccaaaataatcagcaattaaatcagcaattacttctttttgttcttgtgatgaccgatgttctatatcgtttcgaactaacaaaaaatctcgataatcttctaccgtaaatggtttaaaacgatgaacaccatcaggtaatttacaacgaataatgtttgccatagatgctccttttattctatttataaatatgataaataaaggagctaaatatgtatgaatacaaatttgacgtgagagttggttctaaaataatcaactgtcgtgcatttactcttaaagaatatctagaacttattactgccaaaaataatggttccgtagaagtaattgttaaaaagctaatcaaagactgcacaaatgcaaaagatttaaaccgccaagaatcagaactattactgattcatttatgggcgcattctcttggagaagttaatcacgaaaactcctggaagtgcacctgtggaactgaaataccaacccatataaatctattacatacacaaatagatgcaccagaagacctctggtatacactgggtgacattaaaattaaattccattaccctaaaatttttgatgataaaaatatagcccacatgatagtatcatgtatagaaacgattcatgctaacggtgaaagcattccagttgaagacttaaatgaaaaagaattggaagatttatattctatcatcacagagtcagatattgtagctataaaagatatgcttttaaagcctactgtttatttggctgttccaattaaatgttcagagtgtggaaaaacccacgctcatgtaataagaggactcaaagaattctttgagctattgtaatggcaaatattaataagctttattctgatattgacccagagatgaaaatggattggaacaaagacgtttcaagatcacttggattaaggtcaattaaaaatagtcttttaggaattattacaacaagaaaaggttcaagaccgtttgaccctgaatttggatgcgatttatctgaccaactttttgagaatatgactcctcttactgctgacacggttgagcgtaatatcgaaagcgcagtaagaaactatgagccacgtattgataaattagcagttaatgtaataccggtttatgatgattatactctgatagtagaaatacgcttttcggtcatcgataatcctgatgatattgagcagataaaactgcaactggcttccagtaatagggtataatgcttcacgttaaaacgtggtataatgaatctaagtccatccaataacaattgaatagagaacaatatgagattagaagatcttcaagaagaattgaagaaagatgtgtttatagattcgactaaattacagtatgaagcagctaataatgtgatgttatatagtaaatggcttaataagcattcaagtattaaaaaggaaatgcttagaattgaagcacagaaaaaagttgctcttaaagctaaattagattactactcgggacgaggagatggtgatgaatttagtatggatcgttacgagaaatcagaaatgaaaacagttctatcagctgataaggatgttttaaaggttgatacctcgttacaatattgggggattttattggatttctgtagcggagctcttgatgctattaaatcacgtggatttgctattaagcatattcaagacatgcgagcatttgaggctggaaaataatgagatatagcattgatgatgcttttaattatgaagaagaatttgaaacggaaattcaattcttaatgaaaaagcataatcttaagcgtcaggatattcgtatcctggccgaccacccatgtggtgaagatgtcctttatattaaaggaaaatttgccggatatcttgatgaatatttttattctaaagatatgggcattgatatgcatatgagagatatggggtaaatatgacagattataaagaagtatttcgactattagcagagatggaagcttgcttaatccgtgtacaagaaatgcggattaaaatgggtttataaatattactatattcaaaaggagacaatcatgtcagataagatttgtgttgtctgtaaaactccaatcgattctgcattggttgttgaaacagacaaaggtcctgtacatcctgggccttgctataattacattaaagaactaccagtttcagaaagttcggaagaacaattaaatgaaacacaacttttgctatagtgtgacctttagtctatagttttggcccttccttttcggttgggccttttttaatttaaaagctttcttctacttcatcatcgtctgaatcttctaattcagctctttttcctgccaaagcatctctcacagagatgtcatcagtatcttttaattcagtttctttgactcttttcttataataagcttcaagttcttctaaaccttctaatgtttgacaagaggcaattttacccataaattcatcaatagaagcttcataaagaaattgtttaaattctagtaacatctttttctccaaagggccgaagcccttataaattaactgttttcattacgtaattgaatttttcatctgcgtagcgctgaatacgatcaatgccgtgttttaaaagatagttcaaatgaacatatttctttttcgtattagcagattttggcttaacaccacaatcatctatgaggtcccagactgttgctattgtcttagaaccatgcttacgtaatacacgaccgattgtttgtaatacaatgatttttgatttaacaccgtgcgctaaaacaacgtgatgcagatttttaactgaaataccagtagaaaatacaccataactagctactataattattcctttaccattttcagctaaggttttcattatattgcgggtttcggtatcaacttcccctgatacgtaataaactttatcgtattcatttttaattaaatcgaaaatagctttaccgtgcgatacatgtttaaacatgacaaaagcgttttcatctttttgcgcaagcttaatagctaatttagcgatccatttatttcttttactaagccccgtaataatttttatttcttcttggtaagtttttccctttaatttagtagtgaactcatcaggataacgaagaaaaatactattaatttttagctcagttacttgtccatcttccattaacttagaagtcgttactggcttaaatatttcgccaaacattccaacatactgcatgatattggctttgccgtcacgtaatgaaccagatagaccaaatttgaacatgcagttatttaaacctgatatgatagatgaaatactttttcctgtagcaagatgacattcatcattcatcatcattccaaactgtgagaaccattcttttggttgttttactacagtttgccatgtaccaacaacgactggtgcatcatttttatatttatcatcttttgatgctccgccaccaattttctttatcattgcgtgactgaataaacgataatcaacaaagtcatcagccatctgagttgtcagagcagttgttggaacaatgataagaattttaccttcataattttccaaataatatcgcgcaagcagagcctgaattaaagatttacctgcagatgttggaagattaagaattctacgacgattaactaatccttcgaacactgcatctttttgataccagtgtggttcaattcttttatttcctgaatagatttctaatttagaaagccattcatcaaaatcttttcttgataattcttctttttcgttaatttgtgggtcaatccaggctttatagccaaagttatcacagaactttttaatttgcccgactaagccgaatggaagaaggcgattataatctaaaagacggattcgtccatcccagtggccatatttgtatttcggattaaaacgatacccatcagcttcaaagctaaagaaatcacggagttcatgaaatgtactttcttcacaatcaattctcacgtgactaaaatcataaaaatgtactttaatgtcagtcatgtctaaataccatgtaataaatatatctatatttatactgaggaaatattatgatagataaagattatattgcagagctgaaggctcttgatgataataaagaagctaaagctaaattagctgaatacgctgaacagtttggtataaaggtcaaaaagaataaatcttttgataatatcgttgttgatattgaagaagccctccagaagctcgctagtgaacctatgccagagactgatgggttatctattaaagacttaattgatgctgctgatgctgcagagggattaaaatatgacgatgaagaagttaatccagaagcagcacttctgattgattctccagttaaatctgatattaaaattgaagtggtagaaacggataaaattcctgaaaatactgacgttttgattgaagacactccttttgttgaagaaaagtttgaacaagctgtagctgagattattgaatctgaaaagccgtctgtatttactcttccggaaaactttagcccgaatcttcaattattggggaaaaatccaggattctgtacggttccttggtggatttatcaatggatcgctgaaactcctgattggaaatctcatccaactagttttgaacacgcgtcagcacaccaaactttgtttagcttaatttattacattaatcgcgacggatcagttttaattcgtgaaacacgcaattcttctttcgtaacattaaaataaggataacttatggcttttacagttgatataactcctaaaacaccgtcaggagttattgatgaaaccaagcagtttactgctgcacctagtggtgaaactggaggtggaactattacttatgcttggagtgtagataatgctccacaagatgggtctgaagcaacttttgattatgtattaaaaggtcctgctggtcaaaagactattaaagttgttgcaacaaatacagttccagaatctgaagctgaaacagcagaagctactacaactatcacagtcaaaaacaaaactcagacgactaccttagccgtaactcccaatagtcctccagcaggagtaattggaacagctgttaaatttaccgctgctttagcttcgcagccatcaggtgcatctgctacgtatcaatggtacgttgatggttctcctgtagacgaagcgacttctgctacattcaattacactcctgccacaagcggagttaaaaaaattaagtgtgtagctcaagtaactgcgacagattatgatacaaagaccgttacttctgacgaagtgtcgctgactgttaacaagaaaacccaagcgactactttagttgtaactcctaatagtccttcagctggagtaattggaaccccagttcaatttactgctgccttagcttctcaacctgatggagcgtctgctgcatataagtggtatgtagatgattctcaaattggtggagaaactaactctacatttaactatactccaactacaagtggagtaaaaagaattaaatgtgtagcgcaagtaactgctgaaaattacaatgaaaaggaagttacttccaatgaagtatcattaacggttaataagaagacaatgaatccacaggttacattgactcctccttctattaacgttcagcaagatgcttcggctacatttactgctaatgtcactgatgctccagaagaagcgcaaattacttattcatggaagaaagattcttctcctgtagaagggtcaactaacgtatatactgtcgatacctcatctgttggaagtcaaactattgaagttactgctgtcgttacagcaactgattatgatagcaaaacagttaaaacaacaggtcaagttcaggtaactgataaagttgctccagaaccagaaggtgaattgccttatgttcatcctcttccgcaccgtacttcagcttatatctggtgcggttggtgggttatggatgaaatccaaaaaatgactgaagaaggtaaagattggaaaactgacgacccagatagtaagtactacttgcatcgttatacgcttcagaagatgatgaaagattatccagaagttgatgtccaagaatcgcgtaatggatacatcattcacaaaacagctttagaaactggtatcatctatacctatccataatcgtaaggggcttcggcccctttcttcattttgaaagcacacaaaacacaatcagaaaatgatgtatataatggcaccaactcgataacatgagattgattatgagaactgaagttgtggtgtttacacttcatgagtctggaaagtcattcattgaaattgctcgtgaattaaacttacaggcgaaagaagtggctgtattatgggctcgagctatgactgcaaagaataaatttgagactcgagaaaaagttgtctatagaaaaagacacattaataaaaaggtgaaaaatggaacagtatgatctttatgaaaatgaatcttttgctaatcaattacgtgaaaaagcattaaaaagtaaacagtttaagctagagtgttttattaaagatttttcagaacttgctaataaagcagctgaacaaggtaaaacatattttagttattattgtactgctcgcgataaattgattactgaagaaattggtgattggttgagaaaagaaggattcagctttaaagttaatagtgatcagcgtgatggtgattggttagaaattacattttgaggattaattatgtttaaaaagtatagcagtcttgaaaatcattataactctaaatttattgaaaaactttatagcttgggattgactggtggggagtgggtagctcgtgaaaagattcatggtacaaatttctcattgattattgagcgtgataaagtaacttgcgctaaacgtactggccctattcttccagcagaagatttttatggctatgaaatagtactcaaaaagtatgataaagctattaaaggcgttcaagaagtcatggacagtatttcaacttcagtgccggtgacatatcaagtctttggtgaattcgctggtggtggaattcagaaaggtgttgactacggtgaaaaagatttctatgtattcgatatcatcataaacacggaaagcggtgatacttattacatgagtgattacgaaatgcaagatttctgtaatacgtttggttttaaaatggcaccaatgcttggacgtggaacatttgatgctcttatcatgattccaaacgaccttgattccgtcttagccgcttataacgttacggcatcggaagaccttgtcgaagcaaataattgtgtatttgatgctaatgttattggtgataatactgcagaaggttatgttctgaagccttgtttccctaaatggctgccaaacggtgctcgtgtagcgattaaatgcaagaactctaaatttagtgaaaagaaaaagtctgataagcctattaaagctaaagttgagctatcagaagctgataacaaattggttggaattttagcttgttacgttacactaaaccgcgtaaataacgttatttctaaaattggcgaaattggtccaaaggattttggaaaggtgatggggctaactgttcaagatattttggaagaaacttctcgtgaaggtattactctaactcaagcagataatccttctttgattaaaaaggaattagttaaaatggtacaagatgtacttcgtccagcttggattgagttggtgagctaaataaaaagggacctttcggtccctttgttttattcatcaataataatttttggtaatttaacacctaataaaacagacaaatctgaacgacccgccattttatccatgtctccaccgtcaattattcttgcttctttttcatcttttgctacagtataaggatttgctgataaagcatatctaactaataaaccgatagacggttgtaagctttctggatcaacaacaactttaaatgcacctacatgttcagggtcatctaagtcaagaccttctgtatacggagcatagaaaattgatccaacaatttctttttcaccgatattttctactacgccaacgattacataatctaatgggctgttagtatcgcaataaagcggtaaaccattagctaagaacccgtaggcattttgtgaaagatatttgtcatcttctggtttatgttttaaccaacctgatgcagcaagaatcgcagcggcacgagcagaagcaacacagaacgttgctgtataagttgattctttttggatatgcgaaaccatttcacataccattcggtataatgaacgaccagcttcaggagcagatgcataactcaaatcgatgaatccagtatcagtaattcctgtaactttatagcgttttgacactgtaatcaaagactgcagaatatctttattgatttcatccgccatttcagttgcaagcaaatcttccaagaaattaggagcatcgaatccgtttgcttctaaatcttgtgctaattcaactgtgatgccagttttaagtttacgagatttaactgcggtttgccatttattaatctggaatctagcatccgcaatttcgctatcagagttttcaaatttgcttgttgacgctgcgtcagaaaatagacgaacctttaaaagaacaattgcaatctgaagagctaattctaaatcactttcttcaatatcagcaaatggtgtatcttctaatactttataaacgatattattatatttgaataaatcgcctttattgagagttaatttagactcttctgttaattctgtgattttttctcggtctacatatccagcttcgccagcataagtagcaccagttttaaatgtaaattcgttgtctgggttaaggtatttgataccataaaaagcagcaacaggttgattagttctttgcgttgctacaatgtcagaatatattaatttagtggtagcgcgagtcaaagcaacgagatttgggcgaccgattgagttgctattcgttgtggttgattcgcgcagaagttcgttgattttagccattgcgctttcctttcagtttatatgatttatttataccataaaaacaactaaagggacccgaaggtcccttaaatcgtcaaagattagatacctttaacatatacacggcggaagtaagcgttcttaccaaggctgttcaggatagaaggcataccgctctggatgcgagaagccggagcctgagcagcggattctgcaaatgggttgataccgataccgtaacgagttttgaatcccattactggttggaagttcttcggatcagaaccacgcaatggagtcagagctacatatggagcgtagtaaataccagcatccatttcgttcggacctttataacctacagtgaaataatcctgtttagcatactggtcgatatatacacggtatttaccacccagaacaccagcaaatactgacttggtagtatcagtgttaaagccggtagccagaccctgtgcagcataagaaatgccggtatcaactgaagccagaacgttaactacgttacgggaagcgataatgaagttaccttcaccacgaccggtctgacgagcaatttcaactgcttctttgtcaatctggaacaacagagctttaaaggattcacccgcccagcgagcaccacgaatatcgattgggtcctggaagtcgaatacaccagctttagaacccggagtcagggtcataccagatttaccaacctgagcggagtagttaatccaatcaacaacttcgcggttgatttccagcataatttcggtagccagaataccagacagttcagcatcagcatccataccatgaacagcgcggaggtcttgtgctaattcaatagagtaagcagctttcagctgacgagatttagcttcgataacttgcttatcgatacggaagcccatttcattccatgggttatcggtagaaccgttaaaaccttcctggagttcagcgatagaagtagccataccttcagcgatttctaccagtacacctgcttccatttgtttcttaatttcagcatctaatttagctgcgtcagatgcgcctgaatcaatttgtttagcttctgtagcttgcagatacacagtaccagtgtcctggaagaagtgggtgtaaatagttccaacttcaagagtatcacttgctttcaaagcagcgaatttcttagcagcaccctgaccagagaacattgcatctggaccatacattgggtggaatgcttctttagcgccggaagcgattgggtctttaccatataccgcacgaagtgcgaatacctgaccagtcgggctgttcatcggctgaacaccgcaaatatcgaaagcaatcaggttaggaatagcacgacgtaccatacccataacagctgggccaatctgagttactgcgccagaagtctgacctgcagcgatgttggtagcattgtaaccgtggtcaccaccgatttcagcttctgttaagaaagaaccgaatgcctgagcaattttttcgtctttatattccggagctgtctggaaatctttttcctggttttcaaagattttagcgataatcgcttgtttgctattagcaatttccggtaaaccttcaccttccagtaatggcttccatttgttcaaaagttcagctttagttttgatagtcatttgtgttaacctttaaaattagaaacgagatgcgactttcgcatatacacttacaatatcttctgcaccttgcgcagatttatcttctacagcttcagtgacgaaattcagtccggctgcttcagtatcaggagtatttatactctcagtaatagtgctttcatctttattagatttcttcaccatttctacgatggcactcaatttgcttgagaatgcatctgaataatcaataccttcgaccagagcagagactttttctttttgagactcagtcagatctttagtactttcgctcaatgccacttcacgctgcacataattgatatatgcgtcacgcttattgagttcttcgaacagacgagctgattcttctttgtgttcttgcagttcttcttccatttcagctacaacatcaactgattcttctggaacaacaacgttgtgttcaacgaagagctctttcaatccaccaagcatggattcaaacagttcggctttgatgcctttatctactgctaatttattttcagtgagccattcttttgcaagatggtcaaggaatttagaagcttgctcagcgattttcttctcagctttttcttctgcttcttctttatttttttctacttcttcttctgctttttcagcaattttagcgatatgagattcagctaatttaacggcgtgctgcttgacggtagcttcgaatacagtgccgaaagtttcttttgcttccggagaaatattaactgattcgaaaatactatcaagagcaacggaagcatcaattttctgcgcttcggcaatcagttgttctttaagcattttgtagtcctgttgtttagataataatatttataacgcttttttcatggcctctgcgagagccatataggcgtcatcggcacttgtatcggcttccgccgtctgtgattcggtaatttccttaggagttacccatgcatctggagcacttggaccccatactgcatcaacacctacagttaatttgaatccttcgtttacgatacgataacctttatttgtgtcagtcaatgaacctaatccacgagaagaaactcctggaatccatccggcacgaatattagctgctaatttatctccaggaccatggtcaccttcaataacacgagctcgtccgtatacgtcatttcctttccaccacatatcttctataatgatagcggcttgcatcgggtcaacattagcgcgtggaggatgatttaattctccgagagcttgtttagttaaaacttgctcattaatatagtcttttaccgctttttctaatatgcgttttggataaagacgtttatttctattgacgacttccgcttgcataaatattccttcgatgtataaaccaggttttaaacctaagtcttttccatcatgagattcaagcattggtacgccatcaataatttcgccaggttgaccccaagtttcaattagtaattggggttcattcattagcttaatcctaatgctttacggcgtttaagagcttttttacgcttacgctgagcacgagattgacctgctggattggcaatcttcgttttggtagctttacgagcaatttgtctacgttttgctttagacaacccggtagtttgaaatgcattacgttcgcgggttttgcgatctttagtgcgagtaatttcaccacgggcagaaacatgtttaacgatgaattcatttaacggcatattttcattaatagaagctaatgcaaccgctaaatcagtttcatcatcaagcatattctcgataattttatttatatcgtctttatttaaagcagaagacaactcgtcaaagcgaccctgtgcttcaggaataagtgcttcgacattctcgagaactaattcatgagtttcagggattagaatcattattcatcatcctcgtcttcgtcagagtctttgtcttttttgtcgtctttatcatcgctatcttcgtcatcatcttcatcttcatcttcaggttcttcaccttcgattaagaaattacgagcgatagtgattttttcttctttgattaaatcagtcgttcttgcagccatggcttcagcaaataatttacgagcggctacgaggtcgtttgatttaatagcttcaattaaaccttccattaaaaatcctcttgttcttggtcggggtcttggaaacgagcctctttagactcttcttcaatttgcttggcttcttgttctatttcttcatcagtcatctgcaaaatatctttcatagcagttctgtgagaaatatatttaccaataaatggttctgccatggttagcatattaattcttcgttccaaaatttctgcttctttgagctcagcaaagtagctatcccgatgaaattctatcttaatattatttatttcatcattccactcatcttctgtgattatacctttaagcaaaagatttgttttaagtggatctaggaaaacttcttcaaacttatgctgcagctcacgaataaatttagcaaacgttaattcatcacgtgtaatgctagttccagaatcaaacatcacaccgccttgttggtcttgcggaatgcgtgaaagaggaacacgtaatgccatgtaaagagcttgtctaaaccagcgaatatcttccatattgccagtattatcagcaccaggaagagtatcaacttctgtcacagctttaccatcgcggcgctgcaaccaatagtcttcggtcatagacatattatgctgttgattttttattttacctgttgatgcatcatatactacacggtttttcatcgtgttcatgacatgttgcatgtgctcggcagctttacgagcaggcatattacctgtgtctacataccaaacacgacgatcaggagcacgagtaatacgataaatgactacagcatcttctagtaattttaattggttagcaggtttaacagcacgatgtaaatacccgatgatatttttaccgcaacaatcgactaatccagaatgggcataaactacggcagctttaggaatttttatttttgtgccagcttcatacattctaccatcacatgcatatgactcatgggcagtatcatatataaaatattctttgtaacctttaactatttttgtgccagcttcagtttcggttataatttcacgaacatactgaacttggcgagggtctaatctacgtaattcttttatgccttcttttggacgttttggatcaatgattttatggaagaaaattcttgaatcgacataccaacgtctaaaatgatcagaaccttttcgttgaaacgatagatgatttaatacatcactaaattcatctaacatcatatttttaatttttgggctaaatttagatttatccaaatttaacgctacgacttcagtatcatcttcatagacgatagcgtctgaaacgatttctgaaactgcattatctacttcatagttattcatgagattacgatatgtatcaataagctcacgagtagttttcattcctggttcatatgaaccaaaaattgtttggaatgcagcattataaggagaagcagcttcattcgagcttacttcaaattctcttgctccatcatcaagctttggggctgtaatggaaacaagatcttctttttcttggtctttaaaatttcgttcgtccattttagcccatggagcaaacaaacttaatacattaaatttcattgtattctccaaatgggaattatagttatatttataatggacttctctgctttaagcaggatggggatttctccccattcattttattcccaataatcgagagcaagagttacttcaaatgtttggatttcattgtttgaatcccaatctaattgaagttcacccacgttagtaggccatagacctttaatttcaatttcttttgttactgttttagcatcacgagcatattgacgaacaatagcgctctttttatactctgcaggttttccaccagtaatttcgtttccttgaccagcagcaatgctttgccaatcaacaaacttctgacgagcatcatgagcttcatcattcatcactgtaacagtccaatcatcgaatgtacggtctcctgctacattaattttacggttcataaatccgactggaattttttctacaataccagctggtaaagcagtagctttacattggaacgtaaaattttggccaagataagaaatctctacttggaataagttaggtcgcgcaaaatcaccagattcaaacgctcgtgttacatcatctacaaacatattagcctctgtagtatttatatccctatgtttaacctagggcatatagaattaaagaattaaggatatagtgtatttatatggcctgtcgaaacaggcctttagaatgcaccgtattaaccagcaagaccagttaactcatcgaaatctgcaccagtagcagtcgcaacgaagttaagctggatataatttatactgcgcgccggttggatgtagaatgttgcaacaaactcatttctatcaattactgacggagtgttatttgttgtatcgcaaactacacgatattcataaattccgccaagagctttaattccttgcaagtactgagcagtttctgtacggaatgaagaacgagtaaacgcgttgtttaattcgaacaaacgatattttgaactacgtccgatattcgttttcaacatattaaacagacgacgaacgttaatacgatcaaatggagaaggaacagaagtagctgttttatcaccatacaatacgtaaccatcaccacctgtaccagttactgggttgatagcttcttggtataaacggtcgcgctgagcctggcgagtttcaatagcaagtttaataacgttaagaatctggccacgattataaccagctggagacatccaagtctgagaaacgttatcggttctcgcgcataaaccagcaatatcagctgctaatggaacccaacgattcacatcattatatttgtcatactgatatttgtagttgccatcgattgctgcataggttgaactgatattaaagttattatcagtgtatgaacctgctgcagttctccaattgactaagttatcaacagcacgggttacaggaattccaactacagtttcacgcggtggtgagcacaatactaagcaatcctggcgagcatcaccaattgaaacgacgtgtttttggacagtagatgctgtttctaaagattcaccagcacaagaacccgcaataaacagttgaacgtcaacagattcacggtcagcaaagaagtcccaagcttccatcaaatctcctgctgttacttcagcatttgatgataatccaccagacagagttaaaattccagagaagccttctggccagttttgtgcagttgcaaaaatatattctgaaccaccttttgcgaaaaagtcatcgatatagatgttactatcgtaaatatctttttcaccacgcttagttgaaagaacaacgctttgaacaatagcatcatttcgacgaactataatagcgtactgtgagtcagtctgcggcccatatccaaatactgccttggcagtagatgcacgagtaccaccacctggataaattggcagtaatgcagaagcgcctttcgcatagtcagctttagatacgatttcaatttcaattttatcgcctaattcgcctggatagagagctactactcctggaattccatatttttcaagatttgcctgaaaatcaactgctgtcatagcagtttcagcgctctcgatttcagctaataaaataccggaatcagtaataatttttccaagagttattactgcagctaaaccggaagaagatgaagaaatttctgcagtccagttagaacctaatgttgggtattcgccaacttctttagctttagcaataatttttgcagtaggaatattaattttcttaatttttccatctgcgtctacttcggtaattttaccttcagtttcaatatcatctgaaacatatttgaccgtgattttatctccaactgcatagttactacctggggtagaaattgtgtattcaatattaccagcaatcggagatgagtttttagcggtatctctatcaacagcacgaactactcgtaagtcatttccgtactgcaagaaattcatcgcagacataaaatagtcagcagtttcagcggttggttgaccaaaagtattaactaaatctacttcatttgtaacctgtttaatctgaaaagcaggaccccactggaatttaccggccaaagctgctgtaccagtagagttattaaccacggtgctttgaaccgtagtttctttgagctcaatgcccggagataataaagtcatttttaatcctctttaatatgctttaatatatttataccattgacataccatgagatactggaacatactcagcagaatgaaccgaatcaacaaatataactggagcgtattcatcacccatatcttgaagctcttttgaaaacacttcagatgctaatcgcatgtcatctttgtcagcataatcaataaattttgattgtgttgataaccatccaaaaattactaaagacattactaaatcgtcgtgataaccttcttcagccgcccaagacacgcctttttcactaaacgttctaaactcttgaatagttgcacggtgatgaataataagcttatctttttcaataaggtcttttaatgtagagcatccaactgctttcgttcgtttagtttgcttcattcctaaatcagtatatgaatcgcaaataacgccttcgtattctaaatccatataaagcgatttagcaactgacacaccagtactatttaattcaatataaactgggcattcattatattctactaaataacgcataacgatgtcaggtagaattaaatgagaaatagtgtttgagtgtaaaacaccaacttgttcccacacatcatcggtaacatcaataatatgtaaagcgtggtaatcttgcccacgaccttctgagcagtctagagttgcaatatattttctatctggttcagggcttttaaatcgatgaaaaccgtgatcatctggagttacttcaatgaaatccataacagctaatttcattcctgaaattaatgtaccagaagtcccttcaaatgctgcggtgtgttcttgacgaaattgagctaaagtagaaccattaatggtttgtatgctccattgccatccatcgtcaaaaatatcttcatcgttataaagacgttctttaactgaattccaaatagcagtgtatggttcaaatccagatttaccttcaacggctgcagtccaaatatcataaaaatgatttaatccattaggagtcgtagtaataataattttcgaacgacgaccagatgaaattactggctgaatagcaagccaggaatcatggaaatttggaataaacgcgcattcgtcaatataaatcattgcgaacgagttaccacgaactgcgtcaggagaggaagcataggctcctattgaggaaccattatcaagttcaatagaccctttattccattcaacaattcctggctgtaaaaagtcaggaagcagttcaattgcttgcttagtacgatctaaaacttccgcagacattgagcctttgtgcgcaagaatacctacagctttatccttgttaaaacatacaaagtgtgcaaggaaaatagctactacagttgttttaccaagctgacgtgatagattacaaacagtcatacgtttagatgacattattttgagcatatcacgttgatagtcacgtaattgaacctttatgacaccatagtcaatatgagtaatagcgcagtatgtttctgcaaaataaacaatatcgtctcggcattttttccattcctcgaccatttcacgagtccattgtgttttaatattagctcgtttcaagttaggaagacccatataccgagatcttttattattcttatctttataagtttgaaataattcaggcttatcagagttgtttggaatttttactattttatgcagacgaaggtaatcactgaatttttcaggataccattttccatcccactgagattttatccaatgaattccgtcttcatcttttctttccgctaaacttgggtgttttattaaaatttttccagcttcatttaacggatggaaatcatttaatgcattaatcggttgttccatttatcaccttctcacgagcttcttgagcttcgtaagcatcaccaatttcgtccattaattctgttggtgaacccatgaatactgtcgcattctgaatattcatttgacctgtaggaacagcgcctttggtgccaacctgctcagatgtaatatctttcatatctttatgaagcttcagtatttctctgttcgtcgtagtcatttgccccataagagttgcaaatacttccatgtgacgaggagaatcagcattctttgctgtctcaagaaaaatcttggccgcgtccattagcatttgttgttgaaaatgcatatttcgacgaactactccataatcatcttctaaatctggagtacggttttgtggattgcttttaacttctactaattgcagaggttcatatactttaatttcctccccgtcaattccggggaggtcagaaatatctaaaagtttgtttatatcaagaccttccataataacctctatgttcttgggccaggaggttctggcggtgttggtctatttacattgttagtgaaagtttgttttactgttccatcccagtcttctgggtcaatatctcgaggaacaacttcattatcaacagattcaaaaacgccttcgccatcaggcaaatctctggtattagcatgaaaatctgtataagtagtacgaattaatccttctgcatcatctactggaggatacatccatccatttacttcaaatgttaatgaccattcaattctacgacgagataaattgtccccatctatagcttcgtctatagcagcagacatcagtacaattttaatatcccttttaaatggaatatcatttccaaactgctcgtacatagttgtattaaaatgaggttgaaaatatggaagaatctgttcaactatttgaaacatatcatcttcgtagcgagtaaagatactcaattcataaatcattttaataggagatggattatactgcgatactacagaagttgtacctttttgcagtaaattctgatttaaaatgtttgttttaaatggagcgttatagctaaaatcaactaaatgcaaatttatacgaggtagaatagtttcaactttagctacatcttcttgtgaatttattgatgtccatttattcaatttcatcataaagtgttcctttgatgcatacgtaataggaacacgtataaacttatcaccagattctaactgacgcttgatttggatatttgaaaacaaatcgcccatcaaggtagcatatcgtctaaaagacgaattataaaaataaccaaacatgatttctcctaatctgggctttaattagtttataatatttattaatccatgaaatcattatcaaatgggctagattcgaaagattttcctctgttattgacaacaacataaggttcaacatattctttagcttcagaattaatttgatctacttcagcatattgatcaatattaatatcatgaataccgttaagattgcgaacaggatttagttctaattcactaaattctggaatgttaattccttcgtttttctgtagaactggattaatttcttctccagaataaatgaatttacctgctgtaattttacgaatagcgttttggcctaattgataaaatgggtcatatggttcaacccagttgatttcaaataagctgttatccataggaaaatatatcaaatcaccttccttgggttcttttccatttacttgatgtttaaacaggtttggattaatggacaaagttacttcatcctgtacttgcataccaaagttaccaaagaacgatttagctccttcatatccttcaaatgaatttaaatatgcagcaaatttccatgctttagtaaatttattttttaggtcttcgccgaataccaaatcaggggaaacatactctcttggaacataatagcattctacacctcgcatttgaatgctttcagctactaatacatcagctagtatttggctgttttgatagtggttaaaatttacataaggatttagtatttctgtttcattggtctgagaataacctgtgcggttttccaatttagcaaagagatttttatcgtaagtagccatattaacctacaaaaattccaaatggagggtcaagtaagtataattcttcgcgcagtgcttctttttctaaacgagcttcttctattaagcgttgaccatcaattgtaacgccgccgggaagcatcattccttggtgacgtgctaaaatttggccatttagttctttagctaaagctgttgcatagtctttcacccaacgattattataagcaccttgcttaacatctggatcttgaccaacaacacggcctactaaattgcggtctggattattatatcgttcagataatgaccagctatcttgtggaccgactgttccatatcctactgtatttccaaccattttgtttgtatcaatgtatgatttagtccagctttctacgataatcaaatcatatttttggaagtttcccatgactttgagttgttcatttgctgaattaaaccaaaagtctggaataggcgaaagcatatcttgcatcattcccatgtaactagtaagctgggtaaaatatcctaaatcagctccaaaggcatttggtccataaaatctattacaagaagttcccatgccaccattaataccagccattcctaaaagaaagtcagtaaaccatggatatgtagcgtttccgtccattgacgttattgatccaatattcgtgcgcaaaatgcgagttactgcaaatacgttagaacctcttaaatcaaatactccagtcttatacttttcttcatcgtctcctacataaaatacatgaaaacctttgttaagtccatcaaaatggtattcaccgtataattctagggcacgctggatgcaatcataaatttgatcgggtgttaactcaacattaataattggagcccctaaacgtcttagaatgacatctttgagttcctttggattctgagaattatatcctgacatttaaaatcctttagggccttgcggccccatgttatgctggtgataaaaaggtagaaagcaatacccattcgccgtctttacgaacgtaagcttgaccatcttttggagcttcaggaatataaccagcttcttgtaaagtctggacatccctctgcaatgaggacacattacctttaatactacgaatctcttgattaattgaagaaatgttagtctcattagcttttattgaattagtcaatccgcgttcttcgacagttgaaccattcggatttgttccatttactaatttatttagtgttacaacttgccctttaattccggaattgttattaccaatttcaacctgtaagttttgaacatcattgttaagtctattaacagaggtttcaacagttgaaactctatttaaaagagaccccggaggagaaggttgacctccgctagaatcagttccaactatctgatttagccatgaaacgttagctcttaaaccggacgaggtgttttcacctacaattccatttaaagattcaatagaagtggtattatctttaatttgacctttaatactagaaggaatatcatctgaaccaatcgatgtttcaataacagttaatcgctgtttaacgccgccacttttatttagttcatcatttaaagctgaaatttcttgactattgacattgacatctgtgattattgaagtatttcctggatatccaatagaagttttgatagatttaatttcggaactaagattaatctgcgattgatctaaattatgcacacgtgaatatatactttcactaaatgaagatggtctttgtccaagttcttgtctaagatttccaacttcaattgtcagtgaacctacatcagattcaataaatttttcttccaaatcacttagacgtattccttgggaagttatagcatctgtattattaatgatacggtgcttcatccccgtgctgctatttcctacaacaggaagaccattaatatcttgcccagcatattgtcctaattcccttttaatccataacaaatcatttctaatcgttctataaactgaatttgcttgcgaattaaatggaccaatatcagaaagaatattatcaaccgtagtatttgtaccatttaatatatcagtatgttcactcgtaagcgtttttaaatttgatatgtcatttttattaacgctgatttgtgatagcgcttctatatctcctgatacatctaaaatgccttgaattgttttaatatcttcctgtgatgtttcaacggcagttttaagaattcctatattttcatcaagaacctcaacatttttaaatacggacacagttggtctattcattgatccatcgtttccatacttagtactagctcctagtatttcttcaccatttttaatccatgaaatacgctcctgaccttcgccaggaacgctatcaacaaaaggtaaatcttttaattcaatcatttgtaatccttagtaatgaattttaataatatagtttaatgatatattccatggtctagtttcataaccaattaatccttcacgattaagtgtaccaagggcatctctcggtccacctaattcaaatccatcattagtgaagtatgaagcattatcccagtcggcatattttctagttccaagatatccttgataaacggacgcgccaaatggagcttcatgtctttggtattctccccaaccgccagcatgtttatggtatgacatctgttgtgcttgaacaccgccaacatgcattccgtcacatcctacaccaagtctatccttcccgtaaccatcttgccctcgttgatttaaaatatgaccaccagtaccagctcctctgacgaaaagacctcgcatatcaggaatgccaggattattccaatcgccaccaaatctcgttccaactgtgtttctataatcaggatattgatctcctgacacagttccgccatggcacataatccaaccaggaggagctgaatctccagcaaacatcataatagttccaactggaaccctttcggacgaatatctttcagtggcaacaggttgatcgccatttttccacattccgccgcgtgaccaaattccatttgctgttaaatgatctaaatttaaagaaccattaatagtttgtccaccgcgagtattaattacatctgcgttccatgctaatgctccagagctatcaccttcaggggcaataccggcttgcgtcgttaattttactaccccacgcatagaaccggtagctcctcgaccatttaatgtttcacctgttacagcaacatctcccaaattactgttaatttccgattgcgttcctaaacgtataacacccttatattcttgtgttgcaacagaattcataaaggtatacggggaaattgcatatccttcgcgaagagttccttgacgagtttgagcgacagttgataactgaactacgcctctaacagattctgatgctgtgtcttctgaaggagctatttgcgaaattaattttattgcaagtttttgtgtttttagcggagtcattgccgtagtatcatctactccagctaaagctgcaggtgtagatgatattttaataaccccgtttgatgactcagacgaatatctattttggaacacatcgtcagtatggtatttcagtttttgcggcgttattgatgaattattatctgagccttctaatgtttcttcatttgttgaatagcgcgttaaaccatacttagtttcagtagcatttggatacaataatcttgttgctaatgtagctggtgtaacgattttagaattattagttccatctaaaacctcttgctctgacgcaatcattgctattcctttaacttccatagttgcatcaggaataccatttacgccgatattacttatttttgataatgcagactgcactgtcgtgacagtgtcaggaaaattcgatcctgtaggatcgaatttaacgtattttgattcatttgatacgtgctgatatgtattgttactcatgctattctctcaaaataataaaataccgtaggttgtgtgtgactatctagcgtgacagtttgcgcacctaataattgccaagttccgtatccaggtttagaaggagaaattatttcttgtttgaaagtaattcctttatcagagtattgttctaagatatgttcttggttatctaaatacttaacttgtattttatttcctatggtaggatggtcttcaaatgaatcaattgcaataaattttgcaaccatttcctgtagagctgttctaacggcactggtaaattcgattgaagtcattccattagtagctttaactggaataccaaacatgttaacaataactgtgtcacctggtttggctgaagtatcattgacagttccttcaaatgaccagtagtctgtttgttgcactccagtaggagatacgccgcttatattaactaatacagctccaacctgttgctctgccatatttctaacatcattgatagcagattctacatttgggtcataaaaaccttttgctatttgtgaaattgttacagcacctactggctgattattcataaccgaaatatcatttttcttagttctaaaaccaagaaaatctgctaagcgggaaataactcccgctttattattaagtaaactcattatgcaatccttatccaacggtatacagtaatagacggttgaatattactaatattagcaggaggagtatgtgaagagtttgttgttgcgtagtcttcacgatattttgtatatataggaccagtttcatctgggtcatattggcaacctccaataataaccgatccattttcgtcttcaattaaaactctttcgtcggttttagttgcaggaagatttgcgttttcaagcgttactgatgttgttccaactgttccaccggcagtatgtgaaggatttccgctagaatctaaatcattattgtttaaagcaaagtttggatccgtaacatcatcattccatcctactaaaacttgtcctttaccaaataatttccatgaaccaaaccccatataagtcaccggattatttgggtttatagcattttcataaattgaaccaatgggataaatgctatcgaaaatagatgatattgaattatatgttctagtataagaatcaactttttcaacatttggccaaccgattttatcaaaatctgttaaggctacatcaccagtaacttcggtagatgatcctttgctaacataacggtcatcagtcaattcaatgatgtcatctttttctaaaagagttcctaaatcattattgaaccacgttataacgattatatcgcctgattcaaattttctatcaaataaaagactgatcggttttccatcttcatattctatagaataatctgtgtttgattctttccattcaccaccaagagatatacatccttcttgtgtatctgaattagcgccttcacataaaaacagaggatatccggctgttccagcttgttgttgtaaaattccattaaaacgaacttcaagagaattaggattaataggttctcctggaattaatccaaatgcggagaatggaattgatttcattgtcgataaatcagtaacgtaaatacttccttccaaagaagtttttgatgttaattttgaatctaatacttttatttgacgtcttgtgtatgaacttctccactgcgatacaccgtccataaatgtttcaatttgaacagtgtcaccaatattacaaggctgtcttaatctaatattaaatccatcaagaggaaccaattctccttcattttcccctggagagccaaagtcactgttttcgctaaaaacatcgccgtaatacaattcattaccgcggtgttttactctgatgttattaacattataactagttccacggaaaatatctaaaaagtctgtttgtccttgaacttcgactaaaaattctttacgagctacattgctaatgtctgaactaataattttgtcaatttgtttatttttgacatattcccaacgacccggagcgcaataaactagctctaaatcactaaattgaacgttaatttcaaccgatgacgatgaacctttgatagtatcaccagaagcggctactaaagtaactggattaacattccatgttgcaaatacatccctagctctaattactttattataatcattaacagttcctttaggaagttgcagtgttactcttccagacgaagtattaatagcgtatgattttccccattcagcttttaatgtttgcccagatgaagcattataagttttccaggcaccggctgaatatggaacattaccatcaccaagttcataataaagctcatcaaagttttcatttatttttataccacctttacgcaggtagtcaccggtaccatcatctacaacattaccgatattaatattttgtttcattattgagccaccccgattttctgcgtagcgataactttaactgctgctctcatgccgacagttgaagaacttatagtcgccgttacgtaattttctttaatactaaatgcaatattagcgatttcgtcttcttctgtttcatttccaactcgcatgacagcatactcagaagaaataacttctgaattaacagtatccacaagaatatttatttctgctgttttaatttttcttccatctactgattggcacgtaactagcaatttagccatattgtattcagtgcgatgaaatagtggaatgtcaactgatccagatgtagaaatattccatgtaccttcagcaggtgattccttttgaccaaacatactttcaatagaataattccaaaccgacgtagaattatcagatgaaatacaacgtaaggtaactttactatatggactagttactactaaattacctgaaacgcctttaattgaatcaatagcttgaatcgttaaaggattagtaactgatattgatccatttgagttaataaattcaacgcagtcgccaagttcgcctctttcaatgataactttaacacctacagtagaggtatcaatatcatgcctagtgccaacttttactggagttgcatactctgcaatagagtgtttttgataatatccagtagcatggataatttgaccatctgctccagtgccatttgctactgccattttacgttgatcgccaaacgcgttataaattgcgttaaaatcactattaattttattaccaccgtcgaataagatatcaccagtagaagcgttaccaatttcgccggtatcaatcaatttctttggttcttgaatgaacatagcggtttccttatgagtttatagtatttataaagaaaaagggagcccatgggctcccttaatttaaaatgtaaacagaatattgatttcttctgtctgatccattgccataataataggtggcctattttccatataaatcatttcgcccgaatgcctcattaaatcttccggatcataataatccttttcagctttaacatttgggtcatttggatgagctttagcttcaagaggattcgtaattattgatatttgtctaaatcctttatttcccggcaatgcagcttcaggaaaataaactgaatctaaatatgctttaaaacggatagtatttgccttaacacggtaaattaatccaaaatcatcttgttgccaagtgagattatcttcatacccccatctagtcgggtcttcttttaattcctcaggccaaggaaccacgatgtattcattcgtacatctatttatagatacatcaggcggaatctcaaataaatattcccaaacatatccatccccaggttcaattgttccttcagcatctcctcgaccttcaggaggagtcattgacctagcagaaggagtccattttccacctaacttaaggcattcatccttattagttaaagatgctattgagcacattccagtatcaggaacatctaaacagcgatacactaaccatcccgcgcctgattcagtagcgttgtaaggagctgagttacacactacaatatcgttaattctaaatgtgtatggatccggatatctagtatctccccaatctcgacgaggaataactgcatcaagcattgatggaagaacttttactgttcccatcatatgagtccacatgtcagttacacctaatacagaatcggtaggataaggtggggcaaagcccacctcattttcatttgatgaccacggttctgatcttccaaatgtgataaagatagtgtttttatccggaccacttccaattgaattataaaaattcaacattttttctgttctaaattttgaagtaactatcgcacgatagataacacttgaatcattcatctattttaacctgtgttggattttcagggtctcttggatttcctatattgtcttttagacgtttattaactaaatctctaaattgcgcaaatgttgttccagatgcatcaaataaaggactcattaatttacgtcgttcagacggcaattgaccttgaaatattgaattgttattttcagcattatagtcatcaggaagaggatattctacaccagccattgggccaggctcataaatcgcttcgcctgttaccgaatcatgctcaatttcaccagttggagtcaatttagctactctatcagcatatttagtaggcaatccagaatcccatttatagtttttgtatttattaattatagtctctgtgtgtttaagagttaaaccaacattaataaacatcgttaaaagtgtaattgctataaatccaaatcctactggatgaacaaaacgaataacgtcagatttccaacgagaagaaggtaaattggatttaattttcattacataatatgatcgacttctattaatatagtctatattattttgaagcaaatcctttcctttaaccccacgaataatttcgccttcaaaactaggaagtctttctgctttaacttcttgtcctgctattaaccgccccaaaagattatgaatagttacggtccattgtaatttaccattagaatagcttctttctatataagtaacattacatcttcctgtagctgtataaatcgtttgtcctactaaatcttctgtaagagaatcagattgaatgattacatcatattcagtacccgctcccgattcaatttcaatttcaacttcttcattataaagaactttaaaaagaaatttatatgatgcttcaattcctttagtagaataaaaatcatagctacgtgattcaaaaaatcttgcaacagcatcgcgtttgtcagcatttaaataaatgtttcttttatatatttctgaccataaatattcccacgcatgctcttctcgcggatattggttacgaattaaatttcgtaaattgttatactgagttccatatccgtcagaaagatattgaatatatgcttcacaaaatgcctcaaaattagaatcttgtaacaaataagaatcaggcatcattgtgccaatcaatggtcttaaatcagggtcagctaatccacgttcttcttctggtgaccaaggagtttcgcgctcttggttttgtaaaaatgcttttaaaaatacttcagttggcttccaaataatttctactgtatctctcacacgatagttaaaatcataataagaaattatttcgccagaagatttgtagaaaagcatcccagatgcgtattttttaaatccagtgaatttaacatttggcattactatagtgcaatcgccatcatcccagtattcatgcactaatctatctggtgacgtttctggaatattttcaataactttggtgtatttcaaatctgcataaactacaacagctttatcagagttgtttatccaacagcgcgtgttagattttttagaccagttaaagaatggttctgcatagtatttcatcggttgaggagtaaaagtctcccaatcagatttttcatccgctataaatgccatcatatgataatgcttatcagctagccattcacgaggaaattcatatttaacagcgccaattaactgatattttgctgcagtttcagggtcattaacaatgttatcacttaaaaatttaaaattactcgaagacagagaaactaatttaccgtcagttgacatattcgcatatccgggttgaatacgtcttctttcttcttcggtattaccaaaaactcttttccatgttttttcgtcatgatttaaaacatatattcctttatcagcagaatcaattatttttgaagttctaggattagcatttaatgtttcaacttcaccaataataagagcaaaaactttatcaccaatagaatccattttatagcatacagctttaggatttccagttatagtcattgtatcaggttcaaaaagtctttccgagtatgttggtgataacgggtcagaatctataggtgcattactcgtttttatatatctaactttatctctggcaacaacgtagatataatcatccgtacaagtaatggcttcagctatacgatatacattcgctggtaaagtcgcataagtagcgaagatttcaacatcgaatcctaaatgaagttgatctcctaatttagcaaatgttatatcctgtgaactgaatctgacatcatctgctgaccatctaacatcagtagatttgcgaccatagaaaatcttgtcgtatcctagaacgtatgttgtgtttgcagattggtaatatacagtcttagataaaggatatcctacacggtcattgaagagctttacagctttccaagtttgtcctttatcatttgatactttaactacgggttgataacgctcaaaaagatatagaatcccttctgattccatcaaataaactcggttaatatccttacatacttgctgaatagaaccttgtatttcatgatactcgttttcaccaataataaaattactgattgatgagacatcagcatacgatgggctaaactgaaatgattcattcatcaacgcagccattattgtgtcattattgaaattgacataattagaattattaagagtaaatttttcctgaatgaatttattggctaattgcatttcaatcatgttttgaaatgtataagcatttgtagcaaaagtttcaaactcttcggtataaatccaatcagactgctcaaaatcttgcgcagctgtagctactctaataacatatgatgtcagtggatcagcatcatcaaaaaagaaactattatttgctgtatatcctaaattaatccaacgatattgattactcgggagaaaatctcccgagtttgattttgtctcagcgatttctacaaagtagtagaaattagcaccaacgtcatcccagcgtacttgcacctgatttgcggataacttggaaattctgagactagtgactgaaggtgcttttactgtcattgtgatataggctccaaatcgatagttaagtattgtggacgtaagtcattttcaaatacaatcagtgaaccatcgcgagtaaagataacatcatcggttgggtcagaatataattcaatagtctgaacttcaaatttttcagatgttaaattaattttagcgatattccaataaattgtatcggctggatagtttatttcaccgataacatagtatttgtcgcgtccatcagaatttgctaatttgttaaaatcgttgcctgtatacggctgaatgttttcattttctgtaacatcgccagaagcaaatggaccaataataactttaccaattcctttagaatctcggtctgttgatactatacgaacgtcatataatacatcttcttctaaaccagtatcaggatttacaacctttcgtccagaattaaatgaaaacgtattagattccatagaacgatcttttatttgattattgtatttaatacctgcttcaggcgttttatagaagttttgtacttcacgaacaatttgaattgtcgctgatgaaccaatgatagaatgatctgcatcatctacgtaagttaacatcttagacttggcaaaagatgagttaaaaatttctacatcttctgtataataacgatcaattttatcaattatttgaccttcaagccactgttcggattcttgtagtttatttaaagcatatgtgacttttaaattagtcttaataaaaagataattgggagaaataattgacggtgtaataggcgctaaattataatctttgagataatttttaatatcttcacgctgtacagtagttaaatacaatcctgatttaggtttagcagcaataaatgcatacccaggtttagtagaatcagtgaaagtctgaacagcttgaataatagaaccaaatctttctgagacaaatgtatcataatcagttgcggttacgcagcgttgctgggtttcgcgtttaatagtacctaattcgcgaatacgctcaatatcttctggatcaccgccgccatctgctccgacaaaatctgggtcgtcgtttggattttcattaatattgatgacagttatatttgttaatgtatctgcatacgaaaatccgactgcgccgttcgcatctgcaccgttagtactgatgtactcaataacaattgtagagttctgggtaggtttaagacctccaatataattagcggtcaatgctccttctgccgcattaacggaaatctcaccttcaccaaaataaaattcagtatttccatcaatagtttcacgcatgtagtaaattgttgatgtagaaccagcatggaccattgactttcttgtccagttaatccattctgctccatcaacatacagcttaacttggtttctatcaatatttttatcataaatgataataggtgttaatttatcataaatgatttcagttcttactatacgtccctgggccaattttaaacgtggaaaatattggttatttttatcacgaatagcaataacgtcttcggtagaaacgaagttatatggattaacagaagtatcttttgcatatgctaaaaagcgagttccgcgaggaattgtgatgtaattcctattcaatgcgtcagtgcatgttaacataatttcggtctgcgcagcggattttgaagtaggtaaatatccgttatcttgtgcagcttgaacaactgaacttcgtaagttagcagtacgcataaagctttcatacacagcagcattaccaaactgctgaatgtacagcgtattataagccaaaaggtcacacagaacgtttaatcttgagccttcaaaatcataatccaaaaattcattttggccattaagccattcaatgaggttttgttttatttcagcaaacgtacccccgacgaatatctcgggaatagcatttgctgttcttgttaattgataatttacaggggtatttgccattttaaatcctatttaatgaatactttagatgatgcctgagccacagtatcaccgcatgatattggatcagccatttgaacagctttctttccagtgacatataccttagaagttctaggttgtgtcactccgccatgtgtttcatatggctttttaatttctgtatgttctgtaattggatcgcctgctactagaacagcaattcctccagtgaatactttactttgtgtagcattcacaactgttggaggccacgcttcatggccagcagtaacacacttatcataacttaatcctgacatttatggcctctcatacacatagcttctcagcttattagcccaacgactccaatttccaactatagttttagtataatttttaacgagagtttttcttactggagcaggaggatctgtcggttcagtagtatcagaagatgacctagaattactgccagaacctccagattcactttgttcttgatagtcatatattaatgttacttcgtaagtgaatgtcttctggagattttgaggagctttccacaaatacaactgagtgtcagaatcagtaggaagttcttcccatgaagcagcagttttaaattcatcacctaaacgatatttcaacgcgtcatttccgaaaccaaatacagattcatatgttccgtataagcgactttcttctactaaaaccccaggagtttcttcgtaactagttatatttatagatactaacgtttcacctgtttctaattgagcggtaaaggtgacgtcgatagaagaaccttccatggattctcctaaatcagcgctcattggaagtatattagccaatgtcaatcctcgatccatcaattgtgtattgaccagatgaaatagaactcatagatgccattttttctgtccaatcaccaccaacgtcccaatcaactgtcccagcaactttccaagaaagatttccatttactgtattagtctgatttccttcaactaaagtagtagcatctcctttaactgtaatgtcagcattaccttcaactacaatagtaacattacctttaactaggatagttccattaccttcaactgtcttagtttcatctccacgcacaaatatcgtattgcttccatctatttggtgaagacgattatccatgttgtaataaatttctgatccaccgacgttagtctttttatcgccggctaccaaaaaattaccatcagcgttggttatatcatacaaattatcgacagtttttcttgttcttcttcctgacggtgatacttcttcataagttccagttggatgaactaatctatatcgttcttgcccaggggtatcatcaaattcctgaatatgaccgctttcagtttccattgtgtgaacatacggatattcgcctttatatgaagaaactggttctttgaataaaattcttgagtcatttggaataggagggtcagctggatcagaagacttcgccacagtagcagccattgccgacaaagacctagctggtgttttaacttcaataccatatgattccaaatttccggtaagaataatcatggtaacacgggatgcacggccttttgtttgttgataccacaacgaatcacgaccagctttatatgctttttcccaatctcctgctaacatagcagttaacattgtgttaaatttagctacaccgccaacacccatctgaaatgccatattttctaatgccatttgacgagaacggttgacagcttgccagactggtcctactttagaatgtgatttaatgtcacgttgcatatcagctaaatcacgttcaaataaagttaccgcttcttccattgtaatagaacctggatttccagtaatttcacgaccaacttgttttgataaaactttattaatttgagccatatcacgaactggctgtttcatgataagatgaccaataccaattgtcggatatccttcagtatcccaataaacttttagtcttaatccttcatcacggcgaagcatttcagccattgacatatttggattatcatcagttggaatctctgatagtggtctatcatcgggatttattgcggtgtctaagttactatcttggataacgttagaagacgaatcatatcctacttctccgccttggtttagtacgttagtatcatttcctaaacgtctaggatattgtccagttggatcagaaaatccttcaagtctattcggtttttcgcgaactattccaccatacgtgccaagaacaattccgttggttttccatttgtctaaaaaatgaccataaactctagttccttctaccggtccagtaacggagcctccaattccagacattgctgcagaagttataggttgaataactgacatccatggtaatttttcagttggaatacccattacatcaccttgtgctctttgaggtggatgcagaccaacaacacgaacacgaacacgacctaattttaatgggtccattctatcttcaacaacaccaacaaaccaattaaggttattacttatcatttccataagatttctccattatacgtataaggtcgttcataaatgaattaatgtctgattttgctattatttttatttgacgaagtttttcattttcaagaacagcagcttcatacgtatcgaccgcagcaagtgctccttcatattgaggatattttctagctttatcacctttgtcataccaaacatatggattatcatcgtatgatactaaattataaaatttttcaccgttctcatttacatgatatactatttggtctccacctacgtttttgtatttttgtatagatgcttgataagcagcttcttgtgaagtaatccatccataatatggatcataattatcattacacatcaataaaacccaatacaactgtggatttccatatatagtatttgctaattcttccgggcgtggtgaacctttgatataataagtacgtaagcggtatcccgcaagagcgcgtttaaaatagtctttatagtttctaaaaatatctgtcataggaatagtcggcgcgtttttattcaccgttttggccgcatattcaatcggatcaaaaaatgtaaagagcatgggccctcctgtttataaatatattatctatttataaggagaatccaatggcatattctggaaaatgggttcctaaaaatatatcaaagtatagaggtgaccctaaaaaaattacgtatagatcaaattgggaaaaattcttttttgaatggttagataaaaatccagaaattattgcatggggtagtgaaacagcagtaattccttatttttgtaatgcagaagggaaaaaacgtagatacttcatggatatttggatgaaagattcttctggacaagaattttttattgaaataaaacctaaaaaagaaacacaaccaccagttaaaccagcacatctaacaactgcagcgaagaaaagatttatgaatgaaatttatacctggtctgtgaacactgacaaatggaaagcagcacaatctttagctgaaaagcgtggaataaaatttagaattctaacagaagatggattacgagctcttggctttaagggggcataatggctatttttcaaataattaatgaaagcactccccaagttccaaaggttaagcaatcattaaacgaaaagaaatggattcaaataggtcttgaatataaaaaggccaaagcaaaaggaatgacaggaaagcaatttgctgaagaaagaggaatcaaatactctacgtttacttcggcaatgtcaaaatatgcttcgggaattaaaacggctgaaaagattcaaaagcttgaatcaaaaccaatgaataagctcaataagcaagaaagacaactgcttatgataaattcattcagacaaacattgcgcgataaaattcgtaatgaaggtgcagcaattaataataaaaccagaaaatggtttgccgaaactattaagcaagtaaaaggacataaagttgttcgcccgcagccgggacgaatatatgcttttgcttatgatgctaaacacaaggaaactcttccttactgggataaatttcctttgataatttaccttggtttaggtaagcataatttaatgtacggattgaacttgcactatattccacctaaagctcgtcagcaatttctagaagagcttttaaagcaatatgcaaatacacctactattactaataaaacgaaattaaaaattgattggagtcaagtgaaaggatttaggggtgcagaccaaatgattaaggcatatatacccggtaatattatgggtagcctcgttgaaatcgccccgaaagactgggcgaacgttgtgttgatgccactccaaagcttctactccaaaggaaaacgtttctctgcaaacaaagtttggagcaatgtacgatgaattatgaaaaaatttataattcactaattgatagggctcgcaatagagcccttactggatataaagaaagacatcatattattccgaaatgtttaggcggtagtaatgatgcttctaacttggttgatttaactccagaagagcattatgttgcacatcagctattagtaaaaatacacccaaattctttaaaattatcttatgcagccataaaaatgacggtttcaactaaatatgttaggagaaataataaagtttatggttggttaaaaagaaaacatgcaaataatatttcaaatacacaaactggacgaaaatattataataatggtaaaagatgtataaaattgcatgatggagatataattcctgagggatttattttaggaagaggatggagtccgacacaaggaacaactcattctagaaaaaatgacacatttaaaaacaaaaagcttcaatctgctttagctaataaacggtgggaaaaagaaaagaaaataatgttagataactttggagttaacactattgatgaagcaaagaaaattgttcttgattttaaatcaacttgtcatccaagatattggcgaaagccaactttagaaaaatttccatttatgacttctgctcgtttaagaagtctaataaattaattctattatcttccattcttttctgttgtttgttctaaatggaattgaatggaagggacttagacccattataccaccaacagttataaagcattatgaggaatatatgtcgcaagctctgcaacaaatttttaaccaagcaaatacaactaactttgtagtatcaataccacatagtaatactacatctgcttttactttaaatgctcagtcagttcctattccaggaattagaatacctgttactgataccgtgactgggccgtttggactgggccgagcacaacgtccaggtgttacatttgagtatgatccactcattgtgagatttatagttgatgaagaacttaagtcatggataggaatgtatgaatggatgctaggaactagcaactatcttacaggtgaaaatactgcccaaaaaacaggtcctgagtacattacgctttacatcttagataatagcaaaactgaaatcgtgatgtcaataaatttttataagccttgggtttctgacctatctgaagtagaatttagctatacggaagattcagacccggctttagtatgtactgcaacgattccttacacctattttcaagtagaaaaagatggaaaaattatagcagaagtttaatgcttcagttttgtgtgttataatcttaattaaatttgaggagaaacacatgaaactaatctttttaagcggtgtaaagcgtagtggaaaagatactactgctgattttatcatgagcaattattctgcagttaaataccaacttgctggtcctattaaggatgcattggcttatgcatggggagtatttgcagcaaacactggctatccttgcttaactcgtaaagagtttgaaggaattgactatgatcgtgagactaatttaaatctgactaaattagaagtaatcacgattatggaacaagcattttgctatcttaatggtaaaagcccaattaagggtgtgtttgtttttgatgacgaaggacaagaatcagttaattccgtagcatttaacaagattattgacgttataaataatattgaagatcaatggtcagtccgtcgtctgatgcaagccctaggtacggatttgattgttaataacttcgaccgcatgtactgggtaaaattatttgctttagattatcttgataaatttaactcaggttatgattattatatcgttcctgatacccgccaagatcatgaaatggatgcggctagggcgatgggtgctacagtaattcatgtagttcgtcctggtcaaaaatccaatgatacacatattacagaagctggattgccaattcgtgatggtgatttagtaattacaaacgatggttctcttgaagaacttttttctaaaattaaaaatacactaaaggtactataatgtctgaacaaactattgaacaaaaactgtctgctgaaatcgtaactctgaaatctcgcattcttgatacgcaggaccaagcggctcgtctgatggaagaatctaaaattctgcaaggaactttggctgaaattgctcgcgcagtaggtatcactggcgatactattaaagttgaagaaatcgttgaagctgtcaagaatcttactgcttcatctgcagatgaagcaaaagatgaagaataatggaatttaaagacttttcaacgggtctttatgtagcagctaagttttcagaattaacacttgatgcgctggaagaacttcagcgctctttacgtgttcctaatccagttcctagagaaaaaattcattcgactatatgttattcaagagtaaatgttccgtatgttccatcaagcggaagttttgaagtagcttcttctggacatttagaagtatggaaaacacaagatggatctactcttgtacttgtactagattctgaatatctgcgctgtcgacacatgtatgcgcgggcgctaggtgctacacatgattttgacgattacacaccgcatataacattgtcttataatgttgggcccttatcatttagcggtgatgtacaaattccggtcgtattagatcgtgaatacaaagagcctcttaaattcgattgggcagatgatttaaaataatttcacaaagttgtttacatgctgatgaggtagtgatactattacctcatcaaaattaattaggaaaataaaatgaaaacttatcaagaattcacttcaaccactaacgcttctactttgactgaagcgacccttacttctgaagttattaaagcaaacaaaggacgtgaaggtaaaccgatgattagcctggttgatggcgaagaagtcaaaggtactgtttacctgggtgatggatggtccgctaagaaagatggtgctacaatcgttatctcccctgctgaagaaactgctttgtttaaagccaagcatatttcagtagctcagcttaaaattattgctaaaaatcttttgtaataaatttttcacaaagttgtttacatagggttttagttgtgatactattaccctatcaactactgaggagaataaaatgaaacgttgtgaattaattcgaaatgttgctattgcaatttctgcttctgcttttagtttttcaatgtttgttggatttatatgcggattattaactacagcagaaaatgtattttcacttgtagtagcatttttaattggtttaattgctatcgttatggataaaatttctaaaggtgaatagaatgtataaagtgtacgcggattatcaggctaatccatctgaaaagccagattgtcaaattggatgggctcatgatactttgcttgaagcagtagatgctgctcaaaaattaggatataaatatgtagagattgttcaaccgacaggaaaagttattactcttgaagagtttaataatattaaaccaaactttttactttttgcgggtgataattattatcctcgcggtgggtatgctgatttaatcgcgaaagccgctactgaagatgaactccgtgacattatcaaagaaaatgagaacaaaccaatgtatggttctaatcgctttgattggtggcaaattgtaaatgcccacactcatactattgttgatgaagggtaataatgattctttatgctaaagtatcatccattgaaaatggatataaatatgatcaagaagcagctaaagccttgattgatgattatggcgttttaacgtgttttgaagttgaaaaggtttatgttgaccgttcatcttctcaagttaaattagtaaaggaagaccgtaaatttaatacagtaaattttgatttctttattgaaacagaaaaaggtcctcttgaatatgatattttcaagaatcctttgggtcttgaatgcatcgtaaatatgtattatcataaatggtaaatatgctttaagaattatttgttattattaactcataccgcactgattaataccctctatcatcaagggctcttatttgagagcctttgttaataattgggaattagccaagttggtaaggcactggattttgattccaggatgcaaaggttcgaatcctttattcccagcgcgagaatggtcaaattggtaaaggcacagcacttaaaatgctgcggaatgatttccttgtgggttcgagtcccacttctcgcaccaaatttgcggatatcgtataatggcattacctcagacttccaatctgatgatgtgagttcgattctcattatccgctccaatttaatttactccgtgtagctcagtttggtagagcgcctgatttgggatcaggaggtccaaggttcaaatccttgtatggagactggaggcgtggcagagtggttgaatgcaccggtcttgaaaaccggcagtcgctccggcgactcataggttcgaatcctatcgcctccgccagttttgctgatttagctcagtaggtagagtacctcacttgtaatgaggatgtcggcggttcgattccgtcaatcagcaccaaggccctgtagctggaaggttcaagcaagcgactcataatcgccagatggtggttcaattccacccagggccaccaaattatgaattaggggagttatcccgtagaggtagcggtgtggactgtaaattcattgtcattgcgactcgggtggttcgactccatcactccccaccaatttggatgtgtagctcaatggcagagcgatcgcctgttaagcgattggttataggttcgaatcctatcacgtccgccaaatttgtgacaactgtcacattaatatctacttgacgcgattataggggttgaactgatcactcttctcttaatttcgtgtgaagacatcatgattgttggagtaggtattaatgtggccgtagttcagttggtagaactcgagattgtgattctcgtagtcatgggttcgactcccatcggtcaccccaattacggggcatagctcagaaggaagagcaaggaccttctaagtcctgggtcgtaggttcgatccctactgcctcgaccaattataagtgaggaaaatatgaaaggtaatgtttatttagtagttcatgatttaacattctattttaatcataatgatactgttatttctgaacgcgtaattaatttgctttatcagcatgcagattatgtttatgtcgaaaacgaatttggtcattggcaatttctcaaaaatcgttcatttggtttagatggttacgaatattttgaacgtaaagaccttttagataaaattccgttatctacacaatatcaaaatcacaagtctttatataaatgccgtccaattcgaaatgctgaatctgcgtatgaagtaattgatttatggcgtaaacgtcgtgaacaggttgatgctttaaaagaatactaagaaaccgggtcgctaccggtaagtcgtcggactgatgttccctggagtatagtttcctcccacagttttactgtgttctggctctttactatcacagcagaaacggcgcaccgaattatcgattcgaggaaatatctttgccgtaaaccgagtagcgtttttgacggaacgttcggatatggtcgagatatggccttttaaaaatattgagtagcgtcaactgcttaataaccgggttcgaatcccggcgtttcgtacaaacacttgccttagcaggtggaaccccgacaaggttgccgcaaggcttagccccgaccgaaaggttggggctttttagtataaataagtatattaaatccacgaattaaaacaggaaataagatgaaatcatatactcaatttttaaatgaagcggtgttaaatgaagcatctagcaccgaaattcaagctgtcgcaaaagcagccattgcagcgggtaaatattcttacaaagatgcttctgatgaatcgcgatttcagttcgcacgcgacatgaaagcggaaggatttacgggaaatgcagttagtatggcctggaaaagtttagttgctactggcgctgcttttgcaaaggcttcgggtaaacctgctcctaaagcagatcctaaagtcgcacaagaaaaaaatatcgttaaaggaattatctccaaatatgaagctatccttaaagagcttttagtaatcaaaactgaaggccaaaagctagctcgtgctaatagctttaaagataacccacatgctcatgacctagagtatgttgaagacatccaaaaaattattaaagaccgcatttggtctgctaaacaaatcaaataacattcttagccccgaccaaaaggttggggctttttggtatctaggcctttccaggcctctctaggcatcatttagtttctaccctttataatatattatcctatcctttgatttcccctccttgccctagaattccccaaaactttttaaaaaatttttcacaaaactgtttacatccctgttcttccatggtactatacaactatcaactactgatacagaaaacaacttggagaatgaaatggataattacggcgaactgttcaacttctttatgaaatgtgtttcagaagattttggtcgtacagtgaatgatattaaagttatcggtcctgatcatccgatgtttgaaacctatgcagtaatgggtaatgaagatggtcagtggtatactgtaaaggttgtgattaacatgttcactgctgaaggttatgttaaactgtcttctaaagtttaccatgataacgacgaaatcgcagaagaatatttcaataatatgaaataagtttacataggctcatgattgagatattatgagcctataatttgaggaaaactgttatgtctctttataccgaactgaatgaaacttccgaaaaatcttttgatggtatcttatcaataatgcaaaagcaggtattagaagagcttagtgaagccgcaagcaatgggtacaaagcttgtcgagttcaaagcccacatcgccgcatgtatgaacgccctcttattaattggctagaaagcgaaggtcttacggttaagatttgccctgctaaatttgatgttccacgtattcttaatatttactgggataaaccggttgagaaaagtgacacttgggttgttaatgtaaaaacatgtgataatggatgctcacttgaggtgtctgctgtaattaaaggcactcatggtgaaaaatcgtggggttggcctggtaaagataaagaaatcgtgttaagtgtaaaagctactagtattagtactcctatagctaaaagcattatcactgccgctgttgaagaagctgaacgaatttgtaaaattaaaaatagtttacatctcttaaatagcatgatataatttttctgtggattttaattttaatcaacctaaggaaatactatgaacacactgaagaaaattgttgagtttattcgcactaaacttggttctgttatggctaaaaatctatctgttgaagaacagtatactgctgcagcagcaaaactacttgataagattaaagacctaaaaactgcttccattaaatctattaacgaagaaaagcgtattcgtgaacttattgtcgaaaagaataagcaggccgaatcaaaagagcgcgaaattcgcaagctcctttctgaaggtcaagatgtaacaatgcatgctaaactcggtttactatatcgtcgaacagctgagcagctgactactaaagctgatggttatgctgaaatgcgaattgaaatcgccaagaaagtagttgagttagatgatgctcgccaagaacttgcagttaaattggaatatatccgtgaaactcgcgccgcgaatgccctcggaattagtactgctgatgatgtagttgaaattgcagcgctgactaaggttgatattgaagatactcttgctcgagttgaaacctttaatggtaatatttctggggttgaaactacctctgccgatgttcaggaatatattaattctctgaaataatgataaggggcttcggcccctattacttggagtaagtaggaatgaaaatgcaaagtgatttcaattcaatgtttgaagagttccaaagacaggttgatgttccagaccaattattaaatgctcttaaacgtatggcagaaggacgtaattattattggggttcttcatatgaaactgttgaaaatctttctggaagattttctagaggtaaaaagtctttaatacgtcctggaatacttattaacagtattgaatcaattcatttattaacatgcgattttgacgttgaatttactgattttgtttctcctgaatggacggtctgttatttaaacgacgattatgattatcttggcgtttatagtttaagtgatgcatggtttaaacgtaatttacaaaagtcaaatttattctatattgatactactgtaaaatttcagggcaagaaatatttctttactcttatagttgactctgaaacaaaatatgaaaataaacgtattcttagtaaaaagaatatcttaactattgtcgatgatctttttgataaattcgtagaaaatcccaattttgaaagcgatttattattagaaaaatttgttaaggaatgtagagaatatgtcaaaatcatcactataccttccaagtaaaccgttgaagtatgaattaaagcgtcagataatttctactgatgtgttaataggtcctgtggtagttgtatcatttgtgattctggtgattgctggaatggttatagacgttatgactgatattgaatctggtgcaatatttggagcaacactaattcttccgttggtagttccgcttttattagtacctataaattggataggatattggtatcaaggaagacactatcgtaaacgtgtacgtgattggaaagctcagtgtaaaaagattaaaaaggaacatcagcttaaacttgctgcgcatgaatttgatgaaattatgaaatttgttaaggaatcacgatgcaaaagccaaaactaaataaagtcaaatattctcttcctgagtcatttttaatttttgctgtagcgttggctgcagctcttgcgggtagtctttttggattgttaattgactgtcttattttaaacatccgtagtaacgtcagcggtataatagatataatagaagtttggagtgaacttcattatactatagcgatttcattattttcattctttggtacaatgttatattttcattatgataattttaaaataaattggcaaagaaaaaaggattacaaaatacaattaaaggaatataatagctacatgtcttatattgaaaaagaatcaatggaagagtttgtgagtgattgtaggaaaattaaatgattttaaaaactcgctggtatgatttagatgatggggatgatggcatttcagttgatagagttgactggaatggttgttccgaagatatgaaaaatttgttgattagggagtttagaatgggatatcaagcagctaagccatctattataacagatgataaattcgtgtgtattcataatggtcgcgctaagttaacgaacgctgattggttcacagataaaattatgattctgtggtatattattagtcttcctgtgacatcattcgtattttacttttttataaaaaatccaatggacagaataggagattggattcttttaaccatacttgttaatatttttactgcagcaatattatctgggatatggtacacgttcattgaaatgccatggcgactacgcagacaacaaaagatttttgatgaaaagaaatatactcaaaatttaaataactttatcactgaatgcaggaaattaaaatgaaaacactatcagctggtattatctttatgacagaagataaagatttatttatgggtcgggttactggttctcgtaagcctggaatgatggcacatcgctgggatattccaaaggggcgtgtagaaagttctgatttgaatgcactggaagctgcaaaaagagaatgcttagaagagaccggttttagtaattataattcagaccttctagaagacctaggtgtatttaaatattctagcaataaagacttacagttattttattatacgattccagtagagcatgagatgtttagaaattgccattgcgagtcttattttgaaaataaagatggtgttatgattccagagatggatgcttttgcccttattcctcgtactcagtggcaatatgtgatgggtccttcactttaccgaataatgaacagcctcttttaatttataaataccttctataaacacttaggaggtattatgaatatatttgaaatgttacgtatagatgaaggtcttagacttaaaatctataaagacacagaaggctattacactattggcattggtcatttgcttactaaaagtccatcattaagtgttgctaaatctgaattagataaagctattgggcgtaattgcaatggtgtaattacaaaagatgaggctgaaaaactttttaatcaggatgttgatgctgctgttcgcggaattttgagaaatgctaaattaaaaccggtttatgattctcttgatgcggttcgtcgttgtgcattgattaatatggttttccaaatgggagaaactggtgtagcaggatttactaattctttacgtatgcttcaacaaaaacgctgggatgaagcagcagttaacttagctaaaagtagatggtataatcaaacacctaaccgcgcaaaacgagtcatcgctacgtttagaactggcacttgggacgcatataaaaatctataaagttgtttactttctcctagaattgtgatagtatattcacagttgcttgaagagacaaattactaattaaaatatttaaaggaaacacatgaaaacatatcaagaatttatcactgaagcagctattaattctcaaattattgctgaatcttttactgatcttttgaaatttaaaaaaggacagaaaattactgctgtattggatgatggtacagaagttgagatggatgtacagggatataattatgcagtagatggaaaactgtataataaatctcatgctaaatttgattcatttgacgactttgttaatacagttgaagatgaaaaaactcgtcgatccattgtaactggtgatgctaaggttcttatggcacatggtcatgaacgtattcgcgctaaacagaataaaatgggtgaagataattttgcattagttggttatcaatctggtaagcaaacttatggctatcagcgtactgctaccatgtataataaaaatggtaaaattgcctttgtaaatagtaaaggttctattcagtacgttaaatcgttcaaataatatgggaacaagctggacctcatgattctgtgagggattcccgccaacctgtaataaggtcgagcccaagtgcggtaatgggtaaatacagaaatggacaattcatgcgccatggaatggcccaaatttagagagaataaaatgagaacatttttaactggtccttatctatccctgatgaatgcttttacacaccattctgacgctagagtagaagaaatttgtaatagtggattttatgtacctgattcgtcgtatttatttaaagaatattgtacacttcgtttagatggtggcagacaatccggtaaatcaactgcagtaactaattttgccgctaattggttgtatgatggtggaacagttattgttctttctaatacttcagcttacgctaaaatttctgtgggccgcattaaaaaagaattttctcgttattctagcgatgatattcgattccgtttatttactgattctgtccgtagttttgttggtaatagaggtagcaagttcagaggtttatcgctttctcgaattttgtatataatcgatgagcctgttaaatctcctgatatggataaaatttacgatattcatattgaaactgtgcaatattgctgcaatagtaaatgctgtattggtggtattactcgtccgcagtttttcgtaattggaatgcaatgatgacagatacgcaacttttcgaatatctttatttttcgccaaaaactattaaaaataaattggtgaatcattttgaaattttggcaaataataacattttgagcgaattttatcccaagcaatacaaattacaaaaaggcgtattcaaaggatgcagagttttgtgcactgctcctaacgcgcagttaatgaataaaattccatattttaccatggaatttattgatggaccttttaaaggactaatcacccaaagtttaatggcatatgattctgagccatttttaattaaagaacaatcttggataaatttattttctaattgaggtttatatgaaagcatatcaaattcttgaaggcacacacaaaggtactatttattttgaagatggtattcaagcacgaattattgtctctaaaacctttaaagaggactcttttgtagacccagaaattttctacggtttgcatgcccgtgaaattgaaattgagcaacagcctacagttaaaattgaaggtggtcagcacctgaacgttaacgttctgcgtcgtgaaactctggaagatgcagttaagcatccggaaaaatatccgcagctgaccatccgtgtatcaggttatgcagttcgctttaactctctgactccggaacagcagcgcgacgttatcgctcgtaccttcaccgagagtttgtaatggcaaagataattattgaaggttctgaagatgtgctaaatgctttcgccgaatggtttagtaattcaggcgaacagcaatttaatgaagcctggaatatgggtgatattgatggaatttatcctacgacagaaatttctgttcaaggatatggcattcatgaacctattcgtttagttgaatatgatttatgtactggtgaggaagtcaaatatgattgaagatattaaaggttataagccacataccgacgaaaaaatcggtaaagtaaatgctattaaagacgctgaagttcgtttaggacttatctttgatgctttatacgatgaattttgggaagcatttgatagctgtgaagatgatgaactcgcgaagaattatgctgaaagcctcgatcagttaactattgctaaaacgaaactcaaagaagccagtatgtgggcttgtcgtgcagtgttcaaaccagaggaaaaatactaatggctcaattaagcgcagggtttggttatgagtattatactgcacctcgtcgtgtatctgccgctcctaagaaaattcaaagtcttgatgacttccaggaagtagtccgtaaagctttccaggattatgcgcgttatcttaaagaagattcacaggactgtctcgaagaagatgaaattgcttactatgagcagcgtcttgaacagcttaaaaacttacatgaggttcgtgcagaagtttcaaagtctatgaataaattgattagatttaaagaataactgtttacttttcctcttgactgtggtataatttttctatcagttaagaggagaataacatgactatcaatacagaagtttttatccgtcgaaataagcttcgtcgtcactttgagtcggagtttcgtcaaattaacaatgagattcgtgaggcatcaaaagcagcaggggtctcatcgtttcatctaaaatattctcagcatcttcttgatcgtgcaattcaacgggagattgatgagacatatgtttttgaattattccataaaataaaagaccatgttttagaagttaatgaattcctgagtatgcctccgcgtcctgacattgacgaggattttattgatggggttgaatatcgtcctggacgtttagaaatcacagatggaaatctttggcttggatttacggtttgtaagcctaatgagaagttcaaaaacccgtcacttcaatgtagaatggcaattatcaacagtcgtcgtttaccaggaaaggcttctaaagcagtaattaaaactcaatgaggtaagcatgagaaaagcactactcgctggtctattggccatttcaatgatggcacatagctccgagcatactttcagtaatgtccaactcgataacatgcgttacgcgtatcaattcggggaacaattttctaaggatggaaaatataaaacacacaaaaacatccacaagagtggattaggtcatataatggctgctattttatggcaagaaagctctggcggagttaatttaaaatctaaaccaaagcatcacgcctacggaatgttccaaaattatttgcctactatgcgagcaagagttaaggaacttggttataatatgaccgatgctgaaataaaaagaatgttgaataaacggtccaattcagcttcctgggcgtacattgaactttcttattggttaaatatacataagggcgatataagaaaagcaatatcctcttataattcgggatggaatgttaaagcaggttctaaatatgcttctgaagtcctagaaaaggctaattaccttaaaaataataaacttttggaaatagtaaatgactaaaattttggttttatgtataggattaatttcattttctgcgttagcagatacatcatatactgaaattagagagtatgtaaaccgcactgcggcagattattgtgggaaaaataaagcatgccaagctgaatttgcacagaaattaatatatgcatacaaagacggagaaagagataaatcaagcagatacaaaaacgatacattattaaaacgatatgctaaaaagtggaatactttagaatgttcagttgcggaggagaaagataaagccgcttgtcattcaatggttgaccgtttggtagattcttataatcgaggattgagtactagatgattgtaaaatatatcaagggcgatattgtcgccctatttcttcaaggtaatattattgcgcacgggtgcaattgttttcatactatgggttcaggtgtagcgggtcaattagcaagagcctatcccaaaattttagaaatagataaaaccactactgagtacggttctcgtgataaattaggcgatatgtctattgtttttaaacataaacctaatggttttggcatatgttataacttgtacacgcaatacgaaccaggtcctaatcttgattatggtgctttagtaaactgcatgatagaattaaatctacaggcagaaacccttttgttcaaaccagtaatttacattccacgcataggttgtggtatcgctggcggcgattgggataaggtttctaaattaattgacatgtttactcctgatattgatttaatagtggtggattatgaaaatacattacccgcatccgtttgatcctaaaaacaaagcagtaattattcgtcaatgggaacgcatttgccgtactaaatgtccaattaatagtccacatgatgtagataaagactacattggaacattcgttgaatatacctttattgataagaaaggtcgtaaacagcacgtagaagaatattgcttaaaggtgacatggttatgagccaaactagtattcttaaaaatgcccactgtgaaaagtgtaagtggccggttgtttttgctttatgtaatgatgaaatggcttgtgatttcgattattggtgctattgttctaataaaggatgcatcaatcataaaggtgaaggattttattcaggattttatccttatcctgatttcgttaaagaaggtaaaccaaaatgaatagttttgagttacaatacgaagtgttacgtgagcttgataatttaattgaactcgctgttaataaaggttttgccattggaatcggtcaaaaagatactagtcatttaactatggaaatatttaagcaaaagcgaattattttaaaactcctggaaattaatatatatgagtctgagtaaagaacaaaaagataaattgtttgagcttatccatgaacttctagatgagcatacagaagcaaacaccttttatgatgaatacggcccgctatctcctgaacagcaggaagaatttgctgatcggtttgataagaaagaaaatgaattaatagcttatgtgaatacgctttaagaaggtgatatggcgagtttaatttttacttatgcagcaatgaatgctggaaaatctgcttctcttttgactgctgcacataattataaagaacgtggaatgagtgtattagttcttaagccggctattgatactcgcgattctgtctgtgaagttgtttctcgcattggaattaaacaggaagcgaatattattacagatgatatggatattttcgagttctataaatgggctgaagcacaaaaagatattcattgcgtatttgtagatgaagcccaatttttaaaaactgaacaggtgcatcaattaagtcgaattgttgatacatataatgttcctgttatggcttatggattaaggactgatttcgctggaaaattgtttgaaggttctaaagaacttttagcgattgcagataaacttattgaactaaaggcagtttgtcattgcggcaaaaaagcgattatgacagctcgattaatggaagatggaacaccagttaaagaaggtaatcaaatctgtattggtgatgaaatttatgtttctttgtgtagaaaacattggaatgaattaactaaaaagctcggttagtgcaaaagttataaataggtttatctaactaaaggggtatatatgctacaattaactgaaaagcaacttcgcaatcttactgtgcttcaattagatgaaattcgtagggaagttggaaatatcatttcagctttgcgtcgagaagtatcactcaaccaatctccggcagactatactagattgcgaaattttgaaaaataccttgataaagttaaggccgtgcatcggcataaagtaaatacaggacaaaaatgataggaggcctttatggccttaaaagcaacggcactttttgccatgctaggattgtcatttgctttatctccaccaattgaagcgaatgtcgatcctcattttgataaatttatggaatctggtattagacatgtttatatgctttttgaaaataaaagcgtagaatcatctgaacagttctatagttttatgagaacgacctataaaaatgacccgtgctcttctgattttgaatgtatagaacgaggcgcggagatggcacaatcatacgctagaattatgaacattaaattggagactgaatgaaattcagcgacttttcacaaagtggaaaaccttcaaaggcagatgaatacttaggtttattaatggctgcacaagcttattttcattctgcgcattttgaaactaaaagttatgctagacacaaagcatacgattttattttctccgagttgccagatttgattgataaatttggtgagcaatatttggggtattctggtagaaaatacacgccttcaattccagatgccagtaaacttcctaccgacacaattaaaatgattgatcgcatactagaccaatctaacagcatttataaagaaatgcctccagccattcaaagcacgatagatgatattactggaatgttttaccagagtaagtatcttctttccctcgaataacattagtctccttcgggagacttttttcattttaccggtttactttccgtttgagctgtgatactatacaactatcagataaagaggagaatatcatgaaaattgaagcactcaatcaagaaggaaatatctacgtcatcattaatggtgattttttcgtcgacatggatgaagttactagtgaagaacttgtagaacttcttaagaaacgttataatatgtgtgatgaagttgcaactcacatggcgtgtgcaatattctctctgtcatatgtggtggaataatgattagtatagagcaagcagataaaatcaaggaattagtggcttcaattcgtaaagcagatgaagagctgattgactttgcttggtattcatcgggtatttcgaataaaggcattaaagactttgaaactaaagtggataatgctatagaagcgttagatatgtttcttgatgaaattattgatcataatacgagagcttaagtatgctaacacgtgaacagtttgaaaaaatcattaaattagcacgtgatattgaaatagattcataccaattagcagttgagcattgtgaaggatattcatacgacggtatagcaacagctaaaaaggatttggataaatctaaagctaagttagttcaatatcttgaaatgattaggtggaataatgaaaactgaaaagcagatgtttttaatgaaactaattgaagaatatgctaatgcagtttctgactatgaatgttcttctcgggaaagaggtacagctttcgccaaagaagaaatgaaaatcatggttgatgctcacacaaagcttcagaattttattgaaaacgccatttaatggtttacaagttggcaaggttatggtatagtaatcttgtcaactgccaaggagaagagaatgaaagttttgtttgttgtgtatgtgatgattcaatataattacccaatgtttacttataatctggtgaataacattattgatatggttcaaaggagtatgtaatgacaagtgagcaggcttttaaattaagagaattaattgagacgtatagtagagctgttcatacatcaacaattattgatgaatcagctttctccggacatgctaacaagattaaatacaaaattcttatggaagaagctaaagtaaatcttgattcttatattgaaactttaattggtaaataatatgaaagctgtaattgaaacaactgatttgttcggtgacctggttatcgaaaaacgtgataatgtatacgttctgacgcaggaagacgatagtattactattcttcctatggaactggctgcgattttatcttatgctccctcaggagttaccggtgtaactaatattacaggcgacctccaagttcgtttttatcgtggcctttacggcggtaatattgaaactgaatttatgtgtctttctatcaacaattgggcaacattcgttgccaaggtgaaagaattcttggaatctgaaacagtcgaaacacccgaaaaggttaaacttcaatgggctaaagatcgcggtgctagcattattaatccagtagctgcaaactatactacgactttggatgtcaagacttcttatgaagatggcgatgtcgtacttgttcgccaaaatgatgattctcgagcacatattgtcacatttaccaaagacgaagctattgcgctaaagaattatcttgattctgtcattccaactatgatttcaaagtgaggaaatatgtttatttcaagtggaagtggtttaattcgtgttgaatttaaaaatgacatctttcttattcaaggagatgatattattaaaatgagttatgacgaaatcaagaaaatttgtcatgctcttgaaagtcatggaaaagtgaacgctgttattgatattggcgatttatgggtaacactttatgaagtatccgaaggatttaacgttgaagatgaaaataacattttagctattgataaaagaactgatttgtttgatgtattaaaagcctatgaacagtcaaatggtggaagaaaagctgtattgatttatcaaaaaccacattcgtgtggaactgcttcaatcatttcaaatattgaaggcgaagctgatacttatatgtgtgttttaaaagctggtggtgatcgtcatccagattttgtttctattcgccaaaacggtgaagaaatttcactatcaaaatcagaagctgaagctatgattaagtatttaacaactgttacaccttcaatgaaaggataattatgattattaatgaaaactcttggcattataaattattcaaaatgtttaacgacgaatggaaacgacctaagacactgtgcgcgtatttttggtctattgttattcctacatttttcgtttcttttttcggatgtactatactcgcaggtctaactatcatctgcgcagaaatcatgcagaaatggcttatttttggtagtttatggactcttattccatcagcatttatacttgctattttgcttgttttacttattattggttcatttgttattcctgcacaactgcatgaaaaatataaagattataaatggaaaaaggattatgctttacatgtagaaaatattgatagggcgtataaaggtttacctcctattcaaccaaagaaatctattatcgtcgaatttttaaaagcgcgtaaagctaaagtatgtcctgttattgaatataaggctgaatgatgaaaacagtaatgaaaagctattttggtagtcatctttatggaacttctactccagaatctgatgtagattttaaagaaatctttgttcctcctgctcgtgatattcttattggaaatgttaaagagcatatgagtaaaaacactaacaacacatcatctaaaaacaccaaagatgatattgaccatgaactatacagtcttaaatatttctttaaattagcagcagatggtgaaaccgtagcattggatatgctccacactccacctgaactggtagttaaatctgatttgcctgatgtatggaagtttattcaagacaatcgttctcgtttttatacgactaacatgaaatcctatttaggatatgttcgcaagcaagcttctaaatacggtgtcaagggttctcgtttggccgcattacgtgatgtattgaaagtagttaatcaaatccctgaccagtgggttgattaccaagagggaattccagatcgtttacgccgaactaaggtagaagatatcaaacaccgactccctgaaaacgagttttgtgaatgggtattccataatcatgagaaaaccggtccacaaacgttctacactgtgttgggtcgtaaatatcagacaacgctttctcttattgagcttaagcagtcactgaacaaattagatgctgaatatggtgaacgtgctcgtaaggccgaagccaatgaaggtattgactggaaagctctgagccatgcttgccgtggtggacttcaactattggaaatttacaaaactggtgacttggtttatccacttcaagatgctccatttattctcgacgtgaagttgggtaaacatccatttaaaacagttcaagagtttttggaagatgtggtcgatcaagtagaagcagcatctactgaagcttctaagaacggtatgcagcaaaaagtagacatgggtttctgggatgacttccttgagaaggtttatcttgaaaaccaccgaagttattataaatgatagggagccttcgggctcccttttttttatttcaaaaattttttcgcaaaattgtttacaatcataaagctttatggtactatacaactatcaactgatacggatttggagaataaaatgaaaactgtaactatcaataagggtatctacttcggtaaagaaatctctggaacttttgagctcttaggcgaatggttcccagataatgctccggtagatgcacaaggagatggtaaagtttttgttgaaattgacggtaaacgtcgcggtgtttgggtttataaatcagacatttcatatgatggtgtaaaagttgaagaagttaaagaatcgtatgaagatatgaaaacccgcattaataaaagatttaatgttatgggaatgatgacgaatggtattattaacggaaacattcgttcattaattatctctggtgcggcgggtattggtaaaacgtattctttagataaagctttaaataaagcaaatgatattggatacattgaatataaaagcattaatggtaaaatctctggtattggtctttatgaacagctttggaataatcgtgaagagaattctgtccttttgattgatgatgtggatgttttctctgatatggacattcttaatcttctgaaagctgctctggacaccggagagacccgtaaagtctgctggagcaccgcatcttcttacttagaagaaaaaggcattgagcgtgagtttgaatttaaaggaacgattgtttttatcacaaacgttgacattgaccgcgaattagaccgtggtactaaacttgctccgcatttacaagcattagtttcccgctcagtttatttggatttgggtgttcacactaatgaagaaattatggtcagggttgaagatgttattctttcaactgacatgatgcaaaaacgcggtctttctgatgaagaaacttataaagcattatcatggatgaaagtcaatgttaatcgtttacgcaatgtttcactgcgtactgcactttatcttgctgactttattatgaccgacaaaaacggttgggaagaaattgctgaggttactcttctgaaataattcataagaggccttctatgacaaaaaggcagttcagaaatagattatatggactgccattaaaaagatgactagaattaaactggtgaatggaggtagtgatgttatactcaaaggctcgtgaaatttatgaaactaaaattaaagacgcggttcatcagttcgcaacgacaatgcgttggacaaatgactgggaatactccaagaatcataagaagcccttggtgacaagaaaggctcatatgttagtgttaatagaccgtgagcagattaaagcccgagaagccctccagaatcataaaaaggctgcctttgaatggtttatggataacactgctcctgagactaagaaagcggtcagcgcatggttcagtggaaaaaattgtgaaagaagtttcttttagttgtttacaagactgttcctctgtggtactatacaactatcaactacggaggaacagaaaatgaatgctaaagatattttcaacctggtaaattacaacgatggtaaatttaaatctgaagcacaaagcaagttctttaatgacatctcaatcggaggtgaaatcactgttgatggaggacaaatttacaaatcccgttggaattggatcgttattattgatgaaattggtattgtagaaatttacaaaaatacgaataaaaatcgtacattacactggtctcgtgatactaacgaacagtacaaaaaggataaagcatctaaattatctcgtgtaactcaagaagatattgagttcatcaagaaagatattttaatgtatgataacttaattgctgaagagcaagctgttattgataaatttgatgagattaaagcttctcgtgaaattcctgattttatgaaagaatcagtaaatgaacgatacactctcatttcagagcgtattgaaacttacaaaaagcaaagagctgaacgccagaacactcttcggaaatttgaagaacggttaaagacggtactcgcataaccgctttataccaaggatggtataatggttctaagcccttttaattgagattattatgaaacagttgataattaaaagattgaatttattgatatgttgtttatgtatagtaattgcatatggttattatgctattaatgattatatgcattataaagattatgatgttactgtagttaataccattacaggaactcaaggaaaggggtctagtttatcgtttattgccgtatatgaactcaaagacggttatagatttagtgaatatatttccccagagatgtattcttcaatagaaaaaggtgataatattactgtaagtttacgtcctttcgacgtaaaacagacattgtttgataatatcgtttggttctttggaatggtattagttcaatctgtatgcggtgcttatatagtcttttccatcatattctgtgtatttagtaaaattgaaattgagtgaggaaaatatgtcagtagtaattaataatgtcaatacagtaattaaatctttagttaataaaaaattgaatgaatggactgtacttcgtcgtggagagccagataaattttttcatagatttaacccaactttggatttgaatgttattgacagagatgttcatgctgaaattttagataaatttaaagttgatattggatttggattagaaaaacatttacagcgaacaaacggatctggaatgagtttatctaatcgcatcatgaaagcccttaataaaattggagcattgtctcgtattaatgcgagtgaaattcttcgtaattataataaaggatatgacctttatggtcgactaatgccaaaattatcatttgaccaaatgattgcagatttgtgggaaaatcaacgacgattattagcattaggcgctcgattagctaaaggtctagataaacaaatgatttttaagaccaataatacagaagaccttaaatgctttaaatttagtactcgtggagatgattattacatcagagctcgctctactgattatgtcaatatgggtcatcatctctgtttagcttttgaagttttaaaagaatctggaacattagaatattcatctggtgcaaaatgtccaattggttcaagttgtattttaatttatcgcccggatgaatccagttcaactaaattgcctacaaaacctgtaccagttcgtagtaacgaaaaacattctgagcaaattgattattttaataagcaaattgaagagctgaatatttctattcaacaatatgacgatgaaatctttagactatctggattgagtagtaaagctaaatctgagcgtgaaaagttaattaaaattgttgatttacttaaatcttaaggaacactatgaaaactcgttctcaaattgaagatatggttcgtaatgccagctatactcgtgatgttatgacatttttgtgccaaaataatttagaccctgatagagttaatagttccattcactttaagtatatgaatagcagtgaatggttacgtcattttagtaaagcgggatatattacacaaatgactgctcgtgagcagctcaccgatttctgtaaaactattgattataaaaatcctctatttgttcaaggtgttggtcagagtaaggttgatttatcatctggatttttcaatcccaatcattatcgtattgaatggagatttattgctctattccgtaaacagttaaagcaaattttgtcgacagctagtcgattaaaaggttctgacattaacttaaagaacctgaaatttgatggttatactcttcagatggaagtaagaccattaaaagaaaataatagaactgcacgaattagctttaaacctaatacaaaaaattctctttcaatttgtgaatgccttaaatcacagttgacagaagcatttaagtatatggatgttgttgccgctgttcagtctaagattttacctcgttttgaacgatttaagttagatacaacatcatatgaacttgatatgatcgtttcatttaaatacgattttttgagaaaggacgaagtcccgcaagagaaaaagcaggaagtacaagataacttaaatttatccaattacttatcaaacgatcctaaattttggatgtatagttcaggtaataatgatgcatggaaattcaataaagtgaattttcttcctgttgaaaatccgagtcttaaacctgttgaaaaatggcacgcggatgcgattgagaagtctatcaaggcagtagatgatgaactcgttaaagcgaccaatgaagtgctagaagctgaaaaggcgttagaacaagctcagtcaagggttcaaaatctgacaaagcaacgttctaaactgaacaatgcattaaatgcactgaactagtttactttgccacaaggatgtggtataatgttcttactttctactgaggagattaatatgactcgtaacgaatatatcaaatcattcaatagggttattgatgataaagttaaacccatgtctagtcagaatgctgttctttctattatcaatcaatggctcagcagtattgatgcaagtattgtttcttccagtaaatttattcatgaagttcgtgaaatttctagccgtgtagataaagatgatattaagataatctttaaagggtctcgtcttctttcatatttagttaatcgggatattcttggtaattttgggaaagaaattaaacgaactaaaggtgtaataggatacaattggttcggtgatgttaattcttatcatcttaatatcaaagaagaccctaagaatatttttactcgtcgttggattagtaatttcagactttttaaaggacaaattctaaaatcagcttctaaattatgttatggcgattatcgtcaaattcatcctttggcttctgatatgattatcgtaaaagaatatgagcttgataaaaataaagtagctatttttgtgaattatgaattttttacaccagaagctaaccaaaagaatattaataaatttttctcaattgctagcactataactcatcaattagagacggctttggattgtatggaaactgtaaaaagtattcatacatatcctttcaaaaatattggtggttgggaaggatataaaattgtaattagtcttcgtgaagtgaaatgtgcttattcactaactgataaagaaatttaccaacaaaaatgcgaagaaattgtgaatactcctaaagaagaaactacccttgaggaactaatggataatcttgacgattcacttgaaccggtagaaattcgtccagaagttgttgcactagaaaaagcttataaagaagttctagaaatttctaataaagcgcagaaagaatatgagaaggctaaaaagatttgggaagaatctgttaatcgcttggatcgtcttgaacaagctttacagttaattaagtaatttaaagccaaggatggctcggagtataaatcattaacaagtgagaagaacatgaaaactcgtaaacattatattgattattttgacagtcttattactaaacatcgtaattatcagatcggacacagagcagtaatcaataatattcttcgtgatttccttcaatatgttggccaggaaaatcatatctgtaaagatactcaaaacgcttattctcattcgttaggcaatttgcttgaatggtttaaacgttctcgtatgttatcttctactatagctcgtgataacattaaaaactttatgaagccgagcttcattaaatctgagacatctataaccgatttggttcaatttactattgttaacgatgttaaaaagacccatttggctgattggttatctaccattcctgaaactaaatttgctgataaatttgcttgtcaattcaatacccaagtgaatatgctttttaagcatgctcgtaaactatttaccgcgggcgatgaccgtacaaatacagttcatgttaaggattgggttattgctgatgaagtaacacacaagccaggtggttcatcagtattaattaatatccaggttccttattattactcacgtaatctcggcaccatgacagctagagaaattaataagcacaacaaaattattcgttcgttgtcttataaactctgcacgatggtggagatgatggacgtagtggaaatgtatgacgaaaccgaagataatggttcaatgctgtacagttcacgcattctaattaaactgaagaaccctaatacatataagcaagttgtaaaagaacctaaagcagaaaaggctgataatctgagtgaagaacgcgaatatctcaatgctcgtttgattgaagttgaagcccaaattgcagaacacaccaaattgttaaaagctcttaatgcaaaagcaaatggtttacgtaatgctattgaggtattgaaatgaaaaagcgtttattagaagacattgcagcttcaagtaattccagtttaattaaaattattatggctggtgaggaagatgatatggaaatgcgtggaaagattcacggctgcgacgatttagattttaaacctccagcatgggatgctattatggctatggttgaacgacgtgaaagagcttctaaaaacgttcctaattgccctgaatgcggtactgaacaagttcagttgattaactggcgtaaaccagagcttgaatataaatgccgtcaatgtaaacataaatttaataagcatgctccggaaatggttaaattgcctgactctactgagttctttaaagaacttgtgagtgttcaaccaatgcctaataatattttggattaaaaatgaccaagcgtaaagaatatatggagactgctaaaaaggcagtccgtgaattagcaatagcttattataatgaacatggtaaatttcctgatagatacagcgtgcttaaatctgctttaactcgttcatataaaaatatgctatcagaagtaagtgatattatatacaaacataaagaacaaacgggccaaagtcttgattacgacgagacttttaaacaagtactaggaattaaggaataatatgtttaaagtatatggttatgatagcaacattcataaatgcgtgtattgcgataatgcaaaacgtcttttgactgtgaagaaacagccgtttgaatttatcaacattatgccggaaaaaggtgtttttgatgatgagaaaatcgctgagcttctgactaaactaggtcgtgatactcaaatcggcttgacaatgcctcaggtatttgctcctgatggaagtcatattggtggatttgaccaactgcgagaatattttaaatgatgctcgaaggaactgactatattcatgattaccgcggaagcgcggtatatgtaggtgatgaagttgcagtttattatgggtatggaactttgatgacagcaaaggttattcaaattaaaaataatcgtgctaaacttgaagtttattattctaatggtgaaaagtctatttctaagtggaaatacggcgattgtatggtcaaactggggtaaatatgatttacgatattaatgtagcaagagctccgtcaatggttactattcctgctgaagaactagatcgtcttcagaaaattgaagaacttctttgggaaattgaatgtgacctgccatcaggactagaatcctggattgatgatgaagaacttaataagcttcggggttaaaccttggtgggcggctagatgggaaactgtagagccagagccggaagaaccggtttacactgatgaagaaacagtatataatgaaccaacgataaatgacttaattgatatggagatgggacatgattacagtagataagtggtttagaattaatcgtgttgatacagggctgtgtaattactggccggaacttagtgcaggtactgtctttaaagttcgtgaacttgcaaaagaatgtgaagatgatatagaacctgatactggaattattgaaattgaactttccgacggaaagattattaacatctacgataagccaattacgtattggtgtttgtggaatactgaatcagtcgaaaatggcgaaattgaagaagttgtagagcgaactaatcaagttgttcagaagcctaaagccgattttcagggtgaacgtatttcatacgcattagctaaattagctgcacaagaaaataacgatggctatgaaggaaatttgatgcaagctgcggcagaatatattgaatggcttgaaacccaaatttctttttctgaccaaaagattcggcaatataagcgattgaatcaaatgttttacaatacttgaaaaaactcaaaattctttttctgacaggacattttaatgaaaaccgaattggtttatactgaaaagttaaatggcggtaaggtttggaaactttttattaaaggacattctacggaccctcatatgaccacttgcgtaggaacctattctcgtcctactaaaaagatgattcgacagtataaacggttgcatcgaatgttttacaatacttgaaaataataaatacccttatctatttaaggtaagggtttattatgttattgactggcaaattatacaaagaagaaaaacagaaattttatgatgcacaaaacggtaaatgcttaatttgccaacgagaactaaatcctgatgttcaagctaatcatcttgatcatgaccatgaattaaatggaccaaaagcaggaaaggtacgtggattgctttgtaatctctgcaatgccgcagaaggtcaaatgaagcataaatttaatcgttctggcttaaagggacaaggtgttgattatcttgaatggttagaaaatttacttacttatttaaaatccgattacacccaaaataatattcaccctaactttgttggagataaatcaaaggaattttctcgtttaggaaaagaagaaatgatggctgagatgcttcaaagaggatttgaatataatgaatctgacactaaaacacaattaatagcttcatttaagaagcagcttagaaagagtttaaaatgacaattgaaaaagaaattgaaggattgattcataaaactaataaagaccttttaaacgagaatgctaataaagattctcgtgtttttccaactcaacgggaccttatggctggtattgtgtctaaacacattgccaaaaatatggtcccgtcttttattatgaaagcgcatgaaagcggaattatccatttccatgatattgattattcccctgctcttccatttactaattgttgtttagtagatttaaaaggaatgcttgagaacggatttaagcttggtaatgcacagattgaaactcctaaatcaatcggtgttgctactgcaattatggcacaaattactgcacaggttgcttcccatcaatacggcggaacgacttttgctaatgtagataaagtactttctccttatgttaaacgcacctatgcaaaacatattgaggatgcagaaaaatggcaaatcgctgatgcgttgaattatgctcaatctaaaacagaaaaagacgtatacgatgcattccaagcttatgaatatgaagtaaatactctctttagttcaaacggacaaacaccttttgtaacaattacatttggtacgggaactgactggactgaacgaatgattcagaaagcaattctgaaaaatcgtattaaaggtcttggtcgtgatggaataactcctattttccctaagcttgttatgttcgttgaagaaggcgttaatctttataaagacgatccgaattacgacattaagcaacttgctttagagtgcgcaagcaaaaggatgtatcctgatattatttcagctaagaataataaagctatcactggttcatctgttcctgtttctccaatgggttgccgtagtttcttgagcgtatggaaagattcgactggtaatgaaattcttgatggacgtaataatcttggtgttgtaacactgaatcttcctcgcatcgcgttagattcttatattggaacacagttcaatgaacagaaatttatcgaattgttcaatgaacgaatggatttatgttttgaagctttgatgtgcagaattagttccttaaaaggagttaaagcgactgttgctcctattctttaccaagaaggtgcattcggggttcgtcttaaacctgatgacgacataattgagttatttaaaaacggtagaagttcagtgtctttaggatacattggtattcacgaattgaatattcttgtcggtcgtgatattggacgagaaattttaactaaaatgaatgctcatcttaaacagtggactgaaagaaccggatttgcttttagtttgtattctactcctgctgaaaacctttgctatcgcttctgtaaacttgatacagaaaaatatggaagtgtaaaggatgttactgataaaggctggtacactaacagtttccacgtttcagtagaagaaaatgttactccgtttgaaaagatttctcgtgaagcgccatatcatttcattgcgacaggtggtcacatttcttatgttgaacttcctgatatgaaaaataacttaaaaggtcttgaggctgtatgggattatgccgcacaacatttagattattttggtgttaatatgccagtagataaatgttttacatgtggaagtacccatgaaatgactcctactgaaaacggatttgtttgttctatttgtggagaaactgatcctaaaaagatgaatacaataagaagaacatgcggttatttgggaaatccgaacgaacgcggatttaatctcggtaaaaataaagaaatcatgcatagggttaaacaccaatgaattatgatagattttatccttgcgattttgttaatggtcctgggtgcaggaccgttctttttgttactggatgtttgcataaatgcgaagggtgttataataaatcaacgtggaatgctagaaatggtgttccattcactggagagacactagaacaattaattgaatgtttgaataatgattatatagaaggattaactataactgggggtgaccctctttatcctgataacagagacgtgattcactctgtggttcaaactattaaaaatctttatcccaataaaagcatttggttgtggacaggatataagtttgaagatattaaacaactagaaatgcttaaatatgttgatgttattattgatgggaagtatgagaaaaatcttccgaccaaaaagctgtggcgaggatcagataatcagcgactttggtcaaataccgatggggtgtggaaacatgattaaattgaattacattatggatactataaatgatatgatttttcattttggtccagaattttattcgcaatatagtttagtgcttatcaatgcttggttaatcaattaagggtaaaatatgtataaatttcgtaaaggtttagctgattttcttacaactgtaacgttctttctgtttatggcagttggagctattttccttattccttttattgttgtatttttcgtgattagtttaatttctccagaaaagggcttatcttctagtgagttcaatgagcgtctggataaaattactaacaagctgaatgctgttcttgataaaaaggcttaattatgattagttttgagcggtatgtagtagagagttggaatggttttgatatgttcggtaatgactattatttctatgaatgtagtctgaacccaagcttctgggctggacgtgaacaagacctcgaagaaatcaacgctcgtgccgatttgttaggtgaactgcctactacttatttcacctttgatgaatccggcttcgttatccaggtttattttcctgaagaaaactctggtgaggattctgttaatcctccttattgggcttaccaaggaattatttctcgtggaacaaaactcgaacttaaagaataagattgaagtctatggaattccagatgaagtaggtcgttgtcctggatgtcaatcagttacaaaacttctaaaggagctcaatgctccttttactttctataaagttcttacaaataatggtaagattgagtatgatcgtccactgattgtatctcttgccaaacgcgccggattcacatctcttaacattcgttatccagtcattttcattaatgattctagacaaaagaacattaaacacttcaaagaaactctcatttcacttggatatgatagagatatcatagaagactaagacgggccctctgggcctttctttctcacattctgtatattaccattctaagctatcgttcacttcttatcattccctaaaataattttcacaaagttgtttacaagaagttcaaaccgtggtattattaacatatgaattacctttgaggaattgatatggttgataaagagattaaaattgaccagtactatcgtgttaacggacatatcgttattgttacagcagtcgatgatttagatgttcattatactgtgattaaatggaacaaacgtatgatttgtgatcgtgctgtatttagttcagttgctaaggaaattaaactccatgggtaaaacgtatcgtcgtaaagacttaaaagtgcgtgattatgactattttggaaagcgtaaagctccagacggtgtaagtcataaagatatggttgaaaacatttttcgctcagataaatggcgtagaatgaaaggtattgattcagaagttaaagatgagctaaatcgccaattacgtggtgaagtaagaaagttgaaaaaatcagtttacattgacgatgattttgattataatacttctcaactagttgctaagcgtaaatcaaacgagtgctatcgttacagctgaggaaaatatgaatatcaaacgaatgctttttaagcaagggctatatactttaaatgttactccaaaaggcgatacaactaagtggtcagtaaatgactggattaaatttattgatgaaaacggtaactggaaaatttaaatgaatcctgaatctaaattatcgcagcgaattgccgaagctcgtcgtcagttctttgaaaatatgatagcccaaggtattgatgacgaagtttttctgaactggttttggaatcacaaatattcaagttgctcaggagcaatattaatatcagccgcaatgatgtacgaaggctggaagggtgccaaacagtttagctaagggcttcggcccttttttggataataaaattttaacgcaattgaggataatgtatgactattcaaattaaaaacgccatcaattcttatgcatatgataaagtagtttctctgctagaaaaaggcgatattgtaactcctcaaattttggataaatgggaaaaagagcttcatcagacgatgaaacagaatgatcagaagattggacgaaatactgtccgtgaattgttggttcaatatatcttgtcagaatttgatgttaaagcttttggtgtagaatctaaagcttatcaaaaacatgaaatttccgataaaactattcgtcgtatgaaaaatcaacgcaagaaaaaatttgctgacctgaaaattactaaggtataattatgaacgaagctcttattaacgatttgcgtcttgccggatatgaagtaaatacaaacggcaatggtttaactcaaattgaaggaaatggattcattcttgagtatgaatttagccaatggtggttatacgctaattacggcgaattgattgaatacgttgaccaatttgattcactagatgcagctcttggagcggctaagctgatgaatgtatgaa

(19) United States
(12) Patent Application Publication
(10) Pub. No.: US 2025/0164483 A1
(43) Pub. Date: May 22, 2025

(54) LYTIC PHAGE WITH HIGH SPECIFICITY TOWARDS PATHOGENIC ESCHERICHIA COLI

(71) Applicant: The Curators of the University of Missouri, Columbia, MO (US)

(72) Inventors: Azlin Mustapha, Columbia, MO (US); Kai Wen Choo, Columbia, MO (US)

(21) Appl. No.: 18/951,339

(22) Filed: Nov. 18, 2024

Related U.S. Application Data
(60) Provisional application No. 63/599,693, filed on Nov. 16, 2023.

(51) Int. Cl. G01N 33/569; C12N 7/00; G01N 33/543

(52) U.S. Cl. CPC G01N 33/56916; C12N 7/00; G01N 33/54387; C12N 2795/10022; C12N 2795/10031; G01N 2333/01

(57) ABSTRACT

Herein are presented methods for incorporating a novel bacteriophage, CAM-21, that exhibits lytic activity against pathogenic E. coli, into food, food packaging, films, and quantum dot-based biosensors. The bacteriophage contains no genes associated with toxins, virulence factors, antibiotic resistance, lysogeny, or allergens. Methods are provided for encapsulating the bacteriophage with proteins and/or carbohydrate materials to enhance stability. Various films in which the bacteriophage are incorporated, and which similarly exhibit suppression of pathogenic E. coli, are also provided. Additionally, when conjugated with N-doped graphene quantum dots, CAM-21 enables biosensor development for E. coli detection in food products through altered photoluminescence emission spectra, and methods for making and using such biosensors are provided.

### SEQUENCE LISTING

This application contains a sequence listing, the contents of the electronic sequence listing (2024-11-18 UMCO-40489U1-17193-197.xml; Size: 166,000 bytes; Date of Creation: Nov. 13, 2024) is hereby incorporated by reference in its entirety.

### FIELD

The present teachings relate to detection and control of pathogenic bacteria using bacteriophages, and particularly to incorporation of bacteriophages into food packaging and bacteria detection systems.

### BACKGROUND

The statements in this section merely provide background information related to the present disclosure and may not constitute prior art. The present disclosure relates generally to a novel bacteriophage and methods of preparation thereof, and particularly to the incorporation of a novel bacteriophage that demonstrates lytic effect against Escherichia coli (“E. coli”) into food packaging, films, and quantum dot biosensors.

Foodborne illnesses caused by pathogenic bacteria are a significant public health concern, with E. coli being one of the most prevalent and dangerous bacterial pathogens found in food. Shiga toxin-producing E. coli (STEC) strains, in particular, can cause severe illness and potentially life-threatening complications. The Centers for Disease Control and Prevention (CDC) estimates that E. coli infections cause approximately 97,000 illnesses annually in the United States alone.

Traditional attempts to prevent or mitigate bacterial contamination in food products have largely relied on chemical preservatives, pasteurization, and various packaging technologies. However, these conventional methods have notable draw-backs. Chemical preservatives and heat treatments, for example, can negatively affect a consumer's perception of a food's taste and/or appearance. Considered in the context of the rising popularity of minimally processed foods, the drawbacks of conventional methods engender a need for novel antimicrobial strategies that can ensure food safety while satisfying consumer needs.

One such novel strategy considers the use of bacteriophages, which are viruses that specifically target and eliminate bacteria. Bacteriophages offer several advantages over traditional methods as they can directly target specific bacteria, replicate solely in response to the presence of those bacteria, and otherwise not interfere with the taste and appearance of the food product. However, it has not traditionally been clear how one can maintain bacteriophage viability during food processing and storage, and it has furthermore not been clear how these bacteriophages can then be used as detection systems.

Current food packaging designs have yet to take advantage of the potential of bacteriophages as antimicrobial agents, perhaps due to the multiple competing constraints placed on any candidate bacteriophage: the bacteriophage in question must be stable under transport, processing and storage conditions; the bacteriophage must not be toxic, mutagenic, or otherwise harmful to a consumer; the bacteriophage must be specific to one or more common food-borne pathogens; the bacteriophage must be sufficiently potent as to counteract pathogenic bacteria even at low multiplicities of infection; and the bacteriophage must be amenable to incorporation into pack-aging or other useful complexes.

Furthermore, the rapid and accurate detection of E. coli contamination in food products remains a significant challenge. Traditional detection methods include techniques like polymerase chain reaction (PCR) analyses. Such techniques are time-consuming and require specialized laboratory facilities. Pathogenic bacterial infection of food products, by contrast, can occur very quickly, and thus rapid detection and decision-making is essential for preventing the distribution of contaminated products. Additionally, many existing biosensor technologies suffer from limitations in sensitivity, specificity, or ease of use, and are difficult to integrate into food supply chains and storage systems.

Therefore, there is pressing need for novel approaches that can address multiple aspects of pathogenic bacteria control and detection in food products.

### SUMMARY

In various embodiments, the present disclosure discusses methods of making and using bacteriophages for antimicrobial control of E. coli. The present disclosure also discusses the make and use of films incorporating bacteriophages, principally as food packaging materials, as well as the make and use of quantum dots conjugated with bacteriophages for the detection of target bacteria.

In various forms, described herein is a bacteriophage with an inhibitory effect on E. coli. In various forms, the bacteriophage is a bacteriophage called CAM-21, deposited under GenBank accession number OP611477, or is a variant that has the same phenotypic characteristics of bacteriophage CAM-21, or is some combination thereof, and has lytic activity against pathogenic E. coli. In various forms, the bacteriophage has lytic activity against pathogenic E. coli even at a multiplicity of infection value of 0.001. In various forms, a latent period for the bacteriophage is approximately 20 minutes. In various forms, the bacteriophage has a burst size of approximately 69 plaque forming units (PFU) per infected cell. In various forms, the bacteriophage has a genome that comprises no genes known to be associated with known toxins, virulence factors, antibiotic resistances, lysogeny, or allergens. In various forms, the bacteriophage is in the Myoviridae family. In various forms, the bacteriophage is a species in the Tequatrovirus genus. In some forms, the bacteriophage has at least 85, 86, 87, 88, 89, 90, 91, 92, 93, 94, 95, 96, 97, 98, 98.5, 99, 99.1, 99.2, 99.3, 99.4, 99.5, 99.6, 99.7, 99.8, 99.9, or even 100% sequence homology or identity with SEQ ID NO. 1. In some forms, the bacteriophage genome encodes an amino acid sequence having at least at 85, 86, 87, 88, 89, 90, 91, 92, 93, 94, 95, 96, 97, 98, 98.5, 99, 99.1, 99.2, 99.3, 99.4, 99.5, 99.6, 99.7, 99.8, 99.9, or even 100% sequence homology or identity with at least 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 246, 247, 248, 249, 250, 251, 252, 253, 254, 255, 256, 257, 258, 259, 260, 261, 262, 263, 264, or 265 open reading frames from SEQ ID NO. 1. In some forms, the beginning and end points of the sequence encoding the amino acid sequences are denoted in Table 3 and are relative to SEQ ID NO. 1. In some forms, the bacteriophage genome has at least 2, 3, 4, 5, 6, 7, 8, 9, 10, or 11 tRNA genes in common with SEQ ID NO. 1. In some forms, the beginning and end points of the tRNA genes are denoted in Table 2 and are relative to SEQ ID NO. 1.

In various forms, also described herein is a method of preparing the bacteriophage described above. The method generally comprises obtaining an agricultural waste sample, performing a viral plaque assay on the agricultural waste sample using a target bacterial strain, iteratively transferring plaque samples to a buffer to form a purified phage solution, and then cultivating the purified phage solution in a mixture with a saturated bacterial culture. In various forms, the method further comprises centrifuging and filtering the mixture to remove bacterial debris and precipitating the bacteriophage. In various forms, the mixture comprises tryptic soy broth as a medium for the bacterial culture. In various forms, the mixture is incubated at 37° C. In various forms, the mixture is cultured in an incubator for 24 hours. In various forms, the precipitated bacteriophage is resuspended in a saline magnesium buffer.

In various forms, also described herein is a food product resistant to E. coli contamination comprising a food substance and a plurality of bacteriophages incorporated into or onto the food substance. The bacteriophage is CAM-21 deposited under GenBank accession number OP611477, or is a variant that is described herein, and/or has the same phenotypic characteristics of bacteriophage CAM-21, and/or is some combination thereof, and has lytic activity against pathogenic E. coli.

In various forms, also described herein is a method of making a food product resistant to E. coli contamination. In various forms, the method generally comprises providing a food substance and providing a plurality of bacteriophages. The bacteriophage is CAM-21 deposited under GenBank accession number OP611477, or is a variant that is described herein, and/or has the same phenotypic characteristics of bacteriophage CAM-21, and/or is some combination thereof, and has lytic activity against pathogenic E. coli. The method further comprises incorporating the plurality of bacteriophage CAM-21, variant, or combination thereof into or onto the food substance.

For purposes of both the food product and the method described above, in various forms, the food substance is selected from the group consisting of dairy products, meat, fish, fruits, vegetables, and combinations thereof. In various forms, the food substance is a dairy product selected from the group consisting of milk, yogurt, butter, ice cream, cheese and cream. In various forms, the food product is pasteurized or unpasteurized. In various forms, the food substance is selected from the group consisting of dairy products, meat, fish, fruits, vegetables, and combinations thereof. In various forms, the food substance is a meat selected from the group consisting of beef, pork, poultry, lamb, bison, venison, and any combination thereof. In various forms, the meat is ground, chopped, sliced or cut. In various forms, the food substance is a fruit selected from the group consisting of apple, banana, orange, pineapple, kiwi, mango, papaya, peach, plum, pear, grape, lemon, lime, strawberry, raspberry, blueberry, blackberry, cherry, watermelon, cantaloupe, honeydew, grapefruit, tangerine, pomegranate, coconut, avocado, guava, passion fruit, lychee, fig, date, apricot, nectarine, cranberry, elderberry, gooseberry, currant, boysenberry, persimmon, quince, kumquat, dragon fruit, star fruit, durian, jackfruit, rambutan, prickly pear, feijoa, loquat, longan, and any combination thereof. In various forms, the food substance is a vegetable selected from the group consisting of lettuce, spinach, broccoli, cauliflower, carrots, peas, beans, bell peppers, tomatoes, cucumbers, zucchini, kale, Brussels sprouts, cabbage, celery, radish, beetroot, onion, garlic, leek, turnip, parsnip, sweet potato, potato, pumpkin, squash, eggplant, okra, asparagus, artichoke, corn, fennel, Swiss chard, collard greens, mustard greens, bok choy, arugula, watercress, endive, chicory, kohlrabi, rutabaga, jicama, yam, taro, bamboo shoots, beet greens, celeriac, daikon, horseradish, and any combination thereof. In various forms, the food product is a fruit or vegetable that is cut, chopped, sliced, pureed, or mashed. In various forms, the food substance has a pH between 4 and 11.

In various forms, also described herein is a method of making an encapsulated bacteriophage. The method generally comprises providing one or more protein and/or carbohydrate materials, providing a solvent, combining the one or more protein and/or carbohydrate materials and the solvent to form an encapsulant, providing a bacteriophage solution comprising a plurality of bacteriophage, where the bacteriophage is CAM-21 deposited under GenBank accession number OP611477, or is a variant described herein, and/or that has the same phenotypic characteristics of bacteriophage CAM-21, and/or is some combination thereof, and has lytic activity against pathogenic E. coli, mixing the encapsulant with the bacteriophage solution to form a bacteriophage encapsulation material, and extruding the bacteriophage encapsulation material to produce an encapsulated bacteriophage.

In various forms, also described herein is a method of making an encapsulated bacteriophage. The method comprises providing one or more protein and/or carbohydrate materials, providing a solvent, combining the one or more protein and/or carbohydrate materials and the solvent to form an encapsulant, providing a bacteriophage solution comprising a plurality of bacteriophage, where the bacteriophage is CAM-21 deposited under GenBank accession number OP611477, or is a variant described herein, and/or that has the same phenotypic characteristics of bacteriophage CAM-21, and/or is some combination thereof, and has lytic activity against pathogenic E. coli, and spray-drying and electrospinning the bacteriophage with encapsulant to produce an encapsulated bacteriophage.

In various forms as applied to either of the methods of making an encapsulated bacteriophage described above, various exemplary forms pertain. For example, in various forms, the one or more protein and/or carbohydrate materials is selected from the group consisting of soy protein isolate (SPI), whey protein concentrate (WPC), oligosaccharides, starch including modified starch (corn syrup), pullulan, gelatin, acetate cellulose, sodium caseinate, glycerol, sodium alginate (SA), carboxymethylcellulose, cellulose nanofiber, and combinations thereof. In some forms, the oligosaccharides are derived from soybean by-products. In some forms, the starches are from underutilized products like tapioca. In various forms, the one or more protein and/or carbohydrate materials comprises sodium alginate. In various forms, the solvent is selected from the group consisting of distilled water, a buffer, organic acids, alcohols, ethylene glycol, and dimethyl sulfoxide. In various forms, the solvent is water and the one or more protein and/or carbohydrate materials comprise sodium alginate. In various forms, the encapsulant comprises 2, 2.1, 2.2, 2.3, 2.4, 2.5, 2.6, 2.7, 2.8, 2.9, or even 3.0 wt % sodium alginate. In some forms, the encapsulant comprises about 2.5 wt % sodium alginate. In various forms, the bacteriophage solution comprises at least 0.1, 0.25, 0.5, 0.75, 1, 1.5, 2, 2.5, 3, 3.5, 4, 4.5, 5, 5.5, 6, 6.5, 7, 7.5, 8, 8.5, 9, 9.5, or 10 wt % of the plurality of bacteriophage. In various forms, the bacteriophage solution comprises between 0.1 wt % and 10 wt % of the plurality of bacteriophage. In various forms, the bacteriophage solution comprises between 1 wt % and 5 wt % of the plurality of bacteriophage. In various forms, the bacteriophage encapsulation material is extruded through a needle during the extruding. In various forms, the bacteriophage encapsulation material is extruded into a 0.1 to 3 M calcium chloride solution. In various forms, the methods further comprise filtering and/or washing the encapsulated bacteriophage with deionized water. In various forms, the pH of the bacteriophage solution is between 3 and 11.

In various forms, also described herein is a method of making an antimicrobial film, generally comprising the steps of providing one or more proteins and/or carbohydrates, providing a solvent, providing a plurality of encapsulated bacteriophages or a plurality of bacteriophages, where the bacteriophage is CAM-21 deposited under GenBank accession number OP611477, or is a variant described herein, and/or that has the same phenotypic characteristics of bacteriophage CAM-21, and/or is some combination thereof, and has lytic activity against pathogenic E. coli, mixing the one or more proteins and/or carbohydrates, the solvent, and the plurality of encapsulated bacteriophage or the plurality of bacteriophage to form a film-forming solution, casting the film-forming solution onto a substrate, and drying the film-forming solution to form an antimicrobial film. In various forms, the one or more protein and/or carbohydrate materials is selected from the group consisting of soy protein isolate (SPI), whey protein concentrate (WPC), starches including modified starch (corn syrup), oligosaccharides, pullulan, gelatin, acetate cellulose, sodium caseinate, glycerol, sodium alginate (SA), carboxymethylcellulose, cellulose nanofiber, and any combination thereof. In some forms, the oligosaccharides are derived from soybean by-products. In some forms, the starches are from underutilized products like tapioca. In various forms, the one or more protein and/or carbohydrate materials comprise carboxymethylcellulose and/or sodium alginate. In various forms, the one or more protein and/or carbohydrate materials comprise pullulan, sodium alginate, or glycerol. In various forms, the one or more protein and/or carbohydrate materials comprise soy-protein isolate, cellulose nanofiber, or glycerol. In various forms, the solvent is selected from the group consisting of distilled water, a buffer, organic acids, alcohols, ethylene glycol, and dimethyl sulfoxide. In various forms, the pH of the film-forming solution is between 3 and 11.

In various forms, also described herein is an antimicrobial film comprising a film and a plurality of bacteriophage incorporated into or onto the film, wherein the bacteriophage is CAM-21 deposited under GenBank accession number OP611477, or is a variant described herein, and/or that has the same phenotypic characteristics of bacteriophage CAM-21, and/or is some combination thereof, and has lytic activity against pathogenic E. coli. In various forms, the film comprises one or more proteins and/or carbohydrate materials. In various forms, the film comprises one or more proteins and/or carbohydrate materials selected from the group consisting of soy protein isolate (SPI), whey protein concentrate (WPC), oligosaccharides, starch including modified starch (corn syrup), pullulan, gelatin, acetate cellulose, sodium caseinate, glycerol, sodium alginate (SA), carboxymethylcellulose, cellulose nanofiber, and any combination thereof. In various forms, the one or more proteins and/or carbohydrate materials comprise carboxymethylcelluose and/or sodium alginate. In some forms, the oligosaccharides are derived from soybean by-products. In some forms, the starches are from underutilized products like tapioca. In various forms, the one or more proteins and/or carbohydrate materials comprise pullulan, sodium alginate, or glycerol. In various forms, the one or more proteins and/or carbohydrate materials comprise soy-protein isolate, cellulose nanofiber, or glycerol. In various forms, the film comprises at least 0.1, 0.25, 0.5, 0.75, 1, 1.5, 2, 2.5, 3, 3.5, 4, 4.5, 5, 5.5, 6, 6.5, 7, 7.5, 8, 8.5, 9, 9.5, or 10 wt % of the plurality of bacteriophage. In various forms, the film comprises between 0.1 wt % and 10 wt % of the plurality of bacteriophage. In various forms, the film comprises between 3 wt % and 6 wt % of the plurality of bacteriophage. In various forms, the bacteriophages are encapsulated. In various forms, the bacteriophages are encapsulated in a hydrogel, nanogel, carbohydrate, protein, and/or any combination thereof. In various forms, the bacteriophages are encapsulated in sodium alginate. In various forms, the bacteriophages are homogenously incorporated into the film. In various forms, the antimicrobial film is biodegradable. In various forms, the pH of the antimicrobial film is between 3 and 11. In various forms, antimicrobial films in accordance with this disclosure do not differ from films having the same composition but lacking the bacteriophage described herein. In preferred forms, at least one measurement of the color, lightness, redness, yellowness, opacity, tensile strength, flexibility, stretchability, and stability, are at least 95, 95.5, 96, 96.5, 97, 97.5, 98, 98.5, 99, 99.1, 99.2, 99.3, 99.4, 99.5, 99.6, 99.7, 99.8, 99.9, and even 100% identical between films having bacteriophage in accordance with this disclosure and films not including such bacteriophage, but otherwise having the same composition for the film.

In various forms, also described herein is a method of making a packaged food product, generally comprising the steps of providing a food substance, providing an antimicrobial film in accordance with the above description, and packaging the food substance with the antimicrobial film to form a packaged food product. In various forms, the food substance is selected from the group consisting of dairy products, meat, fish, fruits, vegetables, and any combination thereof. In various forms, the food substance is a dairy product selected from the group consisting of milk, yogurt, butter, ice cream, cheese and cream. In various forms, the food product is pasteurized or unpasteurized. In various forms, the food substance is selected from the group consisting of dairy products, meat, fish, fruits, vegetables, and any combination thereof. In various forms, the food substance is a meat selected from the group consisting of beef, pork, poultry, lamb, bison, venison, and any combination thereof. In various forms, the meat is ground, chopped, sliced or cut. In various forms, the food substance is a fruit selected from the group consisting of apple, banana, orange, pineapple, kiwi, mango, papaya, peach, plum, pear, grape, lemon, lime, strawberry, raspberry, blueberry, blackberry, cherry, watermelon, cantaloupe, honeydew, grapefruit, tangerine, pomegranate, coconut, avocado, guava, passion fruit, lychee, fig, date, apricot, nectarine, cranberry, elderberry, gooseberry, currant, boysenberry, persimmon, quince, kumquat, dragon fruit, star fruit, durian, jackfruit, rambutan, prickly pear, feijoa, loquat, longan, and any combination thereof. In various forms, the food substance is a vegetable selected from the group consisting of lettuce, spinach, broccoli, cauliflower, carrots, peas, beans, bell peppers, tomatoes, cucumbers, zucchini, kale, Brussels sprouts, cabbage, celery, radish, beetroot, onion, garlic, leek, turnip, parsnip, sweet potato, potato, pumpkin, squash, eggplant, okra, asparagus, artichoke, corn, fennel, Swiss chard, collard greens, mustard greens, bok choy, arugula, watercress, endive, chicory, kohlrabi, rutabaga, jicama, yam, taro, bamboo shoots, beet greens, celeriac, daikon, horseradish, and any combination thereof. In various forms, the food product is a fruit or vegetable that is cut, chopped, sliced, pureed, or mashed. In various forms, the antimicrobial film has a pH between 3 and 11.

In various forms, also described herein is a biosensor comprising an N-doped graphene quantum dot and a bacteriophage, where the bacteriophage is CAM-21 deposited under GenBank accession number OP611477, or is a variant described herein, and/or that has the same phenotypic characteristics of bacteriophage CAM-21, and/or is some combination thereof, and has lytic activity against pathogenic E. coli, where the bacteriophage is conjugated to the N-doped graphene quantum dot to produce an altered photoluminescence emission spectrum when the biosensor comes into contact with pathogenic E. coli and is excited by light of an excitation wavelength. In various forms, the pathogenic Eschirichia coli is a Shiga toxin-producing E. coli. In various forms, the N-doped graphene quantum dot is an orange emissive N-doped graphene quantum dot.

In various forms, also described herein is a method of producing a quantum dot-bacteriophage biosensor, comprising providing a solution comprising one or more N-doped quantum dots, providing a solution comprising one or more bacteriophages, where the bacteriophage is CAM-21 deposited under GenBank accession number OP611477, or is a variant described herein, and/or that has the same phenotypic characteristics of bacteriophage CAM-21, and/or is some combination thereof, and has lytic activity against pathogenic E. coli, and conjugating and/or crosslinking the one or more N-doped quantum dots to the one or more bacteriophages thereby forming a quantum dot-bacteriophage conjugated biosensor. In various forms, the N-doped graphene quantum dot is an orange emissive graphene quantum dot. In various forms, the method further comprises isolating the one or more quantum dot-bacteriophage conjugated biosensors. In various forms, the isolating is carried out by ultracentrifugation, gel electrophoresis, column chromatography, high pressure liquid chromatography, dialysis, and any combination thereof.

In various forms, also described herein is a method of detecting E. coli in or on a food product by using a quantum dot-bacteriophage biosensor as described herein. The method comprises providing a food product, providing a quantum dot-bacteriophage biosensor as described herein, applying the quantum dot-bacteriophage biosensor onto and/or in the food product, applying light from an excitation source to the quantum dot-bacteriophage biosensor on and/or in the food product, detecting a photoluminescence spectrum emitted by the quantum dot-bacteriophage biosensor, and identifying the presence or absence of E. coli in or on the food product on the basis of an intensity or wavelength of photoluminescence and/or a photoluminescence lifetime.

### DRAWINGS

The drawings described herein are for illustration purposes only and are not intended to limit the scope of the present teachings in any way.

FIG. 1 shows a graph depicting evaluation of lytic activity of Escherichia phage CAM-21 using E. coli O157:H7 strain C7927 as the host bacteria at multiplicity of infection values ranging from 0.0001 to 100.

FIGS. 2A-2D show transmission electron microscopic images of Escherichia phage CAM-21.

FIG. 2A shows the morphology of CAM-21.

FIG. 2B shows the attachment of numerous phages on surface receptors of the bacterial cell.

FIG. 2C shows the formation of progeny phages in the bacterial cell.

FIG. 2D shows the release of progeny phages from infected cells through lysis.

FIG. 3A is a graph showing the optimal multiplicity of infection of CAM-21. Data points denoted with different letters are significantly different (P≤0.05).

FIG. 3B is a graph showing the rate of phage adsorption. Data points denoted with different letters are significantly different (P≤0.05).

FIG. 3C is a graph showing the one-step growth curve of CAM-21. Data points denoted with different letters are significantly different (P≤0.05).

FIG. 4 is a genomic map of Escherichia phage CAM-21. Each color in the figure legend represents predicted open reading frames (ORFs), GC skew and GC content of CAM-21.

FIG. 5A shows a phylogenetic tree based on the complete genome sequence of bacteriophages from the subfamily of Tevenvirinae, including Escherichia phage CAM-21.

FIG. 5B shows a phylogenetic tree based on the major capsid protein of bacteriophages from the subfamily of Tevenvirinae, including Escherichia phage CAM-21.

FIG. 5C shows a phylogenetic tree based on the terminase large subunit of bacteriophages from the subfamily of Tevenvirinae, including Escherichia phage CAM-21.

FIG. 6 shows genomic comparison of Escherichia phage CAM-21 with four other closely related phages, including Escherichia phage wv7, vB_EcoM_BECP11, vB_EcoM_UFV09, and T4, using EasyFig.

FIGS. 7A-7F show the antimicrobial effect of Escherichia phage CAM-21 on the survival of E. coli O157:H7 on refrigerated food samples.

FIG. 7A shows milk at an MOI value of 1,000.

FIG. 7B shows milk at an MOI value of 10,000.

FIG. 7C shows ground beef at an MOI value of 1,000.

FIG. 7D shows ground beef at an MOI value of 10,000.

FIG. 7E shows baby spinach at an MOI value of 1,000.

FIG. 7F shows baby spinach at an MOI value of 10,000.

FIG. 8A is a bar graph showing tensile strength results for various biopolymer films.

FIG. 8B is a bar graph showing elongation at break results for various biopolymer films.

FIG. 9A is a confocal microscopic image of CAM-21 dyed with a fluorescent stain in an SM buffer.

FIG. 9B is a confocal microscopic image of CAM-21 dyed with a fluorescent stain in a soy protein isolate film.

FIG. 10A is a SEM image of soy protein isolate film surfaces comprising 0% bacteriophage at magnification of 1000×.

FIG. 10B is a SEM image of soy protein isolate film surfaces comprising 1% bacteriophage at magnification of 1000×.

FIG. 10C is a SEM images of soy protein isolate film surfaces comprising 4% bacteriophage at magnification of 1000×.

FIG. 11 provides FTIR spectra of carbohydrate biopolymer films incorporated with CAM-21 bacteriophage.

FIG. 12 provides particle size distribution spectra measured for various carbohydrate biopolymer films.

FIG. 13A shows the inhibition of E. coli O157:H7 in tryptic soy broth by soy protein isolate films comprising different concentrations of CAM-21 bacteriophage. Bars labeled with different letters within a single incubation period are significantly different (P≤0.05).

FIG. 13B shows the inhibition of E. coli O157:H7 in peptone water by various carbohydrate biopolymer films. Films labeled with suffix ‘D’ comprise CAM-21 bacteriophage.

FIG. 14A shows the antimicrobial activity of soy protein isolate films incorporated with CAM-21 on E. coli O157:H7 on raw bottom round beef. Bars labeled with different letters within a single storage period are significantly different (P≤0.05).

FIG. 14B shows the antimicrobial activity of soy protein isolate films incorporated with CAM-21 on E. coli O157:H7 on raw beef trimmings. Bars labeled with different letters within a single storage period are significantly different (P≤ 0.05).

FIG. 15 shows the antimicrobial activity of carbohydrate biopolymer films incorporated with CAM-21 on E. coli O157:H7 on spinach leaves.

FIG. 16A shows a photograph of electrophoresis of purified NGQDs under daylight.

FIG. 16B shows a photograph of electrophoresis of purified NGQDs under ultraviolet light.

FIG. 17 shows UV-vis absorption spectra of NGQDs at an excitation wavelength of 365 nm.

FIG. 18A shows an XRD spectrum of NGQDs.

FIG. 18B shows an XPS survey scan of NGQDs.

FIG. 19 shows UV-vis spectra of O-NGQDs at an excitation wavelength of 365 nm.

FIG. 20 shows variations in photoluminescence intensity from O-NGQDs as a function of excitation wavelength.

FIG. 21 shows an FTIR spectrum of exemplary O-NGQD sample.

FIG. 22 shows variations in photoluminescence intensity from O-NGQDs as a function of varying water content in DMSO.

FIG. 23 shows photoluminescence spectra of O-NGQDs in 100% water and in 20% DMSO-water solution.

FIG. 24 shows comparative photoluminescence spectra of O-NGQDs alone and in conjugation with bacteriophage CAM-21.

Corresponding reference numerals indicate corresponding parts throughout the several views of drawings.

### DETAILED DESCRIPTION

The following description is merely exemplary in nature and is in no way intended to limit the present teachings, application, or uses. Throughout this specification, like reference numerals will be used to refer to like elements. Additionally, the embodiments disclosed below are not intended to be exhaustive or to limit the invention to the precise forms disclosed in the following detailed description. Rather, the embodiments are chosen and described so that others skilled in the art can utilize their teachings. As well, it should be understood that the drawings are intended to illustrate and plainly disclose presently envisioned embodiments to one of skill in the art, but are not intended to be manufacturing level drawings or renditions of final products and may include simplified conceptual views to facilitate understanding or explanation. As well, the relative size and arrangement of the components may differ from that shown and still operate within the spirit of the invention.

As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to practice the disclosure and are not intended to limit the scope of the appended claims.

Unless otherwise defined, all technical and scientific terms used herein have the same meaning as commonly understood by one of ordinary skill in the art to which this disclosure belongs. The terminology used herein is for the purpose of describing particular example embodiments only and is not intended to be limiting. As used herein, the singular forms “a”, “an”, and “the” may be intended to include the plural forms as well, unless the context clearly indicates otherwise. The terms “comprises”, “comprising”, “including”, and “having” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps can be employed.

When an element, object, device, apparatus, component, region or section, etc., is referred to as being “on”, “engaged to or with”, “connected to or with”, or “coupled to or with” another element, object, device, apparatus, component, region or section, etc., it can be directly on, engaged, connected or coupled to or with the other element, object, device, apparatus, component, region or section, etc., or intervening elements, objects, devices, apparatuses, components, regions or sections, etc., can be present. In contrast, when an element, object, device, apparatus, component, region or section, etc., is referred to as being “directly on”, “directly engaged to”, “directly connected to”, or “directly coupled to” another element, object, device, apparatus, component, region or section, etc., there may be no intervening elements, objects, devices, apparatuses, components, regions or sections, etc., present. Other words used to describe the relationship between elements, objects, devices, apparatuses, components, regions or sections, etc., should be interpreted in a like fashion (e.g., “between” versus “directly between”, “adjacent” versus “directly adjacent”, etc.).

As used herein the phrase “operably connected to” will be understood to mean two are more elements, objects, devices, apparatuses, components, etc., that are directly or indirectly connected to each other in an operational and/or cooperative manner such that operation or function of at least one of the elements, objects, devices, apparatuses, components, etc., imparts or causes operation or function of at least one other of the elements, objects, devices, apparatuses, components, etc. Such imparting or causing of operation or function can be unilateral or bilateral.

As used herein, the term “and/or” includes any and all combinations of one or more of the associated listed items. For example, A and/or B includes A alone, or B alone, or both A and B.

Although the terms first, second, third, etc. can be used herein to describe various elements, objects, devices, apparatuses, components, regions or sections, etc., these elements, objects, devices, apparatuses, components, regions or sections, etc., should not be limited by these terms. These terms may be used only to distinguish one element, object, device, apparatus, component, region or section, etc., from another element, object, device, apparatus, component, region or section, etc., and do not necessarily imply a sequence or order unless clearly indicated by the context.

Moreover, it will be understood that various directions such as “upper”, “lower”, “bottom”, “top”, “left”, “right”, “first”, “second” and so forth are made only with respect to explanation in conjunction with the drawings, and that components may be oriented differently, for instance, during transportation and manufacturing as well as operation. Because many varying and different embodiments may be made within the scope of the concept(s) taught herein, and because many modifications may be made in the embodiments described herein, it is to be understood that the details herein are to be interpreted as illustrative and non-limiting.

### EXAMPLES

### Example 1: Preparation of CAM-21 Bacteriophage

Ninety bacterial strains, obtained from the University of Missouri, Food Microbiology Lab culture collection (Table 1) were cultured in Tryptic soy broth (TSB) (Difco Laboratories, Detroit, MI, USA) for 24 h at 37° C. In Table 1, “+” denotes the formation of plaques; “−” denotes the absence of plaque, and EHEC denotes Enterohemorrhagic E. coli. All bacterial strains were utilized for the phage host range study, and E. coli O157:H7 strain C7927 was utilized as the host bacterium for phage isolation and purification, and production of a high titer phage stock. This bacterial strain was also used to evaluate the inhibition effect of isolated phages in foods.

Twenty lagoon slurry and fecal samples were collected from Foremost Dairy Research Center (Columbia, MO, USA) for phage isolation. Briefly, 5 g of sample were mixed with saline magnesium (SM) buffer (0.1 M NaCl, 0.008 M MgSO₄, 0.05 M Tris-HCl, 0.01% gelatin, pH 7.5) and centrifuged at 10,000×g for 10 min at 4° C. Then, the mixture was filtered through a polyether sulfone (PES) filter membrane (pore size=0.20 μm; Nalgene, Rochester, NJ, USA). The double-layer agar technique was performed by adding 100 μL of diluted filtrate and 100 μL of log-phase host strain in a test tube containing 5 mL of melted top agar (TSB, 1.25 mM CaCl2, and 0.5% agar). The mixture was then overlaid on a pre-poured tryptic soy agar (TSA) (Difco Laboratories) plate. The melted agar was allowed to solidify before incubation of the sample for 24 h at 37° C. To purify the phages, each plaque was picked with a sterile pipette tip from the agar plate and added into 300 μL of SM buffer. The phage suspension was stored overnight at 4° C. prior to the next experiment, for time efficiency's sake. The double-layer agar technique, as previously mentioned, was performed using 100 μL of the phage suspension. Then, the melted agar was allowed to solidify before incubating for 24 h at 37° C. The purification step was repeated three times. The purified phage was then stored in SM buffer at 4° C. before subsequent analyses.

A high titer phage stock was produced using a polyethylene glycol (PEG) precipitation technique. One hundred microliters of purified phage solution were mixed with 1 mL of saturated bacterial culture (˜109 CFU/mL) in TSB and the mixture allowed to sit for 15 min at 37° C. The phage/bacterial suspension was added into 10 mL of TSB and cultured in a shaker-incubator with shaking at a speed of 200 rpm for 24 h at 37° C. After centrifuging at 11,739×g for 15 min, the suspension was filtered using a PES filter membrane (pore size=0.22 μm; Merck Millipore, Cork, Ireland). Then, 10 wt % of PEG 8000 was added and the solution was left overnight at 4° C. to allow for complete precipitation of the phages (Carroll-Portillo et al., 2021). The phage pellet was precipitated by centrifuging at 16,904×g for 15 min at 4° C. After the removal of the supernatant, 1 mL of SM buffer was added to resuspend the pellet and this was used as the phage stock. The phage count was determined using the double-layer agar method.

For preparation of phage stock specifically for preparation in films as described below, 100 μL of overnight grown culture of E. coli O157:H7 was transferred to 4 mL of Tryptic Soy Broth (TSB), and the culture incubated at 37° C. with shaking at 200 rpm for 6 h. One milliliter of phage stock solution in SM buffer was then added and the mixture was incubated at 37° C. for 15 min. Then, the bacteria/phage mixture was transferred to 150 mL of TSB in a flask and allowed to incubate at 37° C. with shaking (200 rpm) for 20-24 h to promote phage replication. The suspension was then centrifuged at 11,739×g for 15 min to separate out bacterial debris, and the resulting supernatant containing CAM-21 phage was filtered through a 0.22 μm PES membrane filter (Merck Millipore, Cork, Ireland) to achieve a purified phage solution. The phage solution was then centrifuged at 671,328×g for 2 h at 4° C. After removal of the supernatant, 2 mL of SM buffer were added to the pellet and the suspension kept in 4° C. overnight to allow complete resuspension of the phage pellet in SM buffer. The phage count was determined by the double layer agar method.

TABLE 1
- Host Specificity of Escherichia phage CAM-21

| Bacterial | | | | Plaque |
|---|---|---|---|---|
| Species | Source | Strain | Pathotype | Formation |
| E. coli | Clinical | ATCC 25922 | Nonpathogenic | − |
| | Isolate | | | |
| | K-12 wild | ATCC 23716 | Nonpathogenic | − |
| | type | | | |
| E. coli O157:H7 | Human | C7927 | EHEC | + |
| | | 3178-95 | EHEC | + |
| | | G5101 | EHEC | + |
| | | 86-24 | EHEC | + |
| | | 93-111 | EHEC | + |
| | | OK-1 | EHEC | + |
| | | 2886-75 | EHEC | + |
| | Beef | 505B | EHEC | + |
| | | MF 1847 | EHEC | + |
| | Hamburger | EDL933 | EHEC | + |
| E. coli O26:H11 | Not known | H19 | EHEC | + |
| | Human | 97-3250 | EHEC | − |
| E. coli O26 | Human | DEC10C | EHEC | + |
| | | TB285C | EHEC | + |
| | | VP30 | EHEC | + |
| | | DEC9A | EHEC | + |
| | | DEC9F | EHEC | − |
| E. coli O45:H2 | Human | M103-19 | EHEC | − |
| | | MIO1-88 | EHEC | − |
| | | MI05-14 | EHEC | − |
| E. coli O45:HNM | Human | DA-21 | EHEC | − |
| E. coli O45 | Human | DEC11C | EHEC | − |
| | | 5431-72 | EHEC | − |
| | | 4309-72 | EHEC | − |
| | Cow | 88-4110-H | EHEC | − |
| | | D88-28058 | EHEC | − |
| | Pig | 2566-58 | EHEC | − |
| E. coli O103:H2 | Human | MT#80 | EHEC | + |
| E. coli O103:H6 | Human | TB154A | EHEC | + |
| E. coli O103:HN | Human | PT91-24 | EHEC | + |
| E. coli O103:H25 | Human | 8419 | EHEC | + |
| E. coli O103 | Human | DA-41 | EHEC | + |
| | | 6:38 | EHEC | + |
| | | DA-55 | EHEC | + |
| | | 87-2931 | EHEC | + |
| | | GS | EHEC | + |
| | | G5550637 | | |
| E. coli O104:H | Cow | TW 01435 | EHEC | − |
| | Human | ECOR-28 | EHEC | − |
| E. coli O104 | Human | E28 | EHEC | − |
| E. coli O111:H2 | Human | RD8 | EHEC | − |
| E. coli O111:H8 | Human | 3215-99 | EHEC | − |
| E. coli O111:H11 | Human | 0201 9611 | EHEC | − |
| E. coli O111:HNM | Human | 3007-85 | EHEC | − |
| E. coli O111 | Human | CL-37 | EHEC | − |
| | | DEC8B | EHEC | − |
| | | TB226A | EHEC | − |
| | | 928/91 | EHEC | − |
| | | 412/55 | EHEC | − |
| | Cow | DEC8C | EHEC | − |
| E. coli O121:H19 | Human | MT#2 | EHEC | − |
| E. coli O121:H[19] | Human | DA5 | EHEC | − |
| E. coli O121 | Human | 87-2914 | EHEC | − |
| | | DA1 | EHEC | − |
| | Not known | 7927+++ | EHEC | − |
| | | PT91-4 | EHEC | − |
| E. coli O145:HNT | Human | IH-16 | EHEC | + |
| | Unknown | D177 | EHEC | + |
| E. coli O145:HNM | Human | 75-83 | EHEC | − |
| | | GS | EHEC | − |
| | | G5578620 | | |
| E. coli O145 | Human | TB269C | EHEC | + |
| | | MT#66 | EHEC | + |
| | | DEC101 | EHEC | − |
| | Cow | BCL73 | EHEC | + |
| | | B6820-C1 | EHEC | + |
| | Not known | 70300885 | EHEC | + |
| | | 6940 | EHEC | + |
| Salmonella | Turkey | 14-7 | Pathogenic | − |
| Enteritidis | | 14-10 | Pathogenic | − |
| | | 14-11 | Pathogenic | − |
| Salmonella | Not known | | Pathogenic | − |
| Newport | | | | |
| Salmonella | Not known | | Nonpathogenic | − |
| bongori | | | | |
| Salmonella | Not known | | Pathogenic | − |
| Thompson | | | | |
| Salmonella | Not known | | Pathogenic | − |
| Bareilly | | | | |
| Salmonella | Chicken | ATCC 14028 | Pathogenic | − |
| Typhimurium | Turkey | I4 | | − |
| | | 788 | | − |
| Shigella sonei | Not known | ATCC 9290 | Pathogenic | − |
| Shigella flexneri | Feces | ATCC 25929 | Pathogenic | − |
| Listeria innocua | Cabbage | ATCC 51742 | Nonpathogenic | − |
| Listeria | Rabbit | ATCC 15313 | Pathogenic | − |
| monocytogenes | | | | |
| Staphylococcus | Wound | ATCC 29213 | Pathogenic | − |
| aureus | Pleural fluid | ATCC 12600 | Pathogenic | − |
| | Not known | ATCC 29737 | Pathogenic | − |
| Bacillus cereus | Not known | ATCC 10876 | Pathogenic | − |
| Bacillus | Food and | ATCC 7050 | Nonpathogenic | − |
| coagulans | beverage | | | |
| Bacillus | Not known | ATCC | Nonpathogenic | − |
| megaterium | | | | |
| Lysinibacillus | Not known | ATCC 4525 | Nonpathogenic | − |
| sphaericus | | | | |

### Example 2: Host Range Study of CAM-21

The host spectrum of CAM-21 was studied based on a reported plaque assay method with some modifications. Ninety strains of bacteria (Table 1) were selected for the study. Briefly, 100 μL of phage suspension (109 PFU/mL) and 100 μL of log-phase host bacteria were mixed with 5 mL of melted top agar. The melted agar was poured onto pre-solidified TSA plates. The plaque formation on the agar plates was determined after incubation at 37° C. for 24 h. SM buffer was utilized as a control.

CAM-21 exhibited a host specificity towards four STEC serotypes (Table 1), forming plaques with 32 out of 90 total bacterial strains tested. CAM-21 was capable to lyse several strains of STEC serotypes, which include E. coli O157:H7 (10/10), O26 (5/7), O103 (9/9) and O145 (8/11). On the contrary, it did not lyse non-O157 STEC, viz., O45, O104, O111, and O121, non-pathogenic E. coli, and other bacterial genera, including Salmonella, Shigella, Listeria, Staphylococcus, and Bacillus.

### Example 3: Lytic Activity of CAM-21

The lytic activity of CAM-21 was evaluated based on a reported method with modifications. One hundred microliters of phage suspensions (103-109 PFU/mL), diluted from the phage stock (109 PFU/mL), were added into 100 μL of E. coli O157:H7 (107 CFU/mL) at various multiplicity of infection (MOI), from 0.0001 to 100, in a 96-well plate before incubating at 37° C. for up to 24 h. SM buffer was utilized as a control. A BioTek Synergy HT multi-mode microplate reader (BioTek Instruments, Inc., Winooski, VT, USA) was used to measure the optical density at a wavelength of 600 nm (OD600) at various time intervals.

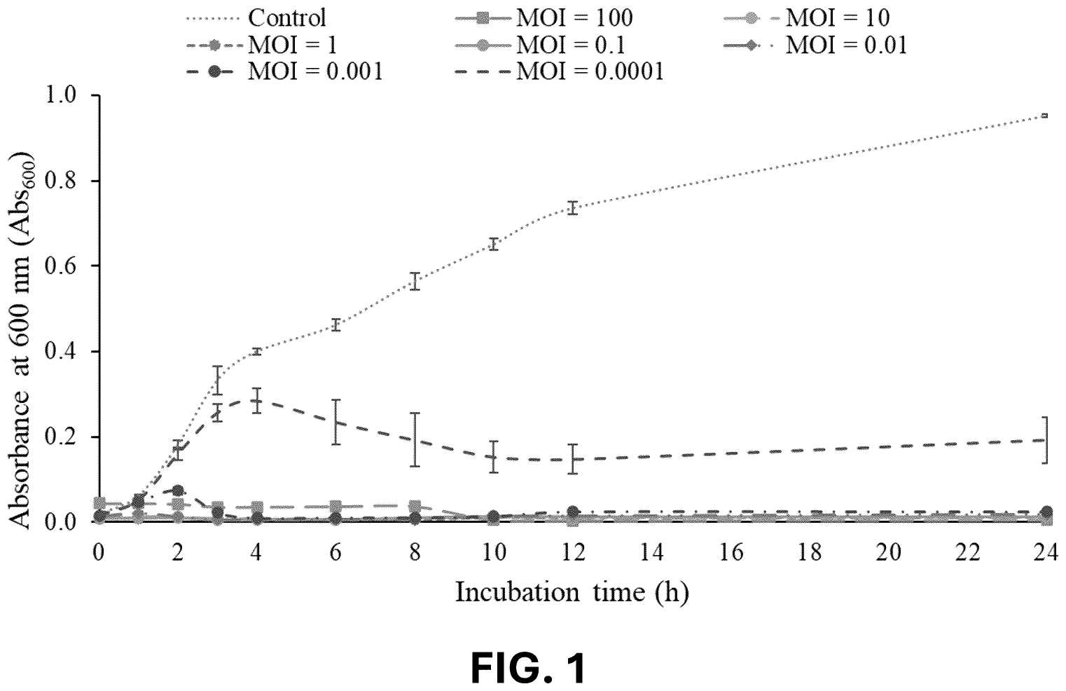

As compared to the control, CAM-21 had a strong inhibitory effect on target bacterial growth at all tested MOI values (FIG. 1). Without exposure to CAM-21, the bacterial cells reached the exponential growth phase after a 2-h incubation, and the OD600 value increased for up to 24 h. With the phage treatment at MOI≥0.001, the bacterial growth was clearly inhibited as all the OD600 values were <0.15 after 24 h of incubation. However, at MOI=0.0001, the OD600 value increased for up to 4 h, followed by a slight reduction to about 0.2, indicating limited growth. However, the OD600 value was still less than that of the phage-free control after 24 h, indicating the strong inhibitory effect of CAM-21 on the target bacterial growth.

### Example 4: TEM Imaging of CAM-21 and Bacterial Lysis

The phage morphology was investigated using transmission electron microscopy (TEM) (JEM-1400, JEOL Ltd., Peabody, MA). Phage particles were concentrated by centrifuging at 46,378×g at 4° C. for 2 h (Model Optima L-90K, Beckman Coulter, Inc., Brea, CA, USA) before resuspending the pellet in SM buffer. A negative staining technique was performed for phage preparation. Five microliters of phage suspension were deposited on copper/carbon grids for 1 min and stained with 2% phosphotungstic acid for 30 s. The grids were air-dried before TEM imaging at 80 kV. The phage size was estimated using an ImageJ software (Version 0.5.3).

To investigate the bacterial lysis by CAM-21, the concentrated phage suspension was first prepared according to the method described. Bacterial cells and phage particles were mixed at a MOI of 1, and incubated for 20 min at 37° C. to allow for the attachment of phage particles to E. coli O157:H7 C7927. Before TEM imaging, the specimen was prepared. Sodium cacodylate (SC) buffer (0.1 M, pH=7.35) containing 2% para-formaldehyde and 2% glutaraldehyde was used to primarily fix the bacterial cells, followed by rinsing with 0.13 M sucrose in SC buffer (0.1M). SC buffer containing 1% OsO₄ was used for a secondary microwave fixation for 1 min, and the sample left for 1 h at 4° C. The specimens were rinsed with SC buffer and distilled water. Aqueous uranyl acetate (1%) was used for en bloc staining before storing the samples overnight at 4° C. The treated samples were rinsed with distilled water, followed by a series of graded dehydration. An Epon resin was used to infiltrate the dehydrated samples for 24 h before polymerization at 60° C. overnight. An ultramicrotome (Ultracut UCT, Leica Microsystems, Germany) and a diamond knife (Diatome, Hatfield, PA, USA) were utilized to prepare thin sections with a thickness of 85 nm before observing with TEM at 80 kV.

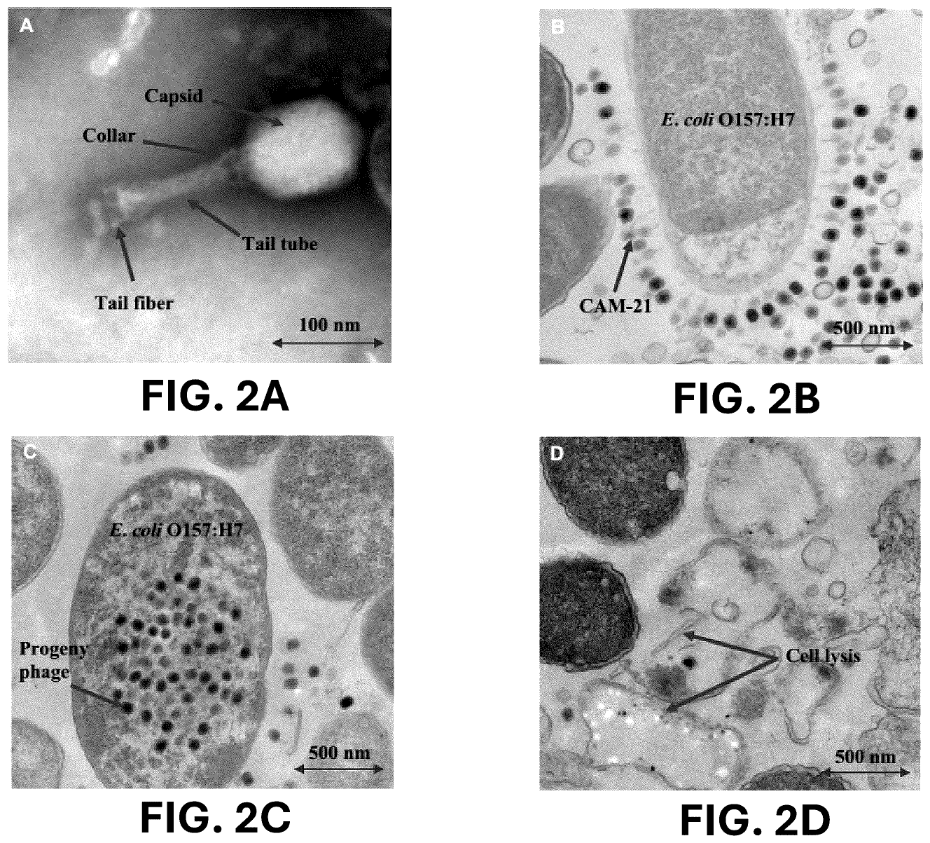

CAM-21 has a polyhedron capsid (diameter=92.83±8.97 nm) and a contractile tail (length=129.75±1.50 nm) (FIG. 2A). Morphological analysis of host lysis showed that infection of target bacteria was initiated by attachment of the phage tail fibers to surface receptors on the cell wall of the bacteria (FIG. 2B). Immature progeny virions were formed within the infected cell (FIG. 2C). Progeny phages were eventually released through bacterial cell lysis (FIG. 2D).

### Example 5: Optimal MOI

The optimal MOI of CAM-21 was evaluated based on an earlier method with some modifications. One hundred microliters of phage suspension and 100 μL of log phase E. coli O157:H7 C7927 (˜10⁸ CFU/mL) were mixed at various MOI values from 0.0001 to 1000. The mixture solutions were added into 800 μL of TSB and incubated in a shaker incubator with a shaking speed of 180 rpm for 4 h at 37° C. The cultures were centrifuged at 9300×g for 15 min before phage titers were evaluated using the double-layer agar technique. The optimal MOI of the phage is the one with the highest phage count.

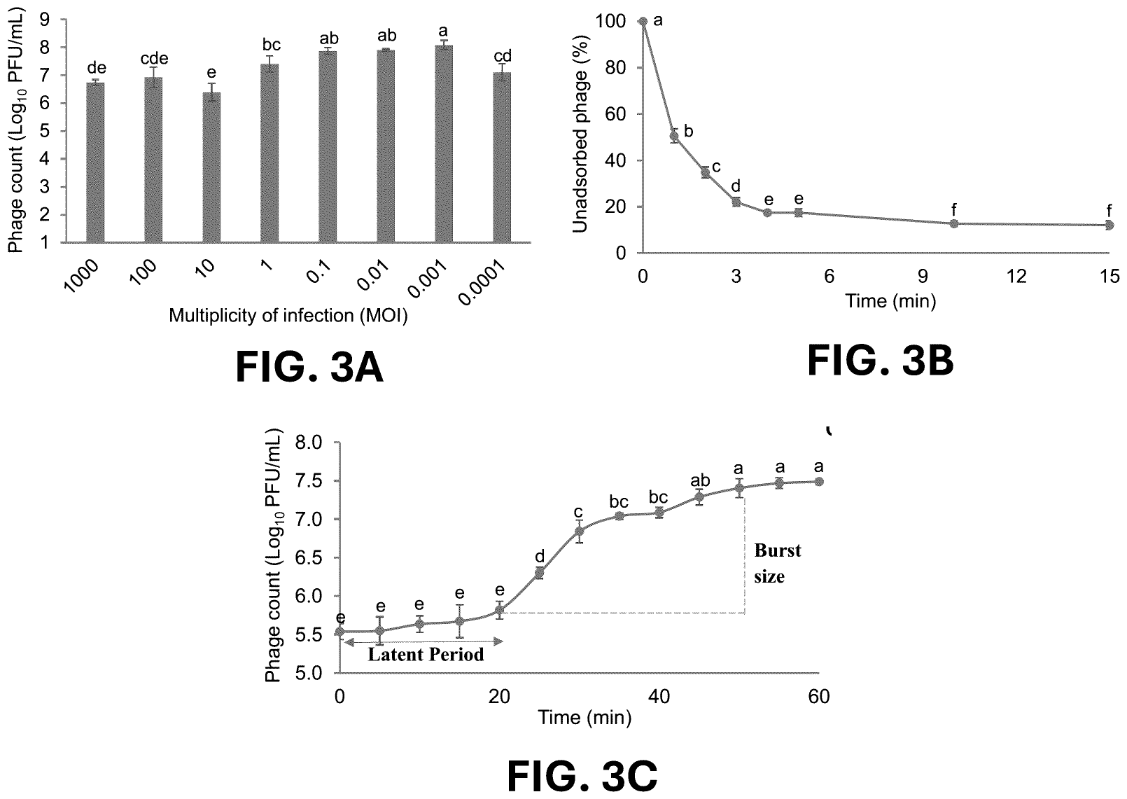

At a MOI of 0.001, CAM-21 produced the highest phage titer, which was 8.08 log PFU/mL, indicating the optimal MOI of this phage (FIG. 3A). However, the phage titer was not significantly different (P>0.05) from those at MOI of 0.1 and 0.01, which were 7.87 and 7.91 log PFU/mL, respectively. The findings revealed that the bacterial infection was still effective even when at a lower phage count.

### Example 6: Phage Adsorption Study and One-Step Growth

The adsorption study of CAM-21 was performed. One milliliter of a freshly grown (3 h) culture of E. coli O157:H7 (10⁸ CFU/mL) was centrifuged at 9300×g for 5 min to obtain a cell pellet that was resuspended in 900 μl of 1× phosphate-buffered saline (PBS). Then, 100 μL of phage suspension (106 PFU/mL) was added, followed by incubation at 37° C. At intervals of 1 min for the first 5 min and 5 min for the next 10 min, a 100 μL aliquot was collected and added into 900 μL of PBS. The suspension was centrifuged at 13,400×g for 2 min. The unabsorbed phage count was determined using the double-layer agar method. The proportion of phage count to the initial phage count at each time interval was expressed as the percentage of unabsorbed phage.

One-step growth of CAM-21 was evaluated. Briefly, 1 mL of a freshly grown (3 h) culture of E. coli O157:H7 (10⁸ CFU/mL) was centrifuged at 9300×g for 5 min to obtain a cell pellet before resuspending it in 1 mL of PBS. Next, 1 mL of phage suspension (105 PFU/mL) was mixed with the bacterial suspension to achieve a MOI of 0.001. The mixture solution was incubated for 15 min at 37° C., followed by centrifugation at 9300×g for 5 min. The cell pellet was washed twice using TSB under the same centrifugation conditions, and resuspended in 10 mL of TSB. The sample was incubated in a shaker incubator while shaking at a speed of 180 rpm at 37° C. For the measurement of phage number, 100 μL of aliquot were collected at every 10 min for 2 h and plated using the double-layer agar method. The latent time and burst size were estimated using the growth curve of CAM-21.

The growth characteristics of CAM-21 were evaluated using adsorption and one-step growth analysis at an optimum MOI of 0.001. After 1 min of incubation, approximately 49% of the phage had adsorbed to the host bacteria, as compared to the original population (FIG. 3B). As the suspension was incubated for a longer period, the percentage of unabsorbed phage was decreased. After 15 min of incubation, about 88% of the initial population was absorbed to the host cells. Based on the one-step growth curve, the burst size and latent time of CAM-21 were approximately 69 PFU/infected cell and 20 min, respectively (FIG. 3C).

### Example 7: Genomic DNA Analysis

CAM-21 genomic DNA extraction was performed using a phage DNA isolation kit (Norgen Biotek, Corp., Thorold, ON, Canada) based on the manufacturer's instructions. Whole genome sequencing (WGS) was conducted using the MiSeq sequencing platform (Illumina, Inc., San Diego, CA, USA). The high-quality filtered reads were assembled using Unicycler. The open reading frames (ORF) were predicted and annotated using the RAST server and BLASTP analysis (https://blast.ncbi.nlm.nih.gov/Blast.cgi). Potential tRNA genes were predicted using tRNAscan-SE. The complete genomic sequence of CAM-21 was deposited in GenBank (accession number OP611477). The potential virulence and antimicrobial resistance factors of CAM-21 were screened using VirulenceFinder-2.0 and ResFinder-4.1, respectively. Screenings for genes related to lysogenic activity and allergenicity were conducted using the databases of PHASTER and AllergenOnline (Version 21), respectively. The CGView server was used to construct a circular genome map of CAM-21. To construct the phylogenetic trees, 30 bacteriophages were chosen from the subfamily, Tevenvirinae, including CAM-21. First, Virus Classification and Tree Building Online Resource (VICTOR) was used to develop a phylogenetic tree based on the whole genome sequences of these phages. The phylogenetic trees were also constructed based on the major capsid protein and terminase large subunit of the bacteriophages by the neighbor-joining technique with a bootstrap value of 1000 after the multiple protein sequence alignment was performed, using the Mega software (Version 11). Using EasyFig (Version 2.2.5), a multiple gene alignment was performed to compare the genome of CAM-21 to its closely related phages, including Escherichia phage wv7, vB_EcoM_BECP11, vB_EcoM_UFV09, and T4.

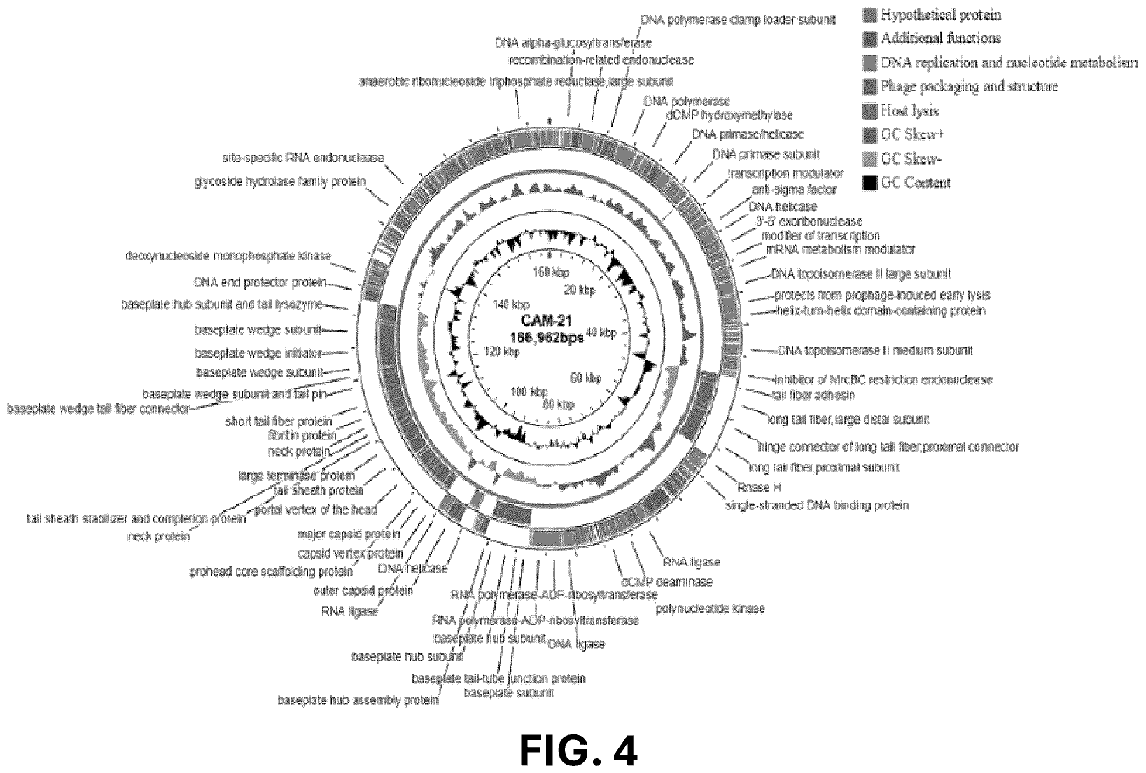

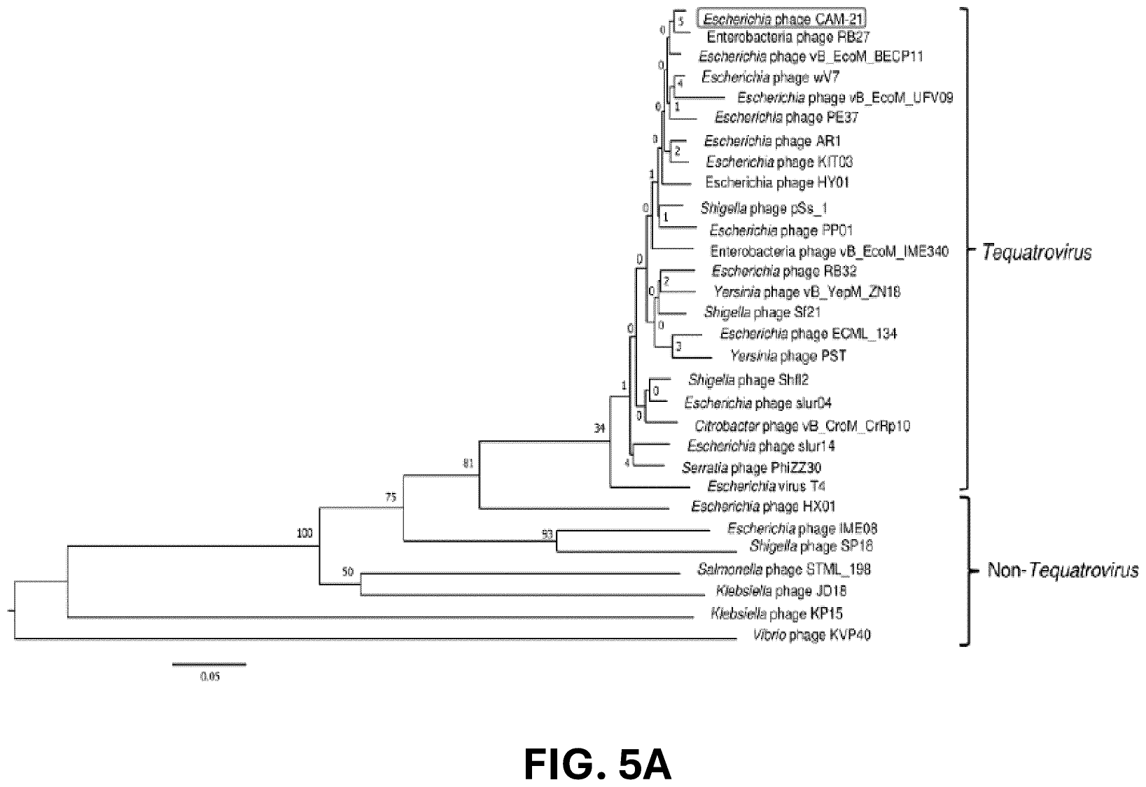

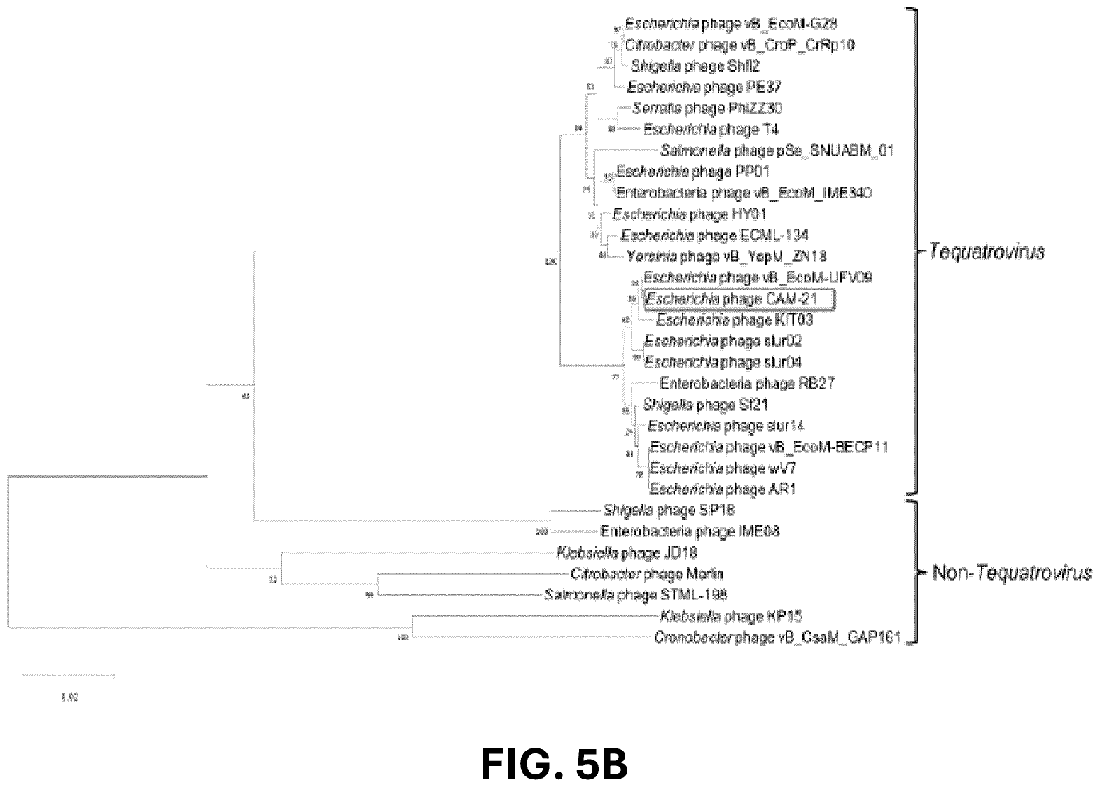

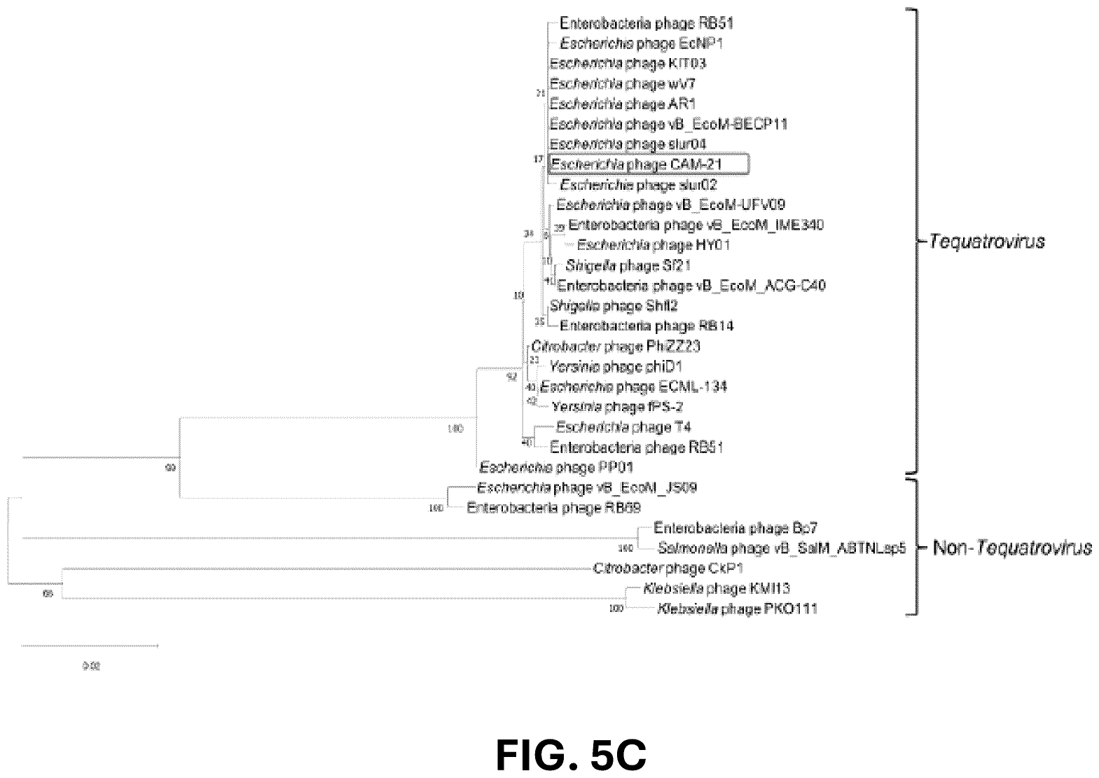

CAM-21 was found to have dsDNA with a genome size of 166,962-bp and a total GC content of 35.49%. The phage genome was predicted to encode 265 ORFs and 11 tRNAs (Table 2). Based on the gene annotation results, 122 ORFs were predicted to be functional, while 143 ORFs were assumed to be hypothetical proteins with unknown functions (Table 3). The putative functional ORFs were classified into four groups, such as host lysis (e.g., tail lysozyme and holin), DNA replication and nucleotide metabolism (e.g., endonuclease, exonuclease, DNA and RNA polymerase, DNA primase, DNA helicase, DNA and RNA ligase, dCMP deaminase, dUTP diphosphatase, and dCMP hydroxymethylase), phage packaging and structure (e.g., head, outer and major capsid, neck, tail fiber, tube and sheath, baseplate hub and wedge, terminase, and fibritin proteins), and additional functions (e.g., thymidylate synthase, thymidine kinase, peptidase, and glutaredoxin) (FIG. 4). From the similarity analysis of amino acid or nucleotide sequences using all the above-mentioned databases, the results indicated that the genome of CAM-21 did not encode toxins, virulence factors, antibiotic resistance, lysogeny and allergens. Based on BLASTN analysis, the gene sequence of CAM-21 showed a high homology with the Myoviridae family phages, which include Escherichia phage vB_EcoM-BECP11 (accession number MW286157; 97% query coverage, and 97% identity), vB_EcoM-UFV09 (accession number MZ291552, 95% query coverage, and 97% identity), and wV7 (accession number HM997020, 96% query coverage, and 98% identity). The classification of CAM-21 was confirmed by a phylogenetic tree analysis. Based on the complete genome, CAM-21 was clustered with Tequatrovirus genus phages, such as RB27, vB_EcoM-BECP11, wV7, vB_EcoM-UFV09, and PE37 (FIG. 5A). Based on FIG. 5B, the major capsid protein of CAM-21 was closely related to those of ten Tequatrovirus phages. For the phylogenetic study based on the large terminase subunit, CAM-21 was clustered with eight Tequatrovirus phages, such as RB51, EcNP1, KIT03, wV7, AR1, vB_EcoM-BECP11, slur04, and slur02 (FIG. 5C), indicating that these phages have a similar DNA packaging process. All references to tRNA start and Bounds end and ORF “Start” and “End” are relative to SEQ ID NO. 1.

TABLE 2
The predicted tRNA genes in the Escherichia phage CAM-21

| No. of tRNA | tRNA start | Bounds end | Type of tRNA | Anti-codon |
|---|---|---|---|---|
| 1 | 135677 | 135749 | glutamine (Gln) | TTG |
| 2 | 135751 | 135837 | leucine (Leu) | TAA |
| 3 | 135843 | 135916 | glycine (Gly) | TCC |
| 4 | 135927 | 136000 | proline (Pro) | TGG |
| 5 | 136003 | 136092 | serine (Ser) | TGA |
| 6 | 136098 | 136173 | threonine (Thr) | TGT |
| 7 | 136175 | 136249 | methionine (Met) | CAT |
| 8 | 136262 | 136348 | tyrosine (Tyr) | GTA |
| 9 | 136353 | 136427 | asparagine (Asn) | GTT |
| 10 | 136542 | 136617 | histidine (His) | GTG |
| 11 | 136622 | 136697 | arginine (Arg) | TCT |

TABLE 3
gp37 and gp38 in CAM-21 & T4-like phages

| | | | | Length | | Iden- | | | | | |
|---|---|---|---|---|---|---|---|---|---|---|---|
| | | | | (no. of | | tity | | | Iden- | | |
| | | | | amino | Gene | with T4 | Putative | Most closely | tity | E | |
| ORF | Strand | Start | End | acid) | name | (%) | function | related protein | (%) | value | Accession no. |
| 1 | − | 2 | 265 | 87 | cam01_001 | — | hypothetical | hypothetical | 100 | 8E−60 | YP_009197386.1 |
| | | | | | | | protein | protein | | | |
| | | | | | | | | AVU02_gp198 | | | |
| | | | | | | | | [Escherichia | | | |
| | | | | | | | | phage slur07] | | | |
| 2 | − | 344 | 901 | 185 | 55 | 100 | RNA | RNA polymerase | 100 | 3E−135 | YP_009197387.1 |
| | | | | | | | polymerase | sigma factor | | | |
| | | | | | | | sigma factor | [Escherichia | | | |
| | | | | | | | | phage slur07] | | | |
| 3 | − | 915 | 1103 | 62 | a-gt.5 | 98 | hypothetical | hypothetical | 100 | 2E−40 | YP_009290332.1 |
| | | | | | | | protein | protein | | | |
| | | | | | | | | BIZ64_gp066 | | | |
| | | | | | | | | [Escherichia | | | |
| | | | | | | | | phage vB_EcoM- | | | |
| | | | | | | | | UFV13] | | | |
| 4 | − | 1105 | 1422 | 105 | a-gt.4 | 99 | hypothetical | a-gt.4 family | 99 | 5E−65 | YP_009167875.1 |
| | | | | | | | protein | protein | | | |
| | | | | | | | | [Escherichia | | | |
| | | | | | | | | phage AR1] | | | |
| 5 | − | 1598 | 1771 | 57 | a-gt.2 | 91 | hypothetical | hypothetical | 97 | 2E−33 | YP_009102267.1 |
| | | | | | | | protein | protein RB27_062 | | | |
| | | | | | | | | [Enterobacteria | | | |
| | | | | | | | | phage RB27] | | | |
| 6 | − | 1838 | 3040 | 400 | a-gt | 91 | alpha- | DNA alpha- | 100 | 7E−304 | YP_009197391.1 |
| | | | | | | | glucosyl- | glucosyltransferase | | | |
| | | | | | | | transferase | [Escherichia | | | |
| | | | | | | | | phage slur07] | | | |
| 7 | − | 3217 | 4236 | 339 | 47 | 99 | metallo- | metallopho- | 100 | 4E−258 | YP_009284129.1 |
| | | | | | | | phosphoesterase | sphoesterase | | | |
| | | | | | | | | [Escherichia | | | |
| | | | | | | | | phage HY03] | | | |
| 8 | − | 4233 | 4496 | 87 | 46.2 | 99 | hypothetical | hypothetical | 100 | 2E−64 | YP_009180833.1 |
| | | | | | | | protein | protein | | | |
| | | | | | | | | AS348_gp078 | | | |
| | | | | | | | | [Escherichia | | | |
| | | | | | | | | phage slur14] | | | |
| 9 | − | 4477 | 4683 | 68 | 46.1 | 100 | hypothetical | DUF5487 family | 100 | 6E−46 | YP_009197394.1 |
| | | | | | | | protein | protein | | | |
| | | | | | | | | [Escherichia | | | |
| | | | | | | | | phage slur07] | | | |
| 10 | − | 4722 | 6362 | 546 | 46 | 95 | recombination- | endonuclease | 97 | 0E+00 | YP_002854390.1 |
| | | | | | | | related | subunit [E. virus | | | |
| | | | | | | | endonuclease | RB14] | | | |
| 11 | − | 6418 | 6606 | 62 | 45.2 | 100 | hypothetical | gp45.2 | 100 | 2E−41 | YP_002854389.1 |
| | | | | | | | protein | hypothetical | | | |
| | | | | | | | | protein [E. virus | | | |
| | | | | | | | | RB14] | | | |
| 12 | − | 6616 | 7005 | 129 | rpbA | 98 | RNA | RNA polymerase | 100 | 2E−90 | YP_007004796.1 |
| | | | | | | | polymerase- | binding protein | | | |
| | | | | | | | binding protein | [Escherichia | | | |
| | | | | | | | | phage wV7] | | | |
| 13 | − | 7061 | 7747 | 228 | 45 | 100 | sliding clamp | gp45 sliding | 100 | 2E−159 | YP_009197398.1 |
| | | | | | | | | clamp DNA | | | |
| | | | | | | | | polymerase | | | |
| | | | | | | | | [Escherichia | | | |
| | | | | | | | | phage slur07] | | | |
| 14 | − | 7799 | 8758 | 319 | 44 | 100 | DNA polymerase | clamp loader, | 100 | 1E−234 | YP_002854386.1 |
| | | | | | | | clamp loader | small subunit [E. | | | |
| | | | | | | | subunit | virus RB14] | | | |
| 15 | − | 8760 | 9323 | 187 | 62 | 99 | DNA polymerase | DNA polymerase | 100 | 6E−134 | YP_002854385.1 |
| | | | | | | | clamp loader | clamp loader | | | |
| | | | | | | | subunit | subunit A [E. virus | | | |
| | | | | | | | | RB14] | | | |
| 16 | − | 9325 | 9693 | 122 | regA | 100 | translational | translational | 100 | 3E−89 | YP_009210240.1 |
| | | | | | | | repressor | repressor RegA | | | |
| | | | | | | | | [Escherichia | | | |
| | | | | | | | | phage slur02] | | | |
| 17 | − | 9695 | 9916 | 73 | cam01_017 | — | hypothetical | hypothetical | 100 | 3E−46 | YP_002854383.1 |
| | | | | | | | protein | protein [E. virus | | | |
| | | | | | | | | RB14] | | | |
| 18 | − | 9995 | 12691 | 898 | 43 | 100 | DNA | DNA polymerase | 100 | 0E+00 | YP_007004790.1 |
| | | | | | | | polymerase | [Escherichia | | | |
| | | | | | | | | phage wV7] | | | |
| 19 | − | 12875 | 13111 | 78 | cam01_019 | — | hypothetical | hypothetical | 97 | 2E−46 | YP_009167860.1 |
| | | | | | | | protein | protein AR1_049 | | | |
| | | | | | | | | [Escherichia | | | |
| | | | | | | | | phage AR1] | | | |
| 20 | − | 13122 | 13502 | 126 | imm.1 | 97 | hypothetical | membrane | 98 | 4E−89 | YP_009965537.1 |
| | | | | | | | protein | protein | | | |
| | | | | | | | | [Escherichia | | | |
| | | | | | | | | phage CF2] | | | |
| 21 | − | 13510 | 13761 | 83 | imm | 99 | superinfection | superinfection | 100 | 1E−51 | YP_009197406.1 |
| | | | | | | | immunity | immunity protein | | | |
| | | | | | | | protein | [Escherichia | | | |
| | | | | | | | | phage slur07] | | | |
| 22 | − | 13915 | 14655 | 246 | 42 | 97 | dCMP | dCMP | 100 | 4E−186 | YP_009102249.1 |
| | | | | | | | hydroxymethylase | hydroxymethylase | | | |
| | | | | | | | | [Enterobacteria | | | |
| | | | | | | | | phage RB27] | | | |
| 23 | − | 14652 | 15494 | 280 | cam01_023 | — | hypothetical | beta-glucosyl- | 99 | 2E−208 | YP_009167855.1 |
| | | | | | | | protein | HMC-alpha- | | | |
| | | | | | | | | glucosyl- | | | |
| | | | | | | | | transferase | | | |
| | | | | | | | | [Escherichia | | | |
| | | | | | | | | phage AR1] | | | |
| 24 | − | 15572 | 16753 | 393 | uvsX | 91 | RecA-like | RecA-like | 100 | 6E−283 | YP_009210233.1 |
| | | | | | | | recombination | recombination | | | |
| | | | | | | | protein | protein | | | |
| | | | | | | | | [Escherichia | | | |
| | | | | | | | | phage slur02] | | | |
| 25 | − | 16746 | 17090 | 114 | 40 | 100 | head vertex | gp40 head vertex | 100 | 6E−74 | YP_009210232.1 |
| | | | | | | | assembly | assembly | | | |
| | | | | | | | chaperone | chaperone | | | |
| | | | | | | | | [Escherichia | | | |
| | | | | | | | | phage slur02] | | | |
| 26 | − | 17100 | 18527 | 475 | 41 | 100 | DNA primase/ | DNA primase- | 100 | 0E+00 | YP_009965544.1 |
| | | | | | | | helicase | helicase subunit | | | |
| | | | | | | | | [Escherichia | | | |
| | | | | | | | | phage CF2] | | | |
| 27 | − | 18586 | 18768 | 60 | dmd | 98 | discriminator | discriminator | 100 | 7E−40 | YP_002854373.1 |
| | | | | | | | of mRNA | of mRNA | | | |
| | | | | | | | degradation | degradation [E. | | | |
| | | | | | | | | virus RB14] | | | |
| 28 | − | 18770 | 19027 | 85 | 61.4 | 96 | hypothetical | hypothetical | 100 | 8E−59 | YP_009288406.1 |
| | | | | | | | protein | protein | | | |
| | | | | | | | | BI058_gp040 | | | |
| | | | | | | | | [Shigella phage | | | |
| | | | | | | | | SHBML-50-1] | | | |
| 29 | − | 19088 | 19381 | 97 | sp | 93 | spackle | spackle | 100 | 3E−66 | YP_009167406.1 |
| | | | | | | | periplasmic | periplasmic | | | |
| | | | | | | | protein | protein | | | |
| | | | | | | | | [Enterobacteria | | | |
| | | | | | | | | phage RB68] | | | |
| 30 | − | 19383 | 20015 | 210 | 61.2 | 79 | hypothetical | hypothetical | 93 | 5E−152 | YP_009102241.1 |
| | | | | | | | protein | protein RB27_036 | | | |
| | | | | | | | | [Enterobacteria | | | |
| | | | | | | | | phage RB27] | | | |
| 31 | − | 20017 | 20373 | 118 | 61.2 | 35 | hypothetical | hypothetical | 100 | 3E−82 | YP_009210226.1 |
| | | | | | | | protein | protein | | | |
| | | | | | | | | AVU04_gp141 | | | |
| | | | | | | | | [Escherichia | | | |
| | | | | | | | | phage slur02] | | | |
| 32 | − | 20375 | 20539 | 54 | 61.1 | 96 | hypothetical | hypothetical | 100 | 3E−31 | YP_009197417.1 |
| | | | | | | | protein | protein | | | |
| | | | | | | | | AVU02_gp167 | | | |
| | | | | | | | | [Escherichia | | | |
| | | | | | | | | phage slur07] | | | |
| 33 | − | 20542 | 21570 | 342 | 61 | 99 | DNA primase | putative DNA | 100 | 4E−260 | YP_009281373.1 |
| | | | | | | | subunit | primase | | | |
| | | | | | | | | [Escherichia | | | |
| | | | | | | | | phage UFV- | | | |
| | | | | | | | | AREG1] | | | |
| 34 | + | 21567 | 21767 | 66 | cam01_034 | — | hypothetical | hypothetical | 99 | 1E−37 | YP_009197419.1 |
| | | | | | | | protein | protein | | | |
| | | | | | | | | AVU02_gp165 | | | |
| | | | | | | | | [Escherichia | | | |
| | | | | | | | | phage slur07] | | | |
| 35 | − | 21839 | 22357 | 172 | 56 | 73 | dUTP | dCTP | 100 | 6E−127 | YP_002853986.1 |
| | | | | | | | diphosphatase | pyrophosphatase | | | |
| | | | | | | | | [Enterobacteria | | | |
| | | | | | | | | phage RB51] | | | |
| 36 | − | 22357 | 22497 | 46 | cam01_036 | — | hypothetical | hypothetical | 94 | 7E−21 | YP_007004772.1 |
| | | | | | | | protein | protein | | | |
| | | | | | | | | F412_gp244 | | | |
| | | | | | | | | [Escherichia | | | |
| | | | | | | | | phage wV7] | | | |
| 37 | − | 22539 | 22775 | 78 | soc | 54 | small outer | capsid and | 100 | 6E−53 | YP_009279024.1 |
| | | | | | | | capsid protein | scaffold protein | | | |
| | | | | | | | | [Shigella phage | | | |
| | | | | | | | | SHFML-26] | | | |
| 38 | − | 22874 | 23080 | 68 | mrh.2 | 99 | hypothetical | hypothetical | 99 | 6E−46 | YP_009197424.1 |
| | | | | | | | protein | protein | | | |
| | | | | | | | | AVU02_gp160 | | | |
| | | | | | | | | [Escherichia | | | |
| | | | | | | | | phage slur07] | | | |
| 39 | − | 23080 | 23421 | 113 | mrh.1 | 97 | hypothetical | hypothetical | 99 | 1E−73 | YP_009167840.1 |
| | | | | | | | protein | protein AR1_029 | | | |
| | | | | | | | | [Escherichia | | | |
| | | | | | | | | phage AR1] | | | |
| 40 | − | 23430 | 23915 | 160 | mrh | 91 | transcription | transcription | 91 | 4E−101 | NP_049641.1 |
| | | | | | | | modulator | modulator under | | | |
| | | | | | | | | heat shock [E. | | | |
| | | | | | | | | virus T4] | | | |
| 41 | − | 23938 | 24093 | 51 | srh | 100 | transcription | transcription | 100 | 4E−29 | YP_006986574.1 |
| | | | | | | | modulator | modulator | | | |
| | | | | | | | | [Escherichia | | | |
| | | | | | | | | phage | | | |
| | | | | | | | | vB_EcoM_ACG- | | | |
| | | | | | | | | C40] | | | |
| 42 | − | 24090 | 24254 | 54 | modA.4 | 94 | hypothetical | hypothetical | 98 | 8E−32 | YP_002853980.1 |
| | | | | | | | protein | protein | | | |
| | | | | | | | | [Enterobacteria | | | |
| | | | | | | | | phage RB51] | | | |
| 43 | − | 24247 | 24717 | 156 | modA.3 | 97 | hypothetical | hypothetical | 98 | 1E−109 | YP_007004405.1 |
| | | | | | | | protein | protein | | | |
| | | | | | | | | F413_gp217 | | | |
| | | | | | | | | [Escherichia | | | |
| | | | | | | | | phage ime09] | | | |
| 44 | − | 24726 | 24908 | 60 | modA.2 | 98 | hypothetical | hypothetical | 100 | 5E−40 | YP_009210213.1 |
| | | | | | | | protein | protein | | | |
| | | | | | | | | AVU04_gp128 | | | |
| | | | | | | | | [Escherichia | | | |
| | | | | | | | | phage slur02] | | | |
| 45 | − | 24976 | 25599 | 207 | modB | 95 | ADP- | NAD-protein | 99 | 1E−145 | YP_009277663.1 |
| | | | | | | | ribosylase | ADP- | | | |
| | | | | | | | | ribosyltransferase | | | |
| | | | | | | | | modA [Shigella | | | |
| | | | | | | | | phage SHFML- | | | |
| | | | | | | | | 11] | | | |
| 46 | − | 25596 | 26198 | 200 | modA | 97 | ADP- | putative ADP- | 99 | 4E−143 | YP_009148470.1 |
| | | | | | | | ribosylase | ribosylase | | | |
| | | | | | | | | [Escherichia | | | |
| | | | | | | | | phage HY011 | | | |
| 47 | − | 26315 | 27061 | 248 | srd | 99 | anti-sigma | anti-sigma factor | 100 | 1E−174 | YP_009965566.1 |
| | | | | | | | factor | [Escherichia | | | |
| | | | | | | | | phage CF2] | | | |
| 48 | − | 27063 | 27374 | 103 | dda.1 | 92 | hypothetical | hypothetical | 100 | 1E−73 | YP_007004760.1 |
| | | | | | | | protein | protein | | | |
| | | | | | | | | F412_gp256 | | | |
| | | | | | | | | [Escherichia | | | |
| | | | | | | | | phage wV7] | | | |
| 49 | − | 27371 | 28690 | 439 | dda | 98 | DNA helicase | DNA helicase | 100 | 0E+00 | YP_009625233.1 |
| | | | | | | | | [Escherichia | | | |
| | | | | | | | | phage slur03] | | | |
| 50 | − | 28697 | 28957 | 86 | cam01_050 | — | hypothetical | hypothetical | 100 | 5E−56 | YP_009619221.1 |
| | | | | | | | protein | protein | | | |
| | | | | | | | | FDJ03_gp141 | | | |
| | | | | | | | | [Shigella phage | | | |
| | | | | | | | | Sf24] | | | |
| 51 | − | 28944 | 29189 | 81 | dexA.2 | 91 | hypothetical | dextranase | 98 | 4E−51 | YP_009210207.1 |
| | | | | | | | protein | [Escherichia | | | |
| | | | | | | | | phage slur02] | | | |
| 52 | − | 29182 | 29424 | 80 | dexA.1 | 93 | hypothetical | hypothetical | 96 | 1E−50 | YP_009098401.1 |
| | | | | | | | protein | protein RB3_015 | | | |
| | | | | | | | | [Escherichia | | | |
| | | | | | | | | phage RB3] | | | |
| 53 | − | 29424 | 30107 | 227 | dexA | 100 | 3′-5′ | exonuclease [E. | 100 | 5E−171 | NP_049629.1 |
| | | | | | | | exoribonuclease | virus T4] | | | |
| 54 | − | 30171 | 30674 | 167 | motB.2 | 91 | hypothetical | hypothetical | 98 | 1E−122 | YP_009614935.1 |
| | | | | | | | protein | protein [Shigella | | | |
| | | | | | | | | phage Sf22] | | | |
| 55 | − | 30687 | 31181 | 164 | motB.1 | 38 | hypothetical | hypothetical | 99 | 2E−119 | YP_009608391.1 |
| | | | | | | | protein | protein | | | |
| | | | | | | | | FDJ03_gp136 | | | |
| | | | | | | | | [Shigella phage | | | |
| | | | | | | | | Sf24] | | | |
| 56 | − | 31254 | 31727 | 157 | motB | 46 | modifier of | modifier of | 99 | 2E−108 | YP_009098397.1 |
| | | | | | | | transcription | transcription | | | |
| | | | | | | | | [Escherichia | | | |
| | | | | | | | | phage RB3] | | | |
| 57 | − | 31737 | 32156 | 139 | motB | 37 | modifier of | modifier of | 100 | 1E−98 | YP 009098396.1 |
| | | | | | | | transcription | transcription | | | |
| | | | | | | | | [Escherichia | | | |
| | | | | | | | | phage RB3] | | | |
| 58 | − | 32264 | 32533 | 89 | cam01_058 | — | hypothetical | hypothetical | 100 | 4E−61 | YP_007004391.1 |
| | | | | | | | protein | protein | | | |
| | | | | | | | | F413_gp267 | | | |
| | | | | | | | | [Escherichia | | | |
| | | | | | | | | phage ime09] | | | |
| 59 | − | 32547 | 32762 | 71 | cef | 97 | modifier of | modifier of | 100 | 9E−50 | YP_002853963.1 |
| | | | | | | | supressor | suppressor T4 | | | |
| | | | | | | | tRNAs | tRNAs | | | |
| | | | | | | | | [Enterobacteria | | | |
| | | | | | | | | phage RB51] | | | |
| 60 | − | 32762 | 33187 | 141 | goF | 84 | mRNA | mRNA | 99 | 2E−92 | YP_009167376.1 |
| | | | | | | | metabolism | metabolism | | | |
| | | | | | | | modulator | moderator | | | |
| | | | | | | | | [Enterobacteria | | | |
| | | | | | | | | phage RB68] | | | |
| 61 | − | 33190 | 33366 | 58 | 39.2 | 70 | hypothetical | hypothetical | 100 | 9E−42 | YP_007004748.1 |
| | | | | | | | protein | protein | | | |
| | | | | | | | | [Escherichia | | | |
| | | | | | | | | phage wV7] | | | |
| 62 | − | 33369 | 33743 | 124 | cam01_062 | — | hypothetical | hypothetical | 99 | 1E−82 | YP_007004747.1 |
| | | | | | | | protein | protein | | | |
| | | | | | | | | F412_gp269 | | | |
| | | | | | | | | [Escherichia | | | |
| | | | | | | | | phage wV7] | | | |
| 63 | − | 33740 | 34009 | 89 | 39.1 | 98 | hypothetical | hypothetical | 99 | 2E−60 | YP_009167816.1 |
| | | | | | | | protein | protein AR1_004 | | | |
| | | | | | | | | [Escherichia | | | |
| | | | | | | | | phage AR1] | | | |
| 64 | − | 34079 | 35896 | 605 | 39 | 98 | DNA | DNA | 100 | 0E+00 | YP_009618817.1 |
| | | | | | | | topoisomerase | topoisomerase II | | | |
| | | | | | | | II large | [Shigella phage | | | |
| | | | | | | | subunit | Sf21] | | | |
| 65 | − | 35951 | 36154 | 67 | rllA.1 | 99 | hypothetical | hypothetical | 100 | 1E−41 | YP_009625491.1 |
| | | | | | | | protein | protein | | | |
| | | | | | | | | FDJ55_gp132 | | | |
| | | | | | | | | [Escherichia | | | |
| | | | | | | | | phage slur04] | | | |
| 66 | − | 36165 | 38342 | 725 | rllA | 98 | protects from | rllA protein | 100 | 0E+00 | YP_007004743.1 |
| | | | | | | | prophage- | [Escherichia | | | |
| | | | | | | | induced early | phage wV7] | | | |
| | | | | | | | lysis | | | | |
| 67 | − | 38354 | 39292 | 312 | rllB | 99 | helix-turn- | helix-turn-helix | 99 | 4E−227 | YP_009149516.1 |
| | | | | | | | helix domain- | domain- | | | |
| | | | | | | | containing | containing protein | | | |
| | | | | | | | protein | [Yersinia phage | | | |
| | | | | | | | | phiD1] | | | |
| 68 | − | 39321 | 39515 | 64 | denB.1 | 98 | hypothetical | hypothetical | 100 | 2E−37 | YP_009098658.1 |
| | | | | | | | protein | protein RB3_272 | | | |
| | | | | | | | | [Escherichia | | | |
| | | | | | | | | phage RB3] | | | |
| 69 | − | 39552 | 39881 | 109 | cam01_069 | — | hypothetical | hypothetical | 99 | 3E−77 | YP_007004648.1 |
| | | | | | | | protein | protein | | | |
| | | | | | | | | F413_gp235 | | | |
| | | | | | | | | [Escherichia | | | |
| | | | | | | | | phage ime09] | | | |
| 70 | − | 39895 | 40371 | 158 | denB | 99 | DNA | endonuclease IV | 100 | 4E−120 | YP_009168090.1 |
| | | | | | | | endonuclease | [Escherichia | | | |
| | | | | | | | IV | phage AR1] | | | |
| 71 | − | 40452 | 40658 | 68 | cam01_071 | — | hypothetical | hypothetical | 100 | 7E−41 | YP_002854606.1 |
| | | | | | | | protein | protein [E. virus | | | |
| | | | | | | | | RB14] | | | |
| 72 | − | 40770 | 40868 | 32 | ndd.5 | 97 | hypothetical | hypothetical | 100 | 9E−17 | YP_009290530.1 |
| | | | | | | | protein | protein | | | |
| | | | | | | | | BIZ64_gp264 | | | |
| | | | | | | | | [Escherichia | | | |
| | | | | | | | | phage vB_EcoM- | | | |
| | | | | | | | | UFV13] | | | |
| 73 | − | 40934 | 41047 | 37 | ndd.4 | 92 | hypothetical | ndd.4 | 97 | 2E−15 | YP_002854603.1 |
| | | | | | | | protein | hypothetical | | | |
| | | | | | | | | protein [E. virus | | | |
| | | | | | | | | RB14] | | | |
| 74 | − | 41055 | 41252 | 65 | ndd.2a | 100 | hypothetical | hypothetical | 100 | 2E−39 | YP_803205.1 |
| | | | | | | | protein | protein | | | |
| | | | | | | | | RB32ORF263c | | | |
| | | | | | | | | [E. virus RB32] | | | |
| 75 | − | 41249 | 41359 | 35 | ndd.2 | 97 | hypothetical | hypothetical | 100 | 7E−21 | YP_009197459.1 |
| | | | | | | | protein | protein | | | |
| | | | | | | | | AVU02_gp125 | | | |
| | | | | | | | | [Escherichia | | | |
| | | | | | | | | phage slur07] | | | |
| 76 | − | 41368 | 41583 | 71 | ndd.1 | 97 | hypothetical | ndd. 1 | 100 | 1E−46 | YP_002854600.1 |
| | | | | | | | protein | hypothetical | | | |
| | | | | | | | | protein [E. virus | | | |
| | | | | | | | | RB14] | | | |
| 77 | − | 41644 | 42102 | 152 | ndd | 98 | Nucleoid | naphthalene 1,2- | 99 | 2E−109 | YP_009102737.1 |
| | | | | | | | disruption | dioxygenase | | | |
| | | | | | | | protein | [Escherichia | | | |
| | | | | | | | | phage ECML- | | | |
| | | | | | | | | 134] | | | |
| 78 | − | 42190 | 42342 | 50 | cam01_078 | — | hypothetical | acridine | 96 | 8E−28 | YP_007005004.1 |
| | | | | | | | protein | resistance protein | | | |
| | | | | | | | | [Escherichia | | | |
| | | | | | | | | phage wV7] | | | |
| 79 | − | 42480 | 43808 | 442 | 52 | 100 | DNA | DNA | 100 | 0E+00 | YP_009210181.1 |
| | | | | | | | topoisomerase | topisomerase II | | | |
| | | | | | | | II medium | medium subunit | | | |
| | | | | | | | subunit | [Escherichia | | | |
| | | | | | | | | phage slur02] | | | |
| 80 | − | 43805 | 43954 | 49 | motA.1 | 100 | hypothetical | hypothetical | 100 | 3E−23 | YP_009210180.1 |
| | | | | | | | protein | protein | | | |
| | | | | | | | | AVU04_gp095 | | | |
| | | | | | | | | [Escherichia | | | |
| | | | | | | | | phage slur02] | | | |
| 81 | − | 44084 | 44719 | 211 | motA | 98 | flagellar | activator of | 100 | 1E−144 | YP_009281594.1 |
| | | | | | | | motor protein | middle period | | | |
| | | | | | | | | transcription | | | |
| | | | | | | | | [Escherichia | | | |
| | | | | | | | | phage UFV- | | | |
| | | | | | | | | AREG1] | | | |
| 82 | − | 44730 | 45059 | 109 | arn.4 | 100 | hypothetical | arn.4 hypothetical | 100 | 3E−78 | YP_002854592.1 |
| | | | | | | | protein | protein [E. virus | | | |
| | | | | | | | | RB14] | | | |
| 83 | − | 45056 | 45517 | 153 | arn.3 | 98 | hypothetical | hypothetical | 99 | 1E−112 | YP_009619063.1 |
| | | | | | | | protein | protein | | | |
| | | | | | | | | FDJ02_gp149 | | | |
| | | | | | | | | [Shigella phage | | | |
| | | | | | | | | Sf21] | | | |
| 84 | − | 45517 | 45813 | 98 | arn.2 | 97 | hypothetical | hypothetical | 100 | 2E−69 | YP_009619062.1 |
| | | | | | | | protein | protein | | | |
| | | | | | | | | FDJ02_gp150 | | | |
| | | | | | | | | [Shigella phage | | | |
| | | | | | | | | Sf21] | | | |
| 85 | − | 45884 | 46015 | 43 | arn.1 | 98 | hypothetical | arn.1 hypothetical | 100 | 4E−28 | YP_002854588.1 |
| | | | | | | | protein | protein [E. virus | | | |
| | | | | | | | | RB14] | | | |
| 86 | − | 46099 | 46377 | 92 | arn | 98 | inhibitor of | inhibitor of MrcBC | 100 | 3E−61 | YP_009965608.1 |
| | | | | | | | MrcBC | restriction | | | |
| | | | | | | | restriction | endonuclease | | | |
| | | | | | | | endonuclease | [Escherichia | | | |
| | | | | | | | | phage CF2] | | | |
| 87 | − | 46374 | 46526 | 50 | asiA.1 | 94 | hypothetical | anti-sigma factor | 98 | 7E−27 | YP_009288615.1 |
| | | | | | | | protein | [Shigella phage | | | |
| | | | | | | | | SHBML-50-1] | | | |
| 88 | − | 46539 | 46811 | 90 | asiA | 100 | anti-sigma 70 | asiA anti-sigma | 100 | 7E−58 | YP_009210171.1 |
| | | | | | | | protein | 70 protein | | | |
| | | | | | | | | [Escherichia | | | |
| | | | | | | | | phage slur02] | | | |
| 89 | + | 46812 | 47468 | 218 | t | 98 | holin lysis | holin [E. virus | 100 | 6E−157 | YP_803187.1 |
| | | | | | | | mediator | RB32] | | | |
| 90 | + | 47501 | 48277 | 258 | 38 | — | tail fiber | receptor- | 99 | 2E−178 | YP_010070631.1 |
| | | | | | | | adhesin | recognizing protein | | | |
| | | | | | | | | [Escherichia | | | |
| | | | | | | | | phage | | | |
| | | | | | | | | vB_EcoM_G4507] | | | |
| 91 | + | 48309 | 51620 | 1103 | 37 | 35 | long tail fiber, | long tail fiber | 97 | 0E+00 | YP_010076447.1 |
| | | | | | | | large distal | subunit, distal | | | |
| | | | | | | | subunit | [Shigella phage | | | |
| | | | | | | | | Sf23] | | | |
| 92 | + | 51629 | 52285 | 218 | 36 | 79 | hinge | hinge connector | 99 | 4E−157 | YP_009168068.1 |
| | | | | | | | connector of | of long tail fiber | | | |
| | | | | | | | long tail fiber, | distal connector | | | |
| | | | | | | | distal | [Escherichia | | | |
| | | | | | | | connector | phage AR1] | | | |
| 93 | + | 52348 | 53463 | 371 | 35 | 97 | hinge | hinge connector | 100 | 2E−265 | YP_009167615.1 |
| | | | | | | | connector of | of long tail fiber, | | | |
| | | | | | | | long tail fiber, | proximal | | | |
| | | | | | | | proximal | [Enterobacteria | | | |
| | | | | | | | connector | phage RB68] | | | |
| 94 | + | 53472 | 57341 | 1289 | 34 | 93 | long tail fiber, | long tail fiber, | 100 | 0E+00 | YP_002854200.1 |
| | | | | | | | proximal | proximal subunit | | | |
| | | | | | | | subunit | [Enterobacteria | | | |
| | | | | | | | | phage RB51] | | | |
| 95 | − | 57446 | 58363 | 305 | rnh | 100 | Rnase H | ribonuclease H | 100 | 2E−225 | YP_009290507.1 |
| | | | | | | | | [Escherichia | | | |
| | | | | | | | | phage vB_EcoM- | | | |
| | | | | | | | | UFV13] | | | |
| 96 | − | 58372 | 58641 | 89 | dsbA | 100 | double- | double-stranded | 100 | 3E−58 | NP_049858.1 |
| | | | | | | | stranded | DNA binding | | | |
| | | | | | | | DNA binding | protein [E. virus | | | |
| | | | | | | | protein | T4] | | | |
| 97 | − | 58619 | 58957 | 112 | 33 | 98 | late promoter | late promoter | 100 | 6E−73 | YP_009102443.1 |
| | | | | | | | transcription | transcription | | | |
| | | | | | | | accessory | accessory protein | | | |
| | | | | | | | protein | [Enterobacteria | | | |
| | | | | | | | | phage RB27] | | | |
| 98 | − | 58954 | 59607 | 217 | 59 | 99 | DNA helicase | DNA helicase | 100 | 2E−154 | YP_009281576.1 |
| | | | | | | | loader | loader and | | | |
| | | | | | | | | ssDNA binding | | | |
| | | | | | | | | [Escherichia | | | |
| | | | | | | | | phage UFV- | | | |
| | | | | | | | | AREG1] | | | |
| 99 | − | 59707 | 60615 | 302 | 32 | 99 | single- | single-stranded | 100 | 6E−219 | YP_007004981.1 |
| | | | | | | | stranded | DNA-binding | | | |
| | | | | | | | DNA binding | protein | | | |
| | | | | | | | protein | [Escherichia | | | |
| | | | | | | | | phage wV7] | | | |
| 100 | − | 60761 | 60991 | 76 | frd.3 | 87 | hypothetical | hypothetical | 100 | 8E−53 | YP_009210159.1 |
| | | | | | | | protein | protein | | | |
| | | | | | | | | AVU04_gp074 | | | |
| | | | | | | | | [Escherichia | | | |
| | | | | | | | | phage slur02] | | | |
| 101 | − | 61039 | 61425 | 128 | frd.2 | 45 | hypothetical | hypothetical | 99 | 4E−92 | YP_007004979.1 |
| | | | | | | | protein | protein | | | |
| | | | | | | | | F412_gp037 | | | |
| | | | | | | | | [Escherichia | | | |
| | | | | | | | | phage wV7] | | | |
| 102 | − | 61427 | 61687 | 86 | cam01_102 | — | hypothetical | hypothetical | 100 | 1E−57 | YP_009168058.1 |
| | | | | | | | protein | protein AR1_247 | | | |
| | | | | | | | | [Escherichia | | | |
| | | | | | | | | phage AR1] | | | |
| 103 | − | 61829 | 62071 | 80 | frd.1 | 99 | hypothetical | hypothetical | 100 | 4E−59 | YP_009168057.1 |
| | | | | | | | protein | protein AR1_246 | | | |
| | | | | | | | | [Escherichia | | | |
| | | | | | | | | phage AR1] | | | |
| 104 | − | 62082 | 62441 | 119 | cam01_104 | — | hypothetical | hypothetical | 100 | 8E−84 | YP_009168056.1 |
| | | | | | | | protein | protein AR1_245 | | | |
| | | | | | | | | [Escherichia | | | |
| | | | | | | | | phage AR1] | | | |
| 105 | − | 62438 | 62683 | 81 | cam01_105 | — | hypothetical | hypothetical | 100 | 2E−57 | YP_009168055.1 |
| | | | | | | | protein | protein AR1_244 | | | |
| | | | | | | | | [Escherichia | | | |
| | | | | | | | | phage AR1] | | | |
| 106 | − | 62683 | 63264 | 193 | frd | 96 | dihydrofolate | dihydrofolate | 100 | 2E−139 | YP_007004974.1 |
| | | | | | | | reductase | reductase | | | |
| | | | | | | | | [Escherichia | | | |
| | | | | | | | | phage wV7] | | | |
| 107 | − | 63284 | 63631 | 115 | cam01_107 | — | hypothetical | hypothetical | 100 | 2E−88 | YP_007004973.1 |
| | | | | | | | protein | protein | | | |
| | | | | | | | | F412_gp043 | | | |
| | | | | | | | | [Escherichia | | | |
| | | | | | | | | phage wV7] | | | |
| 108 | − | 63677 | 64537 | 286 | td | 94 | thymidylate | dTMP | 97 | 7E−217 | YP_009168052.1 |
| | | | | | | | synthase | (thymidylate) | | | |
| | | | | | | | | synthase | | | |
| | | | | | | | | [Escherichia | | | |
| | | | | | | | | phage AR1] | | | |
| 109 | − | 64561 | 64824 | 87 | nrdA.2 | 89 | hypothetical | nrdA.2 | 100 | 2E−61 | YP_002854185.1 |
| | | | | | | | protein | hypothetical | | | |
| | | | | | | | | protein | | | |
| | | | | | | | | [Enterobacteria | | | |
| | | | | | | | | phage RB51] | | | |
| 110 | − | 65095 | 67359 | 754 | nrdA | 99 | ribonucleoside- | ribonucleoside- | 100 | 0E+00 | YP_009102429.1 |
| | | | | | | | diphosphate | diphosphate | | | |
| | | | | | | | reductase | reductase subunit | | | |
| | | | | | | | subunit alpha | alpha | | | |
| | | | | | | | | [Enterobacteria | | | |
| | | | | | | | | phage RB27] | | | |
| 111 | − | 67411 | 68589 | 392 | nrdB | 90 | ribonucleoside- | ribonucleoside- | 100 | 3E−293 | YP_009619035.1 |
| | | | | | | | diphosphate | diphosphate | | | |
| | | | | | | | reductase | reductase, small | | | |
| | | | | | | | subunit beta | subunit [Shigella | | | |
| | | | | | | | | phage Sf21] | | | |
| 112 | − | 68617 | 69027 | 136 | denA | 99 | endonuclease | endonuclease II | 100 | 4E−96 | YP_009210146.1 |
| | | | | | | | II | [Escherichia | | | |
| | | | | | | | | phage slur02] | | | |
| 113 | − | 69080 | 70204 | 374 | rnlA | 99 | RNA ligase | RNA ligase 1 and | 100 | 5E−279 | YP_007004966.1 |
| | | | | | | | | tail fiber | | | |
| | | | | | | | | attachment | | | |
| | | | | | | | | catalyst | | | |
| | | | | | | | | [Escherichia | | | |
| | | | | | | | | phage wV7] | | | |
| 114 | − | 70266 | 70772 | 168 | alc | 98 | inhibitor of | alc inhibitor of | 100 | 1E−117 | YP_009168045.1 |
| | | | | | | | host | host transcription | | | |
| | | | | | | | transcription | [Escherichia | | | |
| | | | | | | | | phage AR1] | | | |
| 115 | − | 70763 | 71116 | 117 | pseT.3 | 97 | hypothetical | hypothetical | 100 | 1E−72 | YP_009168044.1 |
| | | | | | | | protein | protein AR1_233 | | | |
| | | | | | | | | [Escherichia | | | |
| | | | | | | | | phage AR1] | | | |
| 116 | − | 71113 | 71412 | 99 | pseT.2 | 99 | hypothetical | hypothetical | 100 | 1E−70 | YP_009168043.1 |
| | | | | | | | protein | protein AR1_232 | | | |
| | | | | | | | | [Escherichia | | | |
| | | | | | | | | phage AR1] | | | |
| 117 | − | 71409 | 71639 | 76 | pseT.1 | 99 | hypothetical | pseT.1 | 100 | 7E−51 | YP_002854556.1 |
| | | | | | | | protein | hypothetical | | | |
| | | | | | | | | protein [E. virus | | | |
| | | | | | | | | RB14] | | | |
| 118 | − | 71636 | 71938 | 100 | cam01_118 | — | hypothetical | hypothetical | 99 | 6E−66 | YP_007004961.1 |
| | | | | | | | protein | protein | | | |
| | | | | | | | | F412_gp055 | | | |
| | | | | | | | | [Escherichia | | | |
| | | | | | | | | phage wV7] | | | |
| 119 | − | 71935 | 72840 | 301 | pseT | 91 | polynucleotide | polynucleotide | 96 | 3E−223 | YP_009619027.1 |
| | | | | | | | kinase | kinase [Shigella | | | |
| | | | | | | | | phage Sf21] | | | |
| 120 | − | 72840 | 73037 | 65 | cd.5 | 98 | hypothetical | hypothetical | 100 | 1E−44 | YP_009102691.1 |
| | | | | | | | protein | protein | | | |
| | | | | | | | | ECML134_216 | | | |
| | | | | | | | | [Escherichia | | | |
| | | | | | | | | phage ECML- | | | |
| | | | | | | | | 134] | | | |
| 121 | − | 73030 | 73230 | 66 | cd.4 | 98 | hypothetical | hypothetical | 100 | 3E−46 | YP_009168038.1 |
| | | | | | | | protein | protein AR1_227 | | | |
| | | | | | | | | [Escherichia | | | |
| | | | | | | | | phage AR1] | | | |
| 122 | − | 73233 | 73508 | 91 | cd.3 | 99 | hypothetical | hypothetical | 100 | 1E−58 | YP_009197239.1 |
| | | | | | | | protein | protein | | | |
| | | | | | | | | AVU02_gp043 | | | |
| | | | | | | | | [Escherichia | | | |
| | | | | | | | | phage slur07] | | | |
| 123 | − | 73571 | 74098 | 175 | cam01_123 | — | hypothetical | hypothetical | 99 | 4E−126 | YP_009098600.1 |
| | | | | | | | protein | protein RB3_214 | | | |
| | | | | | | | | [Escherichia | | | |
| | | | | | | | | phage RB3] | | | |
| 124 | − | 74092 | 74328 | 78 | cd.2 | 94 | hypothetical | hypothetical | 100 | 2E−49 | YP_009210134.1 |
| | | | | | | | protein | protein | | | |
| | | | | | | | | AVU04_gp049 | | | |
| | | | | | | | | [Escherichia | | | |
| | | | | | | | | phage slur02] | | | |
| 125 | − | 74325 | 74663 | 112 | cd.1 | 98 | hypothetical | hypothetical | 100 | 1E−74 | YP_007004955.1 |
| | | | | | | | protein | protein | | | |
| | | | | | | | | F412_gp061 | | | |
| | | | | | | | | [Escherichia | | | |
| | | | | | | | | phage wV7] | | | |
| 126 | − | 74660 | 75241 | 193 | cd | 99 | dCMP | dCMP deaminase | 100 | 3E−144 | YP_009168033.1 |
| | | | | | | | deaminase | [Escherichia | | | |
| | | | | | | | | phage AR1] | | | |
| 127 | − | 75241 | 75477 | 78 | 31.2 | 95 | hypothetical | hypothetical | 99 | 2E−51 | YP_009180711.1 |
| | | | | | | | protein | protein | | | |
| | | | | | | | | AS348_gp200 | | | |
| | | | | | | | | [Escherichia | | | |
| | | | | | | | | phage slur14] | | | |
| 128 | − | 75478 | 75786 | 102 | 31.1 | 98 | SH3 beta- | phage tail fiber | 99 | 8E−67 | YP_007004586.1 |
| | | | | | | | barrel fold- | protein | | | |
| | | | | | | | containing | [Escherichia | | | |
| | | | | | | | protein | phage ime09] | | | |
| 129 | − | 75843 | 76178 | 111 | 31 | 100 | head | co-chaperone | 100 | 3E−79 | YP_009197246.1 |
| | | | | | | | assembly co- | GroES | | | |
| | | | | | | | chaperone | [Escherichia | | | |
| | | | | | | | GroES | phage slur07] | | | |
| | | | | | | | protein | | | | |
| 130 | − | 76326 | 76574 | 82 | rllI | 100 | lysis inhibition | lysis inhibition | 100 | 4E−53 | YP_002854543.1 |
| | | | | | | | accessory protein | accessory protein | | | |
| | | | | | | | | [E. virus RB14] | | | |
| 131 | − | 76818 | 76994 | 58 | 30.9 | 100 | hypothetical | gp30.9 conserved | 100 | 4E−32 | NP_049823.1 |
| | | | | | | | protein | hypothetical | | | |
| | | | | | | | | protein [E. virus | | | |
| | | | | | | | | T4] | | | |
| 132 | − | 77105 | 77437 | 110 | 30.8 | 97 | hypothetical | gp30.8 | 99 | 7E−78 | YP_002854541.1 |
| | | | | | | | protein | hypothetical | | | |
| | | | | | | | | protein [E. virus | | | |
| | | | | | | | | RB14] | | | |
| 133 | − | 77506 | 77871 | 121 | 30.7 | 99 | hypothetical | gp30.7 | 100 | 1E−90 | YP_002854161.1 |
| | | | | | | | protein | hypothetical | | | |
| | | | | | | | | protein | | | |
| | | | | | | | | [Enterobacteria | | | |
| | | | | | | | | phage RB51] | | | |
| 134 | − | 77912 | 78199 | 95 | 30.6 | 98 | hypothetical | gp30.6 | 100 | 6E−66 | YP_002854539.1 |
| | | | | | | | protein | hypothetical | | | |
| | | | | | | | | protein [E. virus | | | |
| | | | | | | | | RB14] | | | |
| 135 | − | 78199 | 78396 | 65 | 30.5 | 98 | hypothetical | gp30.5 | 100 | 7E−44 | YP_002854538.1 |
| | | | | | | | protein | hypothetical | | | |
| | | | | | | | | protein [E. virus | | | |
| | | | | | | | | RB14] | | | |
| 136 | − | 78393 | 78599 | 67 | 30.4 | 96 | hypothetical | hypothetical | 100 | 8E−49 | YP_007004943.1 |
| | | | | | | | protein | protein | | | |
| | | | | | | | | F412_gp073 | | | |
| | | | | | | | | [Escherichia | | | |
| | | | | | | | | phage wV7] | | | |
| 137 | − | 78592 | 79050 | 152 | 30.3 | 97 | hypothetical | hypothetical | 98 | 9E−108 | YP_009281534.1 |
| | | | | | | | protein | protein | | | |
| | | | | | | | | BI043_gp194 | | | |
| | | | | | | | | [Escherichia | | | |
| | | | | | | | | phage UFV- | | | |
| | | | | | | | | AREG1] | | | |
| 138 | − | 79047 | 79883 | 278 | 30.2 | 97 | hypothetical | hypothetical | 100 | 4E−216 | YP_007004941.1 |
| | | | | | | | protein | protein | | | |
| | | | | | | | | F412_gp075 | | | |
| | | | | | | | | [Escherichia | | | |
| | | | | | | | | phage wV7] | | | |
| 139 | − | 79883 | 80152 | 89 | 30.1 | 98 | hypothetical | hypothetical | 100 | 2E−64 | YP_007004940.1 |
| | | | | | | | protein | protein | | | |
| | | | | | | | | F412_gp076 | | | |
| | | | | | | | | [Escherichia | | | |
| | | | | | | | | phage wV7] | | | |
| 140 | − | 80149 | 81612 | 487 | 30 | 98 | DNA ligase | DNA ligase | 100 | 0E+00 | YP_009281531.1 |
| | | | | | | | | [Escherichia | | | |
| | | | | | | | | phage UFV- | | | |
| | | | | | | | | AREG1] | | | |
| 141 | − | 81609 | 81794 | 61 | alt.1 | 77 | hypothetical | hypothetical | 100 | 2E−38 | YP_009281530.1 |
| | | | | | | | protein | protein | | | |
| | | | | | | | | BI043_gp190 | | | |
| | | | | | | | | [Escherichia | | | |
| | | | | | | | | phage UFV- | | | |
| | | | | | | | | AREG1] | | | |
| 142 | − | 81851 | 83944 | 697 | alt | 71 | RNA polymerase- | ADP- | 100 | 0E+00 | YP_002854531.1 |
| | | | | | | | ADP- | ribosyltransferase | | | |
| | | | | | | | ribosyltransferase | [E. virus RB14] | | | |
| 143 | − | 83948 | 86005 | 685 | alt.-1 | 95 | RNA | RNA polymerase- | 99 | 0E+00 | YP_009288560.1 |
| | | | | | | | polymerase- | ADP- | | | |
| | | | | | | | ADP- | ribosyltransferase | | | |
| | | | | | | | ribosyltransferase | [Shigella phage | | | |
| | | | | | | | | SHBML-50-1] | | | |
| 144 | − | 86066 | 86356 | 96 | alt.-3 | 97 | hypothetical | alt.-3 hypothetical | 100 | 1E−62 | YP_002854150.1 |
| | | | | | | | protein | protein | | | |
| | | | | | | | | [Enterobacteria | | | |
| | | | | | | | | phage RB51] | | | |
| 145 | + | 86385 | 87350 | 321 | 54 | 98 | baseplate | baseplate tail | 100 | 1E−217 | YP_009168010.1 |
| | | | | | | | subunit | tube initiator | | | |
| | | | | | | | | [Escherichia | | | |
| | | | | | | | | phage AR1] | | | |
| 146 | + | 87350 | 88444 | 364 | 48 | 95 | baseplate | baseplate tail | 100 | 2E−248 | YP_009168009.1 |
| | | | | | | | tail-tube | tube cap | | | |
| | | | | | | | junction | [Escherichia | | | |
| | | | | | | | protein | phage AR1] | | | |
| 147 | + | 88453 | 90225 | 590 | 29 | 96 | baseplate | baseplate hub | 99 | 0E+00 | YP_009168008.1 |
| | | | | | | | hub subunit | subunit/tail length | | | |
| | | | | | | | | determinator | | | |
| | | | | | | | | [Escherichia | | | |
| | | | | | | | | phage AR1] | | | |
| 148 | + | 90222 | 90680 | 152 | 28 | 99 | baseplate | baseplate distal | 100 | 8E−111 | YP_009167560.1 |
| | | | | | | | distal hub | hub subunit | | | |
| | | | | | | | subunit | [Enterobacteria | | | |
| | | | | | | | | phage RB68] | | | |
| 149 | + | 90703 | 91875 | 390 | 27 | 98 | baseplate | baseplate hub | 100 | 4E−288 | YP_009168006.1 |
| | | | | | | | hub subunit | subunit | | | |
| | | | | | | | | [Escherichia | | | |
| | | | | | | | | phage AR1] | | | |
| 150 | + | 91872 | 92624 | 250 | 51 | 94 | baseplate | baseplate hub | 100 | 5E−185 | YP_009168005.1 |
| | | | | | | | hub assembly | assembly catalyst | | | |
| | | | | | | | protein | [Escherichia | | | |
| | | | | | | | | phage AR1] | | | |
| 151 | − | 92675 | 93301 | 208 | 26 | 99 | baseplate | baseplate hub | 100 | 1E−154 | YP_009168004.1 |
| | | | | | | | hub subunit | subunit | | | |
| | | | | | | | | [Escherichia | | | |
| | | | | | | | | phage AR1] | | | |
| 152 | − | 93301 | 93699 | 132 | 25 | 100 | baseplate | baseplate wedge | 100 | 7E−91 | YP_006986742.1 |
| | | | | | | | wedge | subunit | | | |
| | | | | | | | subunit | [Escherichia | | | |
| | | | | | | | | phage | | | |
| | | | | | | | | vB_EcoM_ACG- | | | |
| | | | | | | | | C40] | | | |
| 153 | − | 93766 | 94179 | 137 | uvsY | 99 | recombination, | recombination | 100 | 1E−93 | YP_009210105.1 |
| | | | | | | | repair and | mediator protein | | | |
| | | | | | | | ssDNA | UvsY | | | |
| | | | | | | | binding protein | [Escherichia | | | |
| | | | | | | | | phage slur02] | | | |
| 154 | − | 94179 | 94406 | 75 | uvsY.-1 | 100 | hypothetical | hypothetical | 100 | 4E−53 | YP_009030792.1 |
| | | | | | | | protein | protein e112_195 | | | |
| | | | | | | | | [Escherichia | | | |
| | | | | | | | | phage | | | |
| | | | | | | | | vB_EcoM_112] | | | |
| 155 | − | 94530 | 94697 | 55 | uvsY.-2 | 100 | hypothetical | DUF2685 | 100 | 2E−35 | NP_049797.1 |
| | | | | | | | protein | domain- | | | |
| | | | | | | | | containing protein | | | |
| | | | | | | | | [E. virus T4] | | | |
| 156 | + | 94753 | 94986 | 77 | uvsW.1 | 99 | hypothetical | hypothetical | 99 | 4E−46 | YP_007004923.1 |
| | | | | | | | protein | protein | | | |
| | | | | | | | | F412_gp093 | | | |
| | | | | | | | | [Escherichia | | | |
| | | | | | | | | phage wV7] | | | |
| 157 | + | 95012 | 96523 | 503 | uvsW | 99 | DNA helicase | DNA helicase, | 100 | 0E+00 | YP_009965402.1 |
| | | | | | | | | phage-associated | | | |
| | | | | | | | | [Escherichia | | | |
| | | | | | | | | phage CF2] | | | |
| 158 | − | 96574 | 97254 | 226 | inh | 99 | minor capsid | inhibitor of | 100 | 3E−158 | YP_009290447.1 |
| | | | | | | | protein | prohead protease | | | |
| | | | | | | | inhibitor of | [Escherichia | | | |
| | | | | | | | prohead | phage vB_EcoM- | | | |
| | | | | | | | protease | UFV13] | | | |
| 159 | − | 97264 | 98682 | 472 | hoc | 77 | outer capsid | outer capsid | 93 | 1E−303 | YP_009167995.1 |
| | | | | | | | protein | protein Hoc | | | |
| | | | | | | | | [Escherichia | | | |
| | | | | | | | | phage AR1] | | | |
| 160 | − | 98784 | 98987 | 67 | 24.3 | 84 | hypothetical | hypothetical | 100 | 9E−41 | YP_009625072.1 |
| | | | | | | | protein | protein | | | |
| | | | | | | | | [Escherichia | | | |
| | | | | | | | | phage slur03] | | | |
| 161 | − | 98974 | 99252 | 92 | 24.2 | 97 | hypothetical | hypothetical | 100 | 2E−64 | YP_009210096.1 |
| | | | | | | | protein | protein | | | |
| | | | | | | | | AVU04_gp011 | | | |
| | | | | | | | | [Escherichia | | | |
| | | | | | | | | phage slur02] | | | |
| 162 | − | 99262 | 100269 | 335 | rnlB | 82 | RNA ligase | RNA ligase | 100 | 2E−245 | YP_009278901.1 |
| | | | | | | | | [Shigella phage | | | |
| | | | | | | | | SHFML-26] | | | |
| 163 | + | 100299 | 101582 | 427 | 24 | 99 | capsid vertex | precursor of head | 100 | 4E−305 | YP_009098559.1 |
| | | | | | | | protein | vertex subunit | | | |
| | | | | | | | | [Escherichia | | | |
| | | | | | | | | phage RB31 | | | |
| 164 | + | 101666 | 103231 | 521 | 23 | 96 | major capsid | major capsid | 100 | 0E+00 | YP_009288539.1 |
| | | | | | | | protein | protein [Shigella | | | |
| | | | | | | | | phage SHBML- | | | |
| | | | | | | | | 50-1] | | | |
| 165 | + | 103250 | 104059 | 269 | 22 | 98 | prohead core | major prohead- | 100 | 1E−152 | YP_009167988.1 |
| | | | | | | | scaffolding | scaffolding core | | | |
| | | | | | | | protein | protein | | | |
| | | | | | | | | [Escherichia | | | |
| | | | | | | | | phage AR1] | | | |
| 166 | + | 104090 | 104728 | 212 | 21 | 100 | prohead core | prohead core | 100 | 1E−155 | YP_009210091.1 |
| | | | | | | | scaffolding | scaffolding | | | |
| | | | | | | | protein and | protein and | | | |
| | | | | | | | protease | protease | | | |
| | | | | | | | | [Escherichia | | | |
| | | | | | | | | phage slur02] | | | |
| 167 | + | 104728 | 105153 | 141 | 68 | 98 | prohead core | prohead core | 100 | 2E−88 | YP_009167986.1 |
| | | | | | | | protein | protein precursor | | | |
| | | | | | | | | [Escherichia | | | |
| | | | | | | | | phage AR1] | | | |
| 168 | + | 105153 | 105392 | 51 | 67 | 95 | prohead core | prohead core | 98 | 2E−27 | YP_009167985.1 |
| | | | | | | | protein | protein | | | |
| | | | | | | | | [Escherichia | | | |
| | | | | | | | | phage AR1] | | | |
| 169 | + | 105392 | 106966 | 524 | 20 | 100 | portal vertex | portal protein [E. | 100 | 0E+00 | NP_049782.1 |
| | | | | | | | of the head | virus T4] | | | |
| 170 | + | 107050 | 107541 | 163 | 19 | 100 | tail tube | tail protein [E. | 100 | 9E−120 | NP_049781.1 |
| | | | | | | | protein | virus T4] | | | |
| 171 | + | 107658 | 109637 | 659 | 18 | 98 | tail sheath | tail sheath protein | 100 | 0E+00 | YP_007004908.1 |
| | | | | | | | protein | [Escherichia | | | |
| | | | | | | | | phage wV7] | | | |
| 172 | + | 109669 | 111501 | 610 | 17 | 99 | large | putative | 100 | 0E+00 | YP_009197290.1 |
| | | | | | | | terminase | terminase subunit | | | |
| | | | | | | | protein | nuclease and | | | |
| | | | | | | | | ATPase | | | |
| | | | | | | | | [Escherichia | | | |
| | | | | | | | | phage slur07] | | | |
| 173 | + | 111485 | 111979 | 164 | 16 | 100 | small | small terminase | 100 | 1E−116 | YP_009197291.1 |
| | | | | | | | terminase | protein | | | |
| | | | | | | | protein | [Escherichia | | | |
| | | | | | | | | phage slur07] | | | |
| 174 | + | 111988 | 112806 | 272 | 15 | 99 | tail sheath | tail sheath | 100 | 3E−203 | YP_002854120.1 |
| | | | | | | | stabilizer and | stabilizer and | | | |
| | | | | | | | completion | completion | | | |
| | | | | | | | protein | protein | | | |
| | | | | | | | | [Enterobacteria | | | |
| | | | | | | | | phage RB51] | | | |
| 175 | + | 112848 | 113618 | 256 | 14 | 98 | neck protein | neck protein | 100 | 9E−191 | YP_009167978.1 |
| | | | | | | | | [Escherichia | | | |
| | | | | | | | | phage AR1] | | | |
| 176 | + | 113620 | 114549 | 309 | 13 | 98 | neck protein | neck protein | 100 | 9E−236 | YP_009167977.1 |
| | | | | | | | | [Escherichia | | | |
| | | | | | | | | phage AR1] | | | |
| 177 | + | 114581 | 116038 | 485 | wac | 82 | fibritin protein | fibritin neck | 100 | 0E+00 | YP_007004902.1 |
| | | | | | | | | whiskers | | | |
| | | | | | | | | [Escherichia | | | |
| | | | | | | | | phage wV7] | | | |
| 178 | + | 116048 | 117598 | 516 | 12 | 64 | short tail fiber | putative short tail | 100 | 0E+00 | YP_009281494.1 |
| | | | | | | | protein | fiber [Escherichia | | | |
| | | | | | | | | phage UFV- | | | |
| | | | | | | | | AREG1] | | | |
| 179 | + | 117595 | 118254 | 219 | 11 | 67 | baseplate | baseplate wedge | 100 | 3E−157 | YP_007004900.1 |
| | | | | | | | wedge | subunit and tail | | | |
| | | | | | | | subunit and | pin [Escherichia | | | |
| | | | | | | | tail pin | phage wV7] | | | |
| 180 | + | 118254 | 120059 | 601 | 10 | 86 | baseplate | baseplate wedge | 99 | 0E+00 | YP_007004899.1 |
| | | | | | | | wedge | subunit and tail | | | |
| | | | | | | | subunit and | pin [Escherichia | | | |
| | | | | | | | tail pin | phage wV7] | | | |
| 181 | + | 120059 | 120925 | 288 | 9 | 100 | baseplate | baseplate wedge | 100 | 7E−202 | YP_002854113.1 |
| | | | | | | | wedge tail | tail fiber | | | |
| | | | | | | | fiber | connector | | | |
| | | | | | | | connector | [Enterobacteria | | | |
| | | | | | | | | phage RB51] | | | |
| 182 | + | 120989 | 121993 | 334 | 8 | 100 | baseplate | baseplate wedge | 100 | 4E−261 | YP_002854112.1 |
| | | | | | | | wedge | subunit | | | |
| | | | | | | | subunit | [Enterobacteria | | | |
| | | | | | | | | phage RB51] | | | |
| 183 | + | 121986 | 125084 | 1032 | 7 | 98 | baseplate | baseplate wedge | 99 | 0E+00 | YP_007004896.1 |
| | | | | | | | wedge | initiator | | | |
| | | | | | | | initiator | [Escherichia | | | |
| | | | | | | | | phage wV7] | | | |
| 184 | + | 125081 | 127063 | 660 | 6 | 99 | baseplate | baseplate wedge | 100 | 0E+00 | YP_007004895.1 |
| | | | | | | | wedge | subunit | | | |
| | | | | | | | subunit | [Escherichia | | | |
| | | | | | | | | phage wV7] | | | |
| 185 | + | 127072 | 127365 | 97 | 5.4 | 100 | hypothetical | hypothetical | 100 | 3E−68 | NP_049763.1 |
| | | | | | | | protein | protein [E. virus | | | |
| | | | | | | | | T4] | | | |
| 186 | + | 127366 | 127860 | 164 | 5.1 | 98 | hypothetical | hypothetical | 99 | 2E−107 | YP_009102355.1 |
| | | | | | | | protein | protein RB27_150 | | | |
| | | | | | | | | [Enterobacteria | | | |
| | | | | | | | | phage RB27] | | | |
| 187 | + | 127895 | 129622 | 575 | 5 | 99 | baseplate | putative | 100 | 0E+00 | YP_004415044.1 |
| | | | | | | | hub subunit | baseplate hub | | | |
| | | | | | | | and tail | subunit and tail | | | |
| | | | | | | | lysozyme | lysozyme | | | |
| | | | | | | | | [Shigella phage | | | |
| | | | | | | | | Shfl2] | | | |
| 188 | + | 129606 | 130196 | 196 | 53 | 99 | baseplate | baseplate wedge | 100 | 1E−146 | YP_009167965.1 |
| | | | | | | | wedge | subunit | | | |
| | | | | | | | subunit | [Escherichia | | | |
| | | | | | | | | phage AR1] | | | |
| 189 | − | 130244 | 130696 | 150 | 4 | 100 | head | head completion | 100 | 4E−114 | NP_049755.1 |
| | | | | | | | completion | protein [E. virus | | | |
| | | | | | | | protein | T4] | | | |
| 190 | − | 130696 | 131523 | 275 | 2 | 99 | DNA end | DNA end | 100 | 3E−200 | YP_803089.1 |
| | | | | | | | protector | protector protein | | | |
| | | | | | | | protein | [E. virus RB32] | | | |
| 191 | − | 131520 | 132179 | 219 | mobB | 45 | homing | HNH | 100 | 1E−164 | YP_803088.1 |
| | | | | | | | endonuclease | endonuclease [E. | | | |
| | | | | | | | | virus RB32] | | | |
| 192 | − | 132286 | 132816 | 176 | 3 | 100 | tail sheath | tail protein [E. | 100 | 3E−127 | NP_049753.1 |
| | | | | | | | stabilizer and | virus T4] | | | |
| | | | | | | | completion | | | | |
| | | | | | | | protein | | | | |
| 193 | − | 132866 | 133591 | 241 | 1 | 98 | deoxynucleoside | dNMP kinase | 100 | 1E−175 | YP_009030752.1 |
| | | | | | | | monophosphate | [Escherichia | | | |
| | | | | | | | kinase | phage | | | |
| | | | | | | | | vB_EcoM_112] | | | |
| 194 | − | 133591 | 133833 | 80 | 57A | 98 | chaperone for | chaperone for tail | 99 | 3E−45 | YP_007004886.1 |
| | | | | | | | tail fiber | fiber formation | | | |
| | | | | | | | formation | [Escherichia | | | |
| | | | | | | | | phage wV7] | | | |
| 195 | − | 133833 | 134288 | 151 | 57B | 99 | hypothetical | hypothetical | 99 | 4E−109 | NP_049750.1 |
| | | | | | | | protein | protein [E. virus | | | |
| | | | | | | | | T4] | | | |
| 196 | − | 134361 | 134645 | 94 | ipl | 91 | internal head | hypothetical | 100 | 4E−59 | YP_009149385.1 |
| | | | | | | | protein | protein BN81_146 | | | |
| | | | | | | | | [Yersinia phage | | | |
| | | | | | | | | phiD1] | | | |
| 197 | − | 134722 | 134907 | 61 | trna.4 | 100 | hypothetical | trna.4 conserved | 100 | 3E−34 | NP_049748.1 |
| | | | | | | | protein | hypothetical | | | |
| | | | | | | | | predicted | | | |
| | | | | | | | | membrane | | | |
| | | | | | | | | protein [E. virus | | | |
| | | | | | | | | T4] | | | |
| 198 | − | 134909 | 135289 | 126 | trna.3 | 57 | hypothetical | hypothetical | 98 | 9E−93 | YP_009619345.1 |
| | | | | | | | protein | protein | | | |
| | | | | | | | | FDJ03_gp269 | | | |
| | | | | | | | | [Shigella phage | | | |
| | | | | | | | | Sf24] | | | |
| 199 | − | 135292 | 135579 | 95 | trna.2 | 95 | hypothetical | hypothetical | 99 | 3E−67 | YP_009210326.1 |
| | | | | | | | protein | protein | | | |
| | | | | | | | | AVU04_gp266 | | | |
| | | | | | | | | [Escherichia | | | |
| | | | | | | | | phage slur02] | | | |
| 200 | − | 136716 | 137066 | 116 | cam01_200 | — | hypothetical | hypothetical | 99 | 5E−83 | YP_009148578.1 |
| | | | | | | | protein | protein | | | |
| | | | | | | | | ACQ54_gp127 | | | |
| | | | | | | | | [Escherichia | | | |
| | | | | | | | | phage HY01] | | | |
| 201 | − | 137448 | 137921 | 157 | cam01_201 | — | hypothetical | hypothetical | 99 | 2E−102 | YP_002854093.1 |
| | | | | | | | protein | protein | | | |
| | | | | | | | | RB51ORF140 | | | |
| | | | | | | | | [Enterobacteria | | | |
| | | | | | | | | phage RB51] | | | |
| 202 | − | 138163 | 138426 | 87 | e.8 | 99 | hypothetical | hypothetical | 100 | 3E−62 | YP_009210323.1 |
| | | | | | | | protein | protein | | | |
| | | | | | | | | AVU04_gp238 | | | |
| | | | | | | | | [Escherichia | | | |
| | | | | | | | | phage slur02] | | | |
| 203 | − | 138483 | 139001 | 172 | cam01_203 | — | hypothetical | hypothetical | 98 | 5E−122 | YP_002854091.1 |
| | | | | | | | protein | protein | | | |
| | | | | | | | | RB51ORF138 | | | |
| | | | | | | | | [Enterobacteria | | | |
| | | | | | | | | phage RB51] | | | |
| 204 | − | 139044 | 139637 | 197 | e.6 | 99 | hypothetical | hypothetical | 100 | 3E−126 | YP_009149376.1 |
| | | | | | | | protein | protein BN81_137 | | | |
| | | | | | | | | [Yersinia phage | | | |
| | | | | | | | | phiD1] | | | |
| 205 | − | 139679 | 140287 | 202 | e.5 | 93 | hypothetical | hypothetical | 100 | 6E−146 | YP_007004875.1 |
| | | | | | | | protein | protein | | | |
| | | | | | | | | F412_gp141 | | | |
| | | | | | | | | [Escherichia | | | |
| | | | | | | | | phage wV7] | | | |
| 206 | − | 140630 | 141004 | 124 | e.3 | 84 | hypothetical | e.3 hypothetical | 100 | 1E−77 | YP_002854087.1 |
| | | | | | | | protein | protein | | | |
| | | | | | | | | [Enterobacteria | | | |
| | | | | | | | | phage RB51] | | | |
| 207 | − | 141001 | 141489 | 162 | e.2 | 87 | hypothetical | e.2 hypothetical | 100 | 7E−121 | YP_002854086.1 |
| | | | | | | | protein | protein | | | |
| | | | | | | | | [Enterobacteria | | | |
| | | | | | | | | phage RB51] | | | |
| 208 | − | 141486 | 141926 | 146 | nudE | 95 | nudix | nudix hydrolase | 99 | 3E−107 | YP_009965455.1 |
| | | | | | | | hydrolase | [Escherichia | | | |
| | | | | | | | | phage CF21 | | | |
| 209 | − | 141963 | 142457 | 164 | e | 97 | glycoside | glycoside | 100 | 2E−117 | YP_009210314.1 |
| | | | | | | | hydrolase | hydrolase family | | | |
| | | | | | | | family protein | protein | | | |
| | | | | | | | | [Escherichia | | | |
| | | | | | | | | phage slur02] | | | |
| 210 | − | 142546 | 143019 | 157 | cam01_210 | | hypothetical | hypothetical | 100 | 3E−111 | YP_006986677.1 |
| | | | | | | | protein | protein | | | |
| | | | | | | | | D862_gp151 | | | |
| | | | | | | | | [Escherichia | | | |
| | | | | | | | | phage | | | |
| | | | | | | | | vB_EcoM_ACG- | | | |
| | | | | | | | | C40] | | | |
| 211 | − | 143161 | 143703 | 180 | vs.8 | 87 | hypothetical | hypothetical | 99 | 2E−132 | YP_006986676.1 |
| | | | | | | | protein | protein | | | |
| | | | | | | | | D862_gp152 | | | |
| | | | | | | | | [Escherichia | | | |
| | | | | | | | | phage | | | |
| | | | | | | | | vB_EcoM_ACG- | | | |
| | | | | | | | | C40] | | | |
| 212 | − | 143700 | 144029 | 109 | vs.7 | 98 | hypothetical | hypothetical | 100 | 6E−79 | YP_007004866.1 |
| | | | | | | | protein | protein | | | |
| | | | | | | | | F412_gp150 | | | |
| | | | | | | | | [Escherichia | | | |
| | | | | | | | | phage wV7] | | | |
| 213 | − | 144037 | 144399 | 120 | vs.6 | 98 | hypothetical | autonomous | 100 | 2E−83 | YP_009625128.1 |
| | | | | | | | protein | glycyl radical | | | |
| | | | | | | | | cofactor GrcA | | | |
| | | | | | | | | [Escherichia | | | |
| | | | | | | | | phage slur03] | | | |
| 214 | − | 144399 | 144620 | 73 | vs.5 | 94 | hypothetical | hypothetical | 100 | 2E−51 | YP_009277495.1 |
| | | | | | | | protein | protein | | | |
| | | | | | | | | BH804_gp025 | | | |
| | | | | | | | | [Shigella phage | | | |
| | | | | | | | | SHFML-11] | | | |
| 215 | − | 144613 | 144879 | 88 | vs.4 | 93 | hypothetical | hypothetical | 99 | 6E−60 | YP_009625130.1 |
| | | | | | | | protein | protein | | | |
| | | | | | | | | [Escherichia | | | |
| | | | | | | | | phage slur03] | | | |
| 216 | − | 144879 | 145157 | 92 | vs.3 | 97 | hypothetical | hypothetical | 100 | 1E−58 | YP_009210305.1 |
| | | | | | | | protein | protein | | | |
| | | | | | | | | AVU04_gp220 | | | |
| | | | | | | | | [Escherichia | | | |
| | | | | | | | | phage slur02] | | | |
| 217 | − | 145217 | 145678 | 153 | regB | 99 | site-specific | site-specific RNA | 100 | 4E−109 | YP_009167930.1 |
| | | | | | | | RNA | endonuclease | | | |
| | | | | | | | endonuclease | [Escherichia | | | |
| | | | | | | | | phage AR1] | | | |
| 218 | − | 145686 | 146231 | 181 | vs.1 | 100 | hypothetical | hypothetical | 100 | 1E−130 | NP_049725.1 |
| | | | | | | | protein | protein [E. virus | | | |
| | | | | | | | | T4] | | | |
| 219 | − | 146224 | 146565 | 113 | vs | 97 | valyl-tRNA | valyl-tRNA | 100 | 6E−78 | YP_009965467.1 |
| | | | | | | | synthetase | synthetase | | | |
| | | | | | | | modifier | modifier | | | |
| | | | | | | | | [Escherichia | | | |
| | | | | | | | | phage CF2] | | | |
| 220 | − | 146562 | 147041 | 159 | tk.4 | 57 | hypothetical | hypothetical | 98 | 3E−118 | YP_009614768.1 |
| | | | | | | | protein | protein gp16 | | | |
| | | | | | | | | [Shigella phage | | | |
| | | | | | | | | Sf22] | | | |
| 221 | − | 147222 | 147428 | 68 | cam01_221 | — | hypothetical | hypothetical | 100 | 1E−52 | YP_009197343.1 |
| | | | | | | | protein | protein | | | |
| | | | | | | | | AVU02_gp241 | | | |
| | | | | | | | | [Escherichia | | | |
| | | | | | | | | phage slur07] | | | |
| 222 | − | 147425 | 147610 | 57 | cam01_222 | — | hypothetical | hypothetical | 100 | 6E−34 | YP_009167483.1 |
| | | | | | | | protein | protein RB68_114 | | | |
| | | | | | | | | [Enterobacteria | | | |
| | | | | | | | | phage RB68] | | | |
| 223 | − | 147597 | 147782 | 61 | tk.2 | 54 | hypothetical | hypothetical | 100 | 2E−38 | YP_009210297.1 |
| | | | | | | | protein | protein | | | |
| | | | | | | | | AVU04_gp212 | | | |
| | | | | | | | | [Escherichia | | | |
| | | | | | | | | phage slur02] | | | |
| 224 | − | 147792 | 148373 | 193 | tk | 99 | thymidine | thymidine kinase | 100 | 1E−138 | YP_009210296.1 |
| | | | | | | | kinase | [Escherichia | | | |
| | | | | | | | | phage slur02] | | | |
| 225 | − | 148416 | 148628 | 70 | rl.1 | 100 | hypothetical | hypothetical | 100 | 7E−42 | YP_009210295.1 |
| | | | | | | | protein | protein | | | |
| | | | | | | | | AVU04_gp210 | | | |
| | | | | | | | | [Escherichia | | | |
| | | | | | | | | phage slur02] | | | |
| 226 | − | 148641 | 148934 | 97 | rl | 98 | lysis inhibition | lysis inhibition | 100 | 6E−66 | YP_009098495.1 |
| | | | | | | | regulator, | regulator | | | |
| | | | | | | | membrane | membrane | | | |
| | | | | | | | protein | protein | | | |
| | | | | | | | | [Escherichia | | | |
| | | | | | | | | phage RB3] | | | |
| 227 | − | 148931 | 149317 | 128 | rl.-1 | 100 | hypothetical | hypothetical | 100 | 3E−89 | NP_049716.1 |
| | | | | | | | protein | protein [E. virus | | | |
| | | | | | | | | T4] | | | |
| 228 | − | 149413 | 149601 | 62 | mobD.5 | 100 | hypothetical | hypothetical | 100 | 8E−40 | NP_049715.1 |
| | | | | | | | protein | protein [E. virus | | | |
| | | | | | | | | T4] | | | |
| 229 | − | 149601 | 149804 | 66 | mobD.4 | 74 | hypothetical | hypothetical | 100 | 7E−41 | YP_002854062.1 |
| | | | | | | | protein | protein | | | |
| | | | | | | | | [Enterobacteria | | | |
| | | | | | | | | phage RB51] | | | |
| 230 | − | 149807 | 150001 | 64 | mobD.3 | 94 | hypothetical | hypothetical | 97 | 4E−40 | YP_009210290.1 |
| | | | | | | | protein | protein | | | |
| | | | | | | | | AVU04_gp205 | | | |
| | | | | | | | | [Escherichia | | | |
| | | | | | | | | phage slur02] | | | |
| 231 | − | 149991 | 150164 | 57 | mobD.2a | 89 | hypothetical | hypothetical | 100 | 6E−35 | YP_002854060.1 |
| | | | | | | | protein | protein | | | |
| | | | | | | | | [Enterobacteria | | | |
| | | | | | | | | phage RB51] | | | |
| 232 | − | 150225 | 150329 | 34 | mobD.2 | 91 | hypothetical | hypothetical | 100 | 1E−16 | YP_002854059.1 |
| | | | | | | | protein | protein | | | |
| | | | | | | | | [Enterobacteria | | | |
| | | | | | | | | phage RB51] | | | |
| 233 | − | 150329 | 150514 | 61 | cam01_233 | — | hypothetical | hypothetical | 97 | 5E−36 | YP_009030707.1 |
| | | | | | | | protein | protein e112_102 | | | |
| | | | | | | | | [Escherichia | | | |
| | | | | | | | | phage | | | |
| | | | | | | | | vB_EcoM_112] | | | |
| 234 | − | 150516 | 151058 | 180 | mobD.1 | 46 | hypothetical | hypothetical | 98 | 5E−123 | YP_009030706.1 |
| | | | | | | | protein | protein e112_101 | | | |
| | | | | | | | | [Escherichia | | | |
| | | | | | | | | phage | | | |
| | | | | | | | | vB_EcoM_112] | | | |
| 235 | − | 151065 | 151586 | 173 | cam01_235 | — | hypothetical | hypothetical | 98 | 3E−120 | YP_007004840.1 |
| | | | | | | | protein | protein | | | |
| | | | | | | | | F412_gp176 | | | |
| | | | | | | | | [Escherichia | | | |
| | | | | | | | | phage wV7] | | | |
| 236 | − | 151589 | 152050 | 153 | cam01_236 | — | hypothetical | hypothetical | 100 | 2E−109 | YP_007004479.1 |
| | | | | | | | protein | protein | | | |
| | | | | | | | | [Escherichia | | | |
| | | | | | | | | phage ime09] | | | |
| 237 | − | 152050 | 153060 | 336 | nrdC.11 | 96 | hypothetical | hypothetical | 99 | 3E−252 | YP_007004478.1 |
| | | | | | | | protein | protein | | | |
| | | | | | | | | F413_gp153 | | | |
| | | | | | | | | [Escherichia | | | |
| | | | | | | | | phage ime09] | | | |
| 238 | − | 153178 | 154146 | 322 | nrdC.10 | 99 | hypothetical | hypothetical | 100 | 2E−234 | YP_009210283.1 |
| | | | | | | | protein | protein | | | |
| | | | | | | | | AVU04_gp198 | | | |
| | | | | | | | | [Escherichia | | | |
| | | | | | | | | phage slur02] | | | |
| 239 | − | 154248 | 154550 | 100 | nrdC.9 | 98 | hypothetical | hypothetical | 100 | 1E−71 | YP_009102301.1 |
| | | | | | | | protein | protein RB27_096 | | | |
| | | | | | | | | [Enterobacteria | | | |
| | | | | | | | | phage RB27] | | | |
| 240 | − | 154611 | 155138 | 175 | nrdC.8 | 99 | hypothetical | hypothetical | 100 | 3E−119 | YP_009210281.1 |
| | | | | | | | protein | protein | | | |
| | | | | | | | | AVU04_gp196 | | | |
| | | | | | | | | [Escherichia | | | |
| | | | | | | | | phage slur02] | | | |
| 241 | − | 155194 | 155601 | 135 | nrdC.7 | 92 | hypothetical | hypothetical | 99 | 3E−93 | YP_009210280.1 |
| | | | | | | | protein | protein | | | |
| | | | | | | | | AVU04_gp195 | | | |
| | | | | | | | | [Escherichia | | | |
| | | | | | | | | phage slur02] | | | |
| 242 | − | 155609 | 156496 | 295 | nrdC.6 | 98 | hypothetical | nrdC.6 | 98 | 3E−210 | YP_002854426.1 |
| | | | | | | | protein | hypothetical | | | |
| | | | | | | | | protein [E. virus | | | |
| | | | | | | | | RB14] | | | |
| 243 | − | 156505 | 157524 | 339 | nrdC.5 | 96 | hypothetical | hypothetical | 98 | 3E−243 | YP_009197365.1 |
| | | | | | | | protein | protein | | | |
| | | | | | | | | AVU02_gp219 | | | |
| | | | | | | | | [Escherichia | | | |
| | | | | | | | | phage slur07] | | | |
| 244 | − | 157552 | 158583 | 343 | nrdC.4 | 88 | hypothetical | hypothetical | 97 | 2E−239 | YP_009167902.1 |
| | | | | | | | protein | protein AR1_091 | | | |
| | | | | | | | | [Escherichia | | | |
| | | | | | | | | phage AR1] | | | |
| 245 | − | 158635 | 159564 | 309 | nrdC.3 | 60 | hypothetical | nrdC.3 | 99 | 8E−223 | YP_002854423.1 |
| | | | | | | | protein | hypothetical | | | |
| | | | | | | | | protein [E. virus | | | |
| | | | | | | | | RB14] | | | |
| 246 | − | 159561 | 159950 | 129 | nrdC.2 | 78 | hypothetical | nrdC.2 | 99 | 1E−93 | YP_002854422.1 |
| | | | | | | | protein | hypothetical | | | |
| | | | | | | | | protein [E. virus | | | |
| | | | | | | | | RB14] | | | |
| 247 | − | 159953 | 160195 | 80 | nrdC.1 | 99 | hypothetical | nrdC.1 conserved | 99 | 1E−51 | NP_049699.1 |
| | | | | | | | protein | hypothetical | | | |
| | | | | | | | | protein [E. virus | | | |
| | | | | | | | | T4] | | | |
| 248 | − | 160197 | 160460 | 87 | nrdC | 100 | thioredoxin | nrdC thioredoxin | 100 | 7E−63 | NP_049698.1 |
| | | | | | | | | [E. virus T4] | | | |
| 249 | − | 160457 | 160672 | 71 | cam01_249 | — | hypothetical | hypothetical | 100 | 3E−48 | YP 009210272.1 |
| | | | | | | | protein | protein | | | |
| | | | | | | | | AVU04_gp187 | | | |
| | | | | | | | | [Escherichia | | | |
| | | | | | | | | phage slur02] | | | |
| 250 | − | 160651 | 160845 | 64 | cam01_250 | — | hypothetical | hypothetical | 95 | 1E−38 | YP_009284107.1 |
| | | | | | | | protein | protein | | | |
| | | | | | | | | BI016_gp112 | | | |
| | | | | | | | | [Escherichia | | | |
| | | | | | | | | phage HY03] | | | |
| 251 | − | 160968 | 161453 | 161 | pin | 96 | peptidase | host protease | 99 | 8E−118 | YP_009167893.1 |
| | | | | | | | | inhibitor | | | |
| | | | | | | | | [Escherichia | | | |
| | | | | | | | | phage AR1] | | | |
| 252 | − | 161490 | 161666 | 58 | cam01_252 | — | hypothetical | hypothetical | 100 | 8E−40 | YP_803020.1 |
| | | | | | | | protein | protein | | | |
| | | | | | | | | RB32ORF078c | | | |
| | | | | | | | | [E. virus RB32] | | | |
| 253 | − | 161708 | 162181 | 157 | 49 | 100 | recombination | recombination | 100 | 1E−115 | NP_049692.1 |
| | | | | | | | endonuclease | endonuclease VII | | | |
| | | | | | | | VII | [E. virus T4] | | | |
| 254 | − | 162178 | 163995 | 605 | nrdD | 100 | anaerobic | anaerobic | 100 | 0E+00 | YP_002854413.1 |
| | | | | | | | ribonucleoside | ribonucleotide | | | |
| | | | | | | | triphosphate | reductase, large | | | |
| | | | | | | | reductase, | subunit [E. virus | | | |
| | | | | | | | large subunit | RB14] | | | |
| 255 | − | 163992 | 164462 | 156 | nrdG | 79 | anaerobic | nrdG anaerobic | 99 | 2E−125 | YP_009167890.1 |
| | | | | | | | ribonucleoside | NTP reductase, | | | |
| | | | | | | | triphosphate | small subunit | | | |
| | | | | | | | reductase, | [Escherichia | | | |
| | | | | | | | activating | phage AR1] | | | |
| | | | | | | | protein | | | | |
| 256 | − | 164455 | 164568 | 37 | cam01_256 | — | hypothetical | hypothetical | 100 | 2E−22 | YP_009290343.1 |
| | | | | | | | protein | protein | | | |
| | | | | | | | | BIZ64_gp077 | | | |
| | | | | | | | | [Escherichia | | | |
| | | | | | | | | phage vB_EcoM- | | | |
| | | | | | | | | UFV13] | | | |
| 257 | − | 164578 | 164793 | 71 | 55.8 | 93 | hypothetical | hypothetical | 100 | 2E−40 | YP_009167889.1 |
| | | | | | | | protein | protein AR1_078 | | | |
| | | | | | | | | [Escherichia | | | |
| | | | | | | | | phage AR1] | | | |
| 258 | − | 164796 | 165107 | 103 | cam01_258 | — | hypothetical | hypothetical | 100 | 8E−76 | YP_009167888.1 |
| | | | | | | | protein | protein AR1_077 | | | |
| | | | | | | | | [Escherichia | | | |
| | | | | | | | | phage AR1] | | | |
| 259 | − | 165079 | 165402 | 107 | nrdH | 99 | glutaredoxin | glutaredoxin | 100 | 5E−75 | YP_009210263.1 |
| | | | | | | | | [Escherichia | | | |
| | | | | | | | | phage slur02] | | | |
| 260 | − | 165561 | 165737 | 58 | 55.6 | 71 | hypothetical | hypothetical | 100 | 7E−37 | YP_009102278.1 |
| | | | | | | | protein | protein RB27_073 | | | |
| | | | | | | | | [Enterobacteria | | | |
| | | | | | | | | phage RB27] | | | |
| 261 | − | 165730 | 166023 | 97 | 55.5 | 99 | hypothetical | gp55.5 conserved | 99 | 8E−67 | NP_049684.1 |
| | | | | | | | protein | protein of | | | |
| | | | | | | | | unknown function | | | |
| | | | | | | | | [E. virus T4] | | | |
| 262 | − | 166031 | 166162 | 43 | 55.4 | 98 | hypothetical | hypothetical | 100 | 4E−28 | YP_009102276.1 |
| | | | | | | | protein | protein RB27_071 | | | |
| | | | | | | | | [Enterobacteria | | | |
| | | | | | | | | phage RB27] | | | |
| 263 | − | 166163 | 166363 | 66 | 55.3 | 74 | hypothetical | hypothetical | 99 | 3E−44 | YP_009210259.1 |
| | | | | | | | protein | protein | | | |
| | | | | | | | | AVU04_gp174 | | | |
| | | | | | | | | [Escherichia | | | |
| | | | | | | | | phage slur02] | | | |
| 264 | − | 166417 | 166743 | 108 | 55.2 | 100 | hypothetical | gp55.2 | 100 | 9E−71 | NP_049681.1 |
| | | | | | | | protein | hypothetical | | | |
| | | | | | | | | protein [E. virus | | | |
| | | | | | | | | T4] | | | |
| 265 | − | 166746 | 166961 | 71 | 55.1 | 94 | hypothetical | hypothetical | 100 | 7E−47 | YP_009618879.1 |
| | | | | | | | protein | protein | | | |
| | | | | | | | | FDJ02_gp065 | | | |
| | | | | | | | | [Shigella phage | | | |
| | | | | | | | | Sf21] | | | |

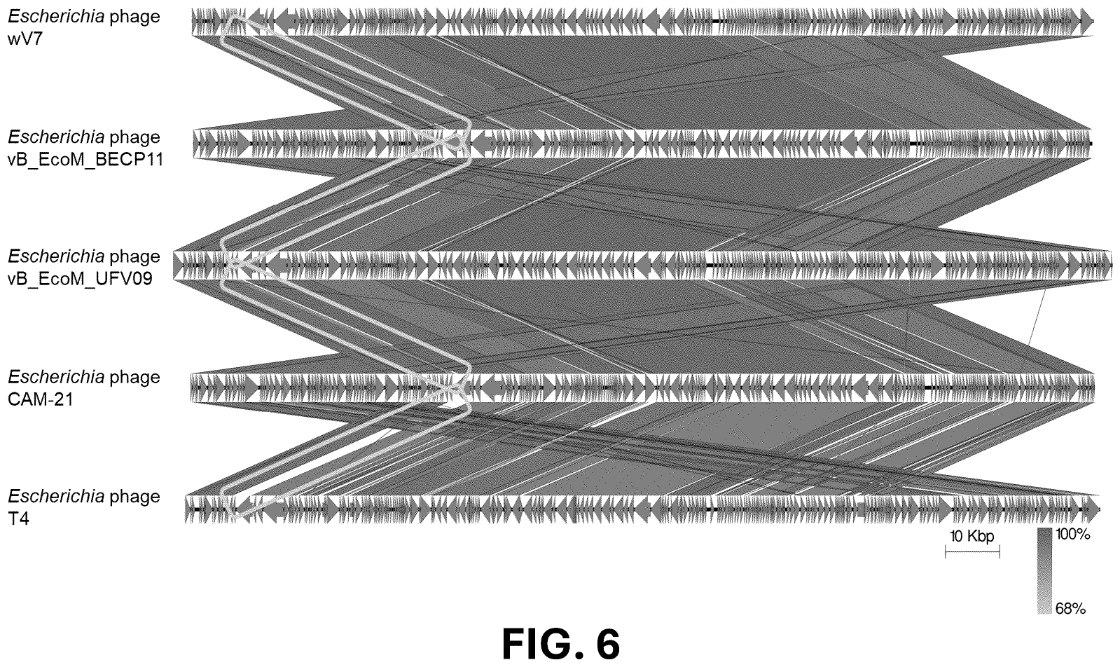

To evaluate the novelty of CAM-21, its phage DNA genome was compared to those of Tequatrovirus phages, such as wV7, vB_EcoM-BECP11, vB_EcoM-UFV09 and T4 (FIG. 6). The gene sequence of CAM-21 showed a conserved gene alignment with T4-like phages, based on the genome comparison. The gene sequences encoding the tail fiber proteins, which are responsible for host recognition were found to have high similarity (>80% identity) between CAM-21 and the selected phages, except for phage T4. As compared with other Tequatrovirus phages above, the long tail fiber subunit (gp37_ORF91) and the tail fiber adhesin (gp38_ORF90) of CAM-21 and vB_EcoM-BECP11 revealed the highest homology, which were 94% and 96%, respectively. Interestingly, the gp37_ORF91 of CAM-21 shared an identity of 35% while the gp38_ORF90 showed no homology, as compared to those of T4 (Table 4).

TABLE 4
gp37 and gp38 in CAM-21 and T4-like Phages

| | Length | | | | Length | | |
|---|---|---|---|---|---|---|---|
| | (no. of | | | | (no. of | | |
| | amino | Gene | Putative | | amino | Identity | |
| ORF | acid) | name | function | Closely related protein | acid) | (%) | Accession no. |
| 91 | 1103 | 37 | long tail | long tail fiber subunit, | 1103 | 97 | YP_010076447.1 |
| | | | fiber, large | distal [Shigella phage | | | |
| | | | [symbol] | Sf23] | | | |
| | | | | hypothetical protein | 1103 | 97 | YP 010070630.1 |
| | | | | [Escherichia phage | | | |
| | | | | vB_EcoM_G4507] | | | |
| | | | | long tail fiber distal | 1103 | 96 | YP_009168069.1 |
| | | | | subunit [Escherichia | | | |
| | | | | phage AR1] | | | |
| | | | | gp37 large distal tail | 1103 | 96 | YP_002854203.1 |
| | | | | fiber subunit | | | |
| | | | | [Enterobacteria phage | | | |
| | | | | RB51] | | | |
| | | | | long tail fiber distal | 1109 | 95 | YP_010099669.1 |
| | | | | subunit [Escherichia | | | |
| | | | | phage MLF4] | | | |
| | | | | large distal long tail | 1103 | 94 | YP_010076863.1 |
| | | | | fiber subunit [Shigella | | | |
| | | | | phage SH7] | | | |
| | | | | tail fibers protein | 1103 | 94 | YP_010073123.1 |
| | | | | [Escherichia phage | | | |
| | | | | PE37] | | | |
| | | | | long tail fiber distal | 1109 | 94 | BBI58091.1 |
| | | | | subunit [Shigella | | | |
| | | | | phage SfPhi01] | | | |
| | | | | hypothetical protein | 1109 | 94 | QQM15651.1 |
| | | | | BECP11_00086 | | | |
| | | | | [Escherichia phage | | | |
| | | | | vB_EcoM-BECP11] | | | |
| | | | | hypothetical protein | 1108 | 86 | QWT76796.1 |
| | | | | [Escherichia phage | | | |
| | | | | vB_EcoM-UFV09] | | | |
| | | | | long tail fiber distal | 1114 | 80 | YP_007004989.1 |
| | | | | subunit [Escherichia | | | |
| | | | | phage wV7] | | | |
| | | | | gp37 | 1026 | 35 | NP_049863.1 |
| | | | | long tail fiber, distal | | | |
| | | | | subunit [E. virus T4] | | | |
| 90 | 258 | 38 | tail fiber | receptor- | 258 | 99 | YP_010070631.1 |
| | | | adhesin | recognizing protein | | | |
| | | | | [Escherichia phage | | | |
| | | | | vB_EcoM_G4507] | | | |
| | | | | receptor- | 259 | 98 | YP_010073122.1 |
| | | | | recognizing protein | | | |
| | | | | [Escherichia phage | | | |
| | | | | PE37] | | | |
| | | | | receptor- | 259 | 97 | YP_009148690.1 |
| | | | | recognizing protein | | | |
| | | | | [Escherichia phage | | | |
| | | | | HY01] | | | |
| | | | | receptor recognition | 259 | 97 | QPP47032.1 |
| | | | | [Shigella phage Sfk20] | | | |
| | | | | receptor- | 258 | 97 | YP_010076448.1 |
| | | | | recognizing protein | | | |
| | | | | [Shigella phage Sf23] | | | |
| | | | | receptor- | 259 | 97 | YP_009281584.1 |
| | | | | recognizing protein | | | |
| | | | | [Escherichia phage | | | |
| | | | | UFV-AREG1] | | | |
| | | | | receptor- | 268 | 97 | YP_009030861.1 |
| | | | | recognizing protein | | | |
| | | | | [Escherichia phage | | | |
| | | | | vB_EcoM_112] | | | |
| | | | | hypothetical protein | 259 | 96 | QQM15650.1 |
| | | | | BECP11_00085 | | | |
| | | | | [Escherichia phage | | | |
| | | | | vB_EcoM-BECP11] | | | |
| | | | | receptor- | 259 | 95 | YP_009168070.1 |
| | | | | recognizing protein | | | |
| | | | | [Escherichia phage AR1] | | | |
| | | | | tail fibers protein | 264 | 81 | QWT76795.1 |
| | | | | [Escherichia phage | | | |
| | | | | vB_EcoM-UFV09] | | | |
| | | | | distal long tail fiber | 264 | 81 | YP_007004990.1 |
| | | | | assembly catalyst | | | |
| | | | | [Escherichia phage | | | |
| | | | | wV7] | | | |
| | | | | gp38 distal long tail | 183 | — | NP_049864.1 |
| | | | | fiber assembly catalyst | | | |
| | | | | [E. virus T4] | | | |

[symbol]

indicates data missing or illegible when filed

### Example 8: Phage Application in Foods

The inhibitory effect of CAM-21 against E. coli O157:H7 strain C7927 was studied using liquid (pasteurized milk) and solid (ground beef and baby spinach) food samples, which were purchased from a local supermarket. First, the bacterial culture was transferred to fresh TSB and incubated overnight at 37° C. To obtain a bacterial culture with high purity, the overnight culture was centrifuged at 9300×g for 5 min and washed with PBS for twice. Then, the culture was inoculated at different concentrations into the food samples. All food samples were pre-plated on MacConkey sorbitol prior to inoculation and found to be negative for this organism.

For the liquid food study, 100 μL of the purified cultures (105 and 106 CFU/mL) were added to each tube containing 9.9 mL of pasteurized milk, followed by the addition of 100 μL of phage suspensions (109 PFU/mL) to give MOI values of 10,000 and 1000, respectively. SM buffer (100 μL) was used as a control. All the samples were stored in a refrigerator at 4° C. After 0, 3, 6, 9, 12, and 24 h of incubation, and aliquots were collected, diluted and plated on MacConkey sorbitol agar plates. After the plates were incubated for 24 h at 37° C., total viable counts of E. coli O157:H7 were determined and expressed in log CFU/mL values. For the solid food study, 10 g of ground beef and a piece of baby spinach leaf (about 1 g) were placed in duplicate respective sterile petri plates. One hundred microliters each of 105 and 106 CFU/mL bacterial cultures were added and spread on the surface of each food sample with a sterile pipet tip. The spiked foods were left in a laminar hood for 30 min to allow for bacterial attachment. Then, a 100 μL of phage suspension (109 PFU/mL) was added to give MOI values of 10,000 and 1000, respectively. SM buffer (100 μL) was used as a control. The food samples were placed in a refrigerator at 4° C. and collected after 0, 3, 6, 9, 12, and 24 h of incubations. On each sampling day, the ground beef and baby spinach samples were placed in sterile stomacher bags, followed by the addition of 90 mL and 9 mL of peptone water (0.1% (w/v)), respectively. The samples were homogenized using a Stomacher™ Circulator Model 400 (Seward, Ltd., UK). The serially-diluted homogenates were plated on MacConkey sorbitol agar plates and incubated for 24 h at 37° C. Total viable counts of the bacteria were determined and expressed in log CFU/g values.

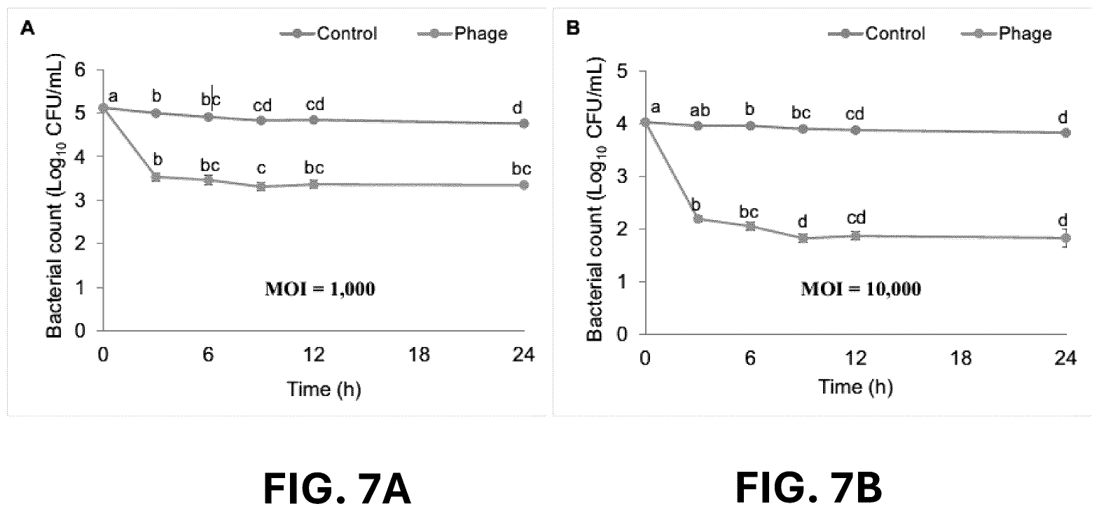

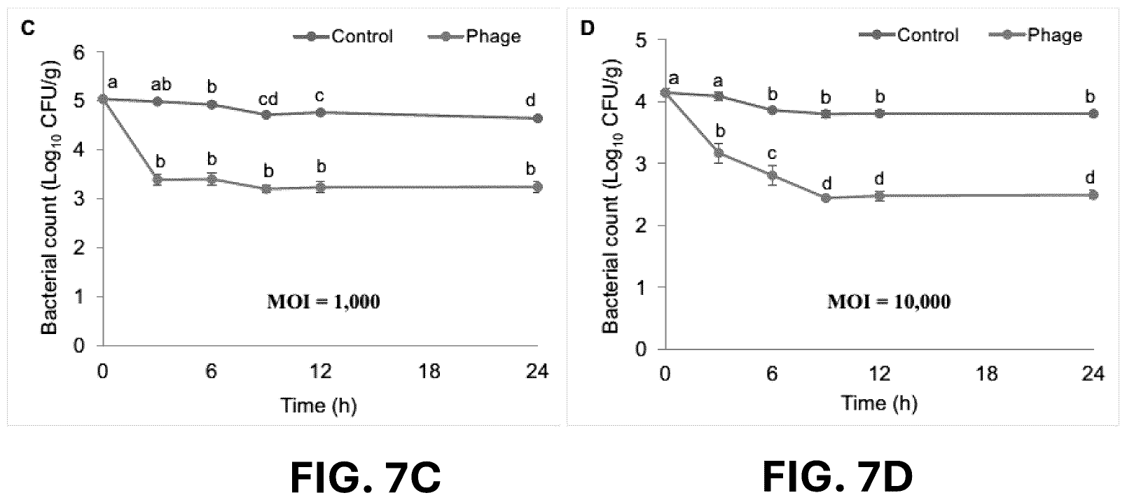

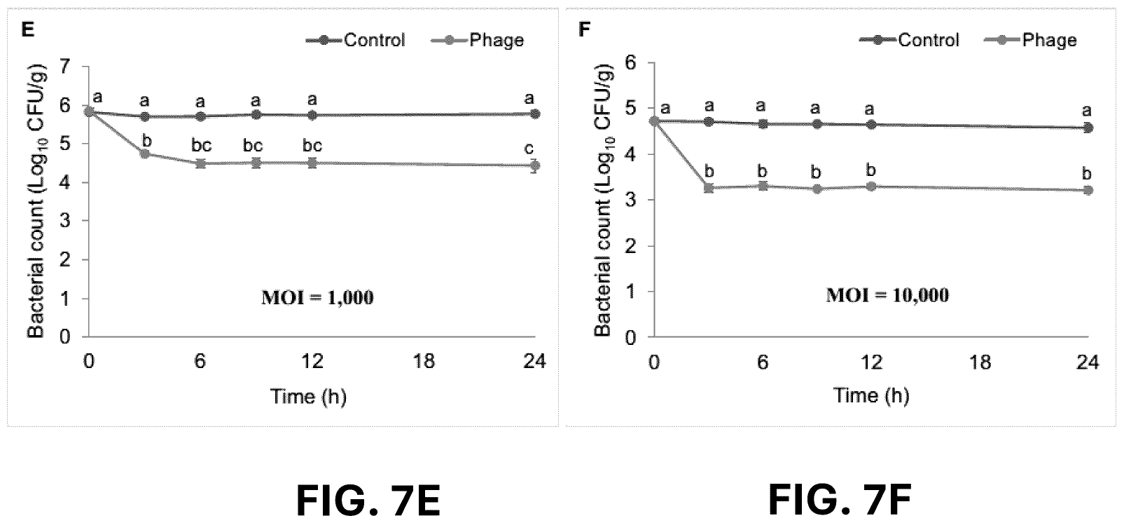

The biocontrol effect of CAM-21 against E. coli O157:H7 was evaluated in milk (FIGS. 7A-7B), ground beef (FIGS. 7C-7D), and baby spinach (FIGS. 7E-7F). The food samples were first spiked with E. coli O157:H7 C7927 before the phage treatment. For the control group, the bacterial counts in all the foods remained almost constant after 24 h of incubation at 4° C., showing only a slight reduction of <0.4 log 10 CFU/mL at the tested MOIs. After 24 h of incubation, the bacterial counts in the treated milk samples were significantly decreased (P≤0.05) by 1.4 and 2.0 log 10 CFU/mL at MOIs of 1000 and 10,000, respectively. Also, the application of CAM-21 on ground beef and baby spinach at MOIs of 1000 decreased the bacterial counts by 1.4 and 1.3 log 10 CFU/g, respectively, after 24 h of storage. At a MOI of 10,000, the phage treatment significantly decreased (P≤ 0.05) the bacterial counts on ground beef and baby spinach by 1.3 and 1.4 log 10 CFU/g, respectively. As shown in FIGS. 7A-7F, treatment with CAM-21 at MOIs of 1000 and 10,000 significantly decreased (P≤0.05) the bacterial counts in all the refrigerated food samples at all the tested time points, as compared to those of controls.

### Example 9A: Film Preparation (Soy Protein Biopolymer)

Pro-Fam® 873 isolated soy protein was sourced from Archer-Daniels-Midland Company (Decatur, IL, USA). Cellulose nanofiber (CNF) in powdered form, produced by bleaching wood pulp, was obtained from the University of Maine, and used to create a suspension. Glycerol was purchased from Fisher Scientific (Waltham, MA, USA). Fresh raw bottom round beef and raw beef trimmings were procured from the Meat Market of the University of Missouri in Columbia, MO, USA. E. coli phage CAM-21 and various strains of E. coli O157:H7, that included C7927, 3178-85, EDL-933, 505B, and MF-1847, were acquired from the University of Missouri's Food Microbiology Lab culture collection. All bacterial strains were used to assess the antimicrobial activity of the films in both broth and beef samples, and E. coli O157:H7 strain C7927 was utilized as the host bacterium to produce the phage stock.

A film-forming solution (FFS) was created by combining 6% (w/w) soy protein isolate (SPI) and 3% (w/w) glycerol in sterile distilled water, with stirring at 800 rpm. The pH of the solution was then raised to 10.0, using a 5 M sodium hydroxide solution, and the solution heated at 85° C. for 30 min while stirring at 800 rpm. Once the solution had cooled to room temperature, 15% (w/w) CNF was added based on the dry weight of SPI. The mixture was stirred for 15 min. Next, 1%, 2%, or 4% (v/w) of CAM-21 was added to the FFS based on its weight, and the solution was gently stirred for 15 min. The film preparation involved using a sterile square-shaped 12 cm² petri dish and a solution-casting technique. The films were dried at 30° C. for 48 h. The experimental setup included different film samples: one SPIfilm with 1% CAM-21 (SC1), one SPI film with 2% CAM-21 (SC2), and one SPI film with 4% CAM-21 (SC4). Additionally, a control film(S) sample did not contain CAM-21.

### Example 9B: Film Preparation (Carbohydrate Biopolymers)

Carbohydrate biopolymers and additives, including carboxymethyl cellulose sodium (CMCS), sodium alginate (SA), gelatin and pullulan (PuI) were purchased from Thermo Fisher Scientific (Waltham, MA). To prepare polymer solutions, these biopolymers were dissolved in deionized water, and glycerol (2% v/v) and Tween 80 (0.01 v/v) were added. Glycerol serves as a plasticizer to increase the flexibility of the films, while Tween 80 functions as an emulsifier, stabilizing the mixture. The solutions were stirred continuously at 80° C. for 18 h to ensure full dissolution and homogeneous distribution of all components.

CAM-21 phage, at a concentration of 10⁸ PFU/mL, was added into the biocomposite film solutions. This concentration was chosen to ensure sufficient phage activity for effective biocontrol against E. coli O157:H7, maximizing the antimicrobial potential of the films. The high CAM-21 MOI of 1000 allows for an intense initial interaction between the bacteriophage CAM-21 and bacterial cells, enhancing the probability of infection and bacterial lysis.

The polymer solutions were allowed to cool to room temperature before the addition of CAM-21 phage at a concentration of 10⁸ PFU/mL. The phage-incorporated solutions were then cast in sterile plastic petri dishes to form three different film formulations: CMCS+Gelatin+SA, CMCS+Gelatin, and Pullulan+Gelatin+SA. The cast film solutions were allowed to dry overnight at room temperature in a laminar flow hood. Once dried, the films were carefully peeled from the petri dishes. Films used for testing were prepared based on the following compositions:
1) CMCS (2% v/v)+Gelatin (2% v/v)+SA (0.6% v/v)+Glycerol (2% v/v)+CAM-21 (8% v/v)
2) CMCS (2% v/v)+Gelatin (2% v/v)+Glycerol (2% v/v)+CAM-21 (8% v/V)
3) PuI (2% v/v)+Gelatin (2% v/v)+SA (0.6% v/v)+Glycerol (2% v/v)+CAM-21 (8% v/v)
4) Control samples were respective films without CAM-21 phage.

### Example 10: Mechanical Properties of Films

The mechanical properties of the films prepared in Examples 9A and 9B were studied. The developed films were smooth, transparent and flexible without bubbles. For the soy protein biopolymers, tensile tests were performed using a texture analyzer (TA-HDplusC, Texture Technologies Corp., South Hamilton, MA, USA). Prior to the tensile measurement, film samples were prepared by cutting them into rectangular strips measuring 1 cm×5 cm. During the test, the crosshead speed and initial grip separation were set to 0.5 mm per second and 25 mm, respectively. The water vapor permeability (WVP) of the film samples was investigated. Initially, 15 g of anhydrous CaCl₂) was placed in a beaker, which was then covered with the film samples and securely sealed using parafilm. This wrapped beaker was placed inside a desiccator maintained at 75% relative humidity (RH) and 25° C., with the desiccator containing saturated NaCl solution. Over a period of up to 12 h, the weight of the beaker was regularly monitored at specific intervals.

The WP was calculated using the following equation:

$$W V P = ( Δ M \times K ) / ( Δ P \times t \times S )$$

where ΔM represents the weight variation of the beaker (in grams), K is the film thickness (in meters), ΔP is the vapor pressure difference across the film (in Pascals), t is the time, and S is the vapor-exposed area under test (in square meters).

For the carbohydrate biopolymers, a texture analyzer (TA-HDplusC, Texture Technologies Corm, South Hamilton, MA) was used to analyze the physical and mechanical properties of the composite films. Films were cut into uniform rectangular strips (2 cm×5 cm) and mounted on the tensile grips, ensuring they were securely clamped without wrinkles or slack. The analyzer was set to the desired test speed and distance, commonly around 1 mm/s for tensile and puncture tests, and the test was conducted, allowing the texture analyzer to record force-displacement data as it stretches or punctures the film. The data for tensile strength, elongation at break, and puncture force were recorded.

The tensile properties of SPI-based films are shown in Table 5 below. After the incorporation of CAM-21 (1-4%), no significant variation (P>0.05) in the TS and Elongation at Break (EAB) of the film samples was observed. The results suggest that the bacteriophage concentration utilized in these films was still insufficient to have any negative impact on the film's strength and flexibility. Prior studies have reported that the incorporation of bacteriophages did not significantly affect (P>0.05) the tensile properties (e.g., puncture strength and puncture deformation) of gelatin films. CAM-21 is a very small particle with dimensions of 92.83 nm in diameter and 129.75 nm in length. Hence, the incorporation of low concentrations of bacteriophages was not expected to disrupt the structure of protein-based films that are strengthened by various types of molecular interactions, such as hydrogen and disulfide bonding, electrostatic forces, and hydrophobic interactions. A high concentration of bacteriophages may still cause structural changes in the polymer matrix due to the disruption of film homogeneity. The minor variation in TS and EAB of the films could be associated with the microstructure of SPI films incorporated with bacteriophages.

TABLE 5
Influence of CAM-21 on SPI Polymer Mechanical Properties

| | | | WVP |
|---|---|---|---|
| Sample | TS (MPa) | EAB (%) | (×10⁻¹⁰ gm⁻¹ s⁻¹ Pa⁻¹) |
| S | 6.35 ± 0.21 | 31.95 ± 0.74 | 2.56 ± 0.05 |
| SC1 | 6.47 ± 0.12 | 33.75 ± 1.64 | 2.66 ± 0.06 |
| SC2 | 6.54 ± 0.17 | 32.56 ± 0.63 | 2.66 ± 0.06 |
| SC4 | 6.53 ± 0.20 | 32.79 ± 1.27 | 2.60 ± 0.12 |

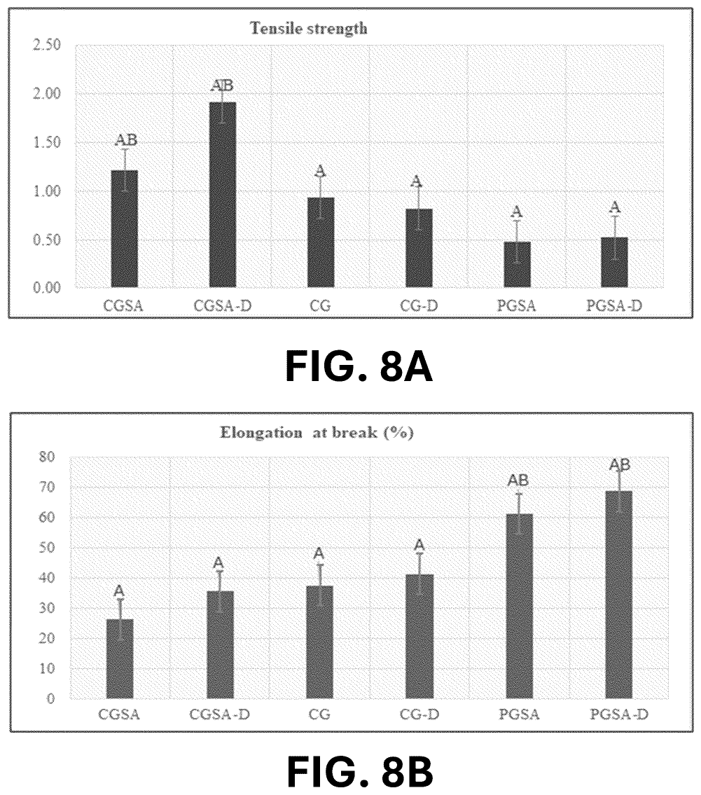

The tensile properties of carbohydrate biopolymers are provided in FIGS. 8A and 8B. The CGSA-D film stands out for its high tensile strength, suggesting enhanced durability and resistance to breakage. In contrast, PGSA-D and PGSA films are the most flexible, with the highest elongation at break values, indicating their suitability for applications where flexibility is essential. The CG and CG-D films offer a balance of moderate tensile strength and low flexibility, suggesting limited stretchability but sufficient structural integrity. Overall, the choice of film composition should be based on the specific mechanical demands of the intended application, with CGSA-D being optimal for strength and PGSA-D for flexibility.

### Example 11: Color and Opacity Analysis of Films

Film color was assessed using a colorimeter (CR-410, Konica Minolta Sensing, Inc., Japan). For SPI films, the total color difference (ΔE) of the films was calculated based on the L*, a*, and b* values obtained from the measurements, with L* representing lightness, ranging from 0 (black) to 100 (white), a* representing the color spectrum from green (negative values) to red (positive values), and b* covering the spectrum from blue (negative values) to yellow (positive values). Triplicate readings were taken to ensure consistency, and the average value was calculated for accuracy. To determine the light transmittance of the films, an UV-Vis spectrophotometer (UV-1900i, Shimadzu Corp., Canby, OR, USA) was used. The transmittance of 0.9 cm×4.0 cm film samples at 600 nm was recorded. A blank test cell without any films served as the reference.

Film opacity was determined for SPI films using the equation:

$$Opacity = - \frac{- log ( T r a n {s}_{600} )}{K}$$

where Trans600 represents the transmittance of the films at 600 nm, and K is the film thickness (in millimeters) determined at five different locations using a digital electronic caliper (Model 35-025; iGAGING, San Clemente, CA, USA).

Film color and opacity are important indicators of food packaging materials. Results for the SPI films are provided in Table 6. As shown in Table 6, the film samples exhibited a yellowish color (large positive b* value), with great film lightness (high L* value). The incorporation of colorless CAM-21 had no significant effect (P>0.05) on the lightness, redness, and yellowness of the S film. The ΔE of phage-containing SPI films were not significantly different (P>0.05) from that of the control films. In addition, all the film samples were visually clear and transparent. The incorporation of bacteriophages in the packaging materials slightly increased the film opacity. However, no significant difference (P>0.05) in the opacity of SPI films containing different concentrations of bacteriophages was observed. These results confirm that the incorporation of bacteriophages did not significantly affect (P>0.05) the optical properties of the SPI films.

TABLE 6
Color and Opacity of Films

| Sample | L* | a* | b* | ΔE | Opacity |
|---|---|---|---|---|---|
| S | 87.69 ± 1.04 | −1.99 ± 0.06 | 20.20 ± 1.68 | 16.93 ± 1.90 | 1.59 ± 0.10 |
| SC1 | 87.97 ± 0.42 | −1.96 ± 0.02 | 20.37 ± 0.92 | 17.00 ± 0.79 | 1.61 ± 0.17 |
| SC2 | 86.77 ± 0.80 | −2.02 ± 0.05 | 22.11 ± 1.25 | 19.01 ± 1.43 | 1.68 ± 0.04 |
| SC4 | 86.15 ± 0.79 | −2.05 ± 0.07 | 22.43 ± 0.89 | 19.54 ± 1.11 | 1.69 ± 0.09 |
| CGSA | 88.945 ± 0.361 | −0.085 ± 0.092 | 5.18 ± 1.499 | 0.75 ± 0.509 | 0.544 ± 0.080 |
| CGSA-D | 88.965 ± 0.488 | 0.155 ± 0.304 | 3.22 ± 1.301 | 0.305 ± 0.049 | 0.573 ± 0.076 |
| PGSA | 88.63 ± 0.693 | 0.485 ± 0.148 | 3.45 ± 2.574 | 0.615 ± 0.46 | 2.793 ± 0.104 |
| PGSA-D | 89.125 ± 0.94 | 0.42 ± 0.269 | 1.66 ± 1.824 | 0.365 ± 0.262 | 2.264 ± 0.298 |
| CG | 89.03 ± 0.127 | −0.065 ± 0.12 | 3.89 ± 0.014 | 0.415 ± 0.106 | 0.665 ± 0.108 |
| CG-D | 89.65 ± 0.665 | 0.15 ± 0.226 | 2.185 ± 1.181 | 0.425 ± 0.12 | 0.883 ± 0.138 |

Lightness values (L) are relatively consistent across treatments, indicating similar brightness, with CG-D showing the highest lightness (89.65), making it the brightest film. The red/green component (a*) reveals slight greenish tendency in CGSA and CG (negative a* values). This green hue is characteristic of the carboxymethyl chitosan-gelatin matrix without bacteriophage addition. PGSA represents the strongest red hue. The yellow/blue component (b*) highlights the highest yellow tone in CGSA (5.18), while PGSA-D (1.66) has the lowest yellow tone, making it the most neutral. The color difference (ΔE) is minimal for CGSA-D (0.305), indicating excellent color stability.

Opacity varies significantly, with PGSA having the highest opacity (2.793), indicating it is the least transparent, while CGSA exhibits the lowest opacity (0.544), making it the most transparent. These findings highlight the influence of composition on the visual properties of films. The opacity values in treatments meet a range of criteria for different packaging film applications. The application depends on whether the film is intended for light-blocking, product visibility, or both.

For carbohydrate-based packaging films, which are often marketed for their environmental benefits, color consistency is still important. While there isn't a specific ISO or ASTM standard focused solely on color values for carbohydrate-based packaging films, general practices in food packaging and biodegradable film production suggest that values should be as close to neutral as possible for aesthetic purposes. Manufacturers typically rely on internal standards or guidelines based on customer requirements. L* value (lightness) is the primary parameter that indicates transparency in packaging films, ranging from 0 (completely black) to 100 (completely white). For transparent films, a higher L* value (closer to 100) signifies greater transparency, as it reflects more light passing through the material with less obstruction. The more light that passes through without scattering or being absorbed, the more transparent the film appears. All of the prepared films showed L* value of around 89 for L value, aligning with consumer acceptance and industry standard.

### Example 12: Compositional Properties of SPI Films

The phage count in the SPI films was assessed using a modified version of a previous technique. Film samples measuring 1.5×1.5 cm² were placed in wells of a sterile 12-well plate. In each well, 1 mL of SM buffer was added, and the plate was agitated in a shaker incubator at 150 rpm for 3 h at room temperature. The phage count was then determined using a double-layer agar technique.

The bacteriophage distribution within the SPI films was studied using a modified version of a previous method. A spectral confocal microscope (Leica TCS SP8, Leica Microsystems Inc., Buffalo Grove, IL) was used to observe the fluorescently labeled samples. One microliter of 100×SYBR™ Gold Nucleic Acid Gel Stain (Invitrogen™, Thermo Fisher Scientific) was added to 0.5 ml of CAM-21 suspensions (˜109 PFU/mL), and the mixture was placed in the dark for 1 h. The labeled phage suspension was spun down using Illustra MicroSpin G-25 columns (GE Healthcare, Chicago, IL, USA) equilibrated with SM buffer to eliminate excess fluorophores. The labeled phages were incorporated into the film-forming solutions and cast in sterile confocal dishes at 30° C. for 48 h. The labeled phage suspension (20 μL) was placed on a glass side and covered with a cover slip. The fluorescent images of labeled phages in the suspension and film samples were obtained using confocal microscopy.

The surface morphology of the SPI films was observed using an environmental scanning electron microscope (Quanta 600 FEG; ThermoFisher Scientific). The films were mounted on aluminum stubs for examination. Prior to SEM imaging, a thin layer of gold was applied to the mounted film samples.

The survival capability of bacteriophages in a polymer matrix is one of the important consideration factors for producing phage-based antimicrobial food packaging materials. CAM-21-containing SPI films were developed by incorporating phage suspensions in a SPI-based FFS containing CNF and glycerol, as additives. The phage titers found in SC1, SC2, and SC4 were 1.02±0.04×10⁷ PFU/cm², 1.41±0.16×10⁷ PFU/cm² and 2.41±0.43×10⁷ PFU/cm², respectively. The results show that the phages remained stable in the polymer matrix because SPI films offer an ideal environment for phages. The stability of phages can be improved by avoiding and reducing oxidative damage to viral nucleic acid components (RNA and DNA) and capsid, and stabilizing the structure of viral capsid proteins. For instance, because of their ordered and compact network structure, protein-based films have good oxygen barrier properties that may reduce oxidative damage. The presence of a plasticizer, such as glycerol, and high protein contents, which may stabilize the viral capsid after film drying, may also contribute to phage survival. Although a 1-2% difference of phage titer in different film formulations may seem negligible on a logarithmic scale, it was observed that even small variations in formulation can potentially influence the film properties. It is suggested that increasing the phage concentration in the film formulation may improve the film performance. For instance, the incorporation of a stock solution consisting of a higher phage concentration (10¹⁰ PFU/mL) resulted in a higher phage titer in the acetate cellulose films.

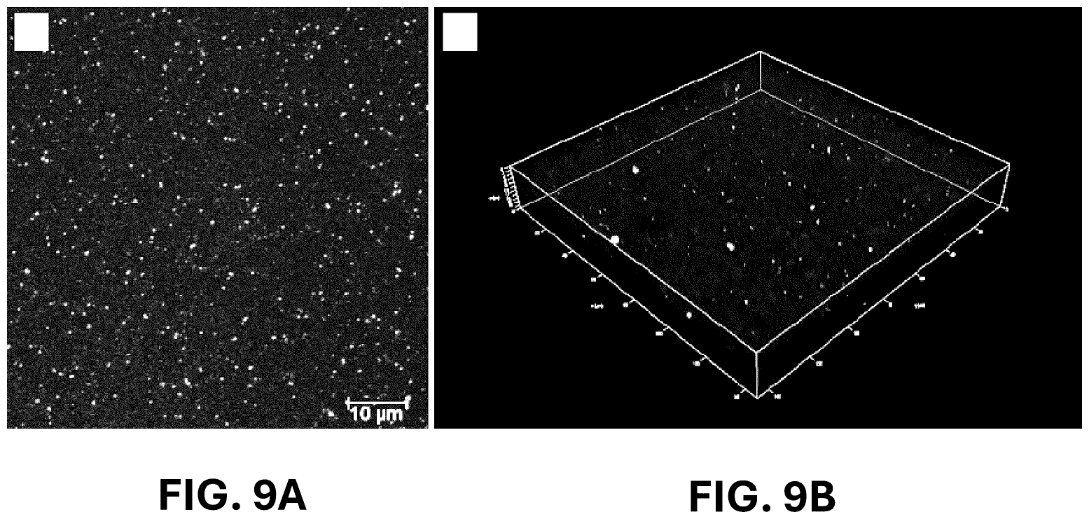

Confocal microscopy was utilized to evaluate the spatial distribution of incorporated phages (white) in the films (FIGS. 9A, 9B). The phage particles were labeled with SYBR™ Gold stain before incorporating in the SPI films (FIG. 9A). The uniform distribution of phages across the entire surface and depth of the polymer matrix is shown in FIG. 9B. Also, phage agglomeration results in the formation of small aggregates, which could be associated with the film drying method or the presence of net opposite charges in the phages' tail and head regions.

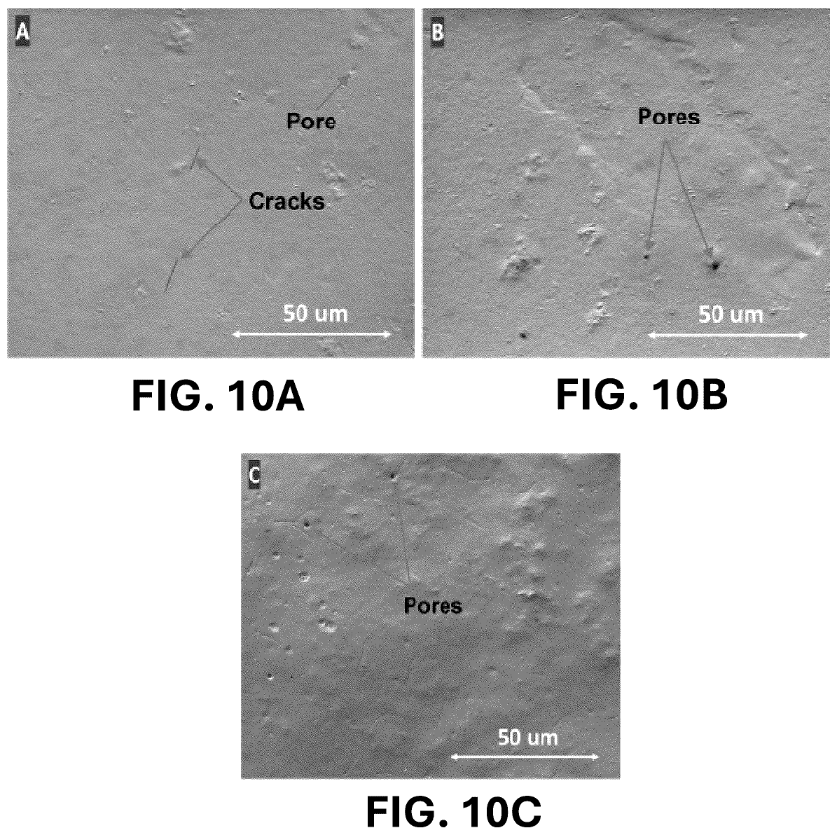

SEM was utilized to evaluate the surface morphology of different film samples (FIGS. 10A-10C). The control films were homogeneous and compact without phase separation, and the film surface was smooth with the presence of some pores and cracks (shown by red arrows, FIG. 10A). Similarly, some pores were also observed when more CAM-21 was added into the SPI films (FIGS. 10B and 10C). The presence of pores could be attributed to the aggregation of CNF during film drying. The incorporation of phage particles in the films did not induce apparent phage separation, suggesting their good compatibility in the polymer matrix. Based on the SEM micrographs, the addition of phage particles had no effect on the film microstructure, which could be associated with the minor variation in WVP and tensile properties of the SPI films.

### Example 13: Compositional Properties of Carbohydrate Biopolymer Films

Fourier Transform Infrared (FTIR) analysis provides insight into the functional groups and chemical interactions within the films, helping to understand how the incorporation of CAM-21 bacteriophage and polymeric components (e.g., chitosan, gelatin, sodium alginate, and pullulan) may alter the chemical structure and properties of each film. To perform FTIR analysis, a Nicolet 380 FTIR spectrometer (Thermo Electron Corp., USA) was used. Carbohydrate biopolymer films were cut into uniform, smooth strips. A background scan was performed prior to each sample measurement to correct for atmospheric interference, specifically from carbon dioxide and water vapor. After the background correction, the FTIR spectrometer was configured to collect spectra across the wavenumber range of 4000-400 cm⁻¹, with a spectral resolution of 4 cm⁻¹.

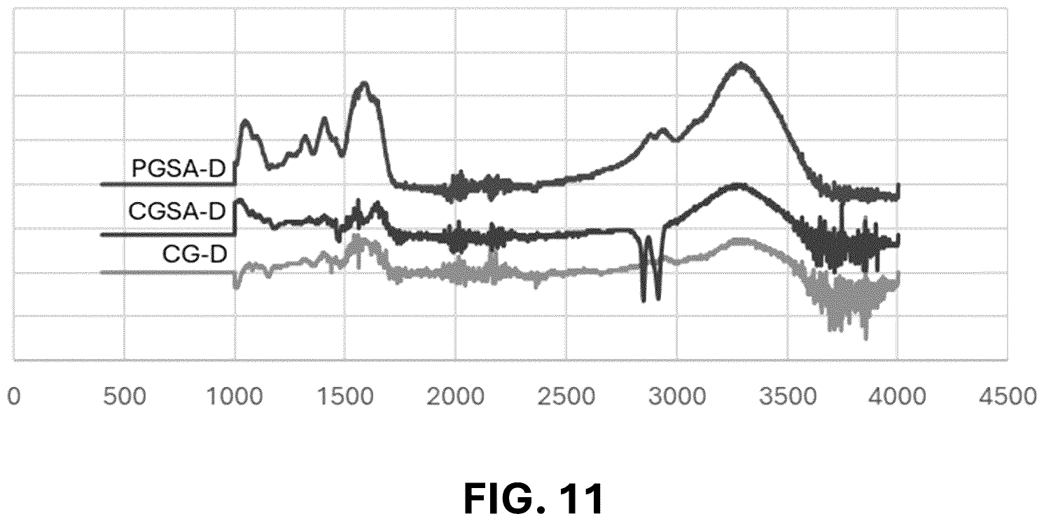

The FTIR spectra, shown in FIG. 11, reveal that each CAM-21-containing film (PGSA-D, CGSA-D and CG-D) show unique functional group interactions influenced by the presence or absence of specific polymers, specifically, sodium alginate, pullulan, and gelatin. PGSA-D shows strong hydrogen bonding and a distinct polysaccharide structure due to pullulan and alginate, which may provide a supportive matrix for CAM-21, especially for applications needing extended release. CGSA-D shows characteristic amide peaks from gelatin and COO-groups from alginate, indicating both protein and polysaccharide interactions, which may contribute to the stability and efficacy of CAM-21 phage on food surfaces. CG-D, lacking alginate, relies on interactions between carboxymethyl chitosan and gelatin, which may create a simpler matrix that still effectively stabilizes the phage but could perform differently in release kinetics compared to PGSA-D and CGSA-D. These FTIR results align with the antimicrobial findings detailed below. The matrix composition affects the stability, interaction, and potential release profile of CAM-21, with each film showing distinct characteristics suited for different applications. PGSA-D and CGSA-D, with sodium alginate's added carboxylate groups, may enhance sustained antimicrobial release, making them potentially more effective in long-term applications, compared to CG-D.

The particle size distribution in the carbohydrate biopolymer film solutions was determined using laser diffraction with a Mastersizer 3000 (Malvern, Worcestershire, UK). Each film solution was prepared by dissolving the film components in water and diluting to a suitable concentration to ensure optimal scattering without multiple scattering effects. The solution was continuously stirred to prevent settling and to maintain uniform particle dispersion. For measurement, the Mastersizer was set up with the dispersant properties (refractive index and absorption) matching those of the solvent used. The instrument's pump and stirrer settings were adjusted to maintain consistent flow and prevent particle aggregation.

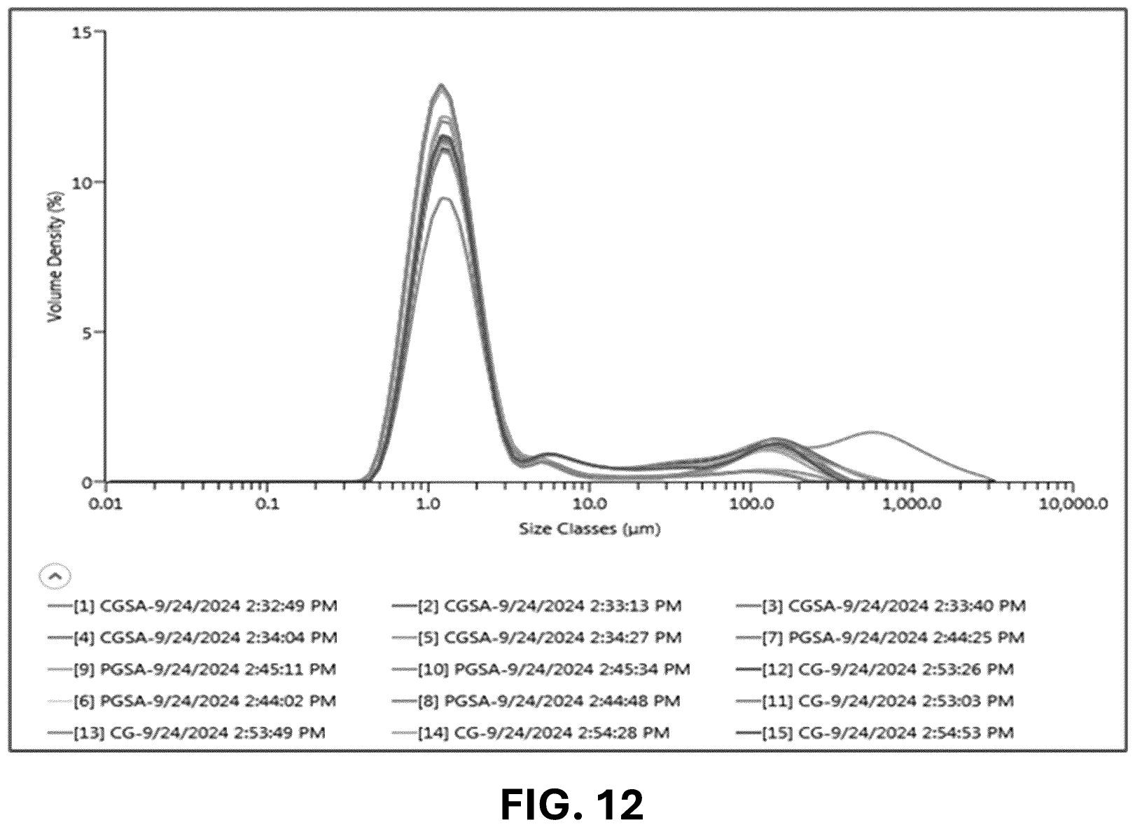

As shown in FIG. 12, the presence of a consistent primary particle size peak at around 1 μm across all samples indicates a high level of uniformity in particle dispersion within each film type, a desirable characteristic for film stability and performance. The slight variations in secondary peaks, particularly in CGSA samples, could reflect differences in polymer compatibility and interactions within each film matrix. The CGSA film matrix shows a broader particle size distribution, possibly due to the combined effects of carboxymethyl chitosan, gelatin, and sodium alginate, which might interact to create larger aggregates. This broader distribution could affect mechanical properties, possibly enhancing tensile strength, but slightly reducing homogeneity. The PGSA and CG films, on the other hand, display narrower distributions with more defined primary peaks, suggesting more homogeneous particle dispersion. This might contribute to their flexibility (noted in the texture analysis for PGSA) and could enhance film uniformity, which is beneficial for consistent antimicrobial action and mechanical stability.

### Example 14: In Vitro Antimicrobial Activity

The antimicrobial effectiveness of the SPI films against E. coli O157:H7 strains 3178-85, EDL-933, C7927, 505B, and MF-1847 was assessed in TSB. The E. coli O157:H7 strains were cultured in TSB and incubated at 37° C. for 24 h. All strains were then combined in equal concentrations to create a five-strain cocktail. The bacterial cocktail was further diluted to achieve a concentration of approximately 10⁴ CFU/mL, and 5 mL of this solution was transferred to sterile test tubes. Two pieces of each film sample, measuring 1 cm×5 cm, were placed in the test tubes containing the diluted bacterial cocktail. As a negative control, a bacterial culture without film was also included. The test tubes were then incubated in a shaker incubator, with continuous shaking at 150 rpm and at a temperature of 37° C. To evaluate the antimicrobial activity of the edible films, plate counts of each diluted sample were performed at 0, 3, 6, 9, 12, and 24 h on MacConkey Sorbitol agar (Difco Laboratories, Detroit, MI, USA). The viable bacterial counts were determined and expressed in log CFU/g values.

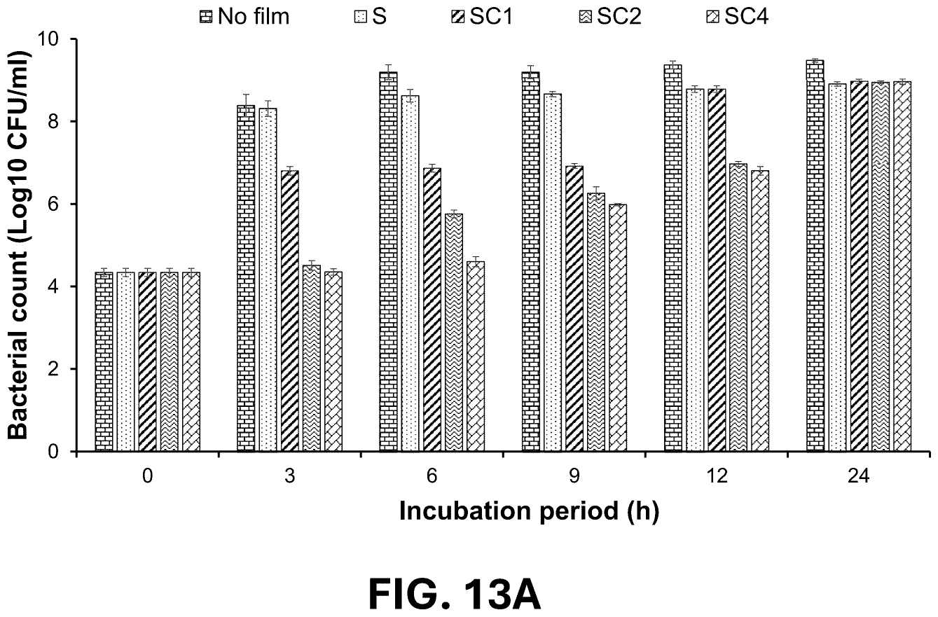

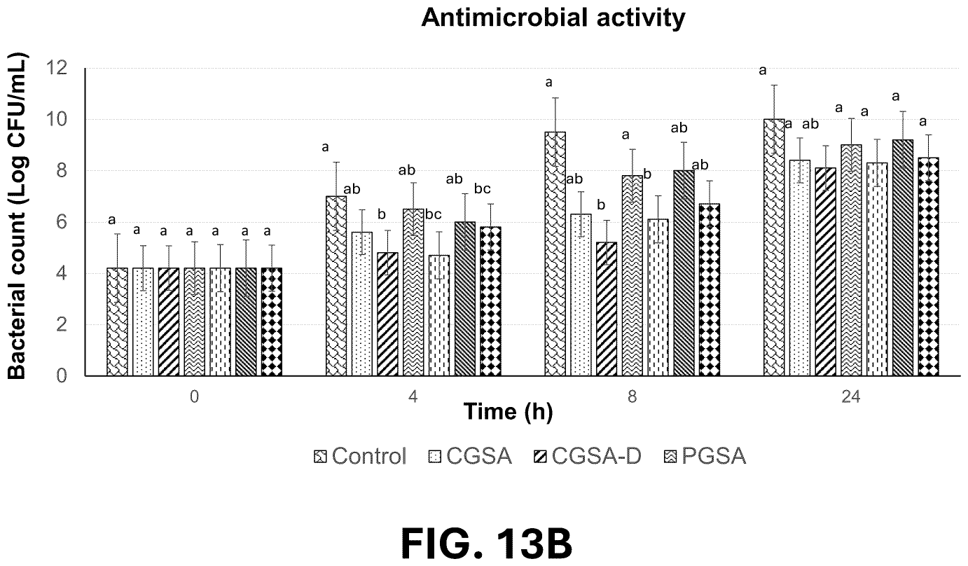

The effects of CAM-21 incorporated into the SPI films on E. coli O157:H7 growth are shown in FIG. 13A. For the control groups without film treatment and with the S film, the bacterial count increased from 4.3 log CFU/mL to about 9 log CFU/mL in 6 h, suggesting that SPI films do not have any inhibitory effect on E. coli O157:H7. After 3, 6, and 9 h of incubation, the bacterial counts in TSB treated with the phage-containing SPI films (SC1, SC2, and SC4) were significantly reduced (P≤0.05) by at least 1.6 log CFU/mL. This could be associated with the short infection period of phages on their host bacteria, that results in bacterial cell lysis after 25-30 min of infection. The best inhibitory effect was presented by the SC4 films, which were incorporated with the highest concentration of CAM-21, and exhibited a maximum reduction of 4.6 log CFU/mL after 6 h of incubation. These results suggest that the antimicrobial activity of the films depends on the concentration of bacteriophages. After 12 h of incubation, the antimicrobial activity of SC2 and SC4 against E. coli O157:H7 was still effective. These findings show that the antimicrobial activity of CAM-21 is preserved during the drying process of the FFS, indicating that the addition of bacteriophages protects their infective capability.

In-vitro antimicrobial activity was also tested in the carbohydrate biopolymer films. To evaluate the antimicrobial activity of bacteriophage-loaded films in broth media, 2×2 cm² of each film (CG-D, CGSA-D, PGSA-D) were cut and placed in tubes containing sterile peptone water inoculated with E. coli O157:H7 at approximately 105 CFU/mL. The tubes were incubated at 37° C. with gentle shaking, and bacterial counts were measured at multiple time points (0, 4, 8 and 24 h) by serially diluting and plating the broth samples on Tryptic Soy Agar (TSA). The reduction in bacterial count was calculated and analyzed statistically to assess the effectiveness of each film. All of the tests were done in at least three replications.

Results for the carbohydrate biopolymers are shown in FIG. 13B. Among the compositions tested, CG-D (carboxymethyl chitosan-gelatin with CAM-21) and PGSA-D (pullulan-sodium alginate with CAM-21) exhibited the most substantial reductions in E. coli O157:H7 count over time, compared to the control, demonstrating enhanced antimicrobial efficacy. Initial bacterial counts were similar across all samples at the 0-h time point. After 4 h, significant reductions in bacterial count emerged for CGSA-D, PGSA-D, and CG-D films, with the effect continuing at 8 h. After 24 h of incubation, bacterial counts in the CGSA treatment remained statistically lower than the control (P≤0.05), while all other treatments showed decreased bacterial counts as compared to the control, although the decrease was not significant (P>0.05). This time-dependent increase in efficacy suggests that the polymeric matrix supports a gradual release and sustained activity of the phage.

The synergistic interactions between CAM-21 bacteriophage and the polymeric matrices (carboxymethyl chitosan-gelatin and pullulan-sodium alginate) likely contributed to these effects by creating an environment conducive to prolonged phage-bacteria interactions. This performance difference may also be influenced by the presence of sodium alginate in CGSA-D, potentially altering the release kinetics of CAM-21. Overall, these results underscore the potential of CAM-21-incorporated films as natural, biodegradable alternatives to conventional food packaging materials, capable of extending shelf life and ensuring food safety through sustained antimicrobial action.

### Example 15: Antimicrobial Effectiveness in Food Studies

The antimicrobial effectiveness of the films against the E. coli O157:H7 strains was assessed using fresh raw bottom round beef and raw beef trimmings as food models. The E. coli O157:H7 strains were cultured in TSB and incubated at 37° C. for 24 h. All strains were then combined in equal concentrations to create a bacterial cocktail. The bacterial cocktail was subjected to centrifugation at 12,745×g (10,000 rpm) for 5 min and washed with peptone water. This washing process was repeated two additional times.

Beef samples were cut into 1.5 cm³ pieces, and 50 μl of 106 CFU/mL bacterial cultures were added and spread on the surface of each food sample using a pipet tip. The spiked foods were left in a laminar hood at room temperature for 15 min to allow for bacterial attachment. Subsequently, 1.5 cm² of films were placed onto the inoculated surface of the food samples. As a control, one food sample was left without any film treatment.

The food samples were then stored in a refrigerator at 4° C. and 25% RH. Samples were collected after 0, 1, 3, 5, and 7 days of storage. On each sampling day, the beef samples were placed in sterile stomacher bags, and 90 mL of peptone water (0.1% w/v) were added to each bag. The samples were homogenized using a Stomacher™ Circulator Model 400 (Seward, Ltd.). The homogenates were serially diluted and plated on MacConkey sorbitol agar plates. The plates were then incubated for 24 hat 37° C. The total viable bacterial counts were determined and expressed in log CFU/g values.

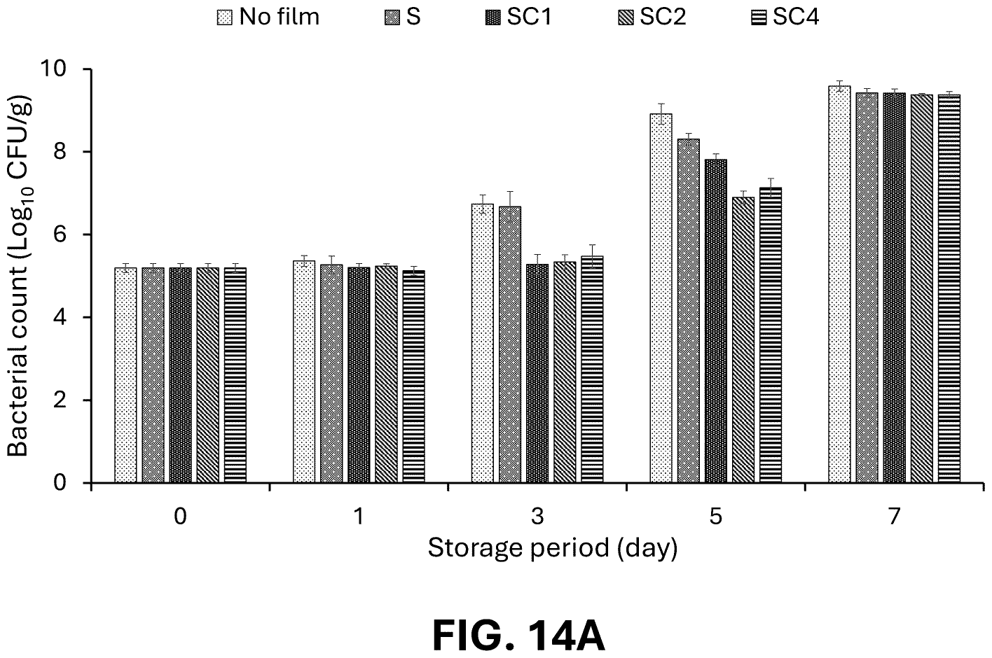

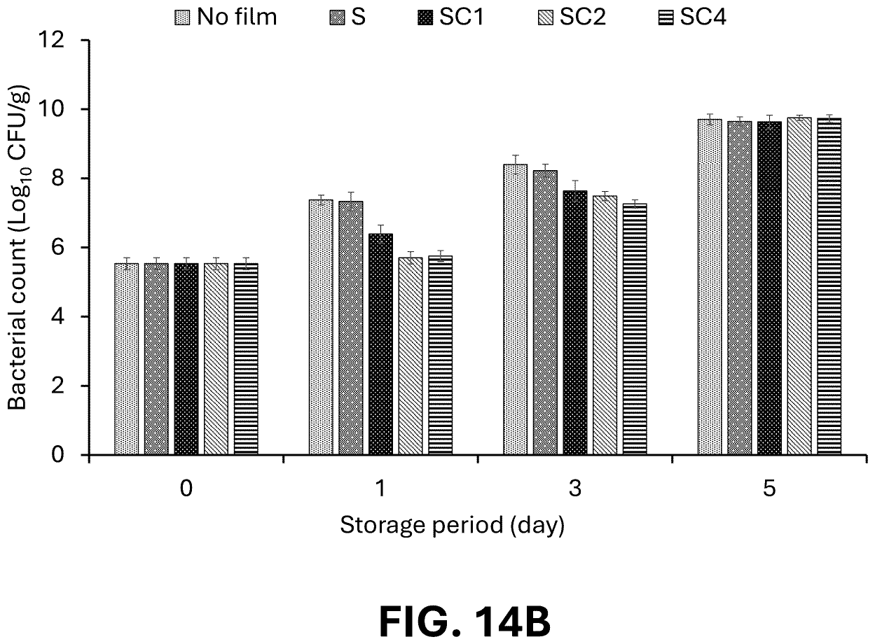

Results from the investigation on contaminated raw bottom round beef are shown in FIG. 14A. Beef samples treated with S films, as well as untreated samples, showed a gradual increase in bacterial counts during 3 days of refrigerated storage, reaching approximately 6.7 log CFU/g, indicating meat spoilage. In contrast, beef samples treated with SPI films incorporating Escherichia phage CAM-21 demonstrated antimicrobial activity against E. coli O157:H7, delaying bacterial growth. The phage containing films (SC1, SC2, and SC4) reduced the bacterial count by a maximum of 1.5 and 2.0 log CFU/g after 3 and 5 days of storage, respectively. However, by day 5, all the beef samples had microbial loads of at least 6.9 log CFU/g, indicating spoilage.

Additionally, the biocontrol effect of phage-containing SPI films was evaluated on raw beef trimmings (FIG. 14B). Both beef samples that are untreated and treated with S films showed an increase in bacterial count of about 1.8 log CFU/g after 1 day of storage. The antimicrobial activity of the SC2 and SC4 films against E. coli O157:H7 was superior to that of the SC1 films on day 1, as they delayed bacterial growth. By day 3 of storage, the microbial load of all beef trimmings exceeded 7 log CFU/g. The variation in antimicrobial effects might be due to the complexity of the food system, including carbohydrate, protein, and fat, as well as water content in beef products. These factors can affect the interaction between phages and E. coli O157:H7, potentially hindering phage transport to the active site of target bacteria. The study concluded that SPI films containing bacteriophages demonstrated significant antimicrobial activity against E. coli O157:H7 in beef products, making them promising candidates for antimicrobial food packaging to preserve the safety of beef products.

To evaluate the antimicrobial activity of the carbohydrate biopolymer films against E. coli O157:H7 on spinach leaves, a bacterial culture was grown in TSB at 37° C. overnight, then centrifuged and washed twice with PBS for purity. Baby spinach leaves, aseptically cut into 4 in² squares were placed and each square placed in sterile petri dishes. A 100 μL aliquot of 107 CFU/mL bacterial suspension was spread on each spinach leaf, and the inoculated spinach leaves were left in a laminar flow hood for 30 min to allow for bacterial attachment. Untreated (without film) samples served as controls. The samples were stored at 4° C. and collected at intervals of 0, 1, 5, and 7 days. On each sampling day, each spinach leaf was suspended in 9 mL PBS and shaken at 200 rpm, 37° C. for 15 min in a shaker incubator. Serially diluted samples were plated on MacConkey sorbitol agar and colonies were enumerated after an overnight incubation at 37° C.

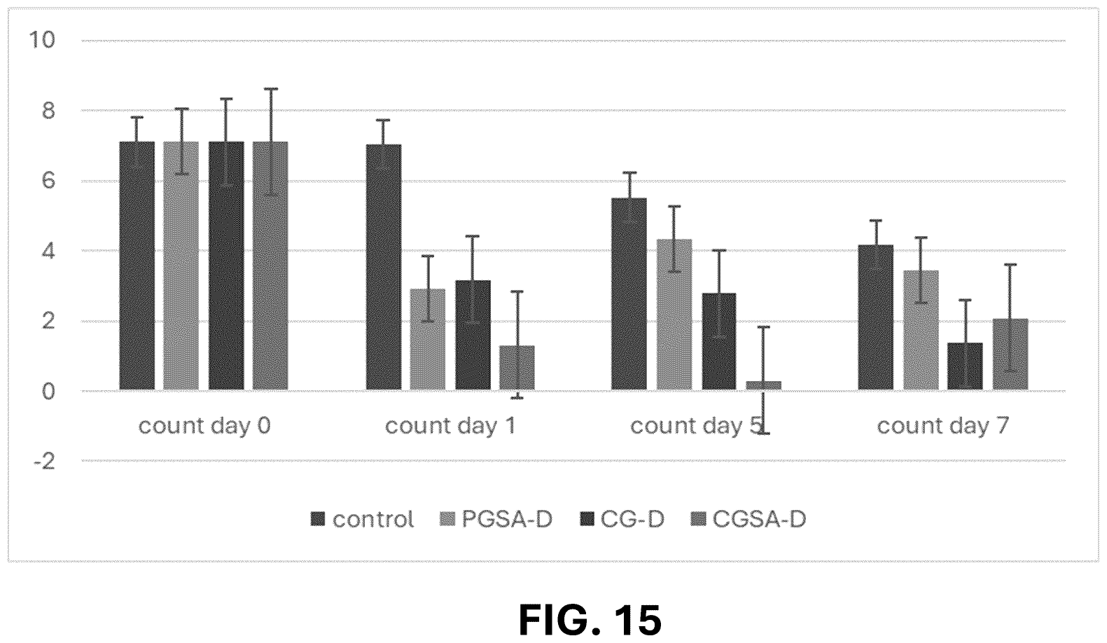

Results are shown in FIG. 15. By day 1, a reduction in E. coli O157:H7 count was observed across all treatments, with CGSA-D showing the most pronounced reduction, followed by CG-D and PGSA-D. By day 5, CGSA-D maintained the lowest bacterial count, indicating an impressive antimicrobial activity, while CG-D also showing effective bacterial inhibition, but to a slightly lesser degree than CGSA-D. In contrast, the control retained the highest bacterial counts, underscoring the importance of CAM-21 incorporation for antimicrobial effects in the biocomposite films. CGSA-D outperformed the other films by day 5, followed by CG-D, while CG-D demonstrated a better effect than CGSA on day 7, which suggests that the addition of sodium alginate in the CGSA matrix may enhance antimicrobial activity on a food surface, possibly by improving adherence or the release profile of CAM-21 in refrigerated, real-food conditions.

### Example 16: Synthesis and Characterization of Quantum Dots for Pathogen Detection

Nitrogen doped graphene quantum dots (N-GQDs) were synthesized using p-phenylenediamine (p-PD) and melamine. Specifically, 0.13 g of p-PD and 0.1 g of melamine were dissolved in 30 mL of ethanol. This mixture was then transferred to a Teflon-lined stainless-steel autoclave and reacted at 180° C. for 20 h. The resulting N-GQDs were purified through continuous dialysis in Milli-Q water, followed by extraction with ethyl acetate. Finally, the N-GQDs were dried at 60° C. using a centrifugal vacuum concentrator. The purified N-GQDs were then examined using gel electrophoresis and compared to the initially synthesized and dialyzed N-GQDs.

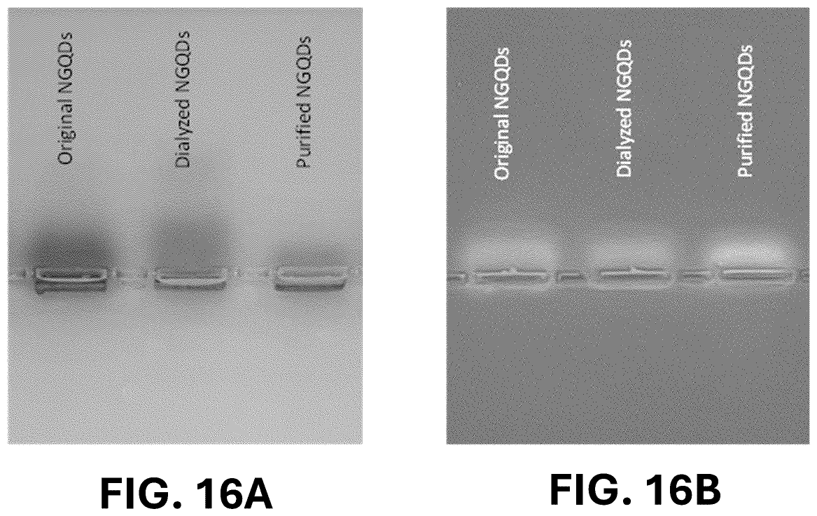

The synthesized NGQDs were purified by a combined dialysis-solvent extraction approach. The results were analyzed by electrophoresis, as shown in FIGS. 16A and 16B. Both the original and dialyzed NGQDs exhibited two bands: one purple and one orange. However, the purified NGQDs displayed only a single orange band. The purple (top) band in the unpurified NGQDs did not fluoresce, while the orange band in all three samples exhibited fluorescence. This indicates that the new purification method is effective for NGQDs.

Fluorescence spectra and UV-vis absorption spectra were employed to investigate the optical properties of the purified N-GQDs. Transmission Electron Microscopy (TEM) and High-Resolution TEM (HRTEM) observations were conducted using transmission electron microscopes (FEI Tecnai F-20 S-Twin, transmission electron microscope, Netherlands). The purified NGQDs were dried and subsequently utilized for chemical composition and structural analysis. Fourier Transform Infrared (FT-IR) spectra were characterized with a Nicolet 380 attenuated total reflectance-FTIR spectrometer (Thermo Electron Corp., USA). X-ray Photoelectron Spectroscopy (XPS) spectra were gathered using a Thermo ESCALAB 250Xi spectrometer. X-ray Diffraction (XRD) patterns were measured using a Rigaku X-ray diffractometer (SmartLab-II, Japan).

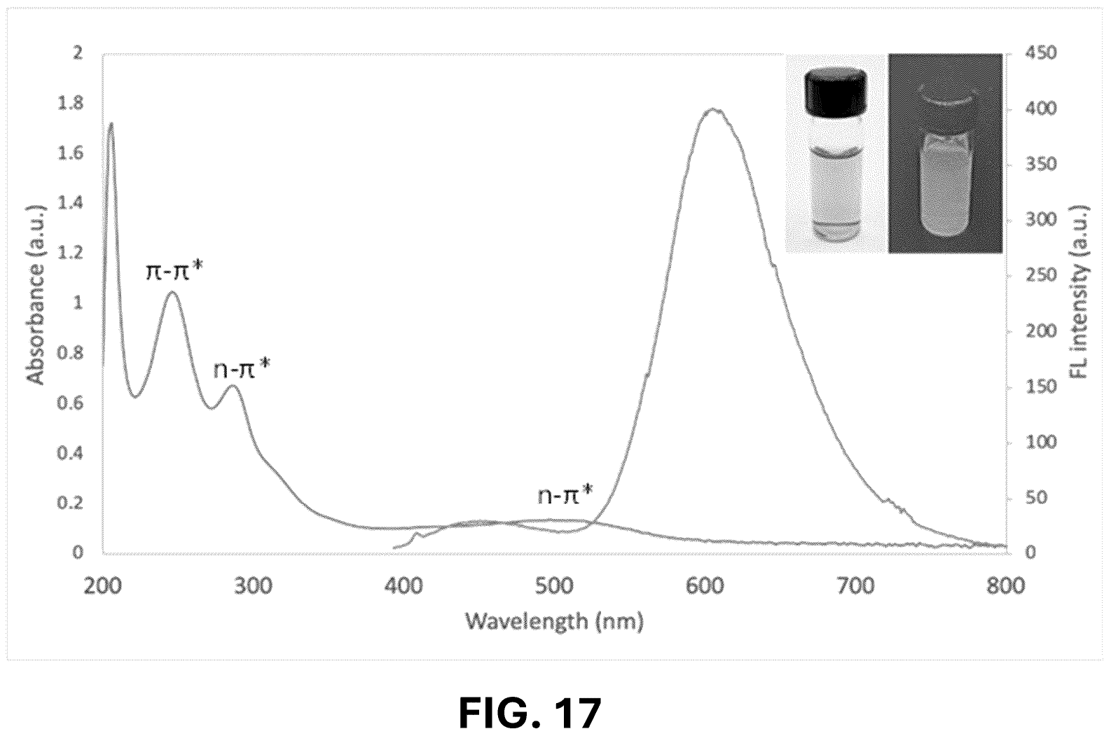

The NGQDs display a peak at 248 nm in the UV-vis spectrum, attributed to the π-π transitions* of the sp² hybridized graphite core. Additional peaks appear around 290 nm and 500 nm, corresponding to the n-IT transitions* characteristic of C—N and C═N groups (FIG. 17). The N-GQDs exhibit an excitation-independent emission spectrum with a maximum emission at 600 nm in ethanol (λexc=365 nm).

The quantum yield of the N-GQDs was measured using a spectrofluorophotometer (Shimadzu RF-6000). Quantum yield is a key parameter for evaluating the luminous intensity and efficiency of fluorescent materials. The RF-6000 spectrofluorophotometer was employed to measure and calculate the quantum yield using the relative method, with Rhodamine B in ethanol (Quantum yield=0.56) as the reference standard. The quantum yield of NGQDs (in water) was calculated according to the following equation:

$$Q = {Q}_{S} \times \frac{F}{{F}_{S}} \times \frac{{A}_{S}}{A} \times \frac{{η}^{2}}{{η}_{S}^{2}}$$

Where Q represents the quantum yield, F is the measured integrated emission intensity, A is the optical density and η is the refractive index of the respective solvents. The subscript “S” refers to a standard with a known quantum yield.

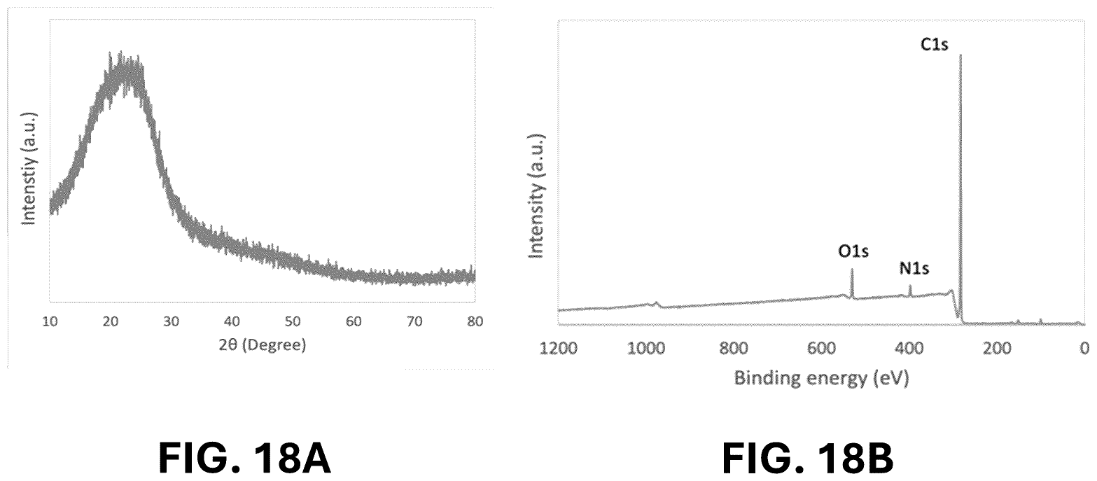

XRD analysis of the NGQDs shows a broad diffraction peak centered at 23.7° (FIG. 18A), indicating their amorphous nature. The elemental composition of the NGQDs was examined using XPS (FIG. 18B), revealing three characteristic signals: C1s at 284 eV, N1s at 399 eV, and O1s at 532 eV, with atomic percentages of 87.14%, 4.8% and 8.06%, respectively. The quantum yield of the N-GQDs was measured under 365 nm excitation, resulting in a quantum yield of approximately 22%.

### Example 17: Synthesis and Characterization of O-NGQDs

Orange emissive N-doped graphene quantum dots were prepared as follows. p-phenylenediamine (0.11 g) was dissolved in absolute ethanol and subsequently transferred into a 100 mL Teflon-lined autoclave. The mixture was heated at 180° C. for 16 h. Following cooling to room temperature, the solution was filtered through a 0.22 μm PES membrane. The resulting N-GQDs were subjected to continuous dialysis (500 D size) for purification, and subsequently dried at 50° C. using a centrifugal vacuum concentrator (CentriVap, Labconco).

For further purification, O-NGQD were dissolved in DMSO and separated using a 1% agarose gel. The band displaying orange fluorescence was excised from the gel. The resulting N-GQD was dried at 50° C. using a centrifugal vacuum concentrator (Acid-resistant CentriVap, Labconco).

UV-Vis spectra and fluorescence spectra were obtained using a Synergy H1 microplate reader (Bio-Tek Instruments Inc.). FI-IR spectra were recorded on a Nicolet 380 (Thermo Fisher) FTIR spectrophotometer.

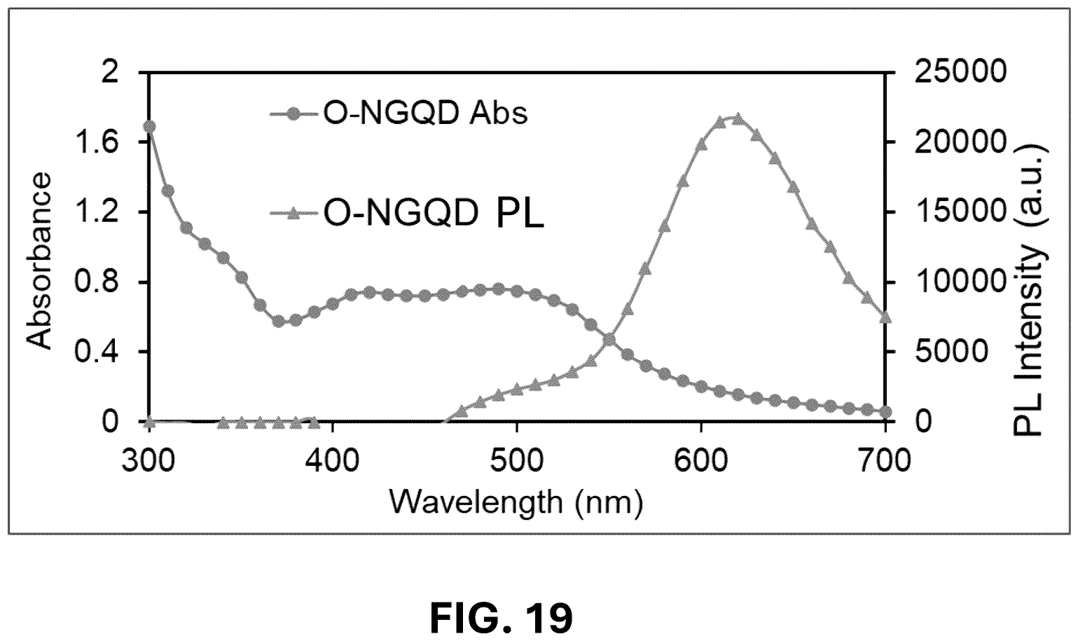

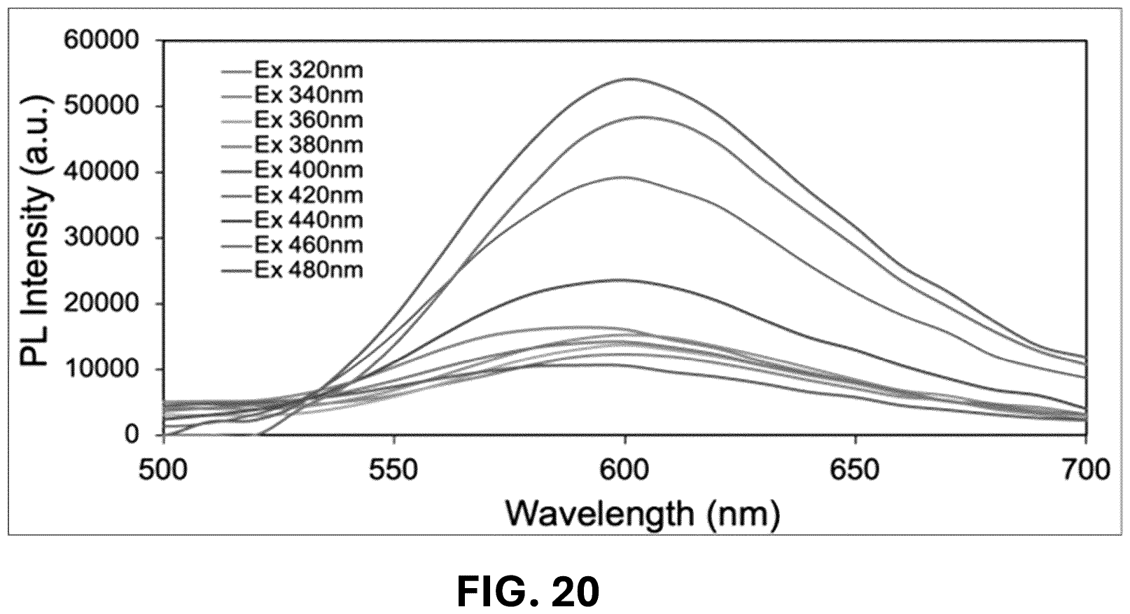

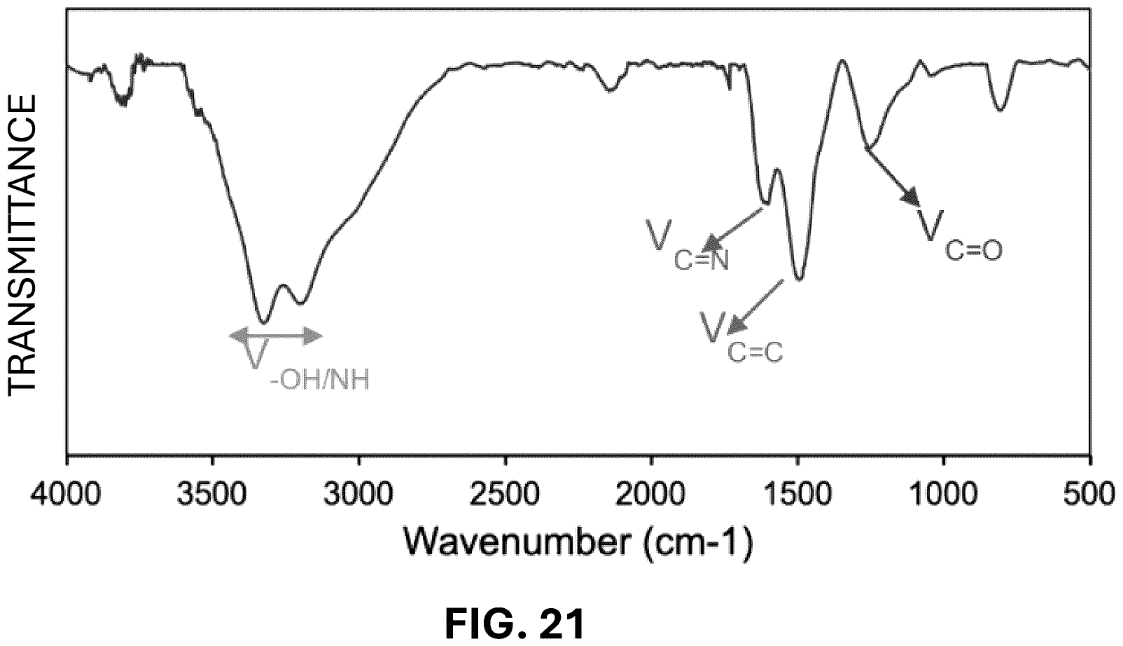

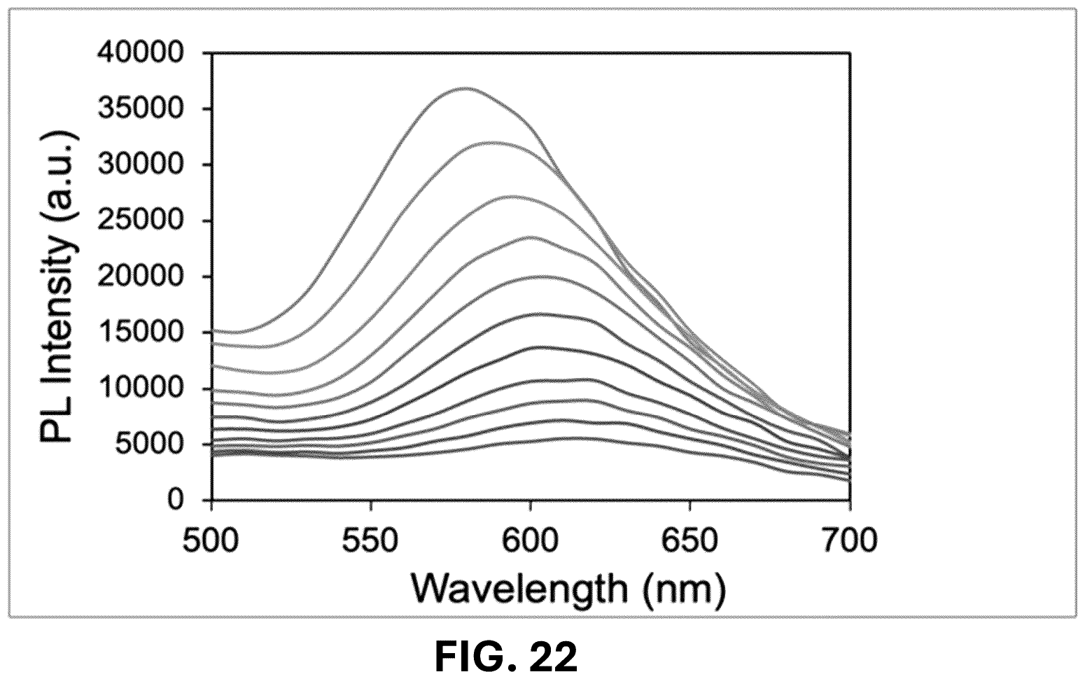

The synthesized O-NGDQ solution, which is red under ambient light, emitted bright orange fluorescence under 365 nm UV light. NGQD typically exhibit peaks in two characteristic regions in the UV-vis spectrum, one between 220 and 280 nm, assigned to the π-π* transitions (of the sp² hybridized graphite core) and the other between 280 and 580 nm, related to the n-π* transitions (associated with functional surface groups). However, the peak around between 220-280 nm is too high to be detected. There should have been peaks in this range. The O-NGQDs also showed peaks around 310 nm and 510 nm, which can be attributed to the n-π* transitions characteristics of C—N and C═N groups (FIG. 19). The O-NGQDs showed an excitation independent emission spectrum, with maximum emission at 610 nm (λexc=360 nm) (FIG. 20). FTIR spectra of the O-NGQDs showed bands related to OH/NH, C═N, C═C and C═O groups (FIG. 21). FIG. 22 shows the fluorescence spectra of R-GQDs dispersed in DMSO solution with varying water content, ranging from 0% to 100%. With an increase in the water content, there is a gradual reduction in the photoluminescence (PL) intensity, accompanied by a minor red-shift in the peak position.

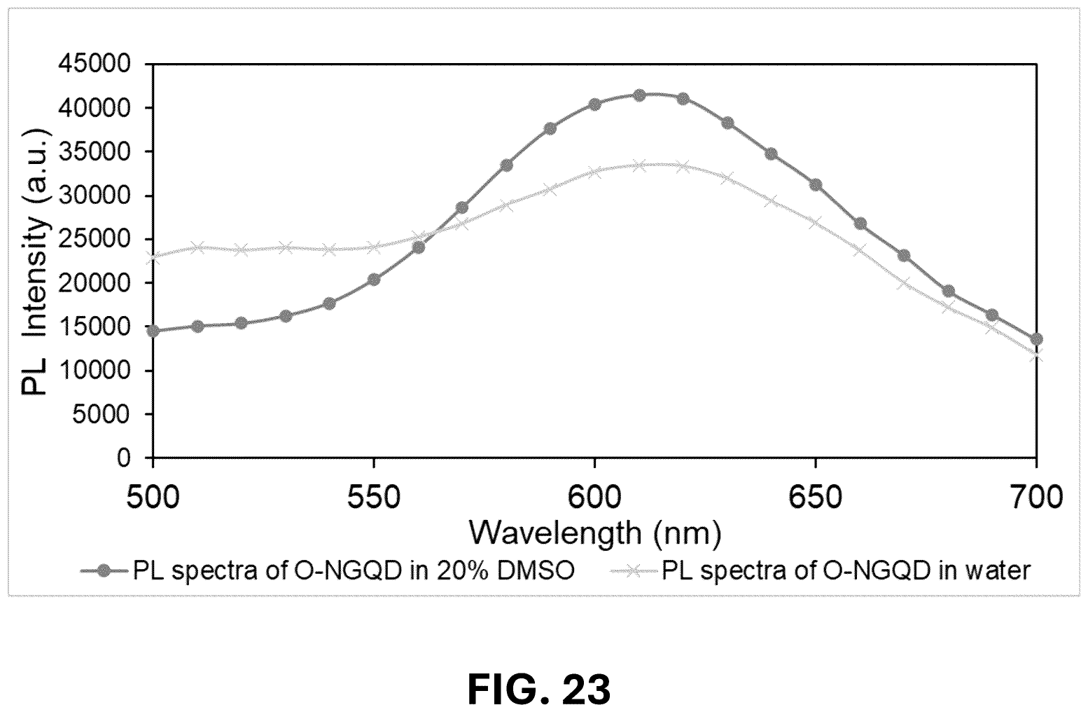

Despite undergoing dialysis for purification, NGQD was still not pure enough. According to some references, NGQD can be further purified by column chromatography. Because this method is time-consuming and expensive, purifying them by agarose gel electrophoresis, which is easier and more economical, was attempted. After purification by electrophoresis, the solubility and fluorescence intensity were significantly increased in water (FIG. 23), compared with FIG. 22.

### Example 18: Conjugation of O-NGQD with CAM-21

N-doped GQD were initially dissolved in DMSO and subsequently diluted in PBS (pH 7.4). N-GQDs was first modified by the crosslinkers, succinimidyl 4-[N-maleimidomethyl]cyclohexane-1-carboxylate (SMCC) and mercaptosuccinic acid (MSA). Subsequently, the surface carboxyl groups of the modified NGQDs were activated using an EDC/NHS method. A CAM-21 phage solution (1011 PFU/mL) was added, and the entire solution was incubated at 4° C. overnight. The phage conjugate was then isolated through ultracentrifugation (20,000 rpm, 4° C.; Sorval) for 2 h. Finally, the fluorescence spectra and phage counts were determined.

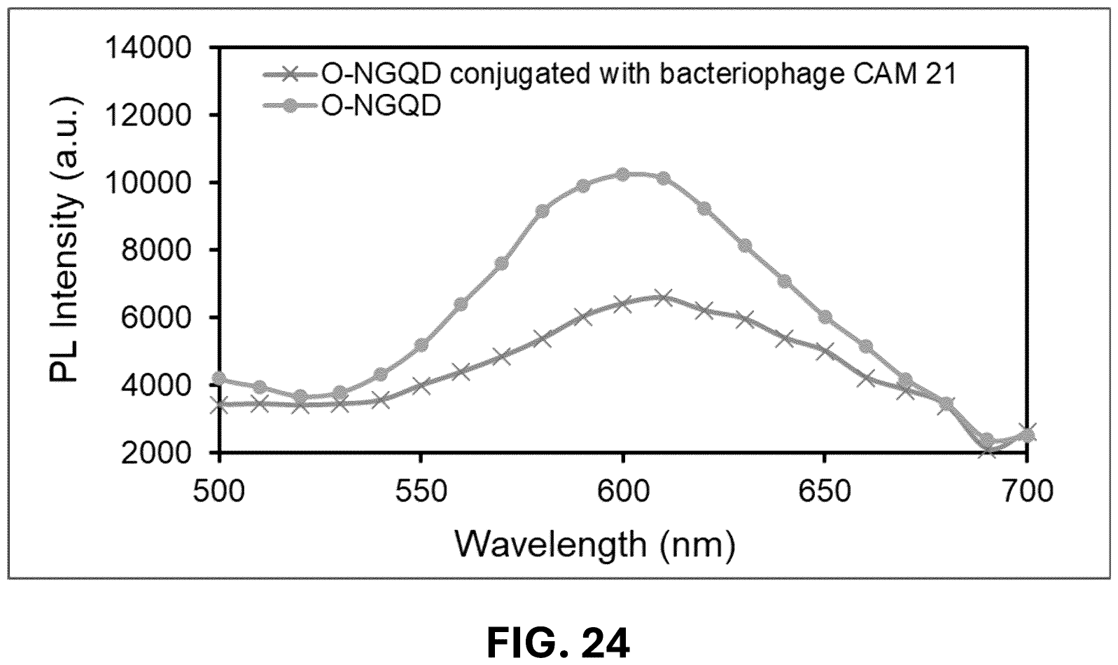

For phage CAM-21 conjugation on the surface of O-NGQD, a crosslinker, SMCC, was introduced to modify the surface functional amine groups of O-NGQDs to reduce their self-conjugation. FIG. 24 reveals that phage CAM-21 was successfully conjugated on the surface of O-NGQD.

In summary, multifunctional O-NGQD were synthesized with a simple one-step hydrothermal carbonization method. The as-synthesized O-NGQDs exhibit orange emission with an excitation wavelength independent property. The FTIR spectrum reveals the presence of abundant oxygen- and nitrogen-containing functional groups. Further, the O-NGQDs exhibited obvious solvent-dependent light emission properties. Notably, as the water content in the solvent increased, a substantial decline was observed in both their solubility and fluorescence intensity. However, following a purification process involving gel electrophoresis, a remarkable enhancement in both solubility and fluorescence intensity was observed. Furthermore, the methods described have effectively conjugated these O-NGQDs with phage CAM-21.

### Example 19: Preparation of NGQD-CAM-21 Probe

Phage CAM-21 suspension was subjected to centrifugation at 671,328×g for 2 h. The resulting pellet was resuspended in NGQD solution (PBS, pH 7.4) and incubated in 4° C. for 48 h. The NGQD-CAM-21 composite was then collected by centrifugation at 671,328×g for 2 h. Following two washes with PBS buffer (pH 7.4), the composite was resuspended in PBS and stored at 4° C. for further use.

### Example 20: Fabrication of LFA Strips and Detection of E. coli O157:H7 Using the Phage-Based LFA

The platform comprised three components: a detection pad, an absorbent pad, and a nitrocellulose membrane. The detection and absorbent pads were prepared by sequentially washing with Milli-Q water, PBST (PBS pH 7.4, Tween20 0.5%) and PBS buffer, followed by drying in an oven at 37° C. overnight. Phage CAM-21 (1010 PFU/mL) and NGQD solution (in ethanol) were used to print the test and control lines. The membrane was then stored at 4° C. overnight, protected with aluminum foil. Prior to use, the detection and absorbent pads were assembled on the prepared membrane, and the strips were cut to 4 mm width.

E. coli O157:H7 C7927 was serially diluted to concentrations ranging from 10¹ to 10⁷ CFU/mL. The bacterial count in each sample was determined by plating on TSA, incubating at 37° C. overnight, and enumerating colonies. Each sample solution (100 mL) was pipetted onto the detection pad, and the strip incubated at 37° C. for 15 min, followed by washing with 100 μL of PBS buffer containing 2% BSA and 0.05% Tween 20. Subsequently, 50 μL of the NGQD-CAM-21 (1011 PFU/mL) composite was added to the detection pad and incubated at 37° C. for 15 min. The result was analyzed using an ESEQuant LR3 lateral flow reader.

To evaluate the effectiveness of the phage-based LFA for detecting E. coli O157 in food samples, the platform was used to test milk and beef samples spiked with the pathogen. Twenty-five grams of beef rump roast and 10 mL of pasteurized milk were inoculated with bacterial dilutions at relevant concentrations and incubated at room temp for 30 min to allow for bacterial attachment. The samples were then homogenized in stomacher bags containing PBS buffer. Serial 10-fold dilutions were prepared and plated on MacConkey-sorbitol agar to determine E. coli O157:H7 counts. The samples were then tested by following the same procedure as described for PBS buffer.

E. coli O157:H7 C7927 samples in PBS buffer with concentrations between 10¹ to 10⁷ were measured with this LFA platform. The fluorescence intensity exhibited a positive correlation with the concentration of E. coli O157:H7, ranging from 10¹ to 10⁷ CFU/mL. The intensity values increased consistently with higher concentrations of E. coli. The limit of detection of the bacterial samples in PBS buffer is around 10 CFU/mL.

To evaluate the test's performance across various food matrices, milk and beef samples were inoculated with different concentrations of the analyte and assessed using the proposed lateral flow strips. The limit of detection for meat and milk was approximately 10⁴ CFU mL⁻¹, significantly higher than in PBS buffer. These findings indicate that the sample matrix influences the lateral flow test's performance, a common occurrence in such assays. This effect may also be linked to changes in the microenvironment, such as the food matrix composition, polarity, hydrogen-bonding capability, local viscosity, pH, and ionic strength of the matrix solution, which can impact the photoluminescent properties of the NGQDs-CAM 21 probe.

What is claimed is:

1. A film product comprising:
a film; and
a quantity of bacteriophage deposited on or incorporated in the film.

2. The film product of claim 1, wherein said film comprises one or more proteins or carbohydrates, or any combination thereof.

3. The film product of claim 2, wherein said one or more protein and/or carbohydrate materials is selected from the group consisting of soy protein isolate (SPI), whey protein concentrate (WPC), starches including modified starch (corn syrup), oligosaccharides, pullulan, gelatin, acetate cellulose, sodium caseinate, glycerol, sodium alginate (SA), carboxymethylcellulose, cellulose nanofiber, and any combination thereof.

4. The film product of claim 2, wherein said one or more proteins or carbohydrates comprises soy-protein isolate, cellulose nanofiber, and glycerol.

5. The film product of claim 1, further comprising a solvent.

6. The film product of claim 5, wherein said solvent is selected from the group consisting of distilled water, a buffer, organic acids, alcohols, ethylene glycol, dimethyl sulfoxide, and any combination thereof.

7. The film product of claim 1, wherein said bacteriophage has lytic activity against Escherichia coli (E. coli).

8. The film product of claim 1, wherein said bacteriophage has at least 90% sequence homology with SEQ ID NO. 1.

9. The film product of claim 1, wherein said bacteriophage is deposited under GenBank accession number OP611477.

10. The film product of claim 1, wherein said film product has at least one measurement or characteristic selected from color, lightness, redness, yellowness, opacity, tensile strength, flexibility, stretchability, and stability, that is at least 95% identical between films having the same composition other than said bacteriophage.

11. The film product of claim 1, wherein the quantity of bacteriophage comprises at least 0.5 wt % of the film product.

12. The film product of claim 1, wherein the bacteriophage is encapsulated with an encapsulant.

13. The film product of claim 12, wherein the encapsulant comprises sodium alginate.

14. A biosensor comprising:
a bacteriophage; and
a graphene quantum dot.

15. The biosensor of claim 14, wherein said bacteriophage has lytic activity against E. coli.

16. The biosensor of claim 14, wherein said bacteriophage has at least 90% sequence homology with SEQ ID NO. 1.

17. The biosensor of claim 14, wherein said bacteriophage is deposited under GenBank accession number OP611477.

18. The biosensor of claim 14, wherein said bacteriophage and said graphene quantum dot are conjugated together.

19. The biosensor of claim 14, wherein said graphene quantum dot is N-doped.

20. A method of detecting E. coli in or on a food product comprising the steps of:
applying the biosensor of claim 14 to a food product;
applying light from an excitation source to said biosensor and said food product;
detecting a photoluminescence spectrum emitted by the biosensor; and
identifying the presence or absence of E. coli in or on said food product on the basis of an intensity and/or wavelength and/or a photoluminescence lifetime.
